SEQUENCE LISTING (90 sequences)

SEQ ID NO: 1 - DNA, 20 bp, synthetic construct
  source 1..20: mol_type = other DNA
  modified_base 1..20: mod_base = OTHER; note = phosphorothioate bond
tccatgacgttcctgatgct

SEQ ID NO: 2 - RNA, 23 nt, synthetic construct
  source 1..23: mol_type = other RNA
  modified_base 2: mod_base = OTHER; note = Uracil
  modified_base 6: mod_base = OTHER; note = Uracil
  modified_base 11: mod_base = OTHER; note = Uracil
  modified_base 17: mod_base = OTHER; note = Uracil
  modified_base 18: mod_base = OTHER; note = Uracil
  modified_base 19: mod_base = OTHER; note = Uracil
  modified_base 22: mod_base = OTHER; note = Uracil
  modified_base 23: mod_base = OTHER; note = Uracil
  modified_base 1..3: mod_base = OTHER; note = phosphorothioate bond
  modified_base 21..23: mod_base = OTHER; note = phosphorothioate bond
atacataaactgcaagtttgctt

SEQ ID NO: 3 - RNA, 21 nt, synthetic construct
  source 1..21: mol_type = other RNA
  modified_base 7: mod_base = OTHER; note = Uracil
  modified_base 8: mod_base = OTHER; note = Uracil
  modified_base 13: mod_base = OTHER; note = Uracil
  modified_base 14: mod_base = OTHER; note = Uracil
  modified_base 15: mod_base = OTHER; note = Uracil
  modified_base 17: mod_base = OTHER; note = Uracil
  modified_base 19: mod_base = OTHER; note = Uracil
  modified_base 21: mod_base = OTHER; note = Uracil
  modified_base 1..3: mod_base = OTHER; note = phosphorothioate bond
gcaaacttgcagtttatgtat

SEQ ID NO: 4 - RNA, 21 nt, synthetic construct
  source 1..21: mol_type = other RNA
  modified_base 2: mod_base = OTHER; note = Uracil
  modified_base 3: mod_base = OTHER; note = Uracil
  modified_base 8: mod_base = OTHER; note = Uracil
  modified_base 12: mod_base = OTHER; note = Uracil
  modified_base 15: mod_base = OTHER; note = Uracil
  modified_base 16: mod_base = OTHER; note = Uracil
  modified_base 20: mod_base = OTHER; note = Uracil
  modified_base 21: mod_base = OTHER; note = Uracil
  modified_base 1..3: mod_base = OTHER; note = phosphorothioate bond
cttacgctgagtacttcgatt

SEQ ID NO: 5 - RNA, 23 nt, synthetic construct
  source 1..23: mol_type = other RNA
  modified_base 3: mod_base = OTHER; note = Uracil
  modified_base 9: mod_base = OTHER; note = Uracil
  modified_base 12: mod_base = OTHER; note = Uracil
  modified_base 18: mod_base = OTHER; note = Uracil
  modified_base 22: mod_base = OTHER; note = Uracil
  modified_base 23: mod_base = OTHER; note = Uracil
  modified_base 1..3: mod_base = OTHER; note = phosphorothioate bond
  modified_base 21..23: mod_base = OTHER; note = phosphorothioate bond
aatcgaagtactcagcgtaagtt

SEQ ID NO: 6 - DNA, 20 bp, synthetic construct
  source 1..20: mol_type = other DNA
cgaaccttacttgagcccac

SEQ ID NO: 7 - DNA, 21 bp, synthetic construct
  source 1..21: mol_type = other DNA
tcacagtggttcgagcttcag

SEQ ID NO: 8 - DNA, 21 bp, synthetic construct
  source 1..21: mol_type = other DNA
gcaaacgagacatcataggca

SEQ ID NO: 9 - DNA, 21 bp, synthetic construct
  source 1..21: mol_type = other DNA
ggtgtccgtgactaactccat

SEQ ID NO: 10 - DNA, 21 bp, synthetic construct
  source 1..21: mol_type = other DNA
tggaaagggtaagaccgtcct

SEQ ID NO: 11 - DNA, 19 bp, synthetic construct
  source 1..19: mol_type = other DNA
tcttgggggttggaggatg

SEQ ID NO: 12 - DNA, 19 bp, synthetic construct
  source 1..19: mol_type = other DNA
cggaggtagaaagggcgtc

SEQ ID NO: 13 - DNA, 23 bp, synthetic construct
  source 1..23: mol_type = other DNA
ctgagatgtcacttcacatggaa

SEQ ID NO: 14 - DNA, 20 bp, synthetic construct
  source 1..20: mol_type = other DNA
gtgcatccccaatgggttct

SEQ ID NO: 15 - DNA, 23 bp, synthetic construct
  source 1..23: mol_type = other DNA
agtacaacgagtaaggagtcact

SEQ ID NO: 16 - DNA, 21 bp, synthetic construct
  source 1..21: mol_type = other DNA
aggccagtatgttgcacatgg

SEQ ID NO: 17 - DNA, 21 bp, synthetic construct
  source 1..21: mol_type = other DNA
tcaggcttacgttgacaaggt

SEQ ID NO: 18 - DNA, 21 bp, synthetic construct
  source 1..21: mol_type = other DNA
cacactttaggcgtgtccatc

SEQ ID NO: 19 - DNA, 21 bp, synthetic construct
  source 1..21: mol_type = other DNA
ttctggtgcttgtctcactga

SEQ ID NO: 20 - DNA, 22 bp, synthetic construct
  source 1..22: mol_type = other DNA
cagtatgttcggcttcccattc

SEQ ID NO: 21 - DNA, 21 bp, synthetic construct
  source 1..21: mol_type = other DNA
ggacttgccttgttccgagag

SEQ ID NO: 22 - DNA, 22 bp, synthetic construct
  source 1..22: mol_type = other DNA
gctgccacataactgatagcga

SEQ ID NO: 23 - DNA, 21 bp, synthetic construct
  source 1..21: mol_type = other DNA
ctggcggacatggctttactt

SEQ ID NO: 24 - DNA, 21 bp, synthetic construct
  source 1..21: mol_type = other DNA
ctcccacttttagcagtcccc

SEQ ID NO: 25 - DNA, 23 bp, synthetic construct
  source 1..23: mol_type = other DNA
ttaaaaacctggatcggaaccaa

SEQ ID NO: 26 - DNA, 23 bp, synthetic construct
  source 1..23: mol_type = other DNA
gcattagcttcagatttacgggt

SEQ ID NO: 27 - DNA, 21 bp, synthetic construct
  source 1..21: mol_type = other DNA
tccttttgggcatcatcttcc

SEQ ID NO: 28 - DNA, 21 bp, synthetic construct
  source 1..21: mol_type = other DNA
tttgtagtggatcgtgcctcg

SEQ ID NO: 29 - DNA, 21 bp, synthetic construct
  source 1..21: mol_type = other DNA
ccaagtgctgccgtcattttc

SEQ ID NO: 30 - DNA, 21 bp, synthetic construct
  source 1..21: mol_type = other DNA
ggctcgcagggatgatttcaa

SEQ ID NO: 31 - DNA, 22 bp, synthetic construct
  source 1..22: mol_type = other DNA
gagcagagaccaaaaggtcaag

SEQ ID NO: 32 - DNA, 21 bp, synthetic construct
  source 1..21: mol_type = other DNA
ctgtgggctcaagtaaggttc

SEQ ID NO: 33 - DNA, 22 bp, synthetic construct
  source 1..22: mol_type = other DNA
caggtctggcaattcttctgaa

SEQ ID NO: 34 - DNA, 23 bp, synthetic construct
  source 1..23: mol_type = other DNA
gtcttgctcatgtgtgtaagtga

SEQ ID NO: 35 - DNA, 21 bp, synthetic construct
  source 1..21: mol_type = other DNA
ctctgttcagctattggacgc

SEQ ID NO: 36 - DNA, 23 bp, synthetic construct
  source 1..23: mol_type = other DNA
cggaatttctgggattcagcttc

SEQ ID NO: 37 - DNA, 23 bp, synthetic construct
  source 1..23: mol_type = other DNA
gttctcagcccaacaatacaaga

SEQ ID NO: 38 - DNA, 19 bp, synthetic construct
  source 1..19: mol_type = other DNA
gtggacgggtcgatgtcac

SEQ ID NO: 39 - DNA, 22 bp, synthetic construct
  source 1..22: mol_type = other DNA
gcaactgttcctgaactcaact

SEQ ID NO: 40 - DNA, 21 bp, synthetic construct
  source 1..21: mol_type = other DNA
atcttttggggtccgtcaact

SEQ ID NO: 41 - DNA, 20 bp, synthetic construct
  source 1..20: mol_type = other DNA
ccaagaggtgagtgcttccc

SEQ ID NO: 42 - DNA, 22 bp, synthetic construct
  source 1..22: mol_type = other DNA
ctgttgttcagactctctccct

SEQ ID NO: 43 - DNA, 22 bp, synthetic construct
  source 1..22: mol_type = other DNA
ctgtgccttggtagcatctatg

SEQ ID NO: 44 - DNA, 23 bp, synthetic construct
  source 1..23: mol_type = other DNA
gcagagtctcgccattatgattc

SEQ ID NO: 45 - DNA, 22 bp, synthetic construct
  source 1..22: mol_type = other DNA
acatccatctcgtgctacttgt

SEQ ID NO: 46 - DNA, 21 bp, synthetic construct
  source 1..21: mol_type = other DNA
gcctctgttttagggagacct

SEQ ID NO: 47 - DNA, 22 bp, synthetic construct
  source 1..22: mol_type = other DNA
gactcttgcgtcaacttcaagg

SEQ ID NO: 48 - DNA, 21 bp, synthetic construct
  source 1..21: mol_type = other DNA
caggctgtcttttgtcaacga

SEQ ID NO: 49 - DNA, 21 bp, synthetic construct
  source 1..21: mol_type = other DNA
gctcttactgactggcatgag

SEQ ID NO: 50 - DNA, 20 bp, synthetic construct
  source 1..20: mol_type = other DNA
cgcagctctaggagcatgtg

SEQ ID NO: 51 - DNA, 22 bp, synthetic construct
  source 1..22: mol_type = other DNA
ctccaagccaaagtccttagag

SEQ ID NO: 52 - DNA, 22 bp, synthetic construct
  source 1..22: mol_type = other DNA
aggagctgtcattagggacatc

SEQ ID NO: 53 - DNA, 22 bp, synthetic construct
  source 1..22: mol_type = other DNA
tgatgagctactactggtcagc

SEQ ID NO: 54 - DNA, 22 bp, synthetic construct
  source 1..22: mol_type = other DNA
gatctcttagcacaaggatggc

SEQ ID NO: 55 - DNA, 21 bp, synthetic construct
  source 1..21: mol_type = other DNA
cagctccaagaaaggacgaac

SEQ ID NO: 56 - DNA, 21 bp, synthetic construct
  source 1..21: mol_type = other DNA
ggcagtgtaactcttctgcat

SEQ ID NO: 57 - DNA, 21 bp, synthetic construct
  source 1..21: mol_type = other DNA
tgtgccgctagaggtgaaatt

SEQ ID NO: 58 - DNA, 19 bp, synthetic construct
  source 1..19: mol_type = other DNA
tggcaaatgctttcgcttt

SEQ ID NO: 59 - DNA, 21 bp, synthetic construct
  source 1..21: mol_type = other DNA
gctgagcactgctggtagaaa

SEQ ID NO: 60 - DNA, 24 bp, synthetic construct
  source 1..24: mol_type = other DNA
ccattttgtgttaagtgactttcc

SEQ ID NO: 61 - DNA, 24 bp, synthetic construct
  source 1..24: mol_type = other DNA
gcatcttactgaaggtgaaatgga

SEQ ID NO: 62 - DNA, 24 bp, synthetic construct
  source 1..24: mol_type = other DNA
ttgggcactgattacagaccatta

SEQ ID NO: 63 - DNA, 21 bp, synthetic construct
  source 1..21: mol_type = other DNA
gccaaaaaactccagtggtga

SEQ ID NO: 64 - DNA, 21 bp, synthetic construct
  source 1..21: mol_type = other DNA
tgactctcacagtcgctgacc

SEQ ID NO: 65 - DNA, 21 bp, synthetic construct
  source 1..21: mol_type = other DNA
gtgtgtgacactgtgcatacg

SEQ ID NO: 66 - DNA, 21 bp, synthetic construct
  source 1..21: mol_type = other DNA
tcagagaagtcagtgacccca

SEQ ID NO: 67 - DNA, 21 bp, synthetic construct
  source 1..21: mol_type = other DNA
gagacagggtttctctgtgta

SEQ ID NO: 68 - DNA, 21 bp, synthetic construct
  source 1..21: mol_type = other DNA
ctttagtcccagcactcggga

SEQ ID NO: 69 - DNA, 21 bp, synthetic construct
  source 1..21: mol_type = other DNA
cggggtacccacctgcctctg

SEQ ID NO: 70 - DNA, 21 bp, synthetic construct
  source 1..21: mol_type = other DNA
aataccacctgcctaacaagt

SEQ ID NO: 71 - DNA, 25 bp, synthetic construct
  source 1..25: mol_type = other DNA
accacatatgtgcattgctcacaga

SEQ ID NO: 72 - DNA, 25 bp, synthetic construct
  source 1..25: mol_type = other DNA
gcctcctgcctcacttctaagtgtt

SEQ ID NO: 73 - DNA, 20 bp, synthetic construct
  source 1..20: mol_type = other DNA
attagaacattcgtcggtac

SEQ ID NO: 74 - DNA, 20 bp, synthetic construct
  source 1..20: mol_type = other DNA
acttgaaccctgtcgtatgc

SEQ ID NO: 75 - DNA, 20 bp, synthetic construct
  source 1..20: mol_type = other DNA
cggctgtcgttctaccacga

SEQ ID NO: 76 - DNA, 20 bp, synthetic construct
  source 1..20: mol_type = other DNA
tctgattaatgacgagctcg

SEQ ID NO: 77 - DNA, 22 bp, synthetic construct
  source 1..22: mol_type = other DNA
gagcagagaccaaaaggtcaag

SEQ ID NO: 78 - DNA, 21 bp, synthetic construct
  source 1..21: mol_type = other DNA
ctgtgggctcaagtaaggttc

SEQ ID NO: 79 - DNA, 23 bp, synthetic construct
  source 1..23: mol_type = other DNA
cattgctgacaggatgcagaagg

SEQ ID NO: 80 - DNA, 22 bp, synthetic construct
  source 1..22: mol_type = other DNA
tgctggaaggtggacagtgagg

SEQ ID NO: 81 - DNA, 20 bp, synthetic construct
  source 1..20: mol_type = other DNA
ctaggggactgtccccatct

SEQ ID NO: 82 - DNA, 20 bp, synthetic construct
  source 1..20: mol_type = other DNA
cttgggaagtccccatggtc

SEQ ID NO: 83 - DNA, 22 bp, synthetic construct
  source 1..22: mol_type = other DNA
gtgctgaggttcacagtgacgt

SEQ ID NO: 84 - DNA, 22 bp, synthetic construct
  source 1..22: mol_type = other DNA
ggttgtagagcctctggatctc

SEQ ID NO: 85 - DNA, 19 bp, synthetic construct
  source 1..19: mol_type = other DNA
gagaagcgggtggacaatg

SEQ ID NO: 86 - DNA, 19 bp, synthetic construct
  source 1..19: mol_type = other DNA
caatgtggcggattcaggc

SEQ ID NO: 87 - DNA, 21 bp, synthetic construct
  source 1..21: mol_type = other DNA
ggccaccctagaccactattc

SEQ ID NO: 88 - DNA, 21 bp, synthetic construct
  source 1..21: mol_type = other DNA
gagttggggtgtcaagggccc

SEQ ID NO: 89 - DNA, 20 bp, synthetic construct
  source 1..20: mol_type = other DNA
tgaagctgcttggtctacgg

SEQ ID NO: 90 - DNA, 20 bp, synthetic construct
  source 1..20: mol_type = other DNA
gacttacatccgaacccggt

(19) United States
(12) Patent Application Publication
(10) Pub. No.: US 2025/0228895 A1
(43) Pub. Date: Jul. 17, 2025

(54) COMPOUNDS TO INHIBIT YTHDF2, T HELPER CELLS LACKING EXPRESSION OF YTHDF2, AND METHODS TO TREAT CANCER

(71) Applicant: City of Hope, Duarte, CA (US)

(72) Inventors: Jianhua Yu, Duarte, CA (US); Michael A. Caligiuri, Duarte, CA (US)

(21) Appl. No.: 19/025,059

(22) Filed: Jan. 16, 2025

Related U.S. Application Data
(60) Provisional application No. 63/621,376, filed on Jan. 16, 2024.

(51) Int. Cl. A61K 35/17; A61K 40/11; A61K 40/31; A61K 47/54; A61P 35/00; C12N 5/0783; C12N 15/113

(52) U.S. Cl. CPC A61K 35/17; A61K 40/11; A61K 40/31; A61K 47/549; A61P 35/00; C12N 5/0636; C12N 15/1138; C12N 2310/14

(57) ABSTRACT

A YTHDF2 inhibitor, such as an antibody, a small molecule, an aptamer, a nucleic acid, a protein, or an enzyme, can be used for inhibiting expression of YTHDF2 in tumor-associated macrophages (TAMs) and in methods of treating cancer. T helper 9 cells that do not express a YTHDF2 protein are useful in inducing an immune response and treating cancer.

### CROSS-REFERENCE TO RELATED APPLICATIONS

This application claims the benefit of priority to U.S. Application No. 63/621,376 filed Jan. 16, 2024, the disclosure of which is incorporated by reference herein in its entirety.

### REFERENCE TO A “SEQUENCE LISTING,” A TABLE, OR A COMPUTER PROGRAM LISTING APPENDIX SUBMITTED AS AN ASCII FILE

The Sequence Listing entitled “048440-854001US-Sequence Listing” is written in XML format, has 98,348 bytes, was created on Dec. 11, 2024, and is incorporated by reference.

### BACKGROUND

Immunotherapy, represented by immune checkpoint inhibitors (ICIs), such as those targeting cytotoxic T lymphocyte-associated protein 4 (CTLA-4) and programmed cell death protein 1/programmed cell death ligand 1 (PD-1/PD-L1), has greatly improved the clinical efficacy of anti-cancer therapy. (Refs 1-3). However, only a minority of patients respond to immune checkpoint therapy (ICT). (Ref 4) Resistance to ICT is derived not only from tumor intrinsic factors such as PD-L1 expression, mutation burden, and mismatch repair deficiency, but also from the complicated interplay between cancer and the tumor microenvironment (TME). (Refs 5-8) The tumor microenvironment (TME) is a complex system where malignant cells interact with immune and nonimmune cells individually or in combination to affect sensitivity to immunotherapy. (Ref 9) Rational combinations of ICIs with TME-targeted therapy will be the next generation of immune-based approaches for cancer treatment. (Ref 9)

Myeloid cells are the most abundant cells in the TME. The tumors recruit and/or reshape myeloid cells to tumor-associated macrophages (TAMs), dendritic cells (DCs), myeloid-derived suppressor cells (MDSCs), and tumor-associated neutrophils (TANs), to have an immunosuppressive environment. (Ref 10) Tumor-infiltrating myeloid cells (TIMs) directly stimulate tumor cell proliferation by cytokines and growth factors and enhance tumor vascularization by angiogenic stimulators. (Ref 11) They also promote cancer progression by suppressing anti-tumor T cell or natural killer (NK) cell function via secreting regulatory cytokines such as transforming growth factor-beta (TGF-β), interleukin-10 (IL-10), and arginase 1 (ARG1) or overexpressing inhibitory ligands such as PD-L1. (Refs 11-13) However, some types of TIMs, including anti-tumorigenic (M1 type) TAMs and CD103⁺ DCs, have anti-tumor functions through cytotoxicity toward tumor cells via phagocytosis or induction of tumor cell elimination by CD8+ T cells through antigen cross-presentation. (Refs 11, 14-15)

The RNA N⁶-methyadenosine (m⁶A) methylation, an epigenetic modification, which is the most common type of RNA methylation that occurs at the N⁶-position of adenosine, has recently been found to play a critical role in shaping the TME. (Ref 16) It has been consistently proven that targeting m⁶A modifiers in tumor cells could induce a strong anti-tumor response and contribute to the efficacy of immunotherapy. For example, depletion of m⁶A RNA methyltransferases, such as methyltransferase-like protein 3/14 (METTL3/14), in tumor cells enhances the immune responses to anti-PD-1 therapy. (Ref 17) Inhibition of m⁶A RNA demethylase fat mass and obesity-associated protein (FTO) or AlkB homolog 5, RNA demethylase (ALKBH5) sensitizes tumor cells to T cell cytotoxicity dependent or independent of anti-PD-1/anti-PD-L1 treatment. (Refs 18-21) Recently, several studies highlighted that the m⁶A modifiers in TIMs play significant roles in shaping tumor immunity and immunotherapy. (Refs 22-25) TAMs are potent regulators of tumor-associated immune suppression in the TME; however, the mechanisms for this effect remain to be fully elucidated, including the roles of readers of the m⁶A modification in this setting. (Ref 10) The disclosure is directed, inter alia, to overcoming and solving this and other issues in the treatment of cancer.

### BRIEF SUMMARY

Provided herein are compounds comprising a tumor-associated macrophage (TAM) targeting agent attached to a YTHDF2 inhibitor. In embodiments, the TAM targeting agent is a ligand for a TAM receptor, wherein the TAM receptor is TLR9, CD163, CD206, CD14, CD16, CD32, CD64, CD68, CD71, CCR5, or CCR2. In embodiments, the TAM targeting agent is a phosphorothioated CpG oligodeoxynucleotide. In embodiments, the YTHDF2 inhibitor is an antibody, a small molecule, an aptamer, a nucleic acid, a protein, or an enzyme. In embodiments, the YTHDF2 inhibitor is an antisense inhibitor, such as shRNA or siRNA.

Provided herein are methods of treating cancer in a patient in need thereof comprising administering to the patient a compound comprising a tumor-associated macrophage (TAM) targeting agent attached to a YTHDF2 inhibitor. In embodiments, the cancer is a solid tumor. In embodiments, the solid tumor is a hot tumor. In embodiments, the solid tumor is a cold tumor. In embodiments, the methods further comprise administering to the patient an effective amount of an immune checkpoint inhibitor, such as a PD-1 inhibitor or a PD-L1 inhibitor.

Provided herein is a YTHDF2 inhibitor of Formula (I) or a pharmaceutically acceptable salt thereof:

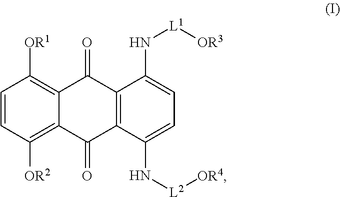

wherein R¹, R², R³, and R⁴ are each independently H or C₁₋₅ alkyl, and L¹ and L² are each independently a 2 to 8 membered heteroalkylene. Provided herein are pharmaceutical compositions containing the YTHDF2 inhibitor of Formula (I) or a pharmaceutically acceptable salt thereof and methods of treating cancer in a patient in need thereof comprising administering to the patient an effective amount of the YTHDF2 inhibitor of Formula (I) or a pharmaceutically acceptable salt thereof. In embodiments, the methods further comprise administering to the patient an effective amount of an immune checkpoint inhibitor, such as a PD-1 inhibitor or a PD-L1 inhibitor. In embodiments of the compound of Formula (I) or the pharmaceutically acceptable salt thereof, R¹, R², R³, and R⁴ are hydrogen, and L¹ and L² are —(CH₂)₂NH(CH₂)₂—.

Provided herein are naïve CD4+ T cells, wherein the naïve CD4+ T cells do not express a YTHDF2 protein. In embodiments, the naïve CD4+ T cells do not express a YTHDF2 protein and comprise a chimeric antigen receptor.

Provided herein T helper 9 cells, wherein the T helper 9 cells do not express a YTHDF2 protein. In embodiments, the T helper 9 cells do not express a YTHDF2 protein and comprise a chimeric antigen receptor.

These and other embodiments of the disclosure are described herein.

### BRIEF DESCRIPTION OF THE DRAWINGS

FIGS. 1A-1H: YTHDF2 deficiency in macrophages suppresses tumor growth. FIGS. 1A-1B: B16-OVA (FIG. 1A) or MC38 (FIG. 1B) tumor growth and tumor weight on day 15 in WT and Ythdf2 cKO mice (n=5 per group). FIGS. 1C-1D: B16F10 lung metastases (c, n=5 per group) and mouse survival (FIG. 1D, n=12 per group) in WT and Ythdf2 cKO mice. FIGS. 1E-IF: Representative plots, percentages, and absolute numbers of tumor-infiltrating macrophages from WT and Ythdf2 cKO mice on day 14 post B16-OVA (e) or MC38 (f) tumor inoculation (n=5 per group). g, B16-OVA tumor growth in WT and Ythdf2 cKO mice treated previously with clodronate liposomes or PBS liposomes (n=4-6 per group). h, B16-OVA (n=6 per group) or MC38 (n=4 per group) tumor growth in immunocompetent CD45.1 recipient mice transplanted with BMDMs from WT or Ythdf2-cKO mice. Data are shown as mean±SD and were analyzed by two-way ANOVA with mixed-effects model (FIGS. 1A, 1B, 1G, 1H) followed by the Holm-Šídík post-test or unpaired two-tailed t-test (FIGS. 1A-1C, 1E, IF) or Kaplan-Meier survival analysis and log-rank test (FIG. 1D). Data in FIGS. 1A-1H are representative of at least two independent experiments (*P<0.05, **P<0.01, ***P<0.001, ****P<0.0001).

FIGS. 2A-2I: YTHDF2 deficiency enhances CD8⁺ T cell-mediated anti-tumor immunity. FIGS. 2A-2B: Representative plots and percentages of tumor-infiltrating IFN-γ-producing CD8⁺ T cells from WT and Ythdf2 cKO mice on day 14 post-B16-OVA (FIG. 2A) or MC38 (FIG. 2B) tumor inoculation (n=5 per group). FIGS. 2C-2D: Representative plots and percentages of tumor-infiltrating effector memory (Tem), central memory (Tcm), and naïve (Tn) CD8⁺ T cells from WT and Ythdf2 cKO mice on day 14 post B16-OVA (FIG. 2C) or MC38 (FIG. 2D) tumor inoculation (n=5 per group). FIGS. 2E-2F: Representative plots and percentages of SIINFEKL-specific CD8⁺ T cells (E) or KSPWFTTL-specific CD8⁺ T cells (F) from WT and Ythdf2 cKO mice on day 14 post-B16-OVA (FIG. 2E) or MC38 (FIG. 2F) tumor inoculation (n=5 per group). FIG. 2G: 3×10⁵ lymphocytes isolated from draining lymph nodes of B16-OVA bearing-mice were stimulated with 10 μg/ml SIINFEKEL for 48 h. IFN-γ production was determined with an ELISPOT assay (n=6 per group). FIG. 2H: B16-OVA tumor growth in WT and Ythdf2 cKO mice treated with anti-IgG or anti-CD8 antibody (n=5-6 per group). i, B16-OVA tumor growth in Rag1^{-/-} mice implanted with BMDMs from WT or Ythdf2-cKO mice together with CD8⁺ T cells from OT-I mice (n=4-5 per group). Data are shown as mean±SD and were analyzed by two-way ANOVA with mixed-effects model and adjusted by Holm-Šídík post-test (FIGS. 2H-2I) or unpaired two-tailed t-test (FIGS. 2A-2G). Data in FIGS. 2A-2I are representative of at least two independent experiments (ns, not significant; *P<0.05, **P<0.01, ***P<0.001, ****P<0.0001).

FIGS. 3A-3G: YTHDF2 deficiency reprograms TAMs by promoting polarization of M1 macrophages. FIGS. 3A-3B: T-distributed stochastic neighbor embedding (t-SNE) plot (FIG. 3A) and the proportions (FIG. 3B) of M1 and M2 macrophages from scRNA-seq. FIG. 3C: Dot plots showing the expression of representative genes of M1 and M2 macrophages. FIGS. 3D-3E: qPCR showing mRNA levels of M1-related genes (Nos2, Il1b, Il6, Il12, Il15, and Il18) (FIG. 3D) or M2-related genes (Arg1, Mrc1, Ym1, and Il10) (FIG. 3E) in TAMs isolated from tumor grafts of B16-OVA-bearing WT and Ythdf2 cKO mice (n=3 per group). FIGS. 3F-3G: Representative histograms and percentages of M1 (CD11b⁺F4/80⁺iNOS⁺) macrophages (FIG. 3F) or M2 (CD11b⁺F4/80⁺Arg1⁺) macrophages (FIG. 3G) in tumor grafts from B16-OVA-bearing WT and Ythdf2 cKO mice (n=6 per group). Data are shown as mean±SD and were analyzed with an unpaired two-tailed t-test (FIGS. 3D-3G). Data in FIGS. 3D-3G are representative of at least two independent experiments (ns, not significant; *P<0.05, **P<0.01, ***P<0.001, ****P<0.0001).

FIGS. 4A-4H: YTHDF2 deficiency enhances cross-presentation ability of M1 macrophages. FIG. 4A: Violin plots showing the enrichment of signatures of early-activated, effector, memory, and memory-precursor CD8⁺ T cells in scRNA-seq. FIG. 4B: Dot plots showing mean strength of selected ligand-receptor pairs of macrophages and effector CD8⁺ T cells. Dot size indicates the mean strength level, and color shows the P-value calculated by the Permutation test. FIG. 4C: Empirical cumulative distribution function (ECDF) plot of enrichment score for the MHC class I gene signature in M1 macrophages from WT and Ythdf2 cKO mice. FIG. 4D: Dot plots showing expression features of selected genes from the MHC class I presentation signature in M1 macrophages from WT and Ythdf2 cKO mice. FIGS. 4E-4H: Representative plots and percentages of IFN-γ production by OT-I CD8⁺ T cells co-cultured with SIINFEKEL peptide (FIGS. 4E-4F) or ovalbumin protein (FIGS. 4G-4H)—loaded M1 BMDM (FIG. 4E: n=3; FIG. 4G: n=4) or TAMs isolated from B16-OVA tumor-bearing mice (FIGS. 4F, 4H n=3). Data are representative of at least three independent experiments in FIGS. 4E-4H, are shown as mean±SD and were analyzed by unpaired two-tailed t-test (FIGS. 4E-4H, *P<0.05, **P<0.01).

FIGS. 5A-5I: YTHDF2 deficiency drives M1 macrophage polarization via IFN-γ-STAT1 signaling. FIGS. 5A-5B: The top six enriched pathways in YTHDF2-deficient M1 macrophages compared to the WT macrophages from GSEA (FIG. 5A). The enrichment of the IFN-□ response in M1 macrophages from Ythdf2 cKO mice compared to those from WT mice is shown (FIG. 5B).

FIG. 5C: A volcano plot of differentially expressed genes in M1 macrophages from scRNA-seq data. FIG. 5D: BMDMs from WT and Ythdf2 cKO mice were treated with IFN-γ (100 ng/ml) for 0, 0.25, 0.5, 1, 2, and 4 h. The cells were collected for immunoblotting using phosphor (p)-STAT1, STAT1, and actin antibodies. FIG. 5E: M1 macrophages from WT or Ythdf2 cKO mice were transfected with Stat1 siRNA or scrambled siRNA for 48 h and then stimulated with IFN-γ for 6 h. mRNA levels of the stimulated cells were determined by qPCR (n=3 per group). FIGS. 5F-5G: B16-OVA tumor growth in immunocompetent C57BL/6 mice transplanted with STAT1 knock-down (FIG. 5F) or STAT1 knock-out (FIG. 5G) BMDMs from WT or Ythdf2 cKO mice (n=5 per group). FIG. 5H: B16-OVA tumor growth in WT and Ythdf2 cKO mice treated with anti-IgG or anti-IFN-γ antibody (n=4-5 per group). FIG. 5I: B16-OVA tumor growth in immunocompetent C57BL/6 mice transplanted with IFNGR1 knock-out BMDMs from WT or Ythdf2 cKO mice (n=5 per group). Data are shown as mean±SD and were analyzed with one-way ANOVA with the Holm-Šídík post-test (FIG. 5E) or two-way ANOVA with mixed-effects model followed by the Holm-Šídík post-test (FIGS. 5F-5I). Data in FIGS. 5D-5I are representative of at least two independent experiments (ns, not significant; *P<0.05, **P<0.01, ***P<0.001, ****P<0.0001).

FIGS. 6A-6G: YTHDF2 decreases stability of Stat1 mRNA in macrophages. FIG. 6A: Integrative Genomics Viewer tracks displaying the distribution of m⁶A peaks and YTHDF2-binding peaks across the Stat1 transcripts. FIG. 6B: Schematic representation of m⁶A positions and mutations within STAT1 3′UTR. FIGS. 6C-6D: RIP using either an m⁶A (FIG. 6C) or a YTHDF2 antibody (FIG. 6D) followed by qPCR to test whether two Stat1 sites in the 3′UTR region were m⁶A methylated and bound by YTHDF2 in M1 macrophages. Rabbit IgG served as control. Enrichment of the indicated genes was normalized to the input level. FIG. 6E: mRNA half-life (t_{1/2}) of Stat1 transcripts in M1 macrophages from WT and Ythdf2 cKO mice. FIG. 6F: Schematic representation of the construction of WT or mutated (GGAC to GGGC) Stat1 3′UTR inserted into the pmirGLO vector. FIG. 6G: Relative luciferase activity of firelfly (F)-Luc/renilla (R)-Luc of pmirGLO-3′UTR-WT, pmirGLO-3′UTR-Mut-1, Mut2, or Mut 1/2 in WT and Ythdf2 cKO BMDMs. Data are shown as mean±SD and were analyzed by unpaired two-tailed t-test (FIGS. 6C-6D and 6G). Data in FIGS. 6C-6E and FIG. 6G are representative of at least two independent experiments (ns, not significant; *P<0.05, **P<0.01).

FIGS. 7A-7G: TLR9-targeted YTHDF2 silencing in TAMs suppresses tumor growth and synergizes with anti-PD-L1 blockade. FIG. 7A: Scheme of therapeutic strategies that use CpG-Ythdf2 siRNA alone or in combination with anti-PD-L1. FIG. 7B: B16-OVA or MC38 tumor growth in mice treated with CpG-Ythdf2 siRNA, CpG-scrambled siRNA, CpG only, or PBS (n=5 per group). FIG. 7C: Percentages of tumor-infiltrating IFN-γ-producing CD8⁺ T cells in B16-OVA (n=5 per group) or MC38 tumor models (n=5 for PBS group; n=3 for CpG only, CpG-Scrambled siRNA, and CpG-Ythdf2 groups) as described in FIG. 7B. For the MC38 model, tumors in the CpG only, CpG-Scrambled siRNA, and CpG-Ythdf2 groups were pooled for flow cytometry due to small tumor size (sample size after pooling n=3). FIG. 7D: B16-OVA tumor growth in mice treated with CpG-Ythdf2 siRNA, CpG-scrambled siRNA, or in combination with anti-PD-L1 (left, n=5-6 per group) and percentages of tumor-infiltrating IFN-γ-producing CD8⁺ T cells (right, n=5 for CpG-scrambled siRNA plus IgG and CpG-scrambled siRNA plus anti-PD-L1 groups, n=3 for CpG-Ythdf2 siRNA plus IgG and CpG-Ythdf2 siRNA plus anti-PD-L1 groups). Tumors from the CpG-Ythdf2 siRNA plus IgG and CpG-Ythdf2 siRNA plus anti-PD-L1 groups were pooled for flow cytometry due to small tumor size (sample size after pooling n=3). FIG. 7E: IFN-γ and granzyme B production by Ly95 T cells when co-cultured with WT or YTHDF2 knock-down macrophages preloaded with NY-ESO_{157-c165} peptide (n=5). FIGS. 7F-7G: Colon cancer sections were immunohistochemically stained with YTHDF2 plus CD68 or CD8 alone. Dash lines outline the edge of tumor zones. Asterisk marks the stroma tissues. Representative YTHDF2⁺CD68⁺ high (Patient #11) and YTHDF2⁺CD68⁺ low (Patient #2) specimens are shown (FIG. 7F). Scale bars=100 μm. Black rows mark YTHDF2⁺CD68⁺ cells. Correlations between YTHDF2⁺CD68⁺ cells and CD8⁺ T cells in the stroma area are shown (FIG. 7G: n=18 patients). Data are shown as mean SD and were analyzed with one-way ANOVA with the Holm-Šídík post-test (FIGS. 7C-7E) or two-way ANOVA with mixed-effects model (FIGS. 7B, 7D) and adjusted by the Holm-Šídík post-test or Spearman correlation (FIG. 7G). Data in FIGS. 7B-7E are representative of at least two independent experiments (*P<0.05, **P<0.01, ***P<0.001, ****P<0.0001).

FIGS. 8A-8F: TCGA database and public single-cell RNA-seq data analysis to determine expression pattern of YTHDF2 in cancers and tumor-infiltrating myeloid cells. FIG. 8A: Boxplots show the YTHDF2 expression in multiple types of cancers and corresponding normal tissues from RNA-seq results of the TCGA database. Red and blue boxes indicate the tumor group and normal group, respectively. FIG. 8B: Scatter plots showing the correlation of YTHDF2 expression and immune scores calculated by the ESTIMATE algorithm from the TCGA database. FIG. 8C: The bubble plots show the correlation of YTHDF2 expression with immune-related markers in each type of cancer. FIGS. 8D-8F: YTHDF2 expression in tumor-infiltrating myeloid cells and corresponding normal tissues from single-cell RNA-seq datasets of GBM (FIG. 8D), COAD (FIG. 8E), and BRCA (FIG. 8F). P values were calculated by Wilcoxon rank-sum test. Abbreviation for TCGA cancer types: BRCA, Breast invasive carcinoma; CHOL, Cholangiocarcinoma; COAD, Colon adenocarcinoma; DLBC, Lymphoid neoplasm diffuse large B-cell lymphoma; GBM, Glioblastoma multiforme; LGG, Brain lower grade glioma; PAAD, Pancreatic adenocarcinoma; READ, Rectum adenocarcinoma; SKCM, Skin cutaneous melanoma; STAD, Stomach adenocarcinoma; TGCT, Testicular germ cell tumors; THYM, Thymoma; UCEC, Uterine corpus endometrial carcinoma; UCS, Uterine carcinosarcoma.

FIGS. 9A-9S: YTHDF2 deficiency does not affect the percentages, absolute numbers, or functions of tumor-infiltrating MDSCs and DCs. FIGS. 9A-9D: Deletion of YTHDF2 in splenic myeloid cells (CD3⁻CD19⁻CD49b⁻CD11b⁺) and bone marrow-derived macrophages (BMDMs) from WT mice and Ythdf2 cKO mice by qPCR (FIGS. 9A, 9C) and immunoblotting (FIGS. 9B, 9D). FIGS. 9E-9G: Gating strategy (FIG. 9E), percentages (FIG. 9F), and absolute numbers (FIG. 9G) of myeloid cells, including dendritic cells (CD11c⁺MHC-II⁺), macrophages (CD11b⁺F4/80⁺), monocytes (CD11b⁺Ly6c⁺), and neutrophils (CD11b⁺Ly6G⁺), in the spleen of normal WT and Ythdf2 cKO mice (n=3 per group). FIGS. 9H-9K: Percentages (FIGS. 9H, 9J) and absolute numbers (FIGS. 9I-9K) of tumor-infiltrating MDSCs (CD11b⁺Gr-1⁺) and DCs (CD11b⁺CD11c⁺MHC-II⁺) from WT and Ythdf2 cKO mice on day 14 post-B16-OVA (FIGS. 9H-9I) or MC38 (FIGS. 9J-9K) tumor inoculation (n=5 per group). FIG. 9L: Percentages and representative plots of IFN-γ production by OT-I CD8⁺ T cells co-cultured with purified tumor-infiltrated MDSCs from WT and Ythdf2 cKO mice in the presence of SIINFEKEL (n=3 per group). FIG. 9M: Percentages and representative plots of IFN-γ production by OT-I CD8⁺ T cells co-cultured with purified tumor-infiltrated DCs from WT and Ythdf2 cKO mice in the presence of SIINFEKEL (n=3 per group). FIGS. 9N-9O: Percentages of IFN-γ production by OT-I CD8⁺ T cells co-cultured with purified tumor-infiltrated MDSCs (FIG. 9N) or DCs (FIG. 9O) from WT and Ythdf2 cKO mice in the presence of ovalbumin protein (n=3 per group). FIGS. 9P-9Q: Expression of YTHDF2 in BMDCs (FIG. 9P) or splenic DCs (FIG. 9Q) from WT mice and Ythdf2 cKO mice by immunoblotting. FIG. 9R, Percentages of tumor-infiltrating macrophages after injection with clodronate liposomes or PBS liposomes in B16-OVA-bearing mice (n=4-6 per group). FIG. 9S: Schematic diagram and representative plots showing co-injection of B16-OVA cells with BMDMs. The data show that the macrophages in the tumor tissues are mainly from co-injected donor BMDMs. Data are shown as mean±SD and were analyzed with an unpaired two-tailed t-test (FIGS. 9A-9O) or one-way ANOVA with the Holm-Šídík post-test (FIG. 9R). Data in FIGS. 9A-9D and 9F-9R are representative of at least two independent experiments. (ns, not significant; **P<0.01, ***P<0.001).

FIGS. 10A-10J: YTHDF2 deficiency does not affect phagocytosis by macrophages and CD4⁺ T and NK cells in the tumor microenvironment. FIGS. 10A-10B: M1 macrophages from WT and Ythdf2 cKO mice were treated with pHrodo Red Zymosan Bioparticles for 3 h at 37° C.; then their phagocytic ability was compared by flow cytometry (n=3 per group). Data shown are median fluorescence intensity (MFI, FIG. 10A) and representative histogram (FIG. 10B) of flow cytometry (n=3 per group). FIG. 10C: B16-OVA tumor growth in Rag1^{-/-} mice transplanted with BMDMs from WT or Ythdf2-cKO mice (n=3 per group). FIG. 10D: B16-OVA tumor growth in Rag1^{-/-} mice that were i.v. injected with CD3⁺ T cells and s.c. transplanted with BMDMs from WT or Ythdf2-cKO mice (n=5 per group). FIGS. 10E-10H: Percentages (FIGS. 10E, 10G) and absolute numbers (FIGS. 10F, 10H) of tumor-infiltrating CD4⁺ T cells, CD8⁺ T cells, and NK cells from WT and Ythdf2 cKO mice on day 14 post-B16-OVA (FIGS. 9E-9F) or MC38 (FIGS. 10G-10H) tumor inoculation (n=5 per group). FIGS. 10I-10J: Percentages and representative plots of tumor-infiltrating Th1 cells (CD4 IFN-γ⁺), Th17 cells (CD4⁺ IL-17A⁺), Treg cells (CD4⁺Foxp3⁺), and granzyme B producing NK cells from WT and Ythdf2 cKO mice on day 14 post B16-OVA (FIG. 101) or MC38 (FIG. 10J) tumor inoculation (n=4 per group). Data are shown as mean±SD and were analyzed with an unpaired two-tailed t-test (FIGS. 10A, 10E-10J) or two-way ANOVA with mixed-effects model and adjusted by the Holm-Šídík post-test (FIGS. 10C-10D). Data in FIGS. 10A-10J are representative of at least two independent experiments (ns, not significant).

FIGS. 11A-11G: scRNA-seq of CD45⁺ tumor-infiltrating immune cells and cross-presentation ability of M1 macrophages in Ythdf2 cKO mice. FIGS. 11A-11B: CD45⁺ tumor-infiltrating immune cells were sorted from WT and Ythdf2 cKO mice on day 14 post B16-OVA tumor inoculation. scRNA-seq was performed using the Chromium System from 10× Genomics. A t-distributed stochastic neighbor embedding (t-SNE) plot shows 15 cell types in the merged (FIG. 11A) and separated groups (FIG. 11B) from scRNA-seq. FIG. 11C: Dot plots showing expression of marker genes in 15 cell types. FIG. 11D: Immunoblotting showing levels of iNOS in M1 or Arg1 levels in M2 BMDMs from WT and Ythdf2 cKO mice. The numbers below a lane indicate expression of target proteins normalized to expression of β-actin. FIG. 11E: Rose diagrams showing the proportions of 15 cell types in WT and Ythdf2 cKO mice. FIG. 11F: Representative histograms and percentages of H-2K^{b} SIINFEKL-positive M1 or M2 macrophages in tumor tissues from WT and Ythdf2 cKO mice on day 14 post-B16-OVA tumor inoculation (n=3 per group). FIG. 11G: mRNA levels of Ifng4 and Ifnb1 from macrophages isolated from B16-OVA tumor-bearing WT or Ythdf2 cKO mice, determined by qPCR. Data are shown as mean±SD and were analyzed by two-tailed t-test (FIG. 11G), one-way ANOVA with the Holm-Šídík post-test (FIG. 11F). Data are in FIGS. 11D, 11F-11G representative of at least two independent experiments (ns, not significant; **P<0.01, ****P<0.0001).

FIGS. 12A-12H: YTHDF2 regulates macrophage reprogramming from M2 to M1 by targeting IFN-γ-STAT1 signaling. FIG. 12A: Ingenuity Pathway Analysis showing that both the IFN signaling pathway and activation of interferon regulatory factor (IRF) by cytosolic pattern recognition receptors are highly enriched in YTHDF2 deficient M1 macrophages. FIG. 12B: BMDMs from WT and Ythdf2 cKO mice were treated with IFN-γ (100 ng/ml) for 6 h. mRNA levels of IFN-γ response genes were determined by qPCR. FIGS. 12C-12D: TAMs sorted from tumor tissues of B16-OVA tumor-bearing WT or Ythdf2 cKO mice were subjected to qPCR to examine mRNA levels of Stat1 (FIG. 12C) and to immunoblotting using phosphor (p)-STAT1, STAT1, and beta-actin antibodies (FIG. 12D). FIG. 12E: Immunoblotting showing levels of STAT1 in BMDMs transfected with Stat1 siRNA or scrambled siRNA. FIG. 12F: Immunoblotting showing levels of STAT1 in BMDMs nucleofected with Stat1 gRNA or control gRNA. FIG. 12G: Representative histogram of IFNGR1 levels in BMDMs nucleofected with Ifngr1 gRNA or control gRNA. FIG. 12H: B16-OVA tumor growth in Rag1^{-/-} mice implanted with BMDMs from WT or Ythdf2-cKO mice together with CD8⁺ T cells from IFN-γ^{-/-} mice (n=4 per group). Data are shown as mean±SD and were analyzed with an unpaired two-tailed t-test (FIGS. 12B-12C) or two-way ANOVA with mixed-effects model and adjusted by the Holm-Šídík post-test (FIG. 12H). The numbers below a lane indicate expression of target proteins normalized to expression of β-actin. Data in FIGS. 12B-12H are representative of at least two independent experiments (ns, not significant; *P<0.05, **P<0.01, ***P<0.001, ****P<0.0001).

FIGS. 13A-13J: Transcriptome-wide identification of YTHDF2-binding targets in macrophages using m⁶A-RIP and YTHDF2 RIP. FIG. 13A: m⁶A motif was detected by the HOMER motif discovery tool. FIG. 13B: Density distribution of m⁶A peaks across the length of mRNA. FIG. 13C: Pie chart depicting the fraction of m⁶A peaks in four transcript segments. FIG. 13D: Volcano plot showing the differentially expressed genes in M1 macrophages from Ythdf2 cKO mice compared with those from WT mice from RNA-seq. FIG. 13E: The enrichment pathways between M1 macrophages of Ythdf2 cKO mice and those from WT mice from GSEA. FIG. 13F: GSEA showing enrichment of IFN-□ response sets in M1 macrophages from Ythdf2 cKO mice compared with those from WT mice. FIG. 13G: Heatmaps of genes representing IFN-γ, TNF-α, IFN-α receptor, IFN regulatory factor 7 (IRF7), Toll-like receptor 4 (TLR4), and STAT1 signaling pathways in WT and Ythdf2 cKO BMDMs from RNA-seq data (n=2). Transcript methylation is depicted by filled (m⁶A-modified) or unfilled (non-m6A-modified) circles. FIG. 13H: Overlapping of high confidence YTHDF2-binding regions between replicate 1 and replicate 2. FIG. 13I: Density distribution of YTHDF2-binding regions across the length of mRNA. FIG. 13J: Pie chart depicting the fraction of YTHDF2-binding regions in four transcript segments.

FIGS. 14A-14G: YTHDF2 is positively regulated by IL-10-STAT3 signaling in TAMs. FIG. 14A: Immunoblotting showing levels of YTHDF2 in macrophages isolated from spleen or tumor tissues. FIG. 14B: Immunoblotting showing levels of YTHDF2 in BMDMs stimulated without or with IL-4, TGF-β, IL-10, or IFN-γ for 24 h. FIG. 14C: Immunoblotting showing levels of YTHDF2 and phospho-Stat3 in BMDMs stimulated with different doses of IL-10 for 24 h. FIG. 14D: Binding sites for STAT3 in the promoter regions of Ythdf2 (predicted from http://jaspar.genereg.net). FIG. 14E: Luciferase reporter assay shows that STAT3 activates Ythdf2 gene transcription. FIG. 14F: Binding of STAT3 to the Ythdf2 promoter in IL-10-treated BMDMs as determined by ChIP-qPCR. FIG. 14G: Immunoblotting showing levels of YTHDF2 and phospho-Stat3 in BMDMs from WT or STAT3 cKO mice stimulated without or with IL-10 (10 ng/ml). Data are shown as mean±SD and were analyzed with an unpaired two-tailed t-test (FIGS. 14E-14F). The numbers below a lane indicate expression of YTHDF2 normalized to expression of β-actin. Data in FIGS. 14A-14C and FIG. 14E-14G are representative of at least two independent experiments (ns, not significant; *P<0.05, **P<0.01, ***P<0.001, ****P<0.0001).

FIGS. 15A-15P: TLR9 expression and CpG-Ythdf2 siRNA design as well as its uptake by macrophages associated with anti-tumor effect in vivo. FIG. 15A: Violin plot showing the Tlr9 expression across 15 cell clusters from scRNA-seq data in this study. FIG. 15B: Sequence of the CpG-linked mouse Ythdf2 siRNA conjugate (CpG-Ythdf2 siRNA). FIGS. 15C-15D: BMDMs were incubated with different doses of Cy3-labeled CpG-Ythdf2 siRNA (FIG. 15C) or with 100 nM Cy3-labeled CpG-Ythdf2 siRNA for the indicated times (FIG. 15D). CpG-Ythdf2 siRNA uptake was examined by flow cytometry. Data shown are representative histograms. FIG. 15E: Cy3-labeled CpG-Ythdf2 siRNA or CpG only was injected into B16-OVA tumors of C57BL/6 mice. Flow cytometric analyses, performed 24 h after the injection, showed efficient uptake of CpG-Ythdf2 siRNA by macrophages. Data shown are representative histograms. FIG. 15F: mRNA levels of Ythdf2 in macrophages sorted from tumor tissues of B16-OVA-bearing mice treated with CpG-Ythdf2 siRNA or CpG-scrambled siRNA (n=3 per group). FIGS. 15G-15H: Representative dot plots and percentages of M1 (FIG. 15G: CD11b⁺F4/80⁺iNOS⁺) or M2 (FIG. 15H: CD11b⁺F4/80⁺Arg1⁺) macrophages in tumor grafts from B16-OVA-bearing mice treated with CpG-Ythdf2 siRNA or CpG-scrambled siRNA (n=5 per group). FIG. 15I: mRNA levels of Ythdf2 in macrophages sorted lung tissues of B16-F10-bearing mice treated with CpG-Ythdf2 siRNA or CpG-scrambled siRNA (n=3 per group). FIGS. 15J-15K: Metastatic nodules (FIG. 15J) and percentages of tumor-infiltrating IFN-γ producing CD8⁺ T cells (FIG. 15K) in the lung of B16F10-bearing mice treated with CpG-Ythdf2 siRNA, CpG-scrambled siRNA, CpG only, or PBS (n=5 per group). FIG. 15L: B16-OVA tumor growth in Batf3 KO mice treated with CpG-Ythdf2 siRNA, or CpG-scrambled (n=5 per group). FIGS. 15M-15N: Violin plots showing the TLR9 expression across cell clusters in scRNA-seq datasets of patients with glioblastoma (FIG. 15M) or kidney cancer (FIG. 15N). FIGS. 15O-15P: Representative histograms showing expression levels of PD-L1 in B16-OVA cells (FIG. 15O) or BMDMs (FIG. 15P) after treatment with IFN-γ for 24 h. Median fluorescence intensity (MFI) is shown. Data are shown as mean±SD and were analyzed with an unpaired two-tailed t-test (FIGS. 15F-15I) or one-way ANOVA with the Holm-Šídík post-test (FIGS. 15J-15K) or two-way ANOVA with the mixed-effects model and adjusted by Holm-Šídík post-test (FIG. 15L). Data in FIGS. 15C-15L and FIGS. 15O-15P are representative of at least two independent experiments (*P<0.05, **P<0.01, ***P<0.001).

FIGS. 16A-16F: The relationship between YTHDF2 expression in TAMs and overall survival in melanoma cancer patients. FIG. 16A: Knock-down of YTHDF2 using siRNA in human monocyte-derived macrophages enhances expression of pro-inflammation cytokines. Data are shown as mean±SD and were analyzed by unpaired two-tailed t-test. FIGS. 16B-16C: Lung cancer tissue microarray was immunohistochemically stained with YTHDF2 plus CD68 or CD8 alone. Representative YTHDF2⁺CD68⁺ low (Patient #2) and YTHDF2⁺CD68⁺ high (Patient #9) specimens are shown (FIG. 16B). Scale bars=100 μm. Black arrows mark YTHDF2⁺CD68⁺ cells. Correlations between YTHDF2⁺CD68⁺ cells and CD8⁺ T cells are analyzed by Spearman correlation (c, n=32 patients). FIG. 16D: Correlation of YTHDF2 expression in tumor infiltrated CD68⁺ macrophages with overall survival of skin cutaneous melanoma (SKCM) patients. P values were calculated using the log-rank test. FIG. 16E: Heatmap of the ordered, z-transformed expression values for YTHDF2 target genes in a patient dataset of SKCM. FIG. 16F: Correlation of a YTHDF2 target gene signature with overall survival of patients with SKCM comparing high and low quartiles. P values were calculated using the log-rank test. Data in FIG. 16A are representative of at least two independent experiments (***P<0.001, ****P<0.0001).

FIG. 17: A working model for how reduction in YTHDF2 reprograms tumor-associated macrophages and elicits potent anti-tumor immunity. TAMs traffic into the TME and respond to IL-10, which upregulates YTHDF2 by IL-10-STAT3 signaling. Ablation of YTHDF2 in TAMs or knock-down of YTHDF2 by CpG-Ythdf2 siRNA enhances the stability of STAT1, reprograms TAMs to induce M1 polarization, thereby facilitating CD8⁺ T cell anti-tumor response, eventually suppressing tumor growth.

FIG. 18: Gating strategy of MDSCs (CD45⁺CD3⁻CD19⁻CD49b⁻CD11b⁺Gr-1⁺), TAMs (CD45⁺CD3⁻CD19⁻CD49b⁻CD11b⁺Gr-1⁻F4/80⁺), and DCs (CD45⁺CD3⁻CD19⁻CD49b⁻CD11b⁺Gr-1⁻F4/80⁻CD11c⁺MHC-II⁺) in the tumor of B16-OVA bearing WT mice.

FIG. 19 shows oligonucleotide primer sequences used in Example 1.

FIG. 20 shows CRISPR RNA sequences used in Example 1.

FIG. 21 shows the top 24 ranked transcription factors associated with YTHDF2, predicated by “ChIPBase” and “AnimalTFDB3”.

FIGS. 22A-22H show genetic deletion of Ythdf2 in tumor cells impairs tumor progression in immunocompetent mice. FIGS. 22A-22B: Wild-type C57BL/6 mice were injected subcutaneously with either 1×10⁶ WT and Ythdf2-KO MC38 cells (FIG. 22A) or 1×10⁶ WT and Ythdf2-KO B16-OVA cells (FIG. 22B). Tumor size was measured (n=5 tumors per group). FIG. 22C: Overall survival (OS) of immunocompetent mice bearing s.c. tumors implanted with WT or Ythdf2-KO B16-OVA cells (n=14-15 tumors per group). FIGS. 22D-22E: WT or Ythdf2-KO B16-OVA tumors were transplanted from Rag1^{-/-} to C57BL/6 mice. Tumor volume (FIG. 22D) and tumor weight (FIG. 22E) were measured on day 14 after the tumor was transplanted (n=4 tumors per group). FIG. 22F: WT or Ythdf2-KO B16-OVA tumor (1×10⁶) growth in C57BL/6 hosts following prior treatment with anti-IgG, anti-NK1.1, anti-CD4, and anti-CD8 antibodies (n=5-6 tumors per group). FIGS. 22G-22H: WT or Ythdf2-KO MC38-OVA (1×10⁶) tumor growth (FIG. 22G) and tumor weight (FIG. 22H) after OT1 CD8⁺ T cell adoptive transfer to Rag1^{-/-} mice (n=5 tumors per group). Data are represented as mean±s.d., and n=number of mice. Statistical analysis was performed using two-way ANONA with a mixed-effects model with P values adjusted by a Holm-Šídík method for multiple comparisons (FIGS. 22A-22B, 22F-22G) or unpaired two-tailed t-test (FIGS. 22D-22E, 22H) or Kaplan-Meier survival analysis and log-rank test (FIG. 22C). The data presented represents one of two or three independent experiments. *p<0.05, **p<0.01, and ****p<0.001.

FIGS. 23A-23O show YTHDF2 deficiency in tumor cells reprograms the immunosuppressive TME. (A) A UMAP plot from scRNA-seq data showing 8 cell clusters of CD45⁺ tumor-infiltrating immune cells sorted from WT and Ythdf2-KO B16-OVA-bearing mice on day 14 after B16-OVA tumor inoculation; pDC, plasmacytoid DC; cDC, Classical Dendritic Cells; TANs, Tumor-associated neutrophils; CAFs, cancer-associated fibroblasts. (B and C) A UMAP plot (B) and the proportions (C) of anti-tumoral and pro-tumoral macrophages from scRNA-seq data, as in A. (D) Representative Gene Ontology clusters of up-regulated genes in anti-tumoral macrophages from scRNA-seq data. (E and F) A UMAP plot (E) and the proportions (F) of T cells subpopulations from scRNA-seq data, as in A. (G) Representative Gene Ontology clusters of up-regulated genes in effector CD8⁺ T cells and Mki67⁺ CD8⁺ T cell subpopulations from scRNA-seq data. (H-J) Representative plots (H), percentage (I), and absolute number (J) of iNOS⁺F4/80⁺ TAMs from WT and Ythdf2-KO B16-OVA (1×10⁶) tumors after tumor inoculation (n=4 tumors per group). (K-M) Representative plots (K), percentage (L), and absolute number (M) of CD8⁺CD3⁺ T cells from WT and Ythdf2-KO B16-OVA (1×10⁶) tumors after tumor inoculation (n=4 tumors per group). (N and O) Representative plots (N) and absolute number (O) of IFN-γ⁺CD8⁺ T cells from WT and Ythdf2-KO B16-OVA (1×10⁶) tumors after tumor inoculation (n=4 tumors per group). Data are represented as mean±s.d. Statistical analysis was performed using unpaired two-tailed t-test (K, L, N, O, and Q). The data presented represents one of two or three independent experiments.

FIGS. 24A-24L show loss of YTHDF2 in tumor cells promotes the recruitment of CX3CR1⁺ macrophage via CX3CL1. (A) Volcano plot showing the differentially expressed genes (DEGs) between WT and Ythdf2-KO MC38 tumor. DEGs with an absolute log-transformed fold change of >0.25 and adjusted P value of <0.05 (determined by two-sided Wilcoxon rank-sum test and adjusted using the Bonferroni correction) were defined as significant. (B) Top 10 Gene Ontology clusters of up-regulated genes in RNA-seq data. (C) Heatmap of differential expression genes in leukocyte migration pathway between WT and Ythdf2-KO MC38 tumor cells. (D) Overlapping analysis of genes identified by RNA-seq (upregulated genes), m6A-seq, and RIP-seq. 81 upregulated differentially expressed genes bound by YTHDF2 and marked with m⁶A are listed on the right. (E) The absolute number of CX3CR1⁺F4/80⁺CD11b⁺ TAMs from WT and Ythdf2-KO B16-OVA tumors after tumor inoculation (n=3 tumors per group). (F) Schematic representation of a transwell coculture assay involving BMDMs treated with WT or Ythdf2-KO tumor supernatant. (G) Migration cell number of BMDMs induced by WT or Ythdf2-KO B16-OVA tumor cells cultured supernatants, measured with a transwell assay (n=3 samples per group). (H) Migration cell number of BMDMs induced by WT or Ythdf2-KO B16-OVA tumor cell culture supernatant with or without AZD8797, measured with a transwell assay (n=3 samples per group). (I) Wild-type C57BL/6 mice were injected s.c. with 1×10⁶ cells WT or Ythdf2-KO B16-OVA cells. C57BL/6 mice received either an intraperitoneal injection of AZD8797 (1 mg/kg) or an equal amount of PBS once per day for 13 days. Tumor size was measured (n=5 tumors per group). (J) Wild-type C57BL/6 mice were injected subcutaneously with 1×10⁶ cells WT or Ythdf2-KO B16-OVA cells. C57BL/6 mice received either an intraperitoneal injection of Liposomal clodronate (200 μL/per mouse) or an equal amount of liposomal PBS solution on days 0 and 7 after tumor inoculation. Tumor size was measured (n=5 tumors per group). Data are represented as mean±s.d. Statistical analysis was performed using two-way ANONA with a mixed-effects model (M, I, and J), one-way ANONA model (H) or unpaired two-tailed t-test (E, F). P values will be adjusted for multiple comparisons Holm-Šídík method. The data presented represents one of two or three independent experiments. **p<0.01, ****p<0.001. ns, not significant.

FIGS. 25A-25H show YTHDF2 decreases the stability of Cx3cl1 mRNA in tumor cells. (A) Distribution of m6A peaks and YTHDF2-binding peaks across Cx3cl1 by Integrative Genomics Viewer. (B and C) qPCR analysis of mRNA levels of Cx3cl1 after intrasample normalization to the levels of reference gene Actb in WT or Ythdf2-KO MC38 (B), and B16-OVA (C) cells (n=3 samples per group). (D and E) ELISA analysis of the levels of CX3CL1 protein secreted from WT and Ythdf2-KO MC38 (D), or B16-OVA (E) tumor cell culture supernatants (n=3 samples per group). (F and G) RIP using either an antibody to m⁶A (F) or to YTHDF2 (G) followed by qPCR in MC38 tumor cells showing that the Cx3cl1 site in the 3′ UTR region were m⁶A methylated and enriched in YTHDF2 binding. Rabbit IgG served as a control. Enrichment of the indicated genes was normalized to the input level (n=3 samples per group). (H) The mRNA half-life (t_{1/2}) of Cx3cl1 transcripts in WT and Ythdf2-KO B16-OVA tumor cells. Paper fits a linear regression model using log(y) vs time, and then transfer back to the nonlinear regression model by exponential function. Two slopes were compared within the full regression model by t test. Data are represented as mean±s.d. Statistical analysis was performed using unpaired two-tailed t-test (B-G). The data presented represents one of two or three independent experiments.

FIGS. 26A-26H show YTHDF2 deficiency promotes the polarization and antigen presentation of anti-tumoral macrophages. FIGS. 26A-26B: FACS analysis of CD86 (anti-tumoral macrophage marker) expression in BMDMs cocultured with WT or Ythdf2-KO B16-OVA (FIG. 26A), and MC38 (FIG. 26B) tumor cell culture supernatant in the presence of mouse IFN-γ at 1 ng/mL (n=3 samples per group). FIGS. 26C-26D: Representative plots (FIG. 26C) and percentage (FIG. 26D) of Ki67⁺ CD8⁺ T cells from WT or Ythdf2-KO B16-OVA tumor-bearing mice on day 14 after tumor inoculation (n=5 tumors per group). FIGS. 26E-26F: Representative plots (E) and percentage (F) of SIINFEKL-specific CD8⁺ T cells from WT and Ythdf2-KO B16-OVA tumor-bearing mice on day 14 after tumor inoculation (n=5 tumors per group). (G and H) FACS analysis of CD8⁺ T cell proliferation (G) and percentage (H) of IFNγ-producing OT1 CD8⁺ T cells. CD8⁺ T cells isolated from OT1 mice were co-cultured with the SIINFEKEL peptide-loaded CD86⁺F4/80⁺CD11b⁺ TAMs isolated from WT or Ythdf2-KO B16-OVA tumor-bearing mice on day 14 after tumor inoculation (n=4 samples per group). Data are represented as mean±s.d. Statistical analysis was performed using unpaired two-tailed t-test (A, B, D, and F) or one-way ANONA model with P values adjusted for multiple comparisons by Holm-Šídík method (G and H). The data presented represents one of two or three independent experiments.

FIGS. 27A-27H show Ythdf2-KO cells directly regulate CD8⁺ T cell effector function. (A) The ability of OT1 CD8⁺ T cells to lyse WT or Ythdf2-KO B16-OVA tumor cells, measured by real-time cell analysis (RTCA). The experiment was performed twice, yielding similar data both times. (B) ELISA analysis of the levels of IFN-γ protein secreted from OT1 CD8⁺ T cells when cocultured with either WT or Ythdf2-KO B16-OVA tumor cells (n=5 samples per group). (C) Cytotoxicity of OT1 CD8⁺ T cells against WT or Ythdf2-KO MC38-OVA tumor cells was evaluated by a standard ⁵¹Cr release assay (n=3 samples per group). (D) Ifng^{+/+} and Ifng^{-/-} C57BL/6 mice were injected s.c. with 5×10⁵ WT and Ythdf2-KO B16-OVA cells. Tumor size was measured (n=5 mice per group). (E) The proliferation of WT or Ythdf2-KO B16-OVA tumor cells when cocultured with OT1 CD8⁺ T cells either with or without an anti-IFN-γ antibody, measured by real-time cell analysis. The experiment was performed twice, yielding similar data both times. (F) The graph represents measurements of oxygen consumption rate (OCR) upon the addition of oligomycin (Oligo), FCCP, and a combination of rotenone and antimycin A (R/A). Measurements were taken from OT1 CD8⁺ T cells after a 24-hour coculture with either WT or Ythdf2-KO B16-OVA tumor cells. (G and H) Quantified basal respiration (G) and maximal respiration (H) of OT1 CD8⁺ T cells after coculture with WT or Ythdf2-KO B16-OVA tumor cells for 24 hours (n=5-7 samples per group). Data are represented as mean±s.d. Statistical analysis was performed using unpaired two-tailed t-test (A, B, G, and H) or two-way ANONA with a mixed-effects model with P values adjusted for multiple comparisons by Holm-Šídík method (C, D, and E). The data presented represents one of two or three independent experiments.

FIGS. 28A-28L show CD8⁺ T cells derived IFN-γ induces autophagic degradation of YTHDF2. (A) Immunoblotting showing the expression of YTHDF2 in B16-OVA tumor cells after coculture with OT1 CD8⁺ T cells for 24 hours. (B and C) Immunoblotting showing the expression of YTHDF2 in B16-OVA and MC38 tumor cells after treatment with 100 ng/mL IFN-γ for 24 hours. (D and E) qPCR analysis of mRNA levels of Ythdf2 after intrasample normalization to the levels of the reference gene Actb in WT B16-OVA (D) and MC38 (E) tumor cells after treatment with 100 ng/mL IFN-γ for 24 hours (n=3 samples per group). (F and G) WT B16-OVA tumor cells treated with CHX for the indicated time periods. YTHDF2 levels were analyzed by immunoblotting. Representative blots (F) and quantification of immunoblotting signals of YTHDF2 to β-actin levels (G) are shown (n=3 samples per group). (H) Immunoblotting showing the expression of YTHDF2 in B16-OVA tumor cells after treatment with 100 ng/mL IFN-γ for 24 hours. MG132 or Lys05 was then added to the IFN-γ treated tumor cells for 6 h prior to collection. DMSO served as vehicle control. (I) Network schematic showing potential protein-protein interactions. Each node represents a protein, the size of each node represents the degree of connectivity, and each edge represents two proteins that have an interaction. (J) Immunofluorescence analysis of p62 (green) and YTHDF2 (red) in 293T cells, B16-OVA cells, and Hela cells. DAPI is used as a nuclear counterstain (blue). Scale bar: 20 μm (upper) or 10 μm (lower). (K) B16-OVA cell lysates were immunoprecipitated with rabbit anti-YTHDF2 or IgG isotype control and immunoblotted with rabbit anti-YTHDF2 or mouse anti-p62. (L) Immunoblotting showing the expression of YTHDF2 in WT and p62-KO B16-OVA tumor cells after treatment with 100 ng/mL IFN-γ for 24 hours. Data are represented as mean±s.d. Statistical analysis was performed using unpaired two-tailed t-test (C, D and E) or two-way ANONA with a mixed-effects model with P values adjusted for multiple comparisons by Holm-Šídík method (F right). The data presented represents one of two or three independent experiments.

FIGS. 29A-29J show inhibition of YTHDF2 by a small molecule degrader suppresses tumor growth. (A) Immunoblotting showing the expression of YTHDF1, YTHDF2, and YTHDF2 in 293T cells after treatment with indicated doses of DF-A7 for 24 hours. PBS served as vehicle control. (B) Structural complex of YTHDF2 bound with DF-A7. YTHDF2 is colored gray; DF-A7 is orange. Hydrogen bonds are indicated with red dashed lines. (C) In vitro quantification of IC50 that examines the degradation impact of DF-A7 on YTHDF2 using an immunoblotting assay (n=2-3 samples per concentration). The Dose-response-Inhibition under the Nonlinear regression model frame was used to fit the IC50. (D and E) Effect of DF-A7 on tumorigenesis of MC38 (D) and B16-OVA (E) cells in C57BL/6 mice. Tumor cells were inoculated s.c. Tumor-bearing mice were injected i.p. with either PBS or 2.5 mg/kg DF-A7 on day1 and day8 (n=5 tumors per group). (F) Wild-type C57BL/6 mice were injected s.c. with 1×10⁶ cells MC38 cells. Tumor-bearing mice were injected i.p. with PBS or 2.5 mg/kg DF-A7 on day 1 and day 8. Animal survival was monitored (n=5-6 mice per group). (G) Representative plots (left) and percentage (right) of iNOS⁺F4/80⁺ TAMs from MC38 (1×10⁶) tumors after tumor inoculation. Tumor-bearing mice were injected i.p. with PBS or 2.5 mg/kg DF-A7 on day 1 and day 8 (n=4 tumors per group). (H) Representative plots (left) and percentage (right) of CD8⁺CD3⁺ T cells from MC38 (1×10⁶) tumors after tumor inoculation. Tumor-bearing mice were injected i.p. with PBS or 2.5 mg/kg DF-A7 on day 1 and day 8 (n=4 tumors per group). (I) Absolute number of IFN-γ⁺CD8⁺ T cells from MC38 (1×10⁶) tumors after tumor inoculation. Tumor-bearing mice were injected i.p. with PBS or 2.5 mg/kg DF-A7 on day 1 and day 8 (n=4 tumors per group). (J) Therapeutic effect of DF-A7 on tumorigenesis of MC38 cells in C57BL/6 mice. Mice were s.c. inoculated with 5×10⁵ MC38 cells, and injected i.p. with 200 μg/per mouse anti-PD-L1 and/or 2.5 mg/kg DF-A7 on days 1 and 8. Data are represented as mean±s.d. Statistical analysis was performed using unpaired two-tailed t-test (G right and H right, and I) or two-way ANONA with a mixed-effects model with P values adjusted for multiple comparisons by Holm-Šídík method (J) or Kaplan-Meier survival analysis and log-rank test (F). The data presented represents one of two or three independent experiments.

FIGS. 30A-30D show increased YTHDF2 expression is associated with decreasing immune cell infiltration and poor survival. (A) Heatmap showing the correlation between immune cells infiltrate estimation value and YTHDF2 expression in multiple cancers from RNA-seq results of the TCGA database. (B) Heatmap showing the expression of YTHDF2 in malignant cells and immune cells from public single-cell RNA-seq dataset in 20 types of cancers. (C) Dotplot showing the association of the YTHDF2 signature with survival among different cancers. (D) Kaplan-Meier survival analysis based on YTHDF2 signature score in multiple cancers. P values were calculated using the log-rank test. Abbreviation for TCGA cancer types: DLBC, Lymphoid neoplasm diffuse large B-cell lymphoma; MESO, Mesothelioma; PAAD, Pancreatic adenocarcinoma; TGCT, Testicular germ cell tumors; LUAD, Lung adenocarcinoma; CHOL, Cholangiocarcinoma; UCEC, Uterine corpus endometrial carcinoma; LGG, Brain lower grade glioma; LIHC, Liver Hepatocellular Carcinoma; KIRC, Kidney Renal Clear Cell Carcinoma; SKCM, Skin cutaneous melanoma; COAD, Colon adenocarcinoma; LAML, Acute myeloid leukemia; GBM, Glioblastoma multiforme; HNSC, Head and neck squamous cell carcinomas; LUSC, lung squamous cell carcinoma; KIRP, Kidney renal papillary cell carcinoma; THCA, Thyroid Cancer; KICH, Kidney Chromophobe; CESC, cervical squamous cell carcinoma; OV, Ovarian cancer; BLCA, Bladder cancer; ESCA, Esophageal Carcinoma; READ, Rectum adenocarcinoma; STAD, Stomach adenocarcinoma; SARC, Sarcoma; UCS, Uterine carcinosarcoma; THYM, Thymoma; ACC, Adrenocortical carcinoma; PCPG, Pheochromocytoma and Paraganglioma; BRCA, Breast invasive carcinoma; PRAD, Pancreatic adenocarcinoma; UVM, Uveal Melanoma.

FIGS. 31A-31N show Ythdf2 deficiency in tumor cells has no discernable effect on proliferative phenotypes or tumor growth in immunodeficient mice. (A) YTHDF1, YTHDF2, YTHDF3 and β-actin immunoblots in MC38 and B16-OVA cells. Images are representatives of at least three independent experiments. *, nonspecific bands. (B and C) Cell proliferation assay (MTT-based) of WT or Ythdf2-KO MC38 (B) and WT or Ythdf2-KO B16-OVA (C) cells. Aliquots of 5,000 cells were seeded into wells of a 96-well plate. Cell proliferation was measured according to the manufacturer's protocol in the indicated time. (D and E) Quantification of annexin V⁺ tumor cells of WT or Ythdf2-KO MC38 (D) and WT or Ythdf2-KO B16-OVA (E) cells. (n=3 samples per group). (F-K) WT and Ythdf2-KO MC38 (1×10⁶) or WT and Ythdf2-KO B16-OVA (1×10⁶) tumor growth in Rag1^{-/-} (F and G), Rag2^{-/-}Il2gc^{-/-} (H and I), NSG (J and K) immunodeficient hosts (n=3-6 tumors per group). (L-N) Percentage of NK cells (L) or CD4⁺ T cells (M), CD8⁺ T cells (N) from WT and Ythdf2-KO B16-OVA (1×10⁶) tumors on day 16 after tumor inoculation in antibody treated mice (n=5-6 tumors per group). Data are represented as mean±s.d. Statistical analysis was performed using unpaired two-tailed t-test (D and E) or two-way ANONA with a mixed-effects model with P values adjusted for multiple comparisons by Holm-Šídík method (F-N). The data presented represents one of two or three independent experiments. ns, not significant.

FIGS. 32A-32G show scRNA-seq reveals the remodeling of WT and Ythdf2-KO TME. (A) Gene expression heatmap for 8 assigned cell clusters from the scRNA-seq data, as in A. Each column group is one cell, and each row represents one gene. Five representative marker genes of each cluster are labeled. (B and C) A UMAP plot (B) and the proportions (C) from scRNA-seq showing 8 cell clusters of CD45⁺ tumor-infiltrating immune cells that were sorted from WT and Ythdf2-KO B16-OVA-bearing mice on day 14 after B16-OVA tumor inoculation. (D) Marker gene expression for each macrophages cluster, with dot color and size representing the averaged scaled expression value and percentage of marker gene expression, respectively. (E) Marker gene expression for each CD8⁺ T cluster, with dot color and size representing the averaged scaled expression value and percentage of marker gene expression, respectively. (F and G) Top 10 Gene Ontology clusters of up-regulated genes in NK cells (E) and cDCs (F) from scRNA-seq data.

FIGS. 33A-33L show Ythdf2 deficiency enhances the infiltration of anti-tumoral macrophages and CD8⁺ effector T cells and does not affect NK cells, MDSCs, and DCs in the tumor microenvironment. (A-C) Representative plots (A), percentage (B), and absolute number (C) of iNOS⁺F4/80⁺ TAMs from WT and Ythdf2-KO MC38 (1×10⁶) tumors after tumor inoculation (n=4 tumors per group). (D-G) Representative plots and percentage of Arg1⁺F4/80⁺ TAMs from WT and Ythdf2-KO B16-OVA (D and E) or MC38 (F and G) tumors after tumor inoculation (n=4 tumors per group). (H and I) Representative plots (H) and absolute number (I) of CD3⁺CD8⁺ T cells from WT and Ythdf2-KO MC38 (1×10⁶) tumors after tumor inoculation (n=4 tumors per group). (J-L) Percentage of NK cells (J), MDSCs (K), and DCs (L) from WT and Ythdf2-KO tumors after tumor inoculation (n=3-5 tumors per group). Data are represented as mean±s.d. Statistical analysis was performed using unpaired two-tailed t-test (B, C, E, G, I-L). The data presented represents one of two or three independent experiments. ns, not significant.

FIGS. 34A-34C show scRNA-seq identifies the expression of Cx3cr1 in CD45⁺ tumor-infiltrating immune cells from WT or Ythdf2-KO B16-OVA tumor cells. (A) Violin plot showing the distribution of mRNA expression for Cx3cr1 expression in different cell populations of the scRNA-seq data. (B) Migration cell number of BMDMs induced by WT or Ythdf2-KO MC38 tumor cell culture supernatants, measured with a transwell assay (n=3 per group). (C) Migration cell number of BMDMs induced by WT or Ythdf2-KO MC38 tumor cell culture supernatants with or without AZD8797, as measured with a transwell assay (n=3 per group). Data are represented as mean±s.d. Statistical analysis was performed using unpaired two-tailed t-test (B and C) or one-way ANONA model (H) with P values adjusted for multiple comparisons by Holm-Šídík method. The data presented represents one of two or three independent experiments.

FIGS. 35A-35E show YTHDF2 deficiency does not affect the phagocytosis of macrophage and Ythdf2-KO tumor supernatant alone does not polarize anti-tumoral macrophage. (A) Phagocytosis ratio of BMDMs. CellTrace™ Violet (CTV) prelabeled WT or Ythdf2-KO MC38 tumor cells were co-cultured with BMDMs for 4 hours. The percentage of macrophages that phagocytosed labeled tumor cells was measured by flow cytometry (n=3 samples per group). (B) Schematic representation of a macrophage polarization coculture assay involving BMDMs treated with WT or Ythdf2-KO tumor cell culture supernatant. (C and D) FACS analysis of iNOS (anti-tumoral macrophage marker) (C) or ARG1 (pro-tumoral macrophage marker) (D) expression in BMDMs cocultured with either WT or Ythdf2-KO tumor cell culture supernatants for 24 hours (n=3 samples per group). (E) The percentage of annexin V⁺ CD8⁺ T cells from WT or Ythdf2-KO B16-OVA tumor-bearing mice on day 14 after tumor inoculation (n=5 tumors per group). Data are represented as mean±s.d. Statistical analysis was performed using unpaired two-tailed t-test (A, C, D, and E). The data presented represents one of two or three independent experiments. ns, not significant.

FIGS. 36A-36E show Ythdf2-KO tumor cells dampen tumor glycolysis via repressing the expression of glycolytic transcripts. (A and B) Representative histograms showing expression levels of PD-L1 (A) and MHC-I (B) in B16-OVA tumor cells or MC38 tumor cells after treatment with IFN-γ for 24 hours. No treatment served as vehicle control. Median fluorescence intensity (MFI) is shown. (C) KEGG results showing the top 10 downregulated pathways enriched in Ythdf2-KO tumor cells compared to WT using RNA-seq data. The P values were calculated by a permutation test. (D) Heatmap of differential gene expression of glycolysis enzymes between WT and Ythdf2-KO groups. (E) Glycolytic stress tests using the Seahorse XF bioanalyzer to measure the glycolytic capacity of WT and Ythdf2-KO B16-OVA cells. Cells were seeded at 20,000/well in XF96 plate (n=5 samples per group).

FIGS. 37A-37H show DF-A7 inhibits the function of YTHDF2 via inducing YTHDF2 degradation. (A) Schematic of the YTHDF2 stability reporter. IRES, internal ribosome entry site. (B) Schematic of the small molecule reporter screen for YTHDF2 stability via flow cytometry. (C) Representative plots of mCherry⁺GFP⁺ Ythdf2-KO 293T cells transduced with full-length YTHDF2^{-/-} eGFP after treatment with different potential small molecules for 24 hours. (D) qPCR analysis of mRNA levels of Cx3cl1 after intrasample normalization to the levels of reference gene Actb in B16-OVA tumor cells after treatment with PBS or DF-A7 for 24 hours (n=3 samples per group). (E) qPCR analysis of mRNA levels of Cx3cl1 after intrasample normalization to the levels of reference gene Actb in MC38 tumor cells after treatment with PBS or DF-A7 for 24 hours (n=3 samples per group). (F) qPCR analysis of mRNA levels of PRR5 after intrasample normalization to the levels of reference gene Actb in HELA cells after treatment with PBS or DF-A7 for 24 hours (n=3 samples per group). (G) Body weight of C57BL/6 mice receiving PBS or DF-A7 treatments. Mice were s.c. inoculated with 5×10⁵ MC38 cells and injected i.p. with 2.5 mg/kg of DF-A7 on day 1 and 8 (n=5 mice per group). (H) HE staining of heart, liver, spleen, lung, and kidney from C57BL/6 mice receiving PBS or DF-A7 treatments. Mice were s.c. inoculated with 5×10⁵ MC38 cells and injected i.p. with 2.5 mg/kg of DF-A7 on days 1 and 8. Data are represented as mean±s.d. Statistical analysis was performed using unpaired two-tailed t-test (D, E, F) or two-way ANONA with a mixed-effects model followed by a Holm-Šídík post test (G). The data presented represents one of two or three independent experiments.

FIG. 38 is a list of primers used in Example 2.

FIG. 39 is a list of immunoblotting antibodies used in Example 2.

FIGS. 40A-40P: YTHDF2 deficiency enhances Th9-cell differentiation in mice. (A and B) Representative plots (A) and percentage (B) of WT (Ythdf2^{+/+}) or Ythdf2^{-/-} Th9 cells on the 5th day after differentiation (n=4). (C) Enzyme-linked immunosorbent assay (ELISA) analysis of the expression of secreted IL-9 protein from Ythdf2^{+/+} or Ythdf2^{-/-} Th9 cell culture supernatants (n=4). (D) qPCR analysis of mRNA expression of Il2, Il9, and Il21 in WT or Ythdf2^{-/-} Th9 cells after intrasample normalization to the reference gene 18S. (E and F) Representative plots (E) and percentage (F) of Ythdf2^{f/f} and Ythdf2^{cKO} Th9 cells on the 5th day after differentiation (n=7). (G) ELISA analysis of the expression of secreted IL-9 protein from Ythdf2^{f/f} and Ythdf2^{cKO} Th9 cell culture supernatants (n=7). (H) qPCR analysis of mRNA expression of Il2, Il9, and Il21 in Ythdf2^{f/f} and Ythdf2^{cKO} Th9 cells after intrasample normalization to the reference gene 18S. (I) Volcano plot showing the differentially expressed genes (DEGs) between Ythdf2^{f/f} and Ythdf2^{cKO} Th9 cells. Genes with a log 2 (fold change) of ≥2 or ≤−2 and P value <0.05 were considered as DEGs. FC, fold change. (J) GO clusters of up-regulated DEGs in RNA-seq data. (K to L) Bar plot of DEGs between Ythdf2^{f/f} and Ythdf2^{cKO} Th9 cells from RNA-seq grouped by cytokines and receptors (K), transcription factors (L), and inhibitory receptors (M). (N to P) GSEA results of the T cell activation involved in immune response gene (N), Natural killer cell activation gene (O), and T cell-mediated immune response to tumor cell gene (P) in mouse Ythdf2^{f/f} and Ythdf2^{cKO} Th9 cells from RNA-seq. NES, normalized enrichment score. Data are represented as means±SD. Statistical analysis was performed using an unpaired two-tailed t-test (B, C, D, F, G, and H). The data presented represent one of two or three independent experiments.

FIGS. 41A-41J: Loss of YTHDF2 promotes Th9 cell differentiation via transcription factor Gata3. (A) Representative plots showing the IL-9 expression by CD4⁺ T cells from Ythdf2^{f/f} and Ythdf2^{cKO} mice under stimulation with different cytokines or their combinations. (B and C) Representative plots (B) and summary graphs (C) showing the IL-9 expression by CD4⁺ T cells from Ythdf2^{f/f} and Ythdf2^{cKO} mice under stimulation with different doses of anti-mouse IL-4 antibody. (D) Venny plot showing an overlapping analysis of genes identified by RNA-seq (up-regulated genes), m⁶A-seq, and RIP-seq. (E and F) Representative plots (E) and summary graphs (F) of IL-9 expression in Gata3 KO Th9 cells from Ythdf2^{f/f} and Ythdf2^{cKO} mice. (G) Distribution of m⁶A peaks and YTHDF2-binding peaks across Gata3 by Integrative Genomics Viewer. (H and I) IP using either an antibody to m⁶A (H) or YTHDF2 (I) followed by qPCR in Th9 cells revealed that the Gata3 site in the 3′UTR region was m⁶A-methylated and enriched in YTHDF2 binding. Rabbit IgG served as a control. Enrichment of the indicated genes was normalized based on the input (n=3). (J) The mRNA half-life (t_{1/2}) of Gata3 transcript in Ythdf2^{f/f} and Ythdf2^{cKO} Th9 cells. The analysis involved fitting a linear regression model using log(y) versus time, followed by the transfer back to the nonlinear regression model through an exponential function. y=exp^{-0.8262t} (R²=0.9594) for Ythdf2^{f/f} Th9 cells and y=exp^{-0.5207t} (R²=0.9323) for Ythdf2^{cKO} Th9 cells. Seven slopes (decay rate) were compared within the full linear regression model using a t-test (n=7). Data are represented as means±SD. Statistical analysis was performed using an unpaired two-tailed t-test (H and I), paired two-tailed t-test (C), or one-way ANOVA models with P values adjusted for multiple comparisons by Holm-Šídík method (F). The data presented represent one of two or three independent experiments.

FIGS. 42A-42D: Ythdf2-deficient Th9 cells show higher anti-tumor efficacy in vivo. (A to C) Tumor growth in C57BL/6 mice that were s.c. inoculated with B16-OVA (A), LLC1-OVA (B), and E0771-OVA (C) tumor cells and then adoptively transferred with OT-II Ythdf2^{f/f} and Ythdf2^{cKO} Th9 cells (n=4 or 5). (D) Tumor growth in C57BL/6 mice that were s.c. inoculated with B16-OVA melanoma cells and received adoptive transfers of either OT-II Ythdf2^{f/f} or Ythdf2^{cKO} Th9 cells and then i.p. injected with anti-IL-9 or anti-IgG antibody every 3 d starting from day 0 (n=4). Data are represented as means±SD. Statistical analysis was performed using two-way ANOVA with a mixed-effects model with P values adjusted for multiple comparisons by Holm-Šídík method (A to D). **P<0.01, ***P<0.001, and ****P<0.0001.

FIGS. 43A-43O: Ythdf2-deficient Th9 cells enhance the infiltration of DCs, cytotoxic CD8⁺ T cells, and NK cells. (A to C) Flow cytometric analysis of tumor-infiltrating DCs in B16-OVA tumor-bearing mice that received adoptively transferred of either Ythdf2^{f/f} or Ythdf2^{cKO} Th9 cells. Data are presented as representative plots (A), summary graphs (B), and the absolute number of per gram tumors (C) (n=4). (D to F) Flow cytometric analysis of tumor-infiltrating CD8⁺ T cells. Data are presented as representative plots (D), summary graphs (E), and the absolute number of per gram tumors (F) (n=4). (G to I) Flow cytometric analysis of tumor-infiltrating IFN-γ⁺CD8⁺ T cells. Data are presented as representative plots (G), summary graphs (H), and the absolute number of per gram tumors (I) (n=4). (J to L) Flow cytometric analysis of tumor-infiltrating NK cells. Data are presented as representative plots (J), summary graphs (K), and the absolute number of per gram tumors (L) (n=4). (M to O) Flow cytometric analysis of tumor-infiltrating IFN-γ⁺ NK cells. Data are presented as representative plots (M), summary graphs (N), and the absolute number of per gram tumors (O) (n=4). Data are represented as means±SD. Statistical analysis was performed using unpaired two-tailed t-tests (B, C, E, F, H, I, K, L, N, and O).

FIGS. 44A-44G: Ythdf2-deficient Th9 cells show higher anti-tumor efficacy depending on both innate and adaptive immune responses. (A and B) Tumor growth in Rag1^{-/-} mice that were s.c. inoculated with B16-OVA (A) and E0771-OVA (B) tumor cells and then adoptively transferred with OT-II Ythdf2^{f/f} and Ythdf2^{cKO} Th9 cells (n=4 or 5). (C) The tumor growth in Rag1^{-/-} mice that were s.c. inoculated with B16F10 melanoma cells and received adoptive transfers of either Ythdf2^{f/f} Th9 cells or Ythdf2^{cKO} Th9 cells and then i.p. injected with anti-NK1.1 or anti-IgG antibody every 7 d starting from day 0 (n=4). (D and E) Representative plots (D) and summary graphs (E) showing the IFN-γ production by NK cells when coculture with B16F10 tumor cells in the presence of either Ythdf2^{f/f} or Ythdf2^{cKO} Th9 cells (n=3). (F) Batf3^{+/+} and Batf3^{-/-} C57BL/6 mice were subcutaneously injected with B16-OVA cells, and then adoptively transferred with OT-II Ythdf2^{f/f} and Ythdf2^{cKO} Th9 cells. Tumor sizes were measured starting from the sixth day after tumor inoculation (n=5). (G) The tumor growth in C57BL/6 mice that were s.c. inoculated with B16-OVA melanoma cells and received adoptive transfers of either OT-II Ythdf2^{f/f} Th9 cells or Ythdf2^{cKO} Th9 cells and then i.p. injected with anti-CD8 or anti-IgG antibody every 7 d starting from day 0 (n=4). Data are represented as means±SD. Statistical analysis was performed using one-way ANOVA models with P values adjusted for multiple comparisons by Holm-Šídík method (E), or two-way ANOVA with a mixed-effects model with P values adjusted for multiple comparisons by Holm-Šídík method (A to C, F, and G). **P<0.01, ***P<0.001, and ****P<0.0001. The data presented represent one of two or three independent experiments.

FIGS. 45A-45L: FIG. 6. YTHDF2 deficiency promotes the differentiation of human Th9 cells. (A and B) Representative plots (A) and percentage (B) of YTHDF2^{+/+} or YTHDF2^{-/-} Th9 cells after differentiation (n=7). (C) ELISA analysis of the expression of secreted IL-9 protein from YTHDF2^{+/+} or YTHDF2^{-/-} Th9 cell culture supernatants (n=7). (D) qPCR analysis of mRNA expression of IL2, IL9, and IL21 in YTHDF2^{+/+} or YTHDF2^{-/-} Th9 cells after intrasample normalization to the reference gene 18S. (E) Volcano plot showing the DEGs between human YTHDF2^{+/+} or YTHDF2^{-/-} Th9 cells. Genes with a log 2 (fold change) of ≥0.2 or ≤−0.2 and adjusted P value <0.05 were considered as DEGs. (F) GO clusters of up-regulated DEGs in RNA-seq data. (G to I) Bar plot of differentially expressed genes between human YTHDF2^{+/+} or YTHDF2^{-/-} Th9 cells from RNA-seq grouped by cytokines and receptors (G), transcription factors (H), and inhibitory receptors (I). (J to L) GSEA results of the adaptive immune response gene (J), positive regulation of innate immune response gene (K), and immune effector process gene (L) in human YTHDF2^{+/+} or YTHDF2^{-/-} Th9 cells from RNA-seq. NES, normalized enrichment score. Data are represented as means±SD. Statistical analysis was performed using paired two-tailed t-test (B and C) or unpaired two-tailed t-test (D). The data presented represent one of two or three independent experiments.

FIGS. 46A-46J: YTHDF2-deficient CAR Th9 cells exhibit enhanced antitumor activity against solid tumors in vivo. (A) Schematic of primary tumor growth assay involving PDAC cancer cells. In total, 3×10⁶ Capan-1 tumor cells plus 1×10⁶ PBMCs in 50 μl of PBS plus 50 μl of Matrigel were s.c. injected into the right flank of immunodeficient NSG-SGM3 mice, and then adoptively transferred with anti-PSCA YTHDF2^{+/+} or YTHDF2^{-/-} CAR-Th9 cells. (B) Tumor growth of established tumor cells, as described in A. Tumor volumes were measured every 4 days. (C) Schematic of primary tumor growth assay involving NSCLC cancer cells. In total, 3×10⁶ A549 tumor cells plus 1×10⁶ PBMCs in 50 μl of PBS plus 50 μl of Matrigel were s.c. injected into the right flank of immunodeficient NSG-SGM3 mice, and then adoptively transferred with anti-EGFR YTHDF2^{+/+} or YTHDF2^{-/-} CAR-Th9 cells. (D) Tumor growth of established tumor cells, as described in C. Tumor volumes were measured every 4 days. (E) Schematic of primary tumor growth assay involving PDAC cancer cells in humanized mice. In total, 10×10⁶ PBMCs in 200 μl PBS were i.v. injected into the NSG-SGM3 mice, and then 3×10⁶ tumor cells in 50 μl of PBS plus 50 μl of Matrigel were s.c. injected into the right flank of established humanized mice. After 13 days of tumor injection, tumor-bearing mice were adoptively transferred with anti-PSCA YTHDF2^{+/+} or YTHDF2^{-/-} CAR-Th9 cells. (F) Tumor growth of established tumor cells, as described in E. Tumor volumes were measured every 4 days, as above. (G and H) Flow cytometric analysis of tumor-infiltrating anti-EGFR CAR-Th9 cells (A) or anti-PSCA CAR-Th9 cells (B), as indicated in A549 or Capan-1 tumor-bearing mice that received adoptively transferred either YTHDF2^{+/+} or YTHDF2^{-/-} CAR Th9 cells. Data are presented as representative plots (left) and summary graphs (right) (n=5). (I and J) Flow cytometric analysis of tumor-infiltrating IFN-γ⁺CD8⁺ T cells, as indicated in A549 or Capan-1 tumor-bearing mice that received adoptively transferred either YTHDF2^{+/+} or YTHDF2^{-/-} CAR Th9 cells. Data are presented as representative plots (left) and summary graphs (right) (n=5). Data are represented as means±SD. Statistical analysis was performed using the unpaired two-tailed t-test (G, H, I, and J), and two-way ANOVA with a mixed-effects model with P values adjusted for multiple comparisons by Holm-Šídík method (B, D, and F). *P<0.05, **P<0.01, and ****P<0.0001.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The term “tumor-associated macrophage” or “TAM” are used in accordance with their plain and ordinary meaning and refer to a type of cell belonging to the macrophage lineage that are found in close proximity or within tumor masses or tumor microenvironments. TAMs may be derived from circulating monocytes or resident tissue macrophages, which form the major leukocytic infiltrate found within the stroma of many tumor types. TAMs may be involved in pro-tumor (e.g. promotion of growth and metastasis through tumor angiogenesis) processes. TAMs may interact with a wide range of growth factors, cytokines and chemokines in the tumor microenvironment which is thought to educate the TAMs and determine their specific phenotype and functional role as the microenvironment varies between different types of tumors. In many tumor types, TAM infiltration level has been shown to be of significant prognostic value.

The term “tumor-associated macrophage targeting agent” or “TAM targeting agent” refers to a compound that has high affinity and specificity for a TAM receptor. Exemplary TAM targeting agents include small molecules, antibodies, aptamers, and nucleic acids.

The term “tumor-associated macrophage receptor” or “TAM receptor” refers to a receptor (protein) that is embedded in the plasma membrane of a tumor-associated macrophage. The TAM receptor can be located in the extracellular domain of a TAM, the transmembrane domain of a TAM, or the intracellular domain of a TAM. The TAM receptor can be an ion channel-linked receptor, an enzyme-linked receptor, or a G protein-coupled receptor. In embodiments, the TAM receptor is CD163, CD206, CD14, CD16, CD32, CD64, CD68, CD71, CCR5, CCR2, or TLR9.

The term “YTHDF2” refers to the gene or variants thereof that code for the YTHDF2 protein. In embodiments, the YTHDF2 variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to the YTHDF2 sequence. In embodiments, the YTHDF2 gene is substantially identical to the nucleic acid sequence identified by NCBI Entrez Gene No. 51441 or a variant or homolog having substantial identity thereto. In embodiments, the YTHDF2 protein is substantially identical to the amino acid sequence identified by UniProt No. Q9Y5A9 or a variant or homolog having substantial identity thereto.

The term “YTHDF2^{-/-} ablated” naïve CD4+ T cell or “YTHDF2^{-/-} ablated” Th 9 cell refers to a naïve CD4⁺ T cell or Th9 cell that does not express a YTHDF2 protein. A naïve CD4⁺ T cell or Th9 cell that “does not express a YTHDF2 protein” refers to a naïve CD4⁺ T cell or Th9 cell that expresses an undetectable amount of YTHDF2 protein and/or that expresses a nonfunctional YTHDF2 protein. Methods for measuring proteins, such as YTHDF2, in vivo and in vitro are well-known in the art. It is also well-known that analytical methods for measuring proteins generally have a detectable limit, i.e., the lowest concentration or amount of a protein that can be reliably detected with a given analytical method. In embodiments, “an undetectable amount of YTHDF2 protein” is a concentration or amount of the YTHDF2 protein that is below a detectable limit. In embodiments, the naïve CD4⁺ T cell or Th9 cell can express a nonfunctional YTHDF2 protein, meaning that the YTHDF2 protein is inactive in vitro or in vivo.

The phrase “modifying expression of a YTHDF2 protein” to produce a naïve CD4⁺ T cell that does not express a YTHDF2 protein refers to modifications to the YTHDF2 gene and modification to the YTHDF2 protein (such as post-translational modifications) that would result in a non-functional YTHDF2 protein. In embodiments, “modifying expression of a YTHDF2 protein in a naïve CD4⁺ cell” refers to “ablating a YTHDF2 gene in a naïve CD4⁺ cell.” “Ablating” refers to removing the gene or altering the expression of the gene, e.g., for the gene to produce a non-functional protein.

The terms “inhibition,” “inhibit,” “inhibiting,” and the like in reference to a protein-inhibitor interaction, nucleic acid-inhibitor interaction, and/or T cell expression of a protein means negatively affecting (e.g. decreasing) the activity or function of the protein, nucleic acid, or T cell relative to the activity or function of the protein, nucleic acid, or T cell in the absence of the inhibitor. In embodiments, inhibition means negatively affecting (e.g. decreasing) the concentration or levels of the protein or nucleic acid relative to the concentration or level of the protein or nucleic acid in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein, nucleic acid, or T cell. In embodiments, inhibition refers to a reduction of activity of a target protein or nucleic acid resulting from a direct interaction (e.g. an inhibitor binds to the target protein). In embodiments, inhibition refers to a reduction of activity of a target protein or nucleic acid from an indirect interaction (e.g. an inhibitor binds to a protein that activates the target protein, thereby preventing target protein activation). In embodiments, inhibition includes inactivating, desensitizing, or down-regulating signal transduction or enzymatic activity or the amount of a protein produced by a T cell.

The terms “inhibitor” or “antagonist” or “downregulator” interchangeably refer to a substance capable of detectably decreasing the expression or activity of a given gene or protein. The inhibitor can decrease expression or activity 10%, 20%, 30%, 40%, 50%, 60%, 70%, 80%, 90% or more in comparison to a control in the absence of the inhibitor. In embodiments, expression or activity is statistically significantly lower than the expression or activity in the absence of the inhibitor.

A “YTHDF2 inhibitor” is an inhibitor of YTHDF2 expression or activity. As such, a YTHDF2 inhibitor is capable of detectably decreasing the expression or activity of a YTHDF2 gene or a YTHDF2 protein. In embodiments, the expression level of the a YTHDF2 gene or a YTHDF2 protein is decreased by about 10%, 20%, 30%, 40%, 50%, 60%, 70%, 80%, 90%, 95%, 96%, 97%, 98% or 99% or lower relative to the expression level of a YTHDF2 gene or a YTHDF2 protein in comparison to a control in the absence of the YTHDF2 inhibitor. In embodiments, the expression level of the a YTHDF2 gene or a YTHDF2 protein is decreased statistically significantly lower than the expression level of a YTHDF2 gene or a YTHDF2 protein in comparison to a control in the absence of the YTHDF2 inhibitor.

The term “DF-A7” refers to a compound of Formula (II):

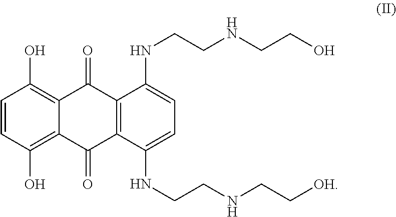 [SMILES: O=C1c2c(O)ccc(O)c2C(=O)c2c(NCCNCCO)ccc(NCCNCCO)c21]

In embodiments, DF-A7 is in the form of a pharmaceutically acceptable salt. In embodiments, DF-A7 is in the form of a dihydrochloride salt. In embodiments, DF-A7 is in the freebase form.

The term “contacting” may include allowing two species to react, interact, or physically touch, wherein the two species may be a YTHDF2 inhibitor as described herein and a gene, nucleic acid, or protein. In embodiments, contacting includes allowing a YTHDF2 inhibitor described herein to interact with a gene, nucleic acid, or protein that is involved in a signaling pathway or that is present on a TAM or encoded by a TAM.

The term “TLR9” or “TLR9 protein,” also known as cluster of differentiation 289 (CD289), includes any of the recombinant or naturally-occurring forms of the toll-like receptor 9 (TLR9) or variants or homologs thereof that maintain TLR9 protein activity (e.g. within at least 80%, 90%, 95%, or 100% activity compared to TLR9). In embodiments, the variants or homologs have at least 90%, 95%, 96%, 97%, 98%, 99% or 100% amino acid sequence identity across the whole sequence or a portion of the sequence (e.g. a 50, 100, 500, 750, 1000, or more continuous amino acid portion) compared to a naturally occurring TLR9 protein. In embodiments, the variants or homologs have about 90%, 95%, or 100% amino acid sequence identity across the whole sequence or a portion of the sequence compared to a naturally occurring TLR9 protein. In embodiments, TLR9 gene is identified by NCBI Gene ID: 54106, homolog or functional fragment thereof. In embodiments, TLR9 protein as identified by UniProt Ref: Q9NR96, homolog or functional fragment thereof.

The term “small molecule” or “small molecule compound” is used in accordance with its well understood meaning and refers to a low molecular weight organic compound that regulates a biological process by functioning as a therapeutic agent. In embodiments, the small molecule is a compound that weighs less than 1,000 daltons. In embodiments, a small molecule is not a protein or a nucleic acid.

A “therapeutic agent” as used herein refer to an agent (e.g., small molecule, aptamer, biologic (e.g., protein, nucleic acid, antibody), pharmaceutical composition) that when administered to a subject will have the intended prophylactic effect, e.g., treating or reducing the progression of a disease (e.g., cancer), or their symptoms or the intended therapeutic effect, e.g., treatment or amelioration of a disease (e.g., cancer), or their symptoms including any objective or subjective parameter of treatment such as abatement; remission; diminishing of symptoms; slowing in the rate of degeneration or decline; making the final point of degeneration less debilitating; or improving a patient's physical or mental well-being.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as, 2′O-methyl, 5′Fluoro, 2′-deoxy-2′fluoro, 2′-deoxy, a universal base nucleotide, a 5-C methyl nucleotide, an inverted deoxybasic residue incorporation, 5-methyl cytidine, or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art). Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

“Unmodified nucleotide” refers to a nucleotide that is not modified from its natural state.

“Modified nucleotide” refers to a nucleotide that is modified from its natural state. The modification to the nucleotide can be to the base, the sugar, the phosphate, or two or more thereof. Nucleotides can be modified, for example, to include 2′-O-aminopropyl group, a 2′-O-ethyl group, a 2′-fluoro group, a 2′-O-methyl group, 2′-deoxy-2′fluoro group, a 2′-O-methoxyethyl group, a 2′-O-allyl group, a 2′-O-propyl group, a 2′-O-pentyl group, and a locked nucleic acid (LNA) modification.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and anomeric sugar-phosphate, backbone-modified nucleotides. In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art. Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

A “siRNA” or “small interfering RNA” refers to a nucleic acid that forms a double stranded RNA, which double stranded RNA has the ability to reduce or inhibit expression of a gene or target gene when present in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular rnRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. The complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein.

The term “recombinant” when used with reference, e.g., to a cell, nucleic acid, protein, or vector, indicates that the cell, nucleic acid, protein or vector, has been modified by: (i) the introduction of a heterologous nucleic acid or protein or (ii) the alteration or deletion of a native nucleic acid or protein, or that the cell is derived from a cell so modified. Thus, for example, recombinant cells: (a) express genes that are not found within the native (non-recombinant) form of the cell, (b) express native genes that are otherwise abnormally expressed, under expressed or not expressed at all, and/or (c) do not express genes that are found within the native (non-recombinant) form of the cell. Transgenic cells are those that express a heterologous gene or coding sequence, typically as a result of recombinant methods.

The terms “isolate” or “isolated”, when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms or by manual alignment and visual inspection (see, e.g., www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The term “F(ab)′₂” is used interchangeably with “Fab dimer.” The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). The term “Fab′ monomer” is used interchangeably with “Fab” and “or an antigen-binding fragment.” While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (e.g., McCafferty et al., Nature 348:552-554 (1990)).

Antibodies are large, complex proteins with an intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat et al, Sequences of Proteins of Immunological Interest, U.S. Department of Health and Human Services, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” and one “heavy” chain. The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

An “antibody variant” as provided herein refers to a polypeptide capable of binding to a receptor protein or an antigen and including one or more structural domains of an antibody or fragment thereof. Non-limiting examples of antibody variants include single-domain antibodies (nanobodies), affibodies (polypeptides smaller than monoclonal antibodies and capable of binding receptor proteins or antigens with high affinity and imitating monoclonal antibodies), antigen-binding fragments (Fab), Fab dimers (monospecific Fab₂, bispecific Fab₂), trispecific Fab₃, monovalent IgGs, single-chain variable fragments (scFv), bispecific diabodies, trispecific triabodies, scFv-Fc, minibodies, IgNAR, V-NAR, hcIgG, VhH, and peptibodies. A “peptibody” as provided herein refers to a peptide moiety attached (through a covalent or non-covalent linker) to the Fc domain of an antibody.

A “single-domain antibody” or “nanobody” refers to an antibody fragment having a single monomeric variable antibody domain. Like a whole antibody, it is able to bind selectively to a specific antigen. In embodiments, the single domain antibody is a human or humanized single-domain antibody.

A single-chain variable fragment (scFv) is typically a fusion protein of the variable regions of the heavy (VH) and light chains (VL) of immunoglobulins, connected with a short linker peptide of 10 to about 25 amino acids. The linker is usually rich in glycine for flexibility, as well as serine or threonine for solubility. The linker can either connect the N-terminus of the VH with the C-terminus of the VL, or vice versa.

Antibodies, e.g., recombinant, monoclonal, or polyclonal antibodies, can be prepared by techniques well known in the art (e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies can be adapted to produce antibodies to polypeptides. Also, transgenic mice, or other organisms such as other mammals, may be used to express humanized or human antibodies. Alternatively, phage display technology can be used to identify antibodies and heteromeric Fab fragments that specifically bind to selected antigens (e.g., McCafferty et al., Nature 348:552-554 (1990); Marks et al., Biotechnology 10:779-783 (1992)). Antibodies can also be made bispecific, i.e., able to recognize two different antigens (e.g., WO 93/08829, Traunecker et al., EMBO J. 10:3655-3659 (1991); Suresh et al., Methods in Enzymology 121:210 (1986)). Antibodies can also be heteroconjugates, e.g., two covalently joined antibodies, or immunotoxins (e.g., U.S. Pat. No. 4,676,980, WO 91/00360, WO 92/200373).

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “aptamer” refers to oligonucleotides (e.g. short oligonucleotides or deoxyribonucleotides), that bind (e.g. with high affinity and specificity) to proteins, peptides, and small molecules. An aptamer may be referred to as an oligonucleotide based target binding moiety. Aptamers may be RNA or DNA. Aptamers may have secondary or tertiary structure and, thus, may be able to fold into diverse and intricate molecular structures. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold Let al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for a protein may be enriched and identified. Aptamers may exhibit many desirable properties for targeted drug delivery, such as ease of selection and synthesis, high binding affinity and specificity, low immunogenicity, and versatile synthetic accessibility. Anticancer agents (e.g. chemotherapy drugs, toxins, and siRNAs) may be successfully delivered to cancer cells in vitro using aptamers.

The term “CpG oligodeoxynucleotide” or “CpG ODN” refers to a 5′ C nucleotide connected to a 3′ G nucleotide through a phosphodiester internucleotide linkage or a phosphodiester derivative internucleotide linkage. In embodiments, a CpG ODN includes a phosphodiester internucleotide linkage. In embodiments, a CpG ODN includes a phosphodiester derivative internucleotide linkage.

The term “Class A CpG ODN” or “A-class CpG ODN” or “D-type CpG ODN” or “Class A CpG DNA sequence” refers to a CpG motif including oligodeoxynucleotide including one or more of poly-G sequence at the 5′, 3′, or both ends; an internal palindrome sequence including CpG motif, or one or more phosphodiester derivatives linking deoxynucleotides. In embodiments, a Class A CpG ODN includes poly-G sequence at the 5′, 3′, or both ends; an internal palindrome sequence including CpG motif, and one or more phosphodiester derivatives linking deoxynucleotides. In embodiments, the phosphodiester derivative is phosphorothioate Examples of Class A CpG ODNs include ODN D19, ODN 1585, ODN 2216, and ODN 2336, the sequences of which are known in the art.

The term “Class B CpG ODN” or “B-class CpG ODN” or “K-type CpG ODN” or “Class B CpG DNA sequence” refers to a CpG motif including oligodeoxynucleotide including one or more of a 6mer motif including a CpG motif, phosphodiester derivatives linking all deoxynucleotides. In embodiments, a Class B CpG ODN includes one or more copies of a 6mer motif including a CpG motif and phosphodiester derivatives linking all deoxynucleotides. In embodiments, the phosphodiester derivative is phosphorothioate. In embodiments, a Class B CpG ODN includes one 6mer motif including a CpG motif. In embodiments, a Class B CpG ODN includes two copies of a 6mer motif including a CpG motif. In embodiments, a Class B CpG ODN includes three copies of a 6mer motif including a CpG motif. In embodiments, a Class B CpG ODN includes four copies of a 6mer motif including a CpG motif. Examples of Class B CpG ODNs include ODN 1668, ODN 1826, ODN 2006, ODN 2007, ODN BW006, and ODN D-SL01, the sequences of which are known in the art.

The term “Class C CpG ODN” or “C-class CpG ODN” “or “C-type CpG DNA sequence” refers to an oligodeoxynucleotide including a palindrome sequence including a CpG motif and phosphodiester derivatives (phosphorothioate) linking all deoxynucleotides. Examples of Class C CpG ODNs include ODN 2395, ODN M362, and ODN D-SLO3, the sequences of which are known in the art.

The term “attached” when referring to two moieties (e.g., a TAM targeting agent attached to a YTHDF2 inhibitor) means the two moieties are bonded, wherein the bond or bonds connecting the two moieties are covalent or non-covalent. In embodiments, the two moieties are covalently bonded to each other (e.g. directly or through a linking group). Exemplary linking groups include a covalent bond, a nucleic acid sequence (e.g., a DNA sequence), substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or combinations of two or more thereof.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable non-cyclic straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) 0, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—OH, —CH₂—NH₂, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —O—CH₃, —CH═CH—N(CH₃)—CH₃, —O—CH₂—CH₃, and —CN. Up to two or three heteroatoms may be consecutive, such as, for example, —CH₂—NH—OCH₃ and —CH₂—O—Si(CH₃)₃.

The term “heteroalkylene,” by itself or as part of another substituent, means, unless otherwise stated, a divalent radical derived from heteroalkyl, as exemplified, but not limited by, —CH₂—CH₂—S—CH₂—CH₂—, —O—CH₂—CH₂—NH—CH₂—, —O—(CH₂)₃—O—PO₃—, —O—(CH₂)—O—PO₃—, —O—(CH₂)₂—O—PO₃—, —O—(CH₂)₄—O—PO₃—, and the like. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted as excluding specific heteroalkyl groups, such as —NR′R″ or the like.

The terms “cycloalkyl” and “heterocycloalkyl,” by themselves or in combination with other terms, mean, unless otherwise stated, cyclic non-aromatic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently (e.g., biphenyl). A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo” means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, □NR′NR″R″′, □ONR′R″, □NR′C═(O)NR″NR″′R″″, —CN, —NO₂, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example:—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, □NR′NR″R″′, □ONR′R″, □NR′C═(O)NR″NR″′R″″, —CN, —NO₂, —R′, —N₃, —CH(Ph)₂, fluoro(C₁-C₄)alkoxy, and fluoro(C1-C₄)alkyl, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R″′, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In embodiments, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties: (A) oxo, halogen, —CF₃, —CN, —OH, —NH₂, —COOH, —CONH₂, —NO₂, —SH, —SO₂Cl, —SO₃H, —SO₄H, —SO₂NH₂, □NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, □NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, heteroaryl, substituted with at least one substituent selected from: (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, heteroaryl, substituted with at least one substituent selected from: (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

As defined herein, the term “activation,” “activate,” “activating,” “activator” and the like in reference to a protein-inhibitor interaction means positively affecting (e.g. increasing) the activity or function of the protein relative to the activity or function of the protein in the absence of the activator. In aspects activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is higher than the expression or activity in the absence of the agonist.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means a range extending to +/−1 of the specified value. In embodiments, about includes the specified value.

“Control” or “control experiment” is used in accordance with its plain ordinary meaning and refers to an experiment in which the subjects or reagents of the experiment are treated as in a parallel experiment except for omission of a procedure, reagent, or variable of the experiment. In embodiments, the control is used as a standard of comparison in evaluating experimental effects. In embodiments, a control is the measurement of the activity of a protein in the absence of a compound as described herein (including embodiments and examples). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The term “cancer” refers to all types of cancer, neoplasm or malignant tumors found in mammals (e.g. humans), including leukemias, lymphomas, carcinomas and sarcomas. the term “cancer” refers to all types of cancer, neoplasm or malignant tumors found in mammals, including leukemias, lymphomas, melanomas, neuroendocrine tumors, carcinomas and sarcomas. xemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or medulloblastoma, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's disease of the nipple, phyllodes tumors, lobular carcinoma, ductal carcinoma, cancer of the pancreatic stellate cells, cancer of the hepatic stellate cells, or prostate cancer. Other exemplary cancers that can be treated with the compounds and pharmaceutical compositions described herein include prostate cancer, lung cancer, colorectal cancer (inclusive of colon cancer and rectal cancer), kidney cancer, bladder cancer, thyroid cancer, endometrial cancer, glioma, glioblastoma, brain cancer, neuroblastoma, pancreatic cancer, medulloblastoma, melanoma, cholangiocarcinoma, cervical cancer, gastric cancer, ovarian cancer, testicular cancer, thymoma, uterine cancer, head and neck cancer, lymphoma (e.g., B-cell lymphoma), brain cancer, and ovarian cancer.

The term “solid tumor” refers to an abnormal mass of tissue that usually does not contain cysts or liquid areas. Examples of solid tumors are sarcomas and carcinomas.

The term “hot tumor” or “inflamed tumor” refers to a tumor wherein there is a considerable presence of anti-tumor immune cells especially tumor infiltrating lymphocytes (e.g., T cells, CD8⁺ T cells), increased interferon-7 (IFN-γ) signaling, expression of PD-L1, and high tumor mutational burden, and thus hot tumors are typically immunostimulatory.

The term “cold tumor” or “non-inflamed tumor” refers to a tumor wherein there is no or minimal presence of anti-tumor immune cells especially tumor infiltrating lymphocytes or instead containing cell subsets within the tumor or tumor microenvironment associated with immune suppression including regulatory T cells (Treg), myeloid-derived suppressor cells (MDSCs) and M2 macrophages. A cold tumor may be characterized by a low number or even absence of infiltration of anti-tumor immune cells that such cells may be present but remain stuck in the surrounding stroma, thus unable to colonize the tumor microenvironment to provide their antitumor functions. In addition, cold tumors are characterized by low mutational load, low major histocompatibility complex (MHC) class I expression, and low PD-L1 expression.

The term “tumor microenvironment (TME)” refers to the environment within an organism (e.g., human) in which a tumor exists, including tumor cells themselves, the surrounding stromal cells and non-cellular components, such as cytokines, chemokines, collagen, elastin, and growth factors. The stromal cells include fibroblasts, epithelial cells, vascular cells, resident and/or recruited inflammatory and immune cells (e.g., macrophages, dendritic cells, granulocytes, lymphocytes, etc.) that modulate tumor cell growth or survival. In early or middle stage of tumor, some tumor cells acquire mutations that allow them to resist immune destruction, but their proliferation and spread are still restricted by immune responses. Major anti-tumor components include natural killer (NK) cells, cytolytic T lymphocytes (CTLs), CD4⁺ helper T cells, M1 macrophages and two major cytokines: interleukin-12 (IL-12) and interferon-T (IFN-γ). However, advanced tumor cells often changes the tumor microenvironment, shifting from immuno-responsive to immunosuppressive, allowing tumor cells to evade from host immunosurveillance and supporting tumor growth, progression and spread. Specifically, in the immunosuppressive tumor microenvironment, although some immune cells, such as cytotoxic CTLs or helper T cells, may still exist, their function is largely inhibited; IL-12 production is also greatly suppressed; and cell subsets associated with immune suppression such as regulatory T cells (Tregs), myeloid derived suppressor cells (MDSC) and M2 macrophages are recruited to the tumor site, leading to inhibition of immune activity; on the other hand, tumor cells produce cytokines, such as tumor necrotic factor-α (TNFα) and cyclooxygenase-2 (Cox-2), which promote chronic inflammation and vascular endothelial growth factor (VEGF) that promotes angiogenesis, both leading to significant tumor growth.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “tumor infiltrating lymphocytes” or “TILs” are populations of immune cells that are associated with tumor tissue. More particularly, TILs are lymphocytes of a subject afflicted with a cancer that have left the blood stream and have associated with a tumor. Therefore, TILs may have tumor specificity and activating TILs may allow for more direct control of the elimination of tumor cells. The main types of TILs include T cells, B cells and natural killer (NK) cells. Cytotoxicity T lymphocytes (CTLs), particularly CD8⁺ cells, can directly attack and kill tumor cells. T helper lymphocytes (Th), particularly CD4⁺ cells, are capable of secreting various cytokines that can activate CTLs. TILs also include myeloid cells such as dendritic cells, particularly CD86⁺ dendritic cells. The presence of TILs, particularly CD8⁺ cells, in tumors is known to be positively associated with good response to immunotherapy particularly using immune checkpoint modulator(s), advantageous in changing the tumor microenvironment from immunosuppressive to immuno-stimulatory, transforming a cold tumor into a hot tumor, and leads to better clinic outcomes. Intratumoral TILs may be defined as lymphocytes inside tumor nests having cell-to-cell contact with no intervening stroma and directly interacting with tumor cells, while stromal TILs may be defined as lymphocytes located dispersed in the stroma between the tumor cells and do not directly contact tumor cells. TILs can be assessed, identified, accounted and/or phenotyped by immunohistochemistry analysis and flow cytometric analysis with antibody labeling.

The term “CD4” refers to cluster of differentiation 4, well-known in the art. The term “CD4” as provided herein includes any of the naturally occurring forms, homologs or variants that maintain the activity of CD4 (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the CD4 protein is the protein as identified by the UniProt accession no. 901730 or NCBI sequence reference NP_000607.1 GI:10835167, homolog or functional fragment thereof. CD4 is a glycoprotein found on the surface of immune cells such as T-helper cells, monocytes, macrophages and dendritic cells.

The term “CD8” refers to cluster of differentiation 8, well-known in the art, and includes any one of the CD8a or CD8b chains. The term “CD8” as provided herein includes any of the naturally occurring forms, homologs or variants that maintain the activity of CD8 (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the CD8a protein is the protein as identified by the NCBI sequence reference NM_001768.6 GI:225007534, homolog or functional fragment thereof. In embodiments, the CD8b protein is the protein as identified by the NCBI sequence reference AAI00912.1 GI:71682667, homolog or functional fragment thereof. CD8 is a transmembrane glycoprotein co-receptor for the T-cell receptor.

The terms “treating” or “treatment” refers to any indicia of clinical success in the therapy or amelioration of a disease (e.g., cancer), including any objective or subjective parameter such as abatement; remission; diminishing of symptoms or making the disease more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination. The term “treating” does not include preventing.

“Patient” or “subject in need thereof” refers to a living organism suffering from or prone to a disease n that can be treated by administration of a compound or pharmaceutical composition herein. Non-limiting examples include humans, other mammals, bovines, rats, mice, dogs, cats, monkeys, goat, sheep, cows, and other non-mammalian animals. In embodiments, a patient is human.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

The term “immune checkpoint inhibitor” refers to a compound (e.g., an antibody) that is capable of binding to an inhibitory receptor or capable of interfering with the interaction between an inhibitory receptor and its ligand, wherein the inhibitory receptor is essential to balance co-stimulatory receptor activity and limit T-cell activation. Thus, immune checkpoint inhibitors target immune system checkpoints such as the PD-1 pathway.

“PD-1 pathway inhibitor” refers to a substance capable of detectably lowering expression of or activity level of the PD-1 signaling pathway compared to a control. An “inhibitor” is a compound or small molecule that inhibits the PD-1 signaling pathway e.g., by binding, partially or totally blocking stimulation of the PD-1 pathway, decrease, prevent, or delay activation of the PD-1 pathway, or inactivate, desensitize, or down-regulate signal transduction, gene expression or enzymatic activity of the PD-1 pathway. In embodiments, the PD-1 pathway inhibitor is a programmed death-ligand 1 (PD-L1) inhibitor or a PD-1 inhibitor. A PD-L1 inhibitor is a substance that, at least in part, partially or totally blocks stimulation, decreases, prevents, or delays activation, or inactivates, desensitizes, or down-regulates signal transduction of PD-1. A PD-1 inhibitor is a substance that, at least in part, partially or totally blocks stimulation, decreases, prevents, or delays activation, or inactivates, desensitizes, or down-regulates signal transduction of PD-1.

Provided herein are compounds comprising a TAM targeting agent attached to a YTHDF2 inhibitor. In embodiments, the TAM targeting agent is a ligand for a TAM receptor. In embodiments, the TAM targeting agent is a ligand for a TAM receptor, wherein the TAM receptor is TLR9, CD163, CD206, CD14, CD16, CD32, CD64, CD68, CD71, CCR5, or CCR2. In embodiments, the TAM targeting agent is a ligand for a TAM receptor, wherein the TAM receptor is TLR9. In embodiments, the TAM targeting agent is a ligand for a TAM receptor, wherein the TAM receptor is CD163. In embodiments, the TAM targeting agent is a ligand for a TAM receptor, wherein the TAM receptor is CD206. In embodiments, the TAM targeting agent is a ligand for a TAM receptor, wherein the TAM receptor is CD14. In embodiments, the TAM targeting agent is a ligand for a TAM receptor, wherein the TAM receptor is CD16. In embodiments, the TAM targeting agent is a ligand for a TAM receptor, wherein the TAM receptor is CD32. In embodiments, the TAM targeting agent is a ligand for a TAM receptor, wherein the TAM receptor is CD64. In embodiments, the TAM targeting agent is a ligand for a TAM receptor, wherein the TAM receptor is CD68. In embodiments, the TAM targeting agent is a ligand for a TAM receptor, wherein the TAM receptor is CD71. In embodiments, the TAM targeting agent is a ligand for a TAM receptor, wherein the TAM receptor is CCR5. In embodiments, the TAM targeting agent is a ligand for a TAM receptor, wherein the TAM receptor is CCR2.

In embodiments of the compounds described herein, the TAM targeting agent is a CpG oligodeoxynucleotide. In embodiments of the compounds described herein, the TAM targeting agent is a phosphorothioated CpG oligodeoxynucleotide. In embodiments, the CpG oligodeoxynucleotide is CpG ODN D19. In embodiments, the CpG oligodeoxynucleotide is CpG ODN 1585. In embodiments, the CpG oligodeoxynucleotide is CpG ODN 2216. In embodiments, the CpG oligodeoxynucleotide is CpG ODN 2336. In embodiments, the CpG oligodeoxynucleotide is CpG ODN 1668. In embodiments, the CpG oligodeoxynucleotide is CpG ODN 1826. In embodiments, the CpG oligodeoxynucleotide is CpG ODN 2006. In embodiments, the CpG oligodeoxynucleotide is CpG ODN 2007. In embodiments, the CpG oligodeoxynucleotide is CpG ODN BW006. In embodiments, the CpG oligodeoxynucleotide is CpG ODN D-SL01. In embodiments, the CpG oligodeoxynucleotide is CpG ODN 2395. In embodiments, the CpG oligodeoxynucleotide is CpG ODN M362. In embodiments, the CpG oligodeoxynucleotide is CpG ODN D-SL03. In embodiments, the CpG oligodeoxynucleotide comprises the sequence: 5′-T*C*C*A*T*G*A*C*G*T*T*C*C*T*G*A*T*G*C*T (SEQ ID NO:1),wherein an asterisk (*) refers to a phosphorothioate internucleotide linkage.

The ability of a TAM targeting agent to bind to a TAM receptor can be described by the equilibrium dissociation constant (K_{D}). The equilibrium dissociation constant (K_{D}) as defined herein is the ratio of the dissociation rate (K-off) and the association rate (K-on) of a TAM targeting agent to bind to a TAM receptor. It is described by the following formula: K_{D}=K-off/K-on. In embodiments, the TAM targeting agent is able to bind to a TAM receptor with an equilibrium dissociation constant (K_{D}) from about 0.5 to about 25 nM. In embodiments, the TAM targeting agent is able to bind to a TAM receptor with an equilibrium dissociation constant (K_{D}) from about 1 to about 25 nM. In embodiments, the TAM targeting agent is able to bind to a TAM receptor with an equilibrium dissociation constant (K_{D}) from about 1.5 to about 25 nM. In embodiments, the TAM targeting agent is able to bind to a TAM receptor with an equilibrium dissociation constant (K_{D}) from about 2 to about 25 nM. In embodiments, the TAM targeting agent is able to bind to a TAM receptor with an equilibrium dissociation constant (K_{D}) from about 2.5 to about 25 nM. In embodiments, the TAM targeting agent is able to bind to a TAM receptor with an equilibrium dissociation constant (K_{D}) from about 3 to about 25 nM. In embodiments, the TAM targeting agent is able to bind to a TAM receptor with an equilibrium dissociation constant (K_{D}) from about 3.5 to about 25 nM. In embodiments, the TAM targeting agent is able to bind to a TAM receptor with an equilibrium dissociation constant (K_{D}) from about 4 to about 25 nM.

In embodiments, the disclosure provides TAM targeting agent having a binding affinity (K_{D}) to a TAM receptor of at least 10⁻⁷ M. In embodiments, the disclosure provides a TAM targeting agent having a binding affinity (K_{D}) to a TAM receptor of at least 10⁻⁸ M. In embodiments, the disclosure provides a TAM targeting agent having a binding affinity (K_{D}) to a TAM receptor of at least 10⁻⁹ M. In embodiments, the disclosure provides a TAM targeting agent having a binding affinity (K_{D}) to a TAM receptor of at least 10⁻¹⁰ M. In embodiments, the disclosure provides a TAM targeting agent having a binding affinity (K_{D}) to a TAM receptor of at least 10⁻¹¹ M. In embodiments, the disclosure provides a TAM targeting agent having a binding affinity (K_{D}) to a TAM receptor from about 10⁻⁷ M to about 10⁻¹¹ M. In embodiments, the disclosure provides a TAM targeting agent having a binding affinity (K_{D}) to a TAM receptor from about 10⁻⁸ M to about 10⁻¹¹ M. In embodiments, the disclosure provides a TAM targeting agent having a binding affinity (K_{D}) to a TAM receptor from about 10⁻⁹ M to about 10⁻¹¹ M.

In embodiments of the compounds described herein, the YTHDF2 inhibitor inhibits mRNA expression or protein expression. In embodiments, the YTHDF2 inhibitor inhibits mRNA expression. In embodiments, the YTHDF2 inhibitor inhibits expression of a YTHDF2 protein. In embodiments, the YTHDF2 inhibitor is an antibody, a small molecule, an aptamer, or a nucleic acid. In embodiments, the YTHDF2 inhibitor is an antibody, a small molecule, an aptamer, a protein, or a nucleic acid. In embodiments, the YTHDF2 inhibitor is an antibody, a small molecule, an aptamer, a protein, an enzyme, or a nucleic acid. In embodiments, the YTHDF2 inhibitor is an antibody. In embodiments, the YTHDF2 inhibitor is a small molecule. In embodiments, the YTHDF2 inhibitor is an aptamer. In embodiments, the YTHDF2 inhibitor is a protein. In embodiments, the YTHDF2 inhibitor is an enzyme. In embodiments, the YTHDF2 inhibitor is a nucleic acid. In embodiments, the nucleic acid is DNA or RNA. In embodiments, the YTHDF2 inhibitor is an antisense inhibitor. In embodiments, the In embodiments, the antisense inhibitor is siRNA or shRNA. In embodiments, the antisense inhibitor is shRNA. In embodiments, the antisense inhibitor is siRNA. In embodiments, the siRNA comprises the sequence: 5′-A*U*ACAUAAACUGCAAGUUUGC*U*U (SEQ ID NO:2), wherein an asterisk (*) refers to a phosphorothioate internucleotide linkage.

In embodiments, the TAM targeting agent is a phosphorothioated CpG oligodeoxynucleotide attached through a covalent linking group to an antisense siRNA. In embodiments, the TAM targeting agent is a phosphorothioated CpG oligodeoxynucleotide covalently bonded to a sense strand RNA, wherein the sense strand RNA is hybridized to an antisense siRNA. In embodiments, the TAM targeting agent is a phosphorothioated CpG oligodeoxynucleotide comprising the nucleic acid having SEQ ID NO:1 attached through a covalent linking group to a sense strand RNA having the sequence: 5′-G*C*AAACUUGCAGUUUAUGUAU-3 (SEQ ID NO:3), wherein the sense strand RNA is hybridized to antisense siRNA having SEQ ID NO:2. In embodiments, the 3′ end of the phosphorothioated CpG oligodeoxynucleotide is attached to the 5′ end of the sense strand RNA. In embodiments, the 3′ end of the phosphorothioated CpG oligodeoxynucleotide is attached to the 3′ end of the sense strand RNA. In embodiments, the 5′ end of the phosphorothioated CpG oligodeoxynucleotide is attached to the 5′ end of the sense strand RNA. In embodiments, the 5′ end of the phosphorothioated CpG oligodeoxynucleotide is attached to the 3′ end of the sense strand RNA.

In embodiments of the compounds described herein, the TAM targeting agent is attached to the YTHDF2 inhibitor through a covalent linking group. In embodiments, the covalent linking group is a bond, a nucleic acid, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a combination of two or more thereof. In embodiments, the covalent linking group is a substituted or unsubstituted heteroalkylene. In embodiments, the covalent linking group is a substituted or unsubstituted 2 to 60 membered heteroalkylene. In embodiments, the covalent linking group is:

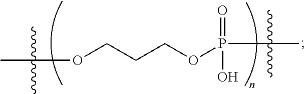

wherein n is an integer from 1 to 10. In embodiments, n is an integer from 2 to 8. In embodiments, n is an integer from 4 to 6. In embodiments, n is 3. In embodiments, n is 4. In embodiments, n is 5. In embodiments, n is 6. In embodiments, n is 7. In embodiments, n is 8.

Provided herein is a YTHDF2 inhibitor of Formula (I) or a pharmaceutically acceptable salt thereof:

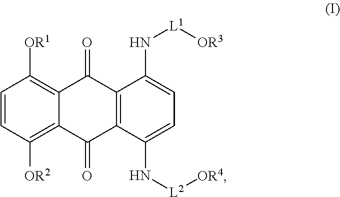

wherein R¹, R², R³, and R⁴ are each independently H or C₁₋₅ alkyl, and L¹ and L² are each independently a 2 to 8 membered heteroalkylene.

In embodiments of the compounds of Formula (I), R¹, R², R³, and R⁴ are each independently H or C₁₋₄ alkyl. In embodiments, R¹, R², R³, and R⁴ are each independently H, methyl, ethyl, or propyl. In embodiments, R¹, R², R³, and R⁴ are each independently H, methyl, or ethyl. In embodiments, R¹, R², R³, and R⁴ are each independently H or methyl. In embodiments, R¹, R², R³, and R⁴ are hydrogen.

In embodiments of the compounds of Formula (I), L¹ and L² are each independently a 2 to 8 membered heteroalkylene. In embodiments of the compounds of Formula (I), L¹ and L² are each independently a 3 to 7 membered heteroalkylene. In embodiments of the compounds of Formula (I), L¹ and L² are each independently a 4 to 6 membered heteroalkylene. In embodiments, L¹ and L² are each independently a 2 to 8 membered heteroalkylene containing one nitrogen atom.

In embodiments of the compounds of Formula (I), L¹ and L² are each independently a 3 to 7 membered heteroalkylene containing one nitrogen atom. In embodiments of the compounds of Formula (I), L¹ and L² are each independently a 4 to 6 membered heteroalkylene containing one nitrogen atom.

In embodiments of the compounds of Formula (I), L¹ and L² are each independently —(CH₂)ₙ₁—NH—(CH₂)ₙ₂—, wherein n1 and n2 are each independently an integer from 1 to 4. In embodiments n1 and n2 are each independently an integer from 1 to 3. In embodiments n1 and n2 are each independently an integer of 1 or 2. In embodiments n1 and n2 are each independently an integer from 2 or 3.

In embodiments, the compound of Formula (I) is a compound of Formula (II) or a pharmaceutically acceptable salt thereof:

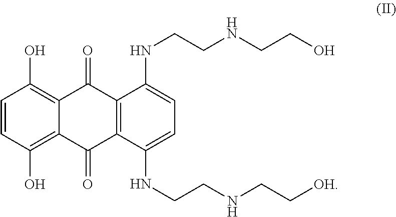 [SMILES: O=C1c2c(O)ccc(O)c2C(=O)c2c(NCCNCCO)ccc(NCCNCCO)c21]

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

### T Cells and Chimeric Antigen Receptors

Provided herein is a naïve CD4⁺ T cell, wherein the naïve CD4⁺ T cell does not express a YTHDF2 protein. In embodiments, the naïve CD4⁺ T cell does not express a YTHDF2 protein and comprises a chimeric antigen receptor. In embodiments, a naïve CD4⁺ T cell the does not express a YTHDF2 protein, and that optionally comprises a chimeric antigen receptor, is referred to as a recombinant naïve CD4⁺ T cell. Provided herein is a process of making a recombinant naïve CD4⁺ T cell comprising modifying expression of a YTHDF2 protein in naïve CD4⁺ T cells, thereby producing naïve CD4⁺ T cells that do not express a YTHDF2 protein. In embodiments, the process further comprises (iii) transducing the naïve CD4⁺ T cell that do not express a YTHDF2 protein with a chimeric antigen receptor, thereby producing naïve CD4⁺ T cells that do not express a YTHDF2 protein and that comprises a chimeric antigen receptor.

Provided herein is a T helper 9 cell, wherein the T helper 9 cell does not express a YTHDF2 protein. In embodiments, the T helper 9 cell does not express a YTHDF2 protein and comprises a chimeric antigen receptor. In embodiments, T helper cell refers to a plurality or population of T helper cells. In embodiments, a T helper cell the does not express a YTHDF2 protein, and that optionally comprises a chimeric antigen receptor, is referred to as a recombinant T helper cell. In embodiments, this recombinant T helper 9 cell secretes higher levels of effector cytokines, such as IL-2, IL-9, and IL-21, when compared to Th9 cells that contain the YTDF2 gene. In embodiments, this recombinant T helper 9 cell expresses higher levels of Gata3, Tbx21, and Batf when compared to Th9 cells that contain the YTDF2 gene. In embodiments, this recombinant T helper 9 cell expresses lower levels of Ctla4, Fas1, Fos, and Tigit, and Tox when when compared to Th9 cells that contain the YTDF2 gene. Provided herein is a method of treating cancer in a patient in need thereof comprising administering to the patient an effective amount of the T helper 9 cells described herein, including embodiments thereof.

Provided herein is a process of making a recombinant T helper 9 cell comprising: (i) modifying expression of a YTHDF2 protein in naïve CD4⁺ T cells, thereby producing naïve CD4⁺ T cells that do not express a YTHDF2 protein; and (ii) culturing and differentiating the naïve CD4⁺ T cells that do not express a YTHDF2 protein in the presence of IL-4, thereby producing T helper 9 cells that do not express a YTHDF2 protein. In embodiments, step (ii) comprises culturing and differentiating the naïve CD4⁺ T cells that do not express a YTHDF2 protein in the presence of IL-4 and TGF-β or in the presence of IL-4 and IL-1β or in the presence of IL-4, TGF-β, and IL-1β. In embodiments, the process further comprises (iii) transducing the T helper 9 cells that do not express a YTHDF2 protein with a chimeric antigen receptor, thereby producing T helper 9 cells that do not express a YTHDF2 protein and that comprises a chimeric antigen receptor.

The term “T helper 9” or “T helper 9 cell” or “Th9” or “Th9 cell” refers to a subpopulation of CD4⁺ T cells that are generally characterized by their cell surface expression of CD4 and CCR6 and the lack of CCR4. Additionally, Th9 cells are defined by their high secretion of interleukin-9. Besides IL-9, Th9 cells also produce IL-2 and and IL-21.

The term “autologous” is meant to refer to any material (e.g., T-cells) derived from the same individual to which it is later to be re-introduced into the individual. The term “allogeneic” refers to a biological material derived from a different animal of the same species. “Xenogeneic” refers to a graft derived from an animal of a different species.

The phrase “chimeric antigen receptor” or “CAR” as generally used in the art refers to a recombinant fusion protein that has an antigen-specific extracellular domain coupled to an intracellular domain that directs the cell to perform a specialized function upon binding of an antigen to the extracellular domain. A wide variety of chimeric antigen receptors (CARs) have been described in the scientific literature. Chimeric antigen receptors are distinguished from other antigen binding agents by their ability to both bind MHC-independent antigen and transduce activation signals via their intracellular domain. The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (KD) between about 0.1 pM to about 10 μM. An antigen-specific extracellular domain suitable for use in a CAR may be any antigen-binding polypeptide, a wide variety of which are known in the art. In some instances, the antigen-binding domain is a single chain Fv (scFv). Other antibody based recognition domains (cAb VHH (camelid antibody variable domains) and humanized versions thereof, IgNAR V_{H} (shark antibody variable domains) and humanized versions thereof, sdAb V_{H} (single domain antibody variable domains) and “camelized” antibody variable domains are suitable for use. In some instances, T-cell receptor (TCR) based recognition domains such as single chain TCR (scTv, single chain two-domain TCR containing VaVP) are also suitable for use. Suitable antigens may include T cell-specific antigens and/or antigens that are not specific to T cells. In embodiments, an antigen specifically bound by the chimeric antigen receptor of a CAR-T cell, and the antigen for which the CAR-T cell is deficient, is an antigen expressed on a malignant T cell, more preferably an antigen that is overexpressed on malignant T cell in comparison to a non-malignant T cell.

The term “antigen” or “cancer antigen” refers to peptides, proteins, or fragments thereof expressed on the surface of a cancer cell that are capable of binding to the antibody binding domain provided herein. In embodiments, a antigen binding domain binds to a tumor-associated antigen. In embodiments, a antigen binding domain binds to a tumor-specific antigen. In embodiments, the antigen binding domain binds to a surface protein expressed by cells present within a malignant tumor.

In general CARs include an extracellular antigen-binding domain (often a scFv derived from variable heavy and light chains of an antibody), a spacer domain, a transmembrane domain and an intracellular signaling domain. The intracellular signaling domain usually includes the endodomain of a T cell co-stimulatory molecule (e.g., CD28, 4-1BB or OX-40) and the intracellular domain of CD3ζ.

An “antibody region” as provided herein refers to a monovalent or multivalent protein moiety that forms part of the protein provided herein including embodiments thereof. A person of ordinary skill in the art would therefor immediately recognize that the antibody region is a protein moiety capable of binding an antigen (epitope). Thus, the antibody region provided herein may include a domain of an antibody or fragment (e.g., Fab) thereof. In embodiments, the antibody region is a protein conjugate. A “protein conjugate” a provided herein refers to a construct consisting of more than one polypeptide, wherein the polypeptides are bound together covalently or non-covalently. In embodiments, the protein conjugate includes a Fab moiety (a monovalent Fab) covalently attached to an scFv moiety (a monovalent scFv). In embodiments, the protein conjugate includes a plurality (at least two) Fab moieties. In embodiments, the polypeptides of a protein conjugate are encoded by one nucleic acid molecule. In embodiments, the polypeptides of a protein conjugate are encoded by different nucleic acid molecules. In embodiments, the polypeptides are connected through a linker. In embodiments, the polypeptides are connected through a chemical linker.

The “central cavity” with respect to the three-dimensional structure of a Fab, refers to the internal cavity of the Fab lined by portions of the heavy and light chain variable and constant regions and including amino acids lining a hole within the cavity. In embodiments, the central cavity including the hole has a structure. In embodiments, where the antibody region includes a Fab, the central cavity thus is lined by residues of the VH, VL, CH1, and CL regions. The central cavity does not include the antigen binding site. Thus, in embodiments the compound that binds to the central cavity does not impact (e.g. measurably impact) the binding of the antibody region to the epitope. In other words, in embodiments, occupancy of this site does not affect antigen binding.

In embodiments, the antibody region is an antibody fragment. In embodiments, the antibody region includes an Fc domain. In embodiments, the antibody region is a humanized antibody region.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein including embodiments thereof are contemplated.

In embodiments, the CAR provided herein includes an intracellular T-cell signaling domain. In embodiments, the intracellular T-cell signaling domain is a CD3 (intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the same. In embodiments, the signaling of the intracellular T-cell signaling domain results in proliferation (cell division) of the T cell expressing the same. In embodiments, the signaling of the intracellular T-cell signaling domain results expression by said T cell of proteins known in the art to characteristic of activated T cell (e.g., CTLA-4, PD-1, CD28, CD69). In embodiments, the intracellular T-cell signaling domain includes the signaling domain of the zeta chain of the human CD3 complex. In embodiments, the intracellular T-cell signaling domain is a CD3 (intracellular T-cell signaling domain.

In embodiments, the CAR provided herein includes an intracellular co-stimulatory signaling domain. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the T cell expressing the same. In embodiments, the intracellular co-stimulatory signaling domain is a CD28 intracellular co-stimulatory signaling domain, a 4-1BB intracellular co-stimulatory signaling domain, a ICOS intracellular co-stimulatory signaling domain, or an OX-40 intracellular co-stimulatory signaling domain. In embodiments, the intracellular co-stimulatory signaling domain includes a CD28 intracellular co-stimulatory signaling domain, a 4-1BB intracellular co-stimulatory signaling domain, a ICOS intracellular co-stimulatory signaling domain, an OX-40 intracellular co-stimulatory signaling domain or any combination thereof.

In embodiments, the CAR provided herein includes a linker domain. In embodiments, the linker domain is between the transmembrane domain and the intracellular T-cell signaling domain. In embodiments, the linker domain is between the intracellular T-cell signaling domain and the intracellular co-stimulatory signaling domain. In embodiments, the linker domain includes the sequence GGCGG or GGG.

In embodiments, the CAR provided herein includes a spacer region. In embodiments, the spacer region is between the transmembrane domain and the antibody region. A “spacer region” as provided herein is a polypeptide connecting the antibody region with the transmembrane domain. In embodiments, the spacer region connects the heavy chain constant region with the transmembrane domain. In embodiments, the binding affinity of the antibody region to an antigen is increased compared to the absence of the spacer region. In embodiments, the steric hindrance between an antibody region and an antigen is decreased in the presence of the spacer region.

In embodiments, the spacer region includes an Fc region. Examples of spacer regions contemplated for the compositions and methods provided herein include without limitation, immunoglobulin molecules or fragments thereof (e.g., IgG1, IgG2, IgG3, IgG4) and immunoglobulin molecules or fragments thereof (e.g., IgG1, IgG2, IgG3, IgG4) including mutations affecting Fc receptor binding. In embodiments, the spacer region is a fragment of an IgG (e.g., IgG4), wherein said fragment includes a deletion of the CH2 domain. The spacer region may be a peptide linker. In embodiments, the spacer region is a serine-glycine linker. In embodiments, the spacer region has the sequence GGSG. In embodiments, the spacer region has the sequence GSGSGSGS. In embodiments, the spacer region is at least 4 amino acids in length. In embodiments, the spacer region is about 4 amino acids in length. In embodiments, the spacer region is between 4 and 250 amino acids in length. The spacer region may include residues capable of extending the half-life in vivo (e.g., plasma) of the proteins provided herein. In embodiments, the spacer region is 10 amino acids in length. In embodiments, the spacer region is 229 amino acids in length. In embodiments, the spacer region is GGGSSGGGSG. The spacer region may be “pasylated.” The term “pasylated” or “pasylation” is used in its customary sense and refers to an amino acid sequences, which due to their high content in proline, alanine and serine form highly soluble biological polymers. Thus, in embodiments, the spacer region includes about 200 proline, alanine and serine residues combined. In embodiments, the spacer region includes from about 10 to about 200 proline, alanine and serine residues combined. In embodiments, the spacer region includes hydrophilic residues. In embodiments, the CAR does not include a spacer region. In embodiments, the nucleic acid does not include a spacer sequence encoding a spacer region. In embodiments, the nucleic acid does not include a spacer sequence encoding a spacer region as described in WO 2015105522 A1.

The CAR described herein can include a spacer region located between the cancer antigen targeting domain (e.g., a CD19 ScFv, e.g., the scFv portion can include the CD19 targeted scFv sequence of a CD19-targeted CAR) and the transmembrane domain. A variety of different spacer regions can be used. Some of them include at least portion of a human Fc region.

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one ore more amino acid modifications, for example, 1, 2, 3, 4 or 5 substitutions, e.g., substitutions that reduce off-target binding.

The CAR is selective for any antigen, for example: CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family member; B-cell maturation antigen (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); interleukin-13 receptor subunit alpha-2; mesothelin; Interleukin 11 receptor alpha (IL-11 Ra); prostate stem cell antigen (PSCA); Protease Serine 21; vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-1 receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromnosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); roplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P4501B1 (CYP1B1); CCCTC-Binding Factor (Zinc Finger Protein)-Like, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fe fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1); and a combination of two or more thereof.

In embodiments, the CAR is an anti-CD19 protein, anti-CD20 protein, anti-CD22 protein, anti-CD30 protein, anti-CD33 protein, anti-CD44v6/7/8 protein, anti-CD123 protein, anti-CEA protein, anti-EGP-2 protein, anti-EGP-40 protein, anti-erb-B2 protein, anti-erb-B2,3,4 protein, anti-FBP protein, anti-fetal acetylcholine receptor protein, anti-GD2 protein, anti-GD3 protein, anti-Her2/neu protein, anti-IL-13R-a2 protein, anti-KDR protein, anti k-light chain protein, anti-LeY protein, anti-L1 cell adhesion molecule protein, anti-MAGE-A1 protein, anti-mesothelin protein, anti-murine CMV infected cell protein, anti-MUC2 protein, anti-NKGD2 protein, anti, oncofetal antigen protein, anti-PCSA protein, anti-PSMA protein, anti-TAA (targeted by mAb IfE) protein, anti-EGFR protein, anti-TAG-72 protein or anti-VEGF-72 protein.

In embodiments, the CAR is an anti-CD19 protein. In embodiments, the CAR is an anti-CD20 protein. In embodiments, the CAR is an anti-CD22 protein. In embodiments, the CAR is an anti-CD30 protein. In embodiments, the CAR is an anti-CD33 protein. In embodiments, the CAR is an anti-CD44v6/7/8 protein. In embodiments, the CAR is an anti-CD123 protein. In embodiments, the CAR is an anti-CEA protein. In embodiments, the CAR is an anti-EGP-2 protein. In embodiments, the CAR is an anti-EGP-40 protein. In embodiments, the CAR is an anti-erb-B2 protein. In embodiments, the CAR is an anti-erb-B2,3,4 protein. In embodiments, the CAR is an anti-FBP protein. In embodiments, the CAR is an anti-fetal acetylcholine receptor protein. In embodiments, the CAR is an anti-GD2 protein. In embodiments, the CAR is an anti-GD3 protein. In embodiments, the CAR is an anti-Her2/neu protein. In embodiments, the CAR is an anti-IL-13R-a2 protein. In embodiments, the CAR is an anti-KDR protein. In embodiments, the CAR is an anti k-light chain protein. In embodiments, the CAR is an anti-LeY protein. In embodiments, the CAR is an anti-L1 cell adhesion molecule protein. In embodiments, the CAR is an anti-MAGE-A1 protein, anti-mesothelin protein. In embodiments, the CAR is an anti-murine CMV infected cell protein. In embodiments, the CAR is an anti-MUC2 protein. In embodiments, the CAR is an anti-NKGD2 protein. In embodiments, the CAR is an anti-oncofetal antigen protein. In embodiments, the CAR is an anti-PCSA protein In embodiments, the CAR is an anti-PSMA protein. In embodiments, the CAR is an anti-TAA (targeted by mAb IfE) protein. In embodiments, the CAR is an anti-EGFR protein. In embodiments, the CAR is an anti-TAG-72 protein. In embodiments, the CAR is an anti-VEGF-72 protein.

### Pharmaceutical Compositions

In embodiments, the disclosure provides pharmaceutical compositions comprising a compound described herein (including embodiments thereof) and a pharmaceutically acceptable excipient. In embodiments, the disclosure provides pharmaceutical compositions comprising Th9 cells described herein (including embodiments thereof) and a pharmaceutically acceptable excipient.

“Pharmaceutically acceptable excipient” refers to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

A “effective amount” is an amount sufficient for a compound to accomplish a stated purpose relative to the absence of the compound (e.g. achieve the effect for which it is administered, treat a disease). An example of an “effective amount” is an amount sufficient to contribute to the treatment of a disease which could also be referred to as a “therapeutically effective amount.” A “reduction” of a symptom or symptoms (and grammatical equivalents of this phrase) means decreasing of the severity or frequency of the symptoms or elimination of the symptoms. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). For any compound described herein, the therapeutically effective amount can be initially determined from cell culture assays. Target concentrations will be those concentrations of active compound that are capable of achieving the methods described herein, as measured using the methods described herein or known in the art. As is known in the art, therapeutically effective amounts for use in humans can also be determined from animal models. For example, a dose for humans can be formulated to achieve a concentration that has been found to be effective in animals. The dosage in humans can be adjusted by monitoring the effectiveness of the compounds or compositions described herein, and adjusting the dosage upwards or downwards. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

The term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intra-tumoral, intravenous, intramuscular, intra-arteriole, intradermal, subcutaneous, intraperitoneal, intraventricular, and intracranial. In embodiments, the therapeutic agents described herein are parenterally administered to a patient. In embodiments, the therapeutic agents described herein are administered intra-tumorally to a patient. Other modes of delivery include, but are not limited to, the use of liposomal formulations, intravenous infusion, transdermal patches, etc. In embodiments, the administering does not include administration of any active agent other than the recited active agent.

The term “locally administering” means administering a therpaeutic agent to a solid tumor. In embodiments, local administration is direct administration of a therapeutic agent into a solid tumor. In embodiments, local administration is intratumoral administration or pulmonary administration.

The term “intratumorally administering” means administering a therapeutic agent into the tumor or the tumor microenvironment. Intratumoral administration can be direct (e.g., injection of a therapeutic agent into a tumor) or indirect (e.g., oral or intravenous administration of a therapeutic agent that contains a tumor-targeting agent). In embodiments, intratumoral administration comprises injecting a therapeutic agent into the tumor or tumor microenvironment.

The term “pulmonary administration” means administering a therapeutic agent into the lungs. In embodiments, “pulmonary administration” is inhalation of a therapeutic agent into the lungs to treat lung cancer, thereby functioning as local administration of a therapeutic agent.

### Methods of Treating Cancer

Provided herein is a method of treating cancer in a patient in need thereof comprising administering to the patient a compound described herein, including embodiments thereof. In embodiments the method of treating cancer comprises administering to the patient a pharmaceutical composition comprising a compound described herein, including embodiments thereof, and a pharmaceutically acceptable excipient. In embodiments, the cancer comprises TAMs. In embodiments, the cancer comprises TAMs having one or more receptors selected from the group consisting of TLR9, CD163, CD206, CD14, CD16, CD32, CD64, CD68, CD71, CCR5, and CCR2. In embodiments, the cancer comprises TAMs having TLR9 receptors.

Provided herein is a method of treating cancer in a patient in need thereof comprising administering to the patient the Th9 cells described herein, including embodiments thereof. In embodiments the method of treating cancer comprises administering to the patient a pharmaceutical composition comprising the Th9 cells described herein, including embodiments thereof, and a pharmaceutically acceptable excipient. In embodiments, the cancer is a solid tumor. In embodiments, the cancer is pancreatic cancer, lung cancer, breast cancer, or melanoma.

In embodiments, the cancer is a solid tumor. In embodiments, the solid tumor is a hot tumor. In embodiments, the solid tumor is a cold tumor. In embodiments, the cancer is melanoma, prostate cancer, lung cancer, breast cancer, colorectal cancer, kidney cancer, bladder cancer, thyroid cancer, endometrial cancer, glioblastoma, glioma, pancreatic cancer, melanoma, cholangiocarcinoma, B-cell lymphoma, gastric cancer, testicular cancer, thymoma, or uterine cancer. In embodiments, the cancer is melanoma. In embodiments, the cancer is prostate cancer. In embodiments, the cancer is lung cancer. In embodiments, the cancer is breast cancer. In embodiments, the cancer is colorectal cancer. In embodiments, the cancer is kidney cancer. In embodiments, the cancer is bladder cancer. In embodiments, the cancer is thyroid cancer. In embodiments, the cancer is endometrial cancer. In embodiments, the cancer is glioblastoma. In embodiments, the cancer is glioma. In embodiments, the cancer is pancreatic cancer. In embodiments, the cancer is melanoma. In embodiments, the cancer is cholangiocarcinoma. In embodiments, the cancer is B-cell lymphoma. In embodiments, the cancer is gastric cancer. In embodiments, the cancer is testicular cancer. In embodiments, the cancer is thymoma. In embodiments, the cancer is uterine cancer.

In embodiments, the solid tumor is a hot tumor. In embodiments, the hot tumor is melanoma, prostate cancer, lung cancer, breast cancer, colorectal cancer, kidney cancer, bladder cancer, thyroid cancer, endometrial cancer, glioblastoma, glioma, pancreatic cancer, melanoma, cholangiocarcinoma, B-cell lymphoma, gastric cancer, testicular cancer, thymoma, or uterine cancer. In embodiments, the prostate cancer is a hot tumor. In embodiments, the lung cancer is a hot tumor. In embodiments, the breast cancer is a hot tumor. In embodiments, the colorectal cancer is a hot tumor. In embodiments, the kidney cancer is a hot tumor. In embodiments, the bladder cancer is a hot tumor. In embodiments, the thyroid cancer is a hot tumor. In embodiments, the endometrial cancer is a hot tumor. In embodiments, the glioblastoma is a hot tumor. In embodiments, the glioma is a hot tumor. In embodiments, the pancreatic cancer is a hot tumor. In embodiments, the melanoma is hot tumor. In embodiments, the cholangiocarcinoma is a hot tumor. In embodiments, the B-cell lymphoma is a hot tumor. In embodiments, the gastric cancer is a hot tumor. In embodiments, the testicular cancer is a hot tumor. In embodiments, the thymoma is a hot tumor. In embodiments, the uterine cancer is a hot tumor. In embodiments, the melanoma is a hot tumor.

In embodiments, the solid tumor is a cold tumor. In embodiments, the cold tumor is melanoma, prostate cancer, lung cancer, breast cancer, colorectal cancer, kidney cancer, bladder cancer, thyroid cancer, endometrial cancer, glioblastoma, glioma, pancreatic cancer, melanoma, cholangiocarcinoma, B-cell lymphoma, gastric cancer, testicular cancer, thymoma, or uterine cancer. In embodiments, the prostate cancer is a cold tumor. In embodiments, the lung cancer is a cold tumor. In embodiments, the breast cancer is a cold tumor. In embodiments, the colorectal cancer is a cold tumor. In embodiments, the kidney cancer is a cold tumor. In embodiments, the bladder cancer is a cold tumor. In embodiments, the thyroid cancer is a cold tumor. In embodiments, the endometrial cancer is a cold tumor. In embodiments, the glioblastoma is a cold tumor. In embodiments, the glioma is a cold tumor. In embodiments, the pancreatic cancer is a cold tumor. In embodiments, the melanoma is cold tumor. In embodiments, the cholangiocarcinoma is a cold tumor. In embodiments, the B-cell lymphoma is a cold tumor. In embodiments, the gastric cancer is a cold tumor. In embodiments, the testicular cancer is a cold tumor. In embodiments, the thymoma is a cold tumor. In embodiments, the uterine cancer is a cold tumor. In embodiments, melanoma is a cold tumor.

In embodiments, the cancer is a hematological malignancy. In embodiments, the hematological malignancy is lymphoma or leukemia. In embodiments, the hematological malignancy is lymphoma. In embodiments, the hematological malignancy is leukemia.

In embodiments of the methods of treating cancer described herein, the compounds, Th9 cells, and pharmaceutical compositions are locally administered to the cancer in the patient. In embodiments, the compounds, Th9 cells, and pharmaceutical compositions are locally administered to the solid tumor cancer in the patient. Locally administering the compounds, Th9 cells, or pharmaceutical compounds includes administering the compounds or Th9 cells within the solid tumor or administering the compounds to the tumor microenvironment. In embodiments, locally administering refers to administering within the solid tumor. In embodiments, locally administering refers to administering within the tumor microenvironment. In embodiments, locally administering the compound to the cancer in the patient comprises intratumorally administering the compound or Th9 cells to the cancer in the patient. In embodiments, locally administering the compound to the cancer in the patient comprises pulmonary administration of the compound or Th9 cells to the cancer in the patient. Pulmonary administration is local when the cancer is lung cancer.

In embodiments of the methods of treating cancer described herein, the methods comprise administering an effective amount of the compounds or Th9 cells described herein, including embodiments thereof either alone or in combination with an anticancer agent. In embodiments, the methods comprise administering an effective amount of the compounds or Th9 cells described herein, including embodiments thereof in combination with an effective amount of an immune checkpoint inhibitor. In embodiments, the methods comprise administering an effective amount of a pharmaceutical composition comprising the compounds or Th9 cells described herein, including embodiments thereof, either alone or in combination with an anticancer agent. In embodiments, the methods comprise administering an effective amount of a pharmaceutical composition comprising the compounds or Th9 cells described herein, including embodiments thereof in combination with an effective amount of an immune checkpoint inhibitor.

In embodiments, the immune checkpoint inhibitor is a PD-1 inhibitor or a PD-L1 inhibitor. In embodiments, the immune checkpoint inhibitor is a PD-1 inhibitor. In embodiments, the PD-1 inhibitor is pembrolizumab, nivolumab, cemiplimab, dostarlimab, camrelizumab, sintilimab, tislelizumab, toripalimab, spartalizumab, retifanlimab, pimivalimab, AMP-224, or MEDI0680. In embodiments, the PD-1 inhibitor is pembrolizumab. In embodiments, the PD-1 inhibitor is nivolumab. In embodiments, the PD-1 inhibitor is cemiplimab. In embodiments, the PD-1 inhibitor is dostarlimab. In embodiments, the PD-1 inhibitor is camrelizumab. In embodiments, the PD-1 inhibitor is sintilimab. In embodiments, the PD-1 inhibitor is tislelizumab. In embodiments, the PD-1 inhibitor is toripalimab. In embodiments, the PD-1 inhibitor is spartalizumab. In embodiments, the PD-1 inhibitor is retifanlimab. In embodiments, the PD-1 inhibitor is pimivalimab. In embodiments, the PD-1 inhibitor is AMP-224. In embodiments, the PD-1 inhibitor is MEDI0680. In embodiments, the immune checkpoint inhibitor is a PD-L1 inhibitor. In embodiments, the PD-L1 inhibitor is atezolizumab, avelumab, durvalumab, envafolimab, cosibelimab, AUNP12, CA-170, or BMS-986189. In embodiments, the PD-L1 inhibitor is atezolizumab, avelumab, durvalumab, envafolimab, or cosibelimab. In embodiments, the PD-L1 inhibitor is atezolizumab. In embodiments, the PD-L1 inhibitor is avelumab. In embodiments, the PD-L1 inhibitor is durvalumab. In embodiments, the PD-L1 inhibitor is envafolimab. In embodiments, the PD-L1 inhibitor is cosibelimab. In embodiments, the PD-L1 inhibitor is AUNP12. In embodiments, the PD-L1 inhibitor is CA-170. In embodiments, the PD-L1 inhibitor is BMS-986189.

### EXAMPLES

### Example 1

The RNA N⁶-methyadenosine (m⁶A) methylation, an epigenetic modification, which is the most common type of RNA methylation that occurs at the N⁶-position of adenosine, has recently been found to play a critical role in shaping the TME. (Ref 16). TAMs are potent regulators of tumor-associated immune suppression in the TME; however, the mechanisms for this effect remain to be fully elucidated, including the roles of readers of the m⁶A modification in this setting. YTH domain-containing family [YTHDF] proteins, including YTHDF1, YTHDF2, and YTHDF3, function as the main m⁶A readers through binding to the methylated RNA and mediating specific functions. (Refs 10,26). For example, YTHDF1 enhances mRNA translation, YTHDF2 regulates mRNA stability, and YTHDF3 promotes the translation and degradation of mRNA. (Refs 27-30). Clearly defining the roles of the readers of m⁶A modification in TAMs will provide us with a new opportunity to target the TME for cancer immunotherapy.

In this study, we show that the anti-tumor immunity of TAMs is controlled by mRNA m⁶A methylation through the m⁶A-reader protein YTHDF2. YTHDF2 deficiency promotes the polarization of anti-tumorigenic macrophages and increases their antigen cross-presentation ability, thereby enhancing CD8⁺ T cell-mediated anti-tumor immunity. Based on these results, we developed an innovative agent, CpG oligodeoxynucleotide (CpG-ODN)-conjugated-Ythdf2 small interfering RNA (siRNA), for targeting YTHDF2 specifically in TAMs via Toll-like receptor 9 (TLR9). Our approach produced profound anti-tumor effects, including when used in combination with anti-PD-L1 therapy. Taken together, our findings uncover a previously unappreciated mechanism by which YTHDF2 in TAMs sabotages anti-tumor innate and adaptive immunity. YTHDF2 inhibition is a promising approach to harness the anti-tumor activity of macrophages and CD8⁺ T cells to enhance cancer immunotherapy.

### YTHDF2 is Upregulated in Tumor-Infiltrating Myeloid Cells

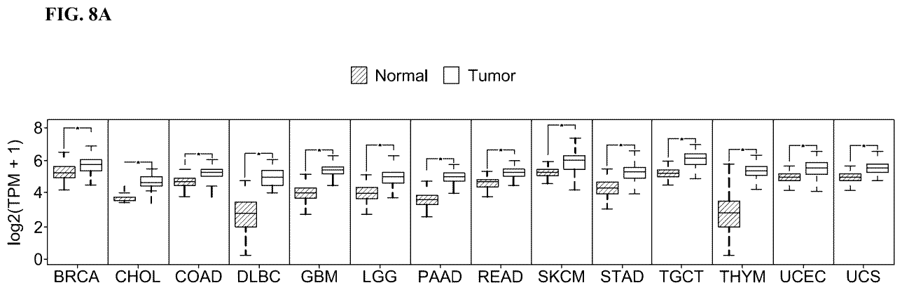

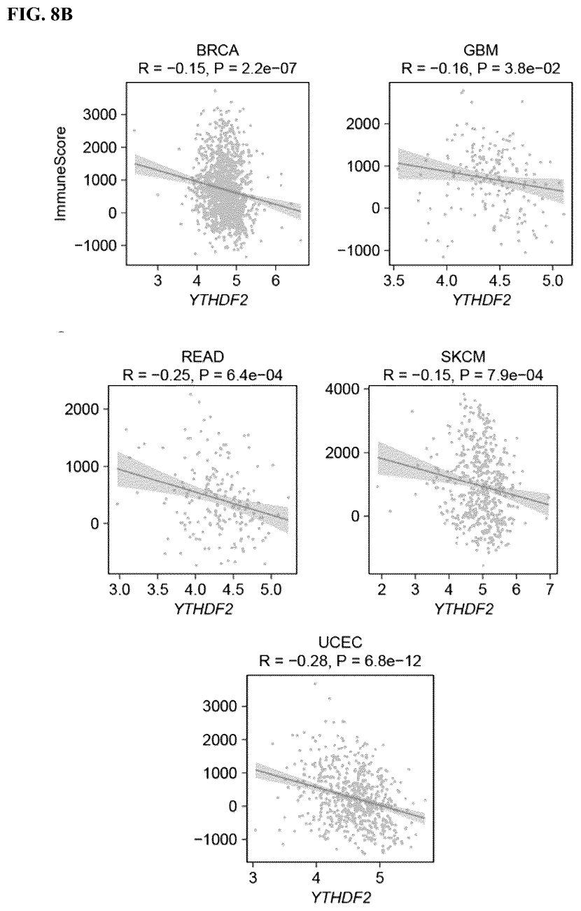

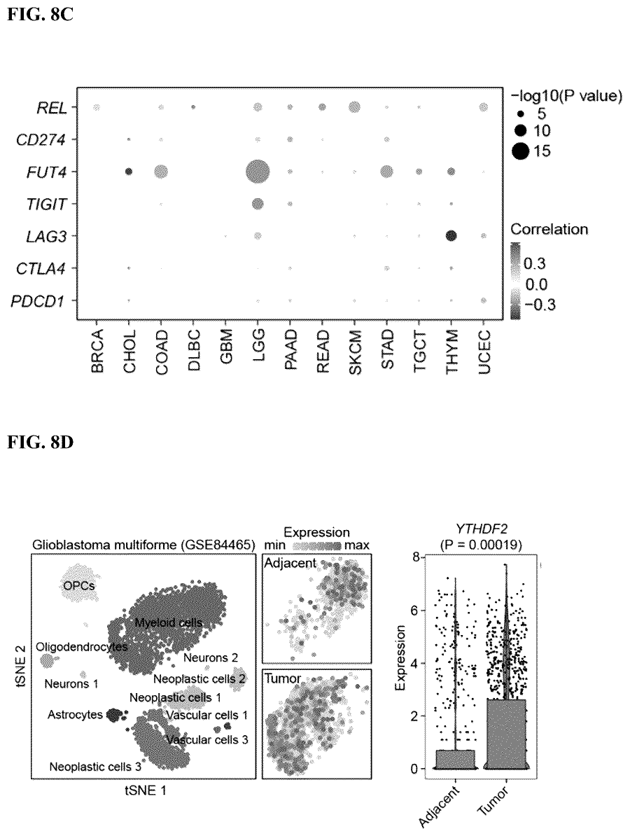

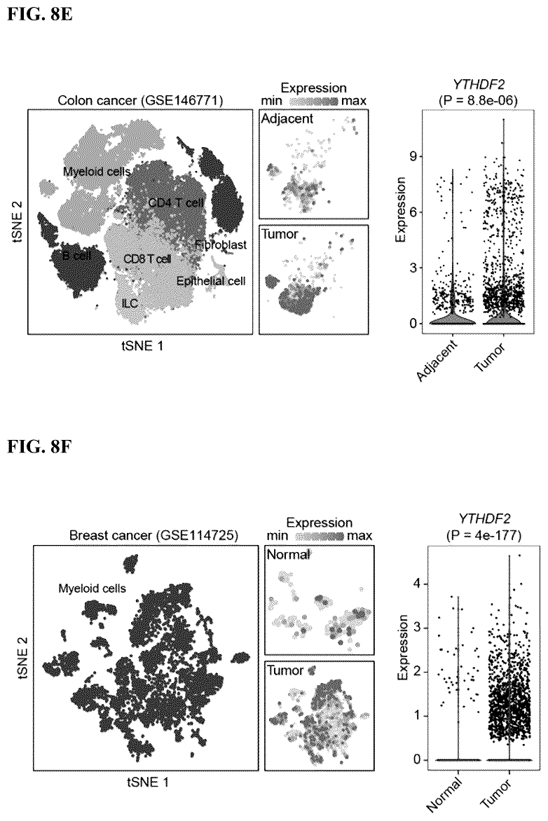

YTHDF2 is one of the most important m⁶A-reader proteins that control the life span and stability of target mRNA. (Ref 28). We screened the YTHDF2 expression in tumor samples and their adjacent normal tissues from RNA sequencing (RNA-seq) results of The Cancer Genome Atlas (TCGA) database and The Genotype-Tissue Expression (GTEx) database. YTHDF2 mRNA expression was significantly upregulated in 14 out of 31 types of tumor tissues, including breast cancer (BRCA), colon cancer (COAD), glioblastoma (GBM), rectum adenocarcinoma (READ), skin melanoma (SKCM), and uterine corpus endometrial carcinoma (UCEC) (FIG. 8A), which was in agreement with the previously published studies. (Refs 31-34). The Estimation of STromal and Immune cells in MAlignant Tumor tissues using Expression data (ESTIMATE) is an algorithm that was developed for estimating the immune and stromal cell infiltration into the TME. (Ref 35). Using this algorithm, we found a negative correlation between YTHDF2 mRNA expression and immune scores in BRCA, GBM, READ, SKCM, and UCEC (FIG. 8B). These data indicate that YTHDF2 is upregulated and can negatively correlate with tumor immune cell infiltration in cancer.

TIMs play a key role in shaping the TME and mediating tumor immune escape¹¹. However, the function of YTHDF2 in TIMs is unclear. By leveraging the TIMER2.0 website, we found that YTHDF2 expression positively correlated with the expression of myeloid-related genes, including REL (encoding C-Rel) and fucosyltransferase 4 (FUT4, encoding CD15) (FIG. 8C). YTHDF2 expression also positively correlated with the expression of immune checkpoint genes, CD274 (encoding PD-L1), TIGIT, LAG3, CTLA4, and PDCD1 (encoding PD-1) in several types of cancers (FIG. 8C). We then analyzed public single-cell RNA sequencing (scRNA-seq) data to evaluate the expression levels of YTHDF2 in TIMs. We found that the expression of YTHDF2 in TIMs was higher than that seen in myeloid cells in adjacent or normal tissues in patients with GBM, COAD, or BRCA (FIGS. 8D-8F). These results indicate that YTHDF2 is upregulated in TIMs, which prompted us to explore the precise role of YTHDF2 in TIMs during tumor development.

### Myeloid YTHDF2 Deficiency Suppresses Tumor Growth and Metastasis

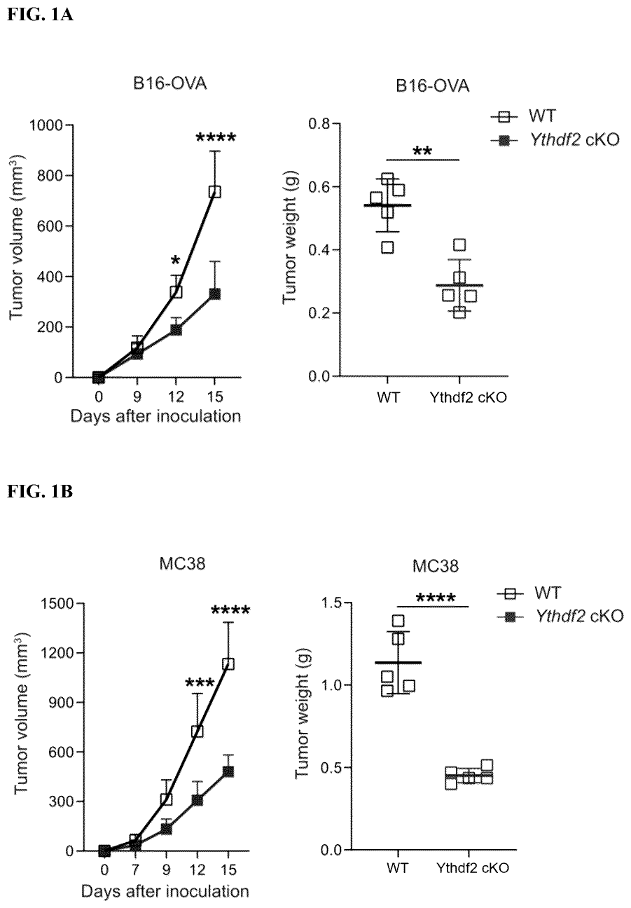

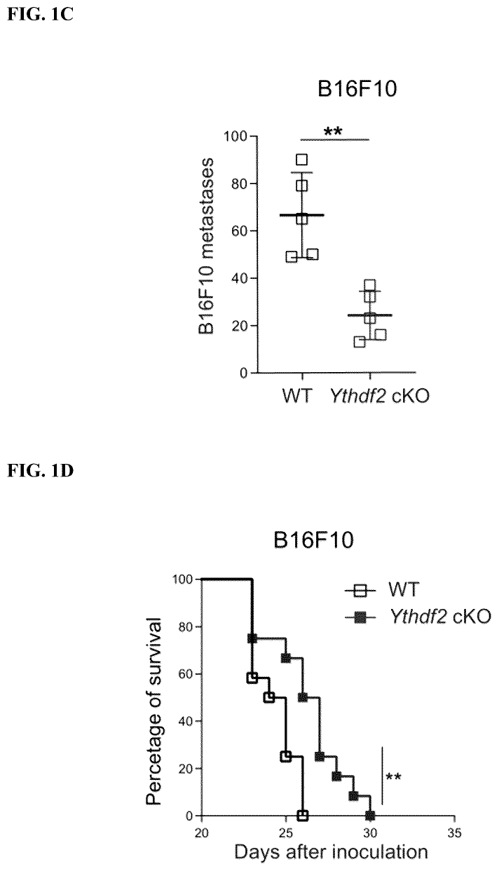

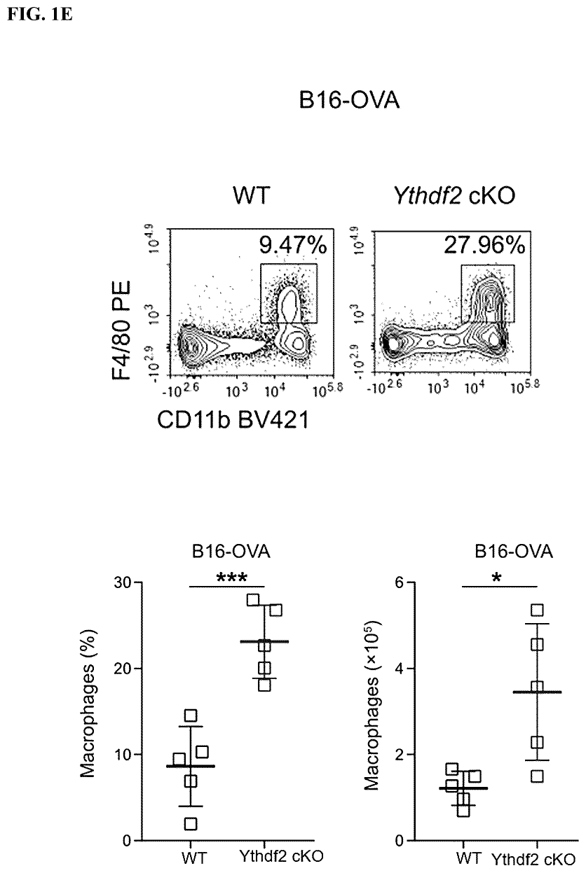

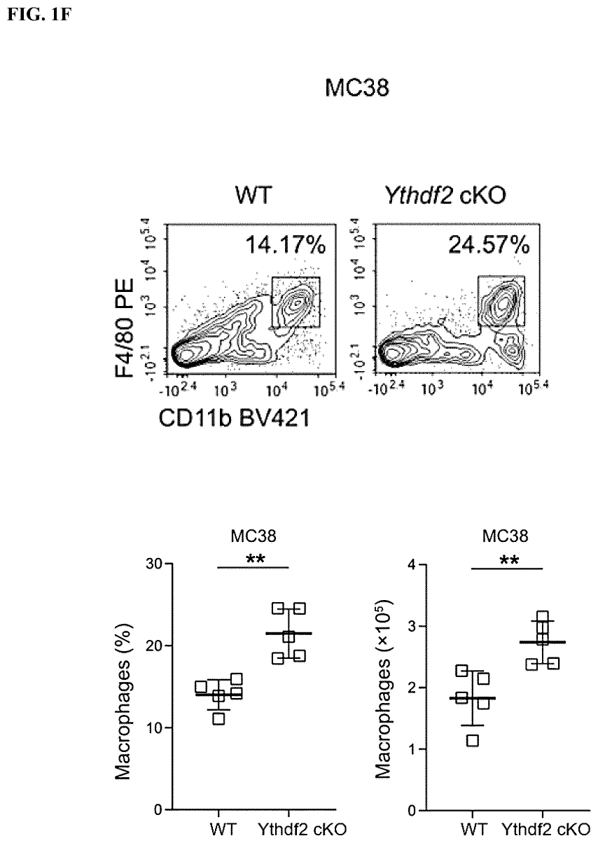

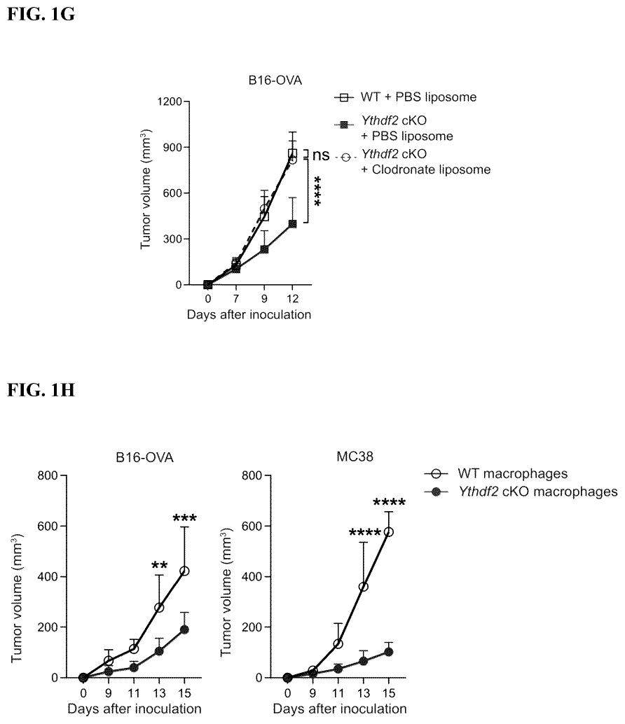

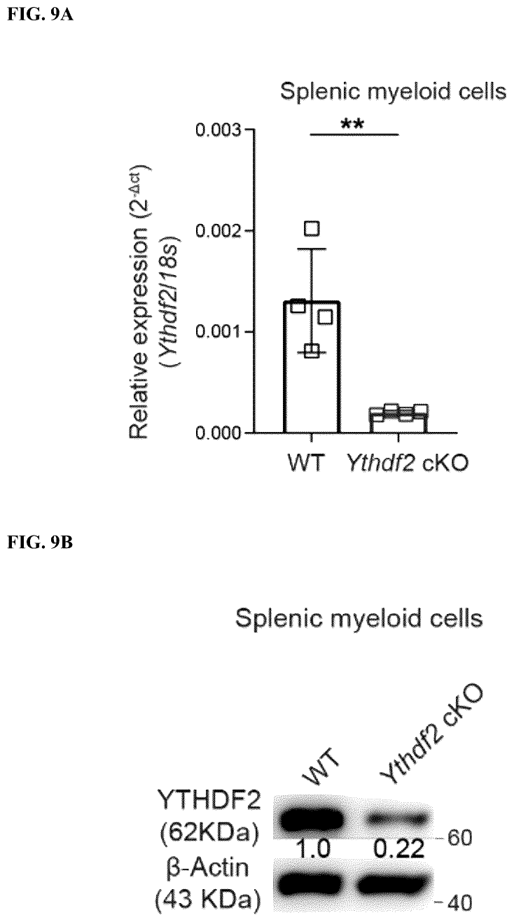

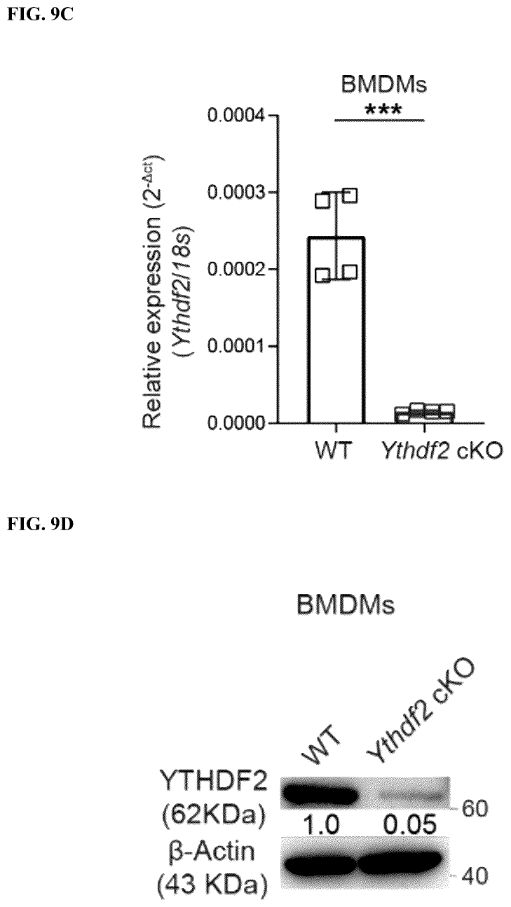

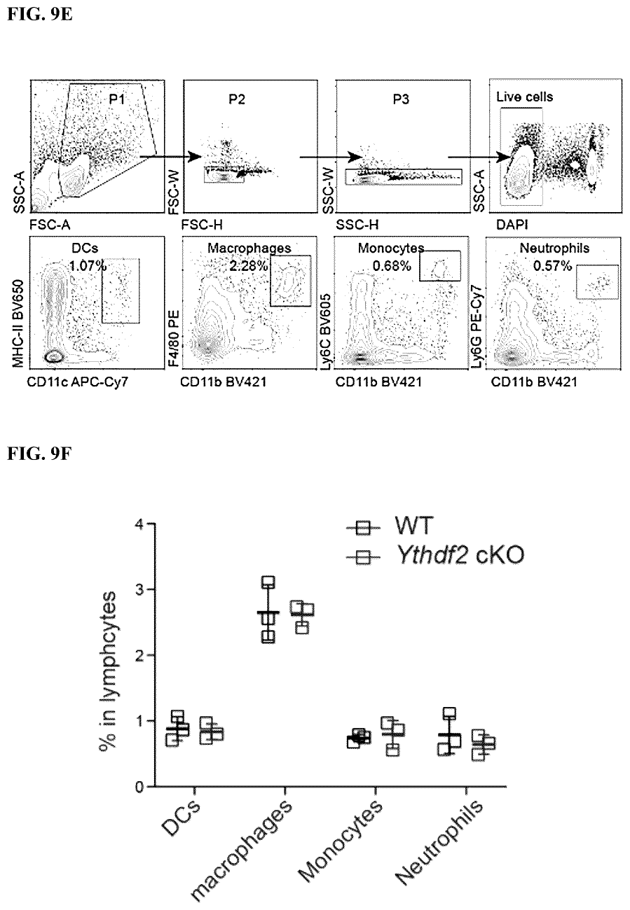

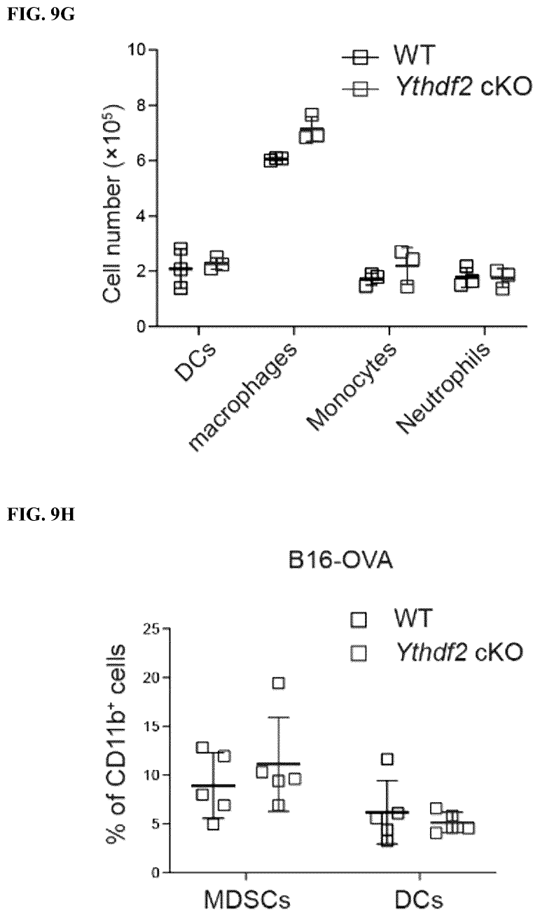

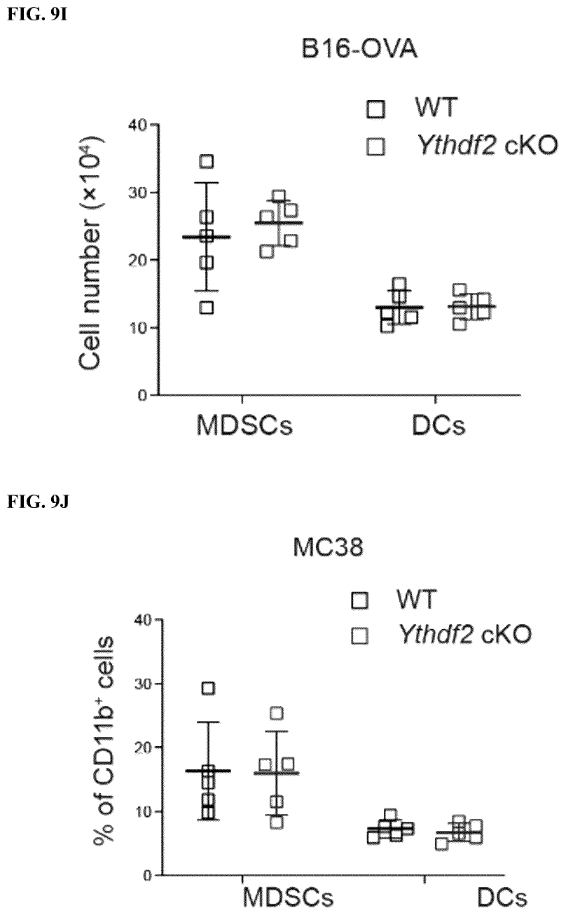

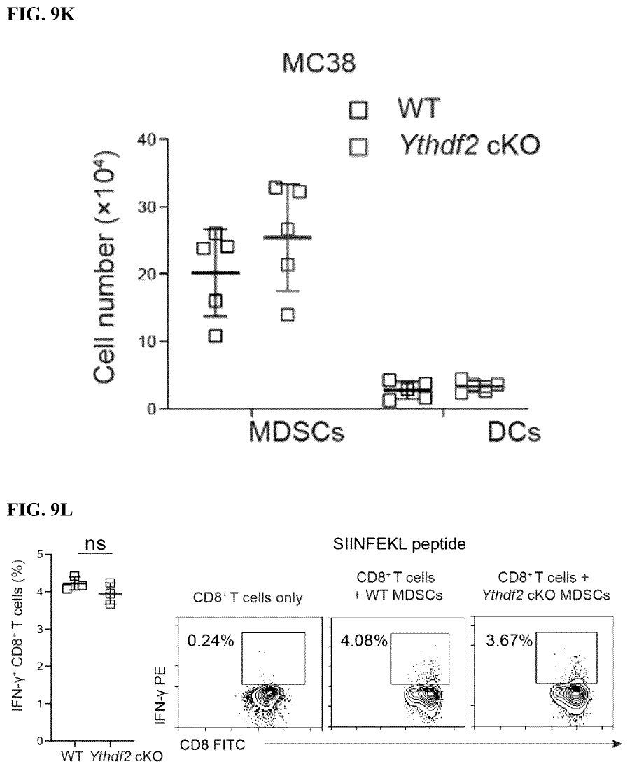

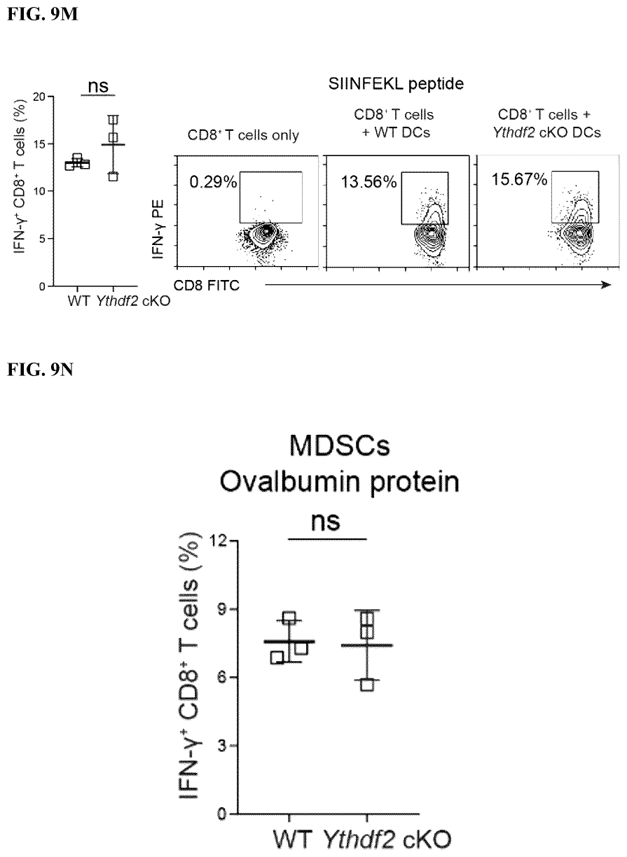

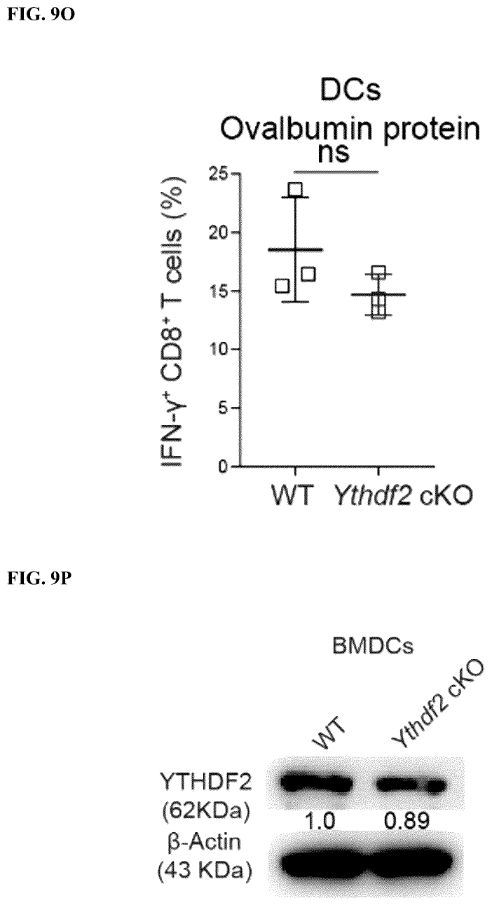

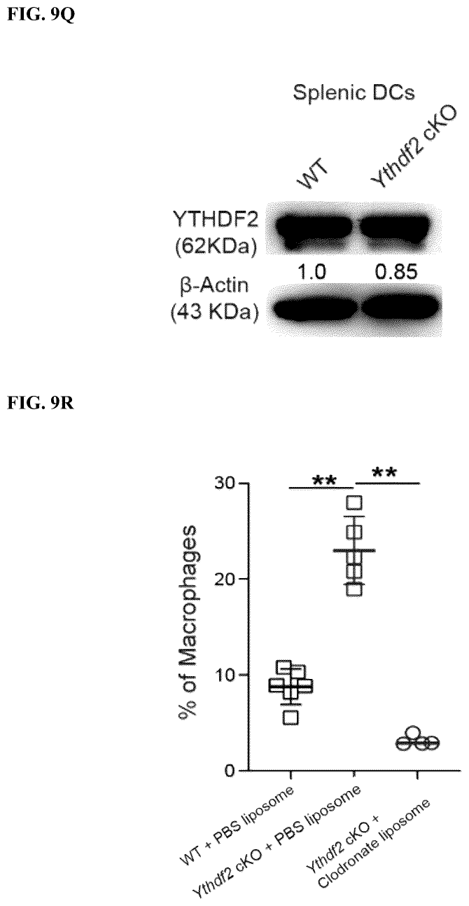

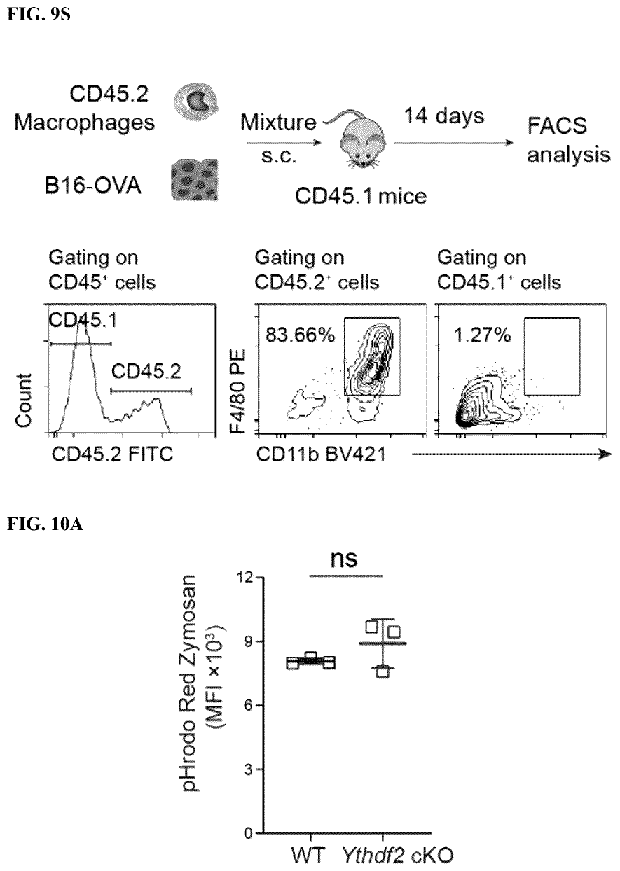

To address the role of myeloid YTHDF2 in tumor development, we generated a mouse model in which the Ythdf2 gene was selectively ablated in the myeloid lineage. This was achieved by crossing our Ythdf2^{f/f} mice with the lysozyme 2 (Lyz2 or LysM) Cre mice to generate Ythdf2^{f/f}Lyz2^{Cre} mice (hereafter Ythdf2^{f/f} denoted as wild type [WT] and Ythdf2^{f/f}Lyz2^{Cre} as Ythdf2 cKO) (Ref 36) (FIGS. 9A-9D). YTHDF2 deficiency did not change the frequency or composition of myeloid cells in the spleen at a steady-state (FIG. 9E-9G). We then established two tumor models by subcutaneous (s.c.) injection of B16-OVA or MC38 cells into the flanks of syngeneic WT and Ythdf2 cKO mice. Tumor growth was significantly delayed in the absence of myeloid YTHDF2 in the two tumor models, as demonstrated by a significant reduction in tumor volume and tumor weight in Ythdf2 cKO mice compared to WT mice (FIGS. 1A-1B). We also established a third tumor model, a melanoma lung metastatic model, by intravenous (i.v.) injection of B16F10 cells into WT and Ythdf2 cKO mice. Ythdf2 cKO mice displayed a much less burden of metastatic nodules than those of WT mice (FIG. 1C). Moreover, B16F10 tumor-bearing Ythdf2 cKO mice had significantly longer survival than WT mice (FIG. 1D). Collectively, these data indicate that YTHDF2 deficiency in myeloid cells suppresses tumor growth and metastasis in mice.

### YTHDF2 Deficiency in Macrophages Contributes to Tumor Inhibition

To identify the subset of myeloid cells that contributes to tumor suppression in YTHDF2 deficiency, we profiled myeloid cell populations in tumor tissues of two tumor models. In both models, WT and Ythdf2 cKO mice had comparable percentages and absolute numbers of tumor-infiltrating MDSCs and DCs (FIG. 9H-9K). When we co-cultured OT-I naïve CD8⁺ T cells, which specifically recognize the SIINFEKL peptide, with MDSCs or DCs isolated from tumors of B16-OVA-bearing mice that were pre-loaded with the SIINFEKL peptide or co-cultured with ovalbumin protein, we found that myeloid YTHDF2 deficiency did not undermine the inhibitory activity of MDSCs or the cross-priming capacity of DCs, measured by IFN-γ production by the T cells (FIG. 9L-9O), indicating that myeloid YTHDF2 deficiency does not affect the percentages and functions of MDSCs and DCs. The Lyz2-Cre-mediated genetic deletion of Ythdf2 is mainly found in myeloid cells, such as monocytes, macrophages, and granulocytes, but rarely in DCs. (Ref 37). Indeed, we found comparable levels of YTHDF2 in bone marrow-derived dendritic cells (BMDCs) and splenic DCs between WT and Ythdf2 cKO mice (FIG. 9P-9Q), indicating that the function of DCs may not be affected by YTHDF2 in our model. Given that DCs, specifically the subset of conventional type 1 DCs (cDC1s), are specialized for priming CD8⁺ T cell responses through antigen cross-presentation, further studies are warranted to explore the role of YTHDF2 in DCs using appropriate Cre models. (Ref 38). In addition, we documented leaky expression of YTHDF2 in both splenic myeloid cells and bone marrow-derived macrophages (BMDMs) (FIG. 9B, 9D), which is consistent with a previous report showing 83-98% deletion efficiency of gene expression in mature macrophages in Lyz2 Cre mice. (Ref 37). Therefore, more dramatic effects may be observed if complete knockout of YTHDF2 is achieved.

By contrast, we observed a significant increase in the percentage and absolute number of macrophages in tumor tissues of Ythdf2 cKO mice compared to those of WT mice (FIGS. 1E-1F), indicating that YTHDF2 deficiency resulted in an increase of macrophages in the TME. To determine whether the myeloid YTHDF2 deficiency resulting in suppression of tumor growth is dependent on macrophages, we depleted macrophages in tumor-bearing mice by i.v. injection of clodronate liposomes on days 0 and 7 after tumor inoculation (FIG. 9R). Depleting macrophages abrogated the differences in tumor growth between WT and Ythdf2 cKO mice (FIG. 1G). In addition, we established a tumor model by injecting a mixture of tumor cells and BMDMs (FIG. 9S). We confirmed that the adoptive-transfer of BMDMs could persist in vivo for at least 2 weeks (FIG. 9S). Co-engrafting B16-OVA or MC38 cells with BMDMs from Ythdf2 cKO mice significantly reduced tumor growth compared to co-engrafting either of these tumors with BMDMs from WT mice (FIG. 1H). Taken together, these data indicate that YTHDF2 deficiency in TAMs contributes to suppression of tumor growth.

### YTHDF2 Deficiency in Macrophages Strengthens CD8⁺ T Cell-Mediated Anti-Tumor Immunity

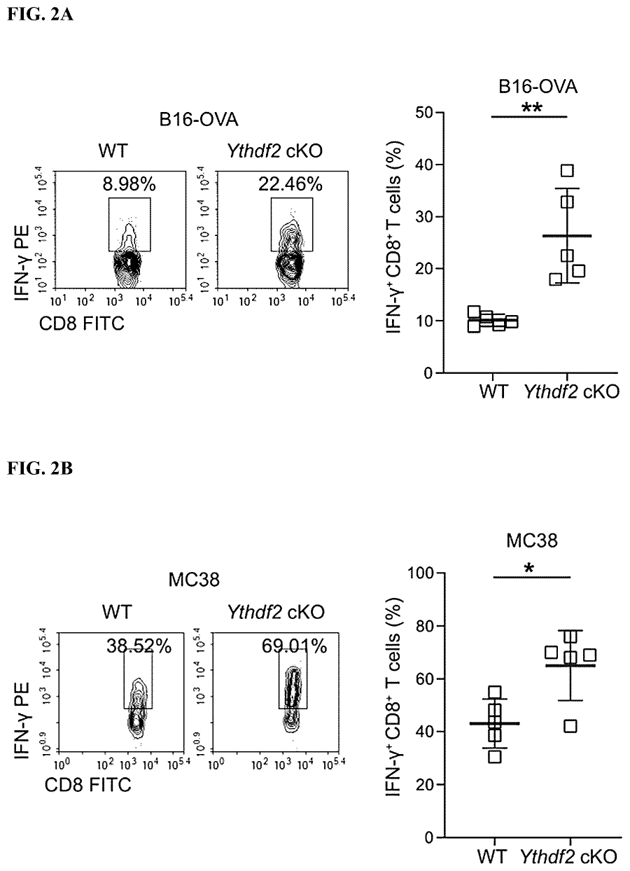

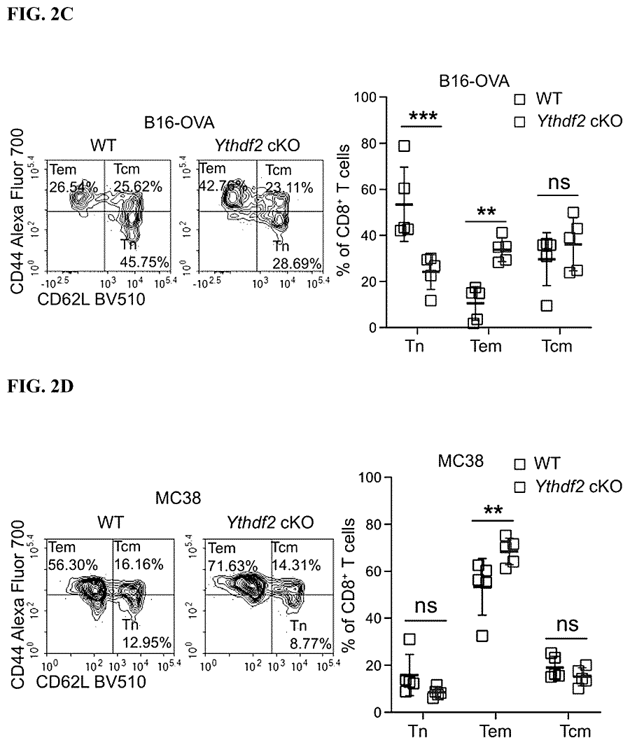

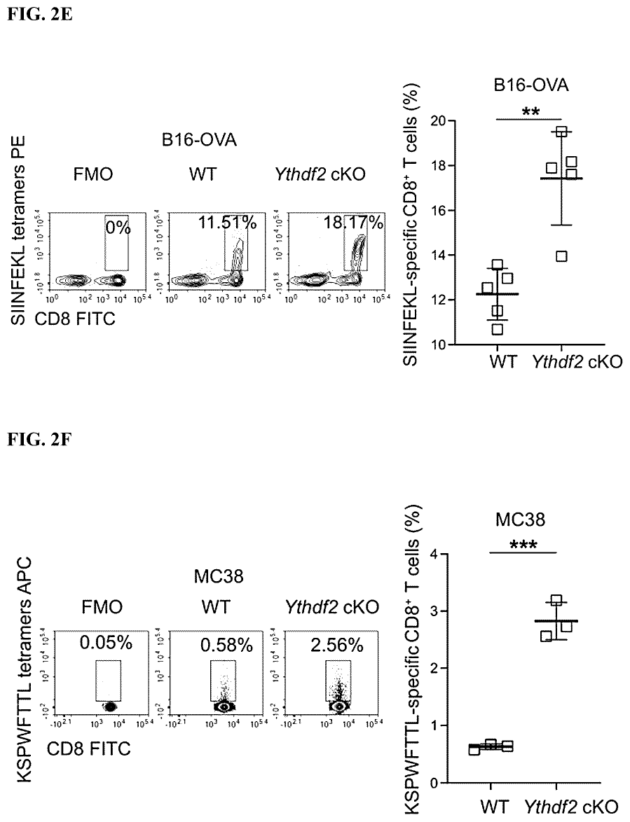

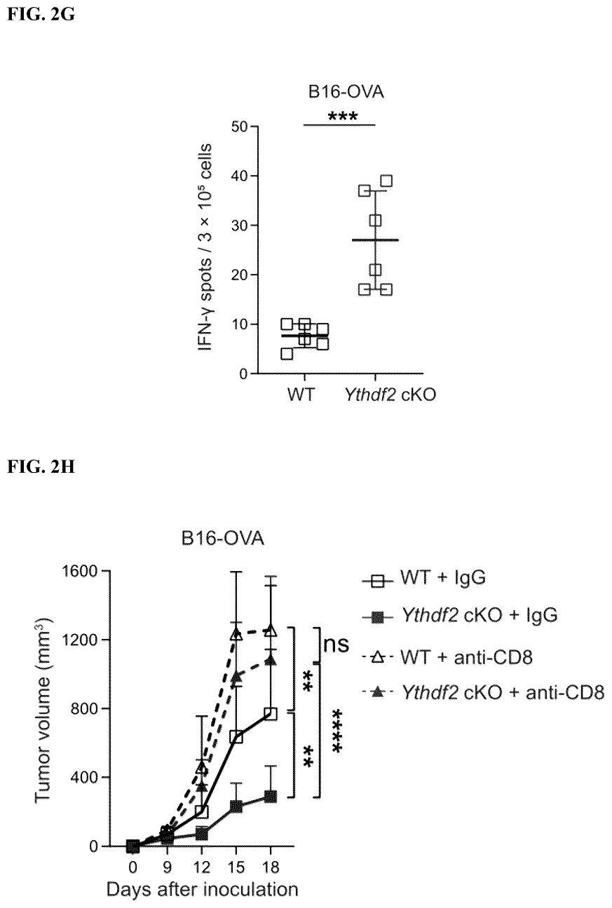

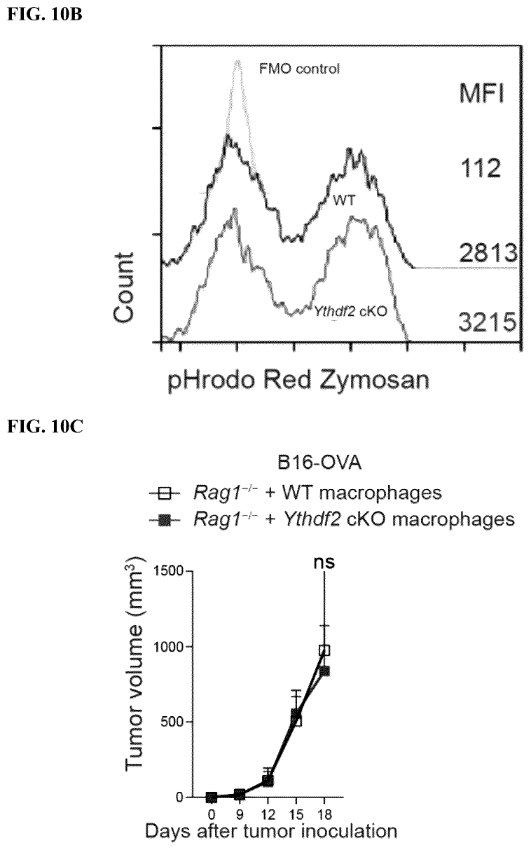

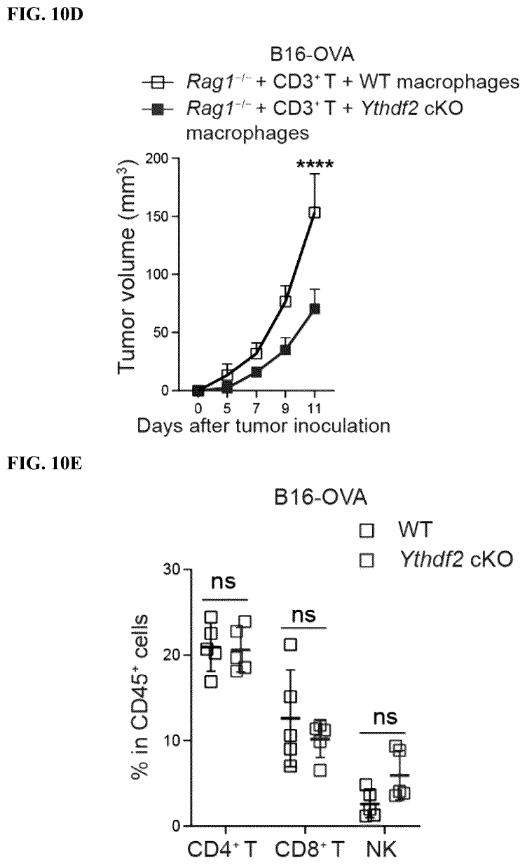

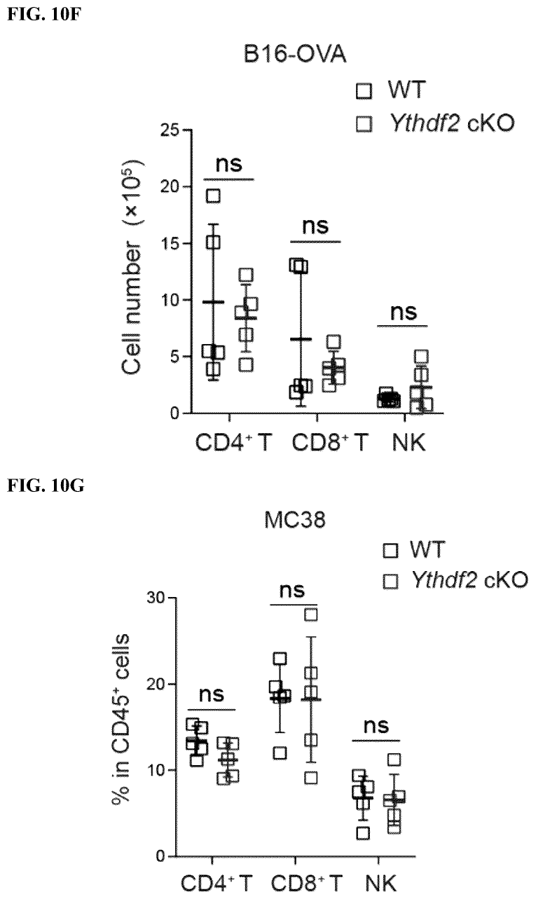

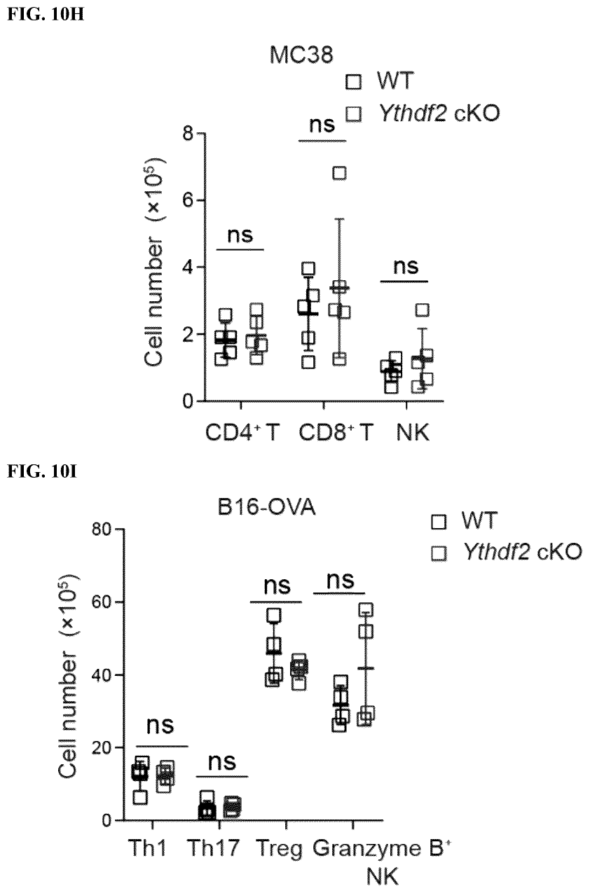

Macrophages alone have been shown to control tumor progression through phagocytosis³⁹, but we found no significant difference in phagocytic ability between WT and Ythdf2 cKO mice (FIGS. 10A-10B). Macrophage YTHDF2 deficiency also failed to suppress tumor growth in Rag1^{-/-} mice that lack T and B cells (FIG. 10C). However, in Rag1^{-/-} mice in which CD3⁺ T cells were adoptively transferred without B cells, YTHDF2 deficiency in macrophages could suppress tumor growth (FIG. 10D), indicating that macrophages deficient in YTHDF2 require assistance from T cells instead of B cells to control tumor growth. We therefore profiled lymphocytes that had infiltrated tumors. There were no significant differences in percentages and absolute numbers of CD4⁺ T cells, CD8⁺ T cells, and NK cells between WT and Ythdf2 cKO mice in either the B16-OVA or the MC38 tumor model (FIGS. 10E-10H). The percentages of CD4⁺ T cell subsets, such as T helper (Th) 1, Th17 effector cells, and regulatory T cells (Treg), and granzyme B-producing NK cells were comparable between WT and Ythdf2 cKO mice in both the B16-OVA and the MC38 tumor model (FIGS. 10I-10J), indicating that total CD4⁺ T cells as well as their subsets and activated NK cells are not involved in enhanced tumor eradication mediated by YTHDF2-deficient TAMs. However, we observed higher levels of IFN-γ-producing CD8⁺ cytotoxic T cells in tumors from Ythdf2 cKO mice compared to those from WT mice (FIGS. 2A-2B). Moreover, we found a significant increase of effector memory CD8⁺ T cells (Tₑₘ, CD62L^{low}CD44^{high}) in tumors from Ythdf2 cKO mice compared to those from WT mice (FIGS. 2C-2D). We then examined antigen-specific CD8⁺ T cell responses. We found that the frequency of tumor-infiltrating SIINFEKL-specific CD8⁺ T cells was significantly upregulated in B16-OVA tumors from Ythdf2 cKO mice compared to those from WT mice (FIG. 2E). We also observed higher levels of tumor-infiltrating KSPWFTTL-specific CD8⁺ T cells in MC38 tumors from Ythdf2 cKO mice compared to those from WT mice (FIG. 2F). Using an IFN-γ enzyme-linked immune absorbent spot assay, we found that SIINFEKL-specific CD8⁺ T cells in the draining lymph modes produced more IFN-γ in Ythdf2 cKO mice than in WT mice (FIG. 2G). To further acknowledge that suppression of tumor growth by YTHDF2 deficiency is dependent on CD8⁺ T cell-mediated anti-tumor immunity, we depleted CD8⁺ T cells using an anti-CD8 antibody. Indeed, the anti-tumor response in YTHDF2-deficient mice was completely abrogated in the absence of CD8⁺ T cells (FIG. 2H). By contrast, Rag1^{-/-} mice with adoptively transferred WT OT-I T cells and YTHDF2-deficient BMDMs had significantly smaller tumors than those that received both WT OT-I T cells and WT BMDMs (FIG. 2I). Collectively, these data indicate that YTHDF2 deficiency in macrophages can suppress tumor progression only in the presence of CD8⁺ T cell-mediated anti-tumor immunity.

### YTHDF2 Deficiency Improves Macrophage Anti-Tumorigenic Polarization

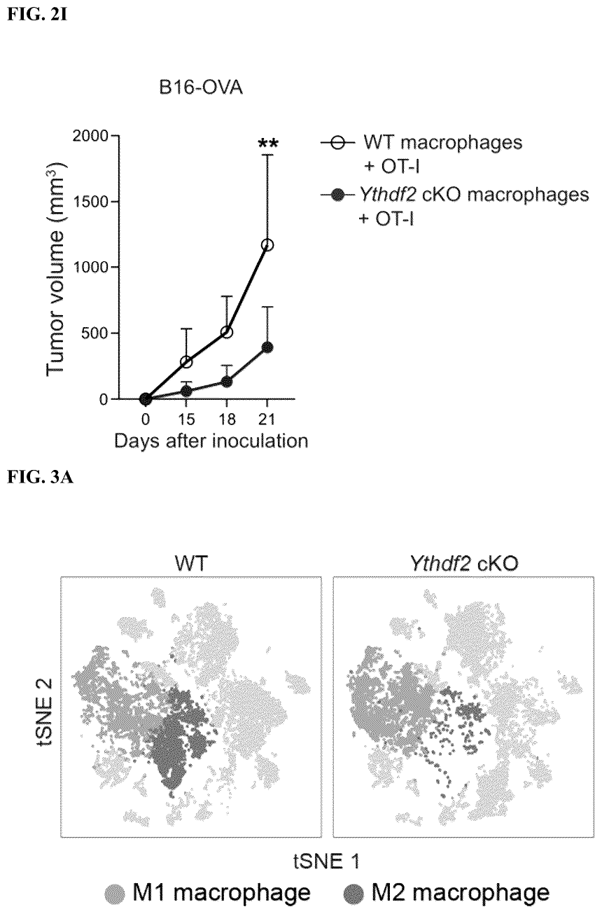

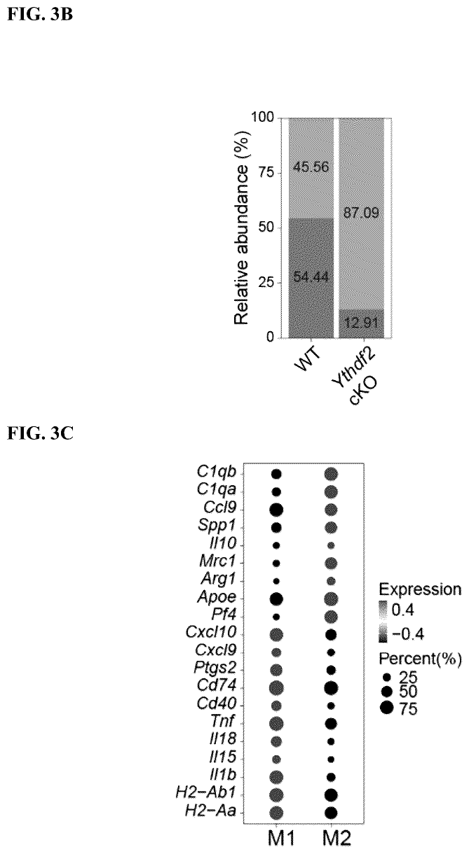

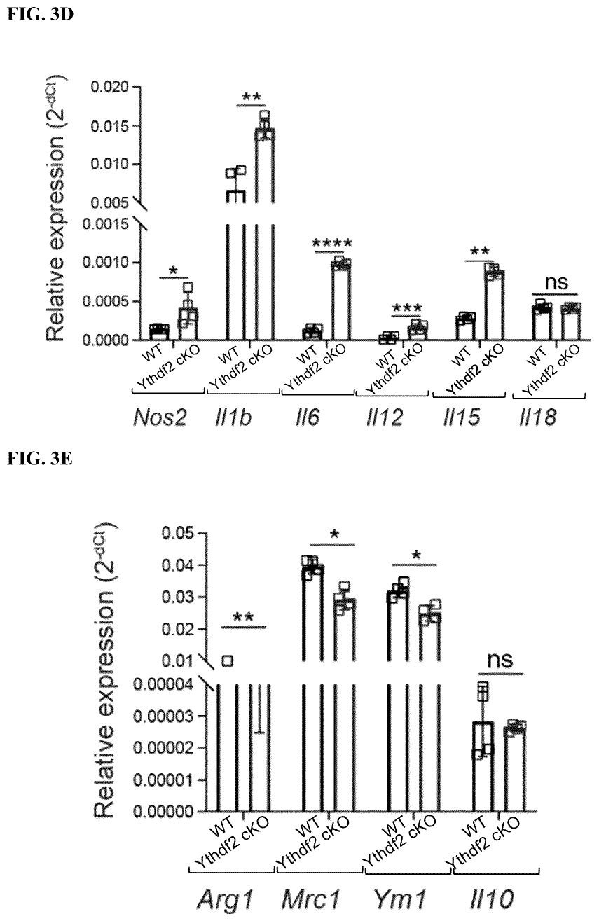

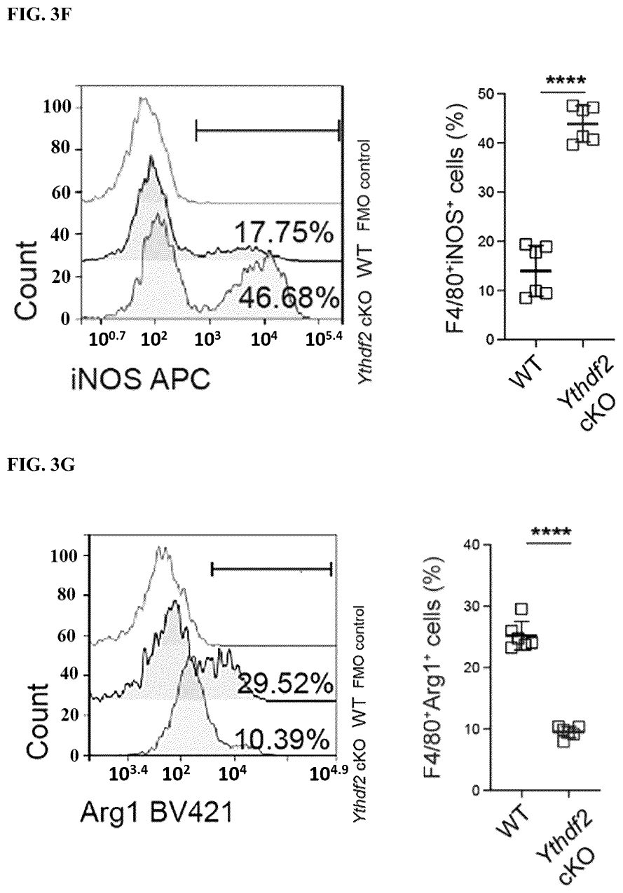

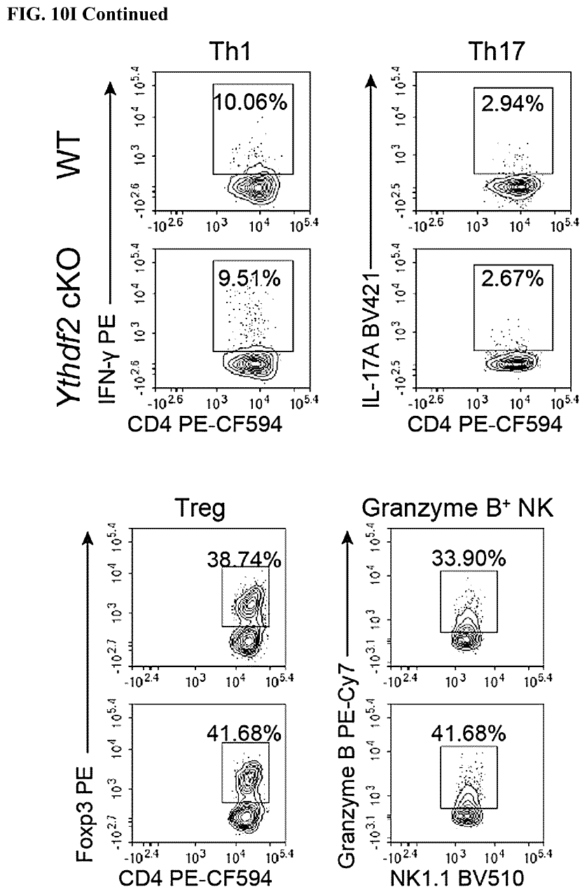

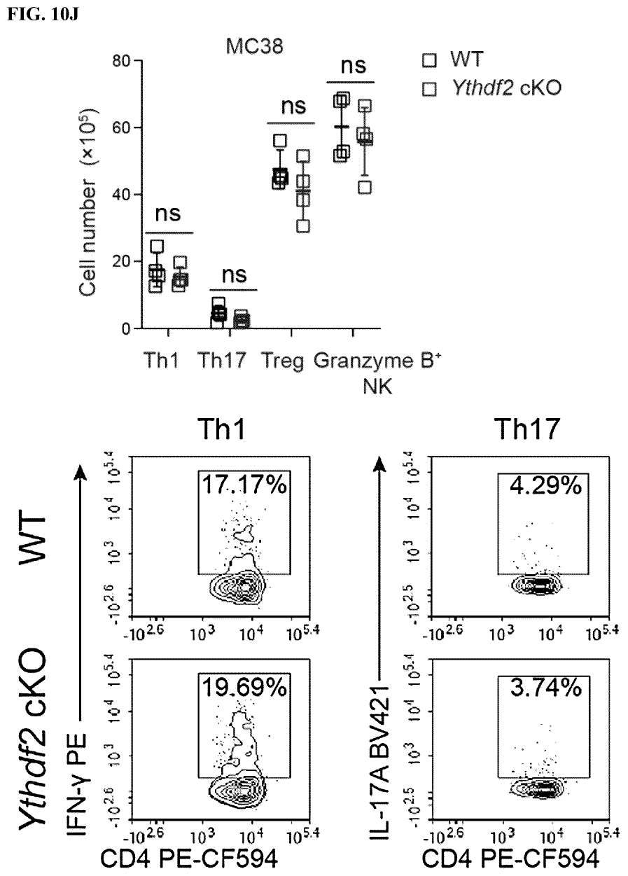

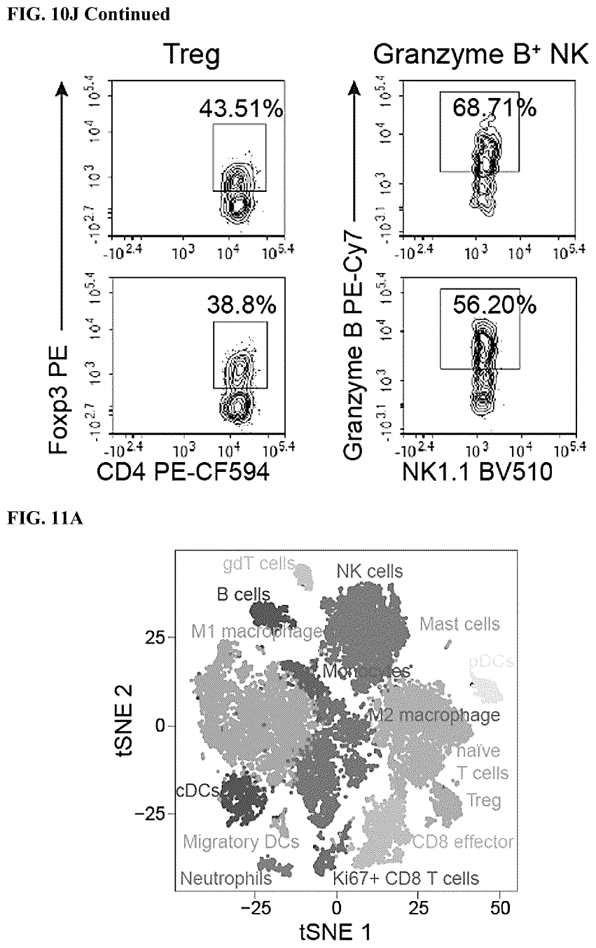

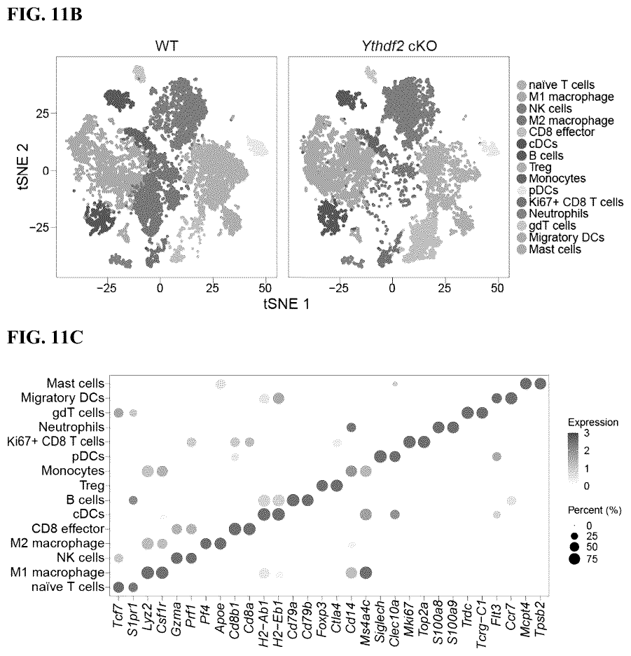

As TAMs comprise both anti-tumorigenic (M1 type) and pro-tumoral (M2 type) cells, we further characterized this heterogeneous population. First, we analyzed CD45-positive tumor-infiltrating cells from WT and Ythdf2 cKO mice by single-cell RNA sequencing (scRNA-seq). Unsupervised cluster analysis using nonlinear dimensionality reduction (t-distributes stochastic neighbor embedding [t-SNE]) identified 15 clusters of cells with unique expression features (FIGS. 11A-11C). Cell types for these clusters were then identified based on the canonical markers, which included three monocyte/macrophage clusters, two CD8⁺ T cell clusters, three DC clusters, and single clusters of B cells, naïve T cells, Treg cells, γδ T cells, NK cells, neutrophils, and mast cells in WT and Ythdf2 cKO mice (FIGS. 11A-11C). Of note, the proportion of M2 macrophages characterized by Pf4, Apoe, Arg1, Mrc1, Il10, Spp1, Ccl9, C1qa, and C1qb was lower in Ythdf2 cKO tumors. In contrast, the proportion of M1 macrophages—characterized by H2-Aa, H2-Ab1, Il1b, Il15, Il18, Tnf, Cd40, Cd74, Ptgs2, Cxcl9, and Cxcl10—was higher in Ythdf2 cKO tumors compared to WT tumors (FIGS. 3A-3C), indicating a switch from pro-tumoral to anti-tumoral macrophages in Ythdf2 cKO tumors. Quantitative (q)PCR analysis validated that the mRNA expression of M1 markers, including Nos2, Tnf, Il1b, Il6, Il12, and Il15 were significantly upregulated in macrophages isolated from tumors from Ythdf2 cKO mice compared to those from WT mice (FIG. 3D). In contrast, the expression of M2 markers, including Arg1, Mrc1, and Chil3 were significantly decreased in macrophages isolated from tumors of Ythdf2 cKO mice compared to those from WT mice (FIG. 3E). Immunoblotting assay confirmed that M1 macrophages from Ythdf2 cKO mice had increased inducible nitric oxide synthase (iNOS) levels (FIG. 11D), whereas M2 macrophages from Ythdf2 cKO mice showed decreased arginase 1 (Arg1) expression compared to those from WT mice (FIG. 11D). Flow cytometry assay further confirmed that the percentage of M1 macrophages (CD11b⁺F4/80⁺iNOS⁺) was significantly increased, whereas the percentage of M2 macrophages (CD11b⁺F4/80⁺Arg1⁺) was markedly decreased in tumor grafts from Ythdf2 cKO mice compared to those from WT mice (FIGS. 3F-3G). Together, our findings reveal that deficiency of YTHDF2 in macrophages reprograms TAMs to become anti-tumorigenic.

### YTHDF2 Deficiency Augments the Antigen Cross-Presentation Ability of Anti-Tumorigenic Macrophages

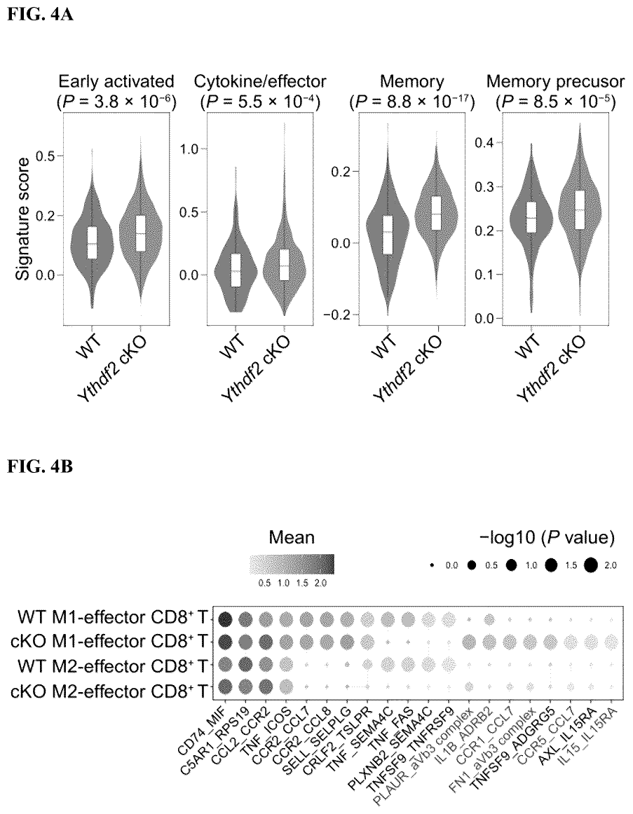

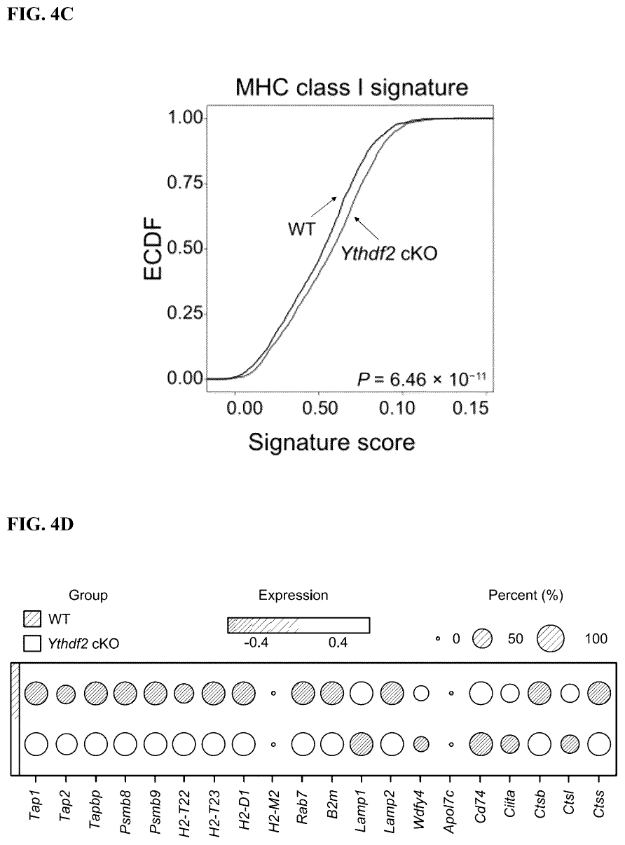

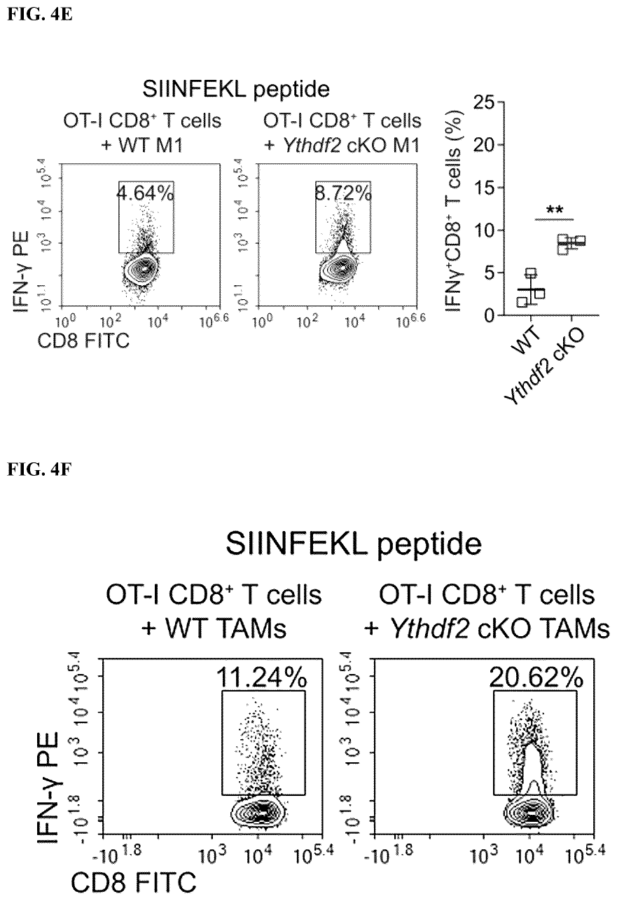

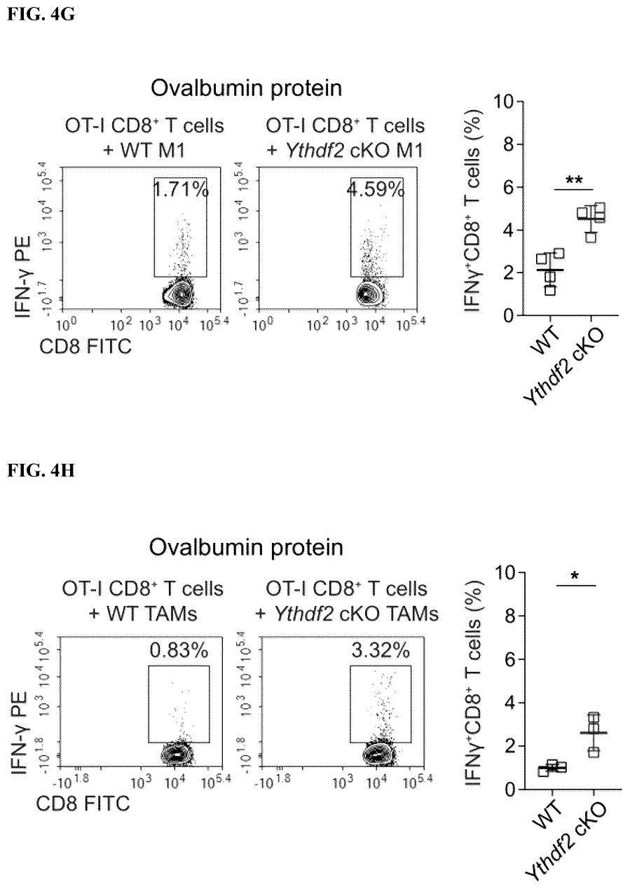

Macrophages can favor T-cell mediated anti-tumor responses not only by secreting proinflammatory cytokines but also by presenting tumor antigens on major histocompatibility complex (MHC) class I and II to CD8⁺ and CD4⁺ T cells, respectively. (Ref 40). We found a larger proportion of effector CD8⁺ T cells (marked by Cd8a, Cd8b1, Cd3g, Ifng, Gzmb, Gzma, and Prf1) in tumors from Ythdf2 cKO mice than in those from WT mice (FIG. 11E). In addition, we observed that the CD8⁺ T cell cluster from Ythdf2 cKO mice expressed higher levels of gene signatures in early activated, cytokine/effector, memory, and memory precursor states (Ref 41) (FIG. 4A), indicating that YTHDF2-deficient macrophages facilitate CD8⁺ T cell activation and effector functions in the TME.

We evaluated the crosstalk between macrophage subsets and tumor-infiltrating effector CD8⁺ T cells using the cell-cell communication analytical tool in CellPhoneDB. (Ref 42). We found some unique interactions between YTHDF2-deficient M1 macrophages and effector CD8⁺ T cells that strongly associated with MHC class I cross-presentation, leukocyte recruitment, and T cell activation (e.g., PLAUR_αvβ3, CCR1_CCL7, IL-1β_ADRB2, and IL-15_IL-15RA) (FIG. 4B). These correlations indicate that YTHDF2 regulates the crosstalk between M1 macrophages and CD8⁺ T cells. Further analysis showed that M1 macrophages from Ythdf2 cKO mice exhibited a higher score of MHC class I gene signature (FIG. 4C), in which several genes related to MHC class I antigen presentation (including Tap1, Tap2, Tapbp, Psmb8, Psmb9, H2-T22, H2-T23, H2-D1, Rab7, B2m, Lamp2, Ctsb, and Ctss) were upregulated (FIG. 4D). (Ref 41). Flow cytometric analysis showed that M1 macrophages had presented more H-2K^{b}-SIINFEKL complexes than M2 macrophages regardless of the WT or the Ythdf2 cKO genotype (FIG. 11F), which is consistent with prior evidence that particularly anti-tumorigenic M1 macrophages are capable of antigen cross-presentation. (Ref 40). Levels of H-2K^{b}-SIINFEKL complexes were also more upregulated in M1 macrophages from Ythdf2 cKO mice compared to those M1 macrophages from WT mice (FIG. 11F). Moreover, an in vitro antigen-presentation assay showed that M1 BMDMs from Ythdf2 cKO mice or TAMs isolated from B16-OVA tumor-bearing Ythdf2 cKO mice were better able to cross-prime CD8⁺ T cells for both the SIINFEKL peptide and the ovalbumin protein, as evidenced by their greater production of IFN-γ when compared to M1 BMDMs or TAMs from WT mice (FIGS. 4E-4H). Type I IFNs (IFN-α/β) are key for the induction of antigen cross-presentation. (Ref 43). TAMs isolated from B16-OVA tumor-bearing Ythdf2 cKO mice had significantly higher mRNA levels of Ifna4 and Ifnb1 compared to those from WT mice (FIG. 11G). Collectively, these results indicate that deficiency of YTHDF2 increases the ability of M1 macrophages to cross-present antigen, thereby enhancing anti-tumor responses of CD8⁺ T cells.

### YTHDF2 Deficiency Reprograms Macrophages Via IFN-γ—STAT1 Signaling

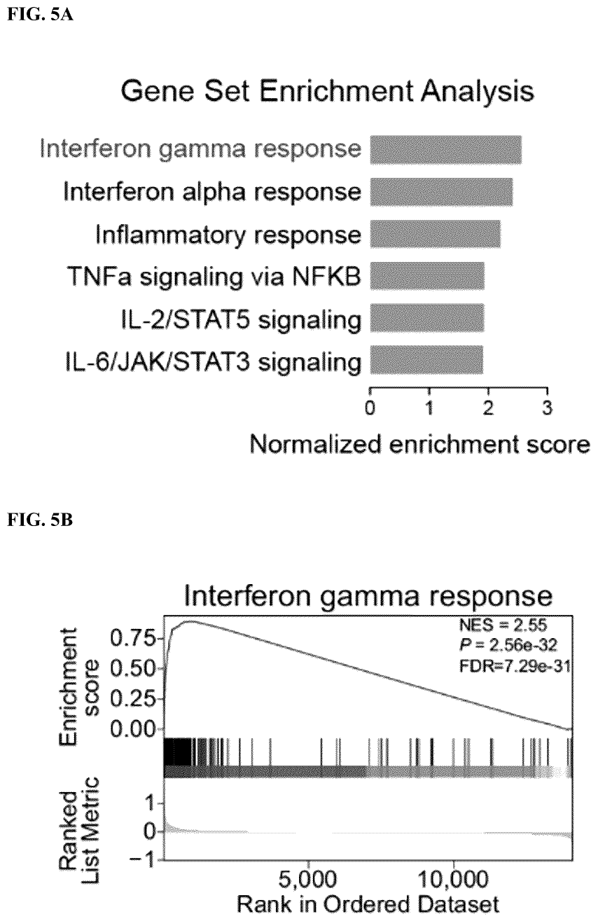

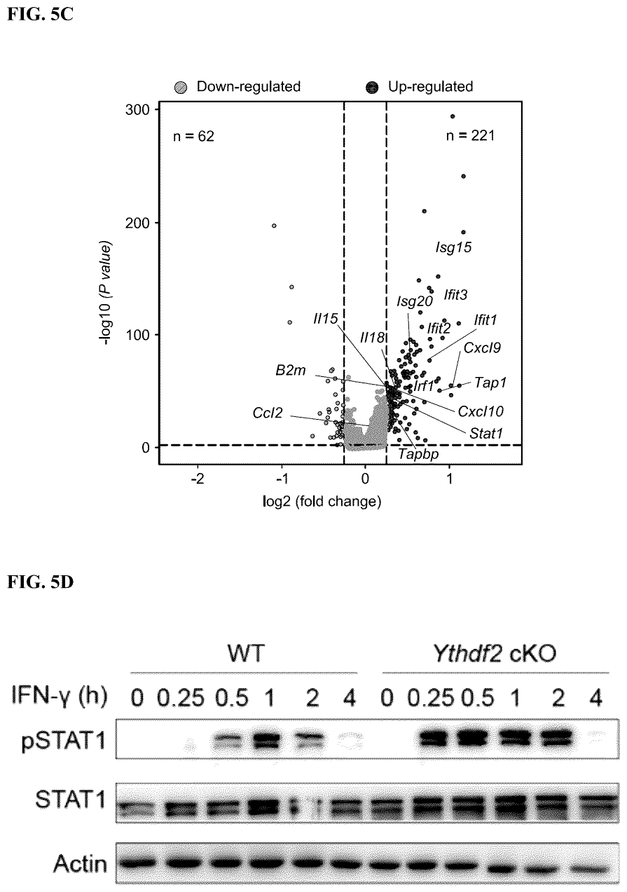

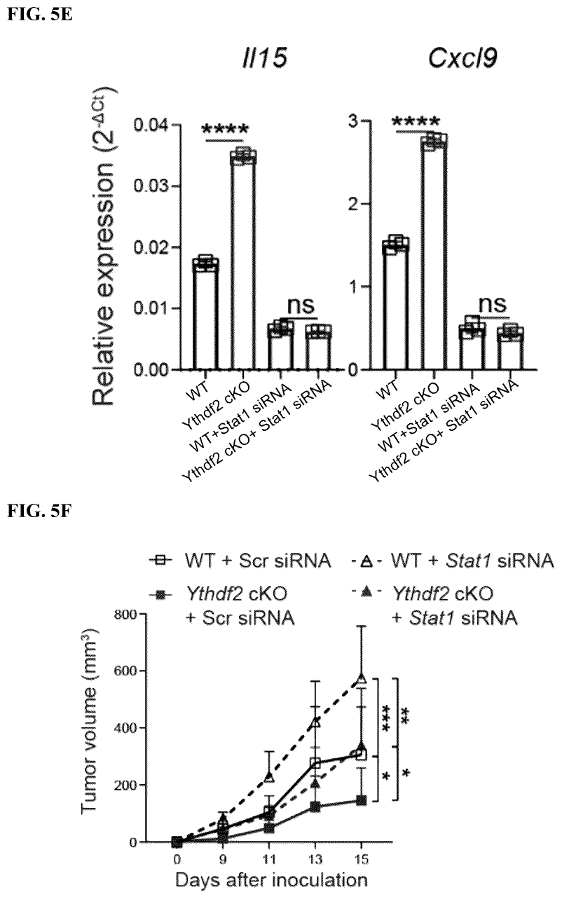

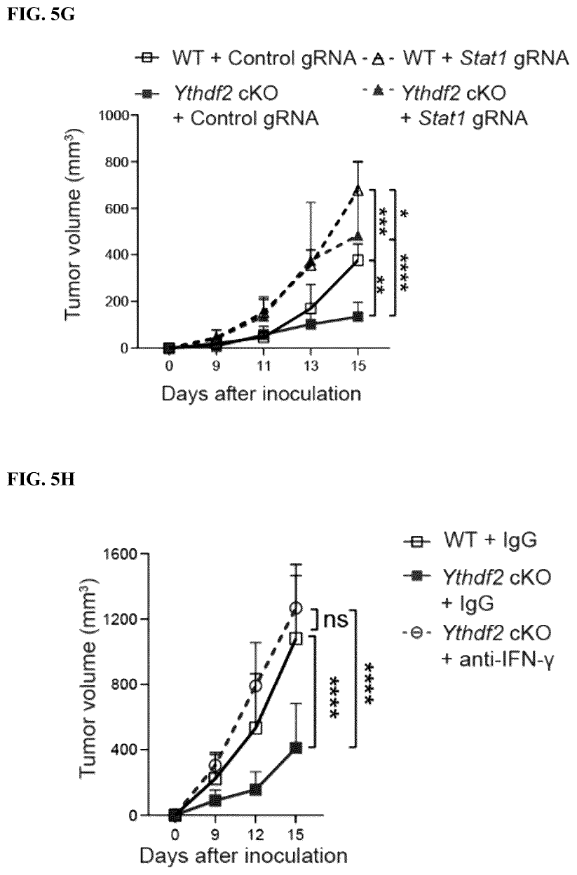

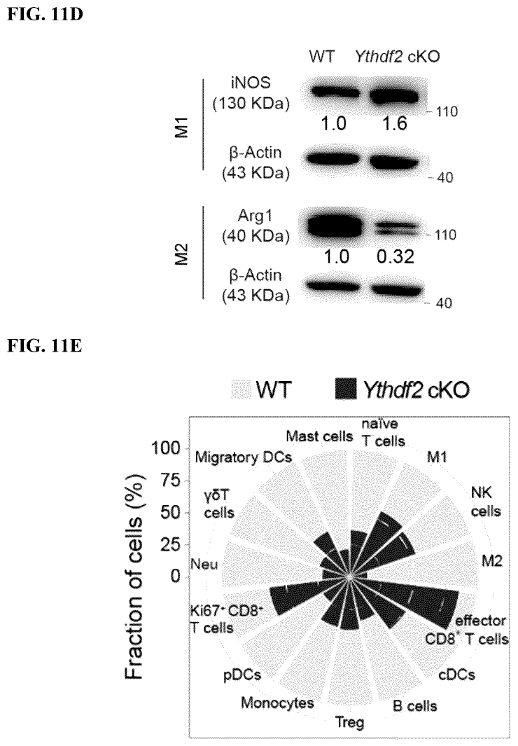

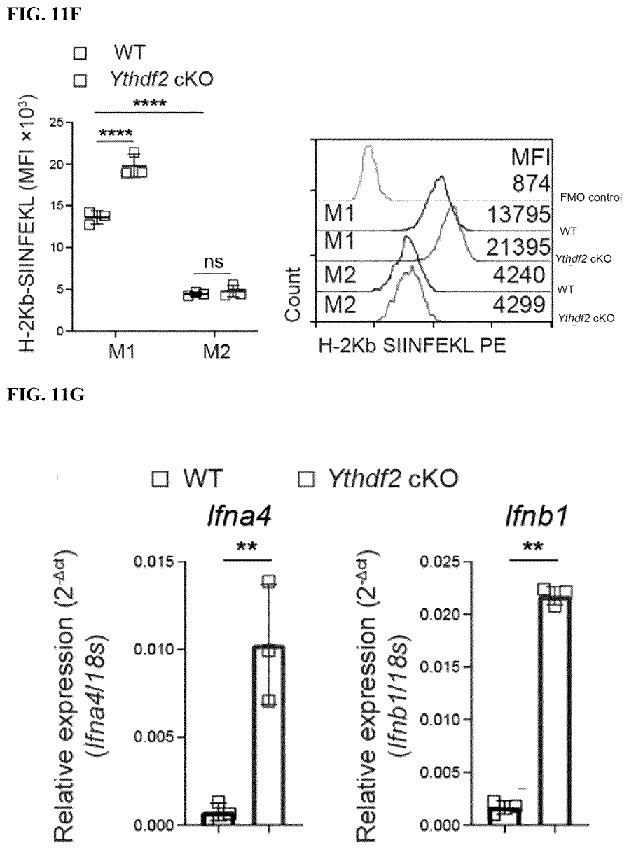

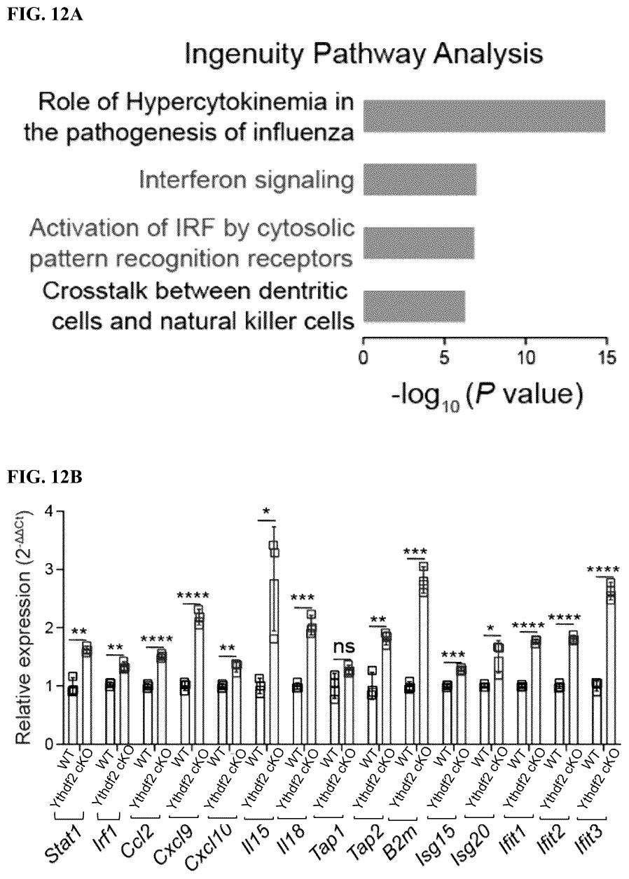

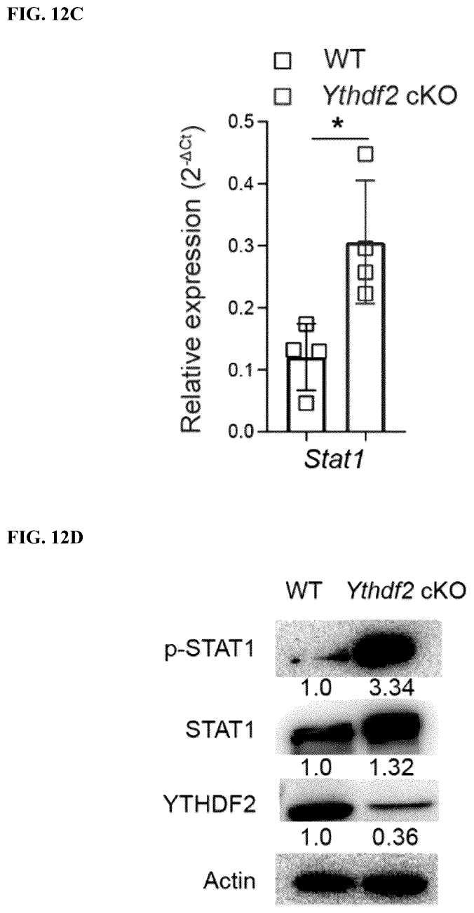

Signal transducer and activator of transcription 1 (STAT1) is downstream of the IFN-γ receptor signaling and nuclear factor κB (NF-κB) is downstream of the LPS/Toll-like receptor 4 (TLR4) signaling. Each contributes to the reprogramming of macrophages to the M1 type. (Ref 44). Ingenuity Pathway Analysis showed that the IFN signaling pathway and activation of IFN regulatory factor by cytosolic pattern recognition receptors were enriched in YTHDF2-deficient M1 macrophages (FIG. 12A). Gene set enrichment analysis (GSEA) in scRNA-seq data revealed that IFN-γ response genes, including transcripts encoding IFN-γ signaling transduction (Stat1 and Irf1), IFN-stimulated genes (Isg15, Isg20, Ifit1, Ifit2, and Ifit3), antigen presentation genes (B2m, Tap1, and Tapbp), and function genes (Cc12, Cxcl9, Cxcl10, Il15, and Il18), were enriched and significantly upregulated in M1 macrophages from Ythdf2 cKO mice when compared to WT mice (FIGS. 5A-5C). A qPCR assay confirmed that most of those genes were significantly upregulated in IFN-γ treated macrophages from Ythdf2 cKO mice compared to those from WT mice (FIG. 12B). In addition, TAMs isolated from tumors in Ythdf2 cKO mice showed higher levels of Stat1 mRNA and STAT1 phosphorylation when compared to those from tumors in WT mice (FIGS. 12C-12D). Moreover, we found that YTHDF2-deficient macrophages had higher STAT1 phosphorylation and responded quicker to IFN-γ stimulation than WT macrophages (FIG. 5D). Knocking down Stat1 by siRNA in YTHDF2-deficient BMDMs (FIG. 12E) abolished the expression difference of 1115 and Cxcl9, two important M1 macrophage signature genes downstream of IFN-γ, seen between WT and YTHDF2-deficient macrophages in vitro (FIG. 5E). We also knocked out Stat1 (Stat1^{-/-}) in both WT and YTHDF2-deficient BMDMs by CRISPR/Cas9 (FIG. 12F). Adoptive transfer of WT and YTHDF2-deficient BMDMs with knock-down of Stat1 by siRNA into B16-OVA-bearing mice eliminated the difference in tumor volume (FIG. 5F); identical results were obtained using the WT and YTHDF2-deficient Stat1^{-/-} BMDMs (FIG. 5G). Neutralizing IFN-γ in vivo or specific deletion of IFNGR1 in TAMs (FIG. 12G) also abrogated the difference in tumor volume (FIGS. 5H-5I). When we co-transferred BMDMs and CD8⁺ T cells from IFN-γ^{-/-} mice into Rag1^{-/-} mice, we observed similar levels of tumor growth in WT and Ythdf2 cKO mice (FIG. 12H). Collectively, these results indicate that YTHDF2 deficiency helps to reprogram macrophages into the M1 state by regulating the IFN-γ—STAT1 signaling pathway.

YTHDF2 Decreases the Stability of Stat1 mRNA in Macrophages

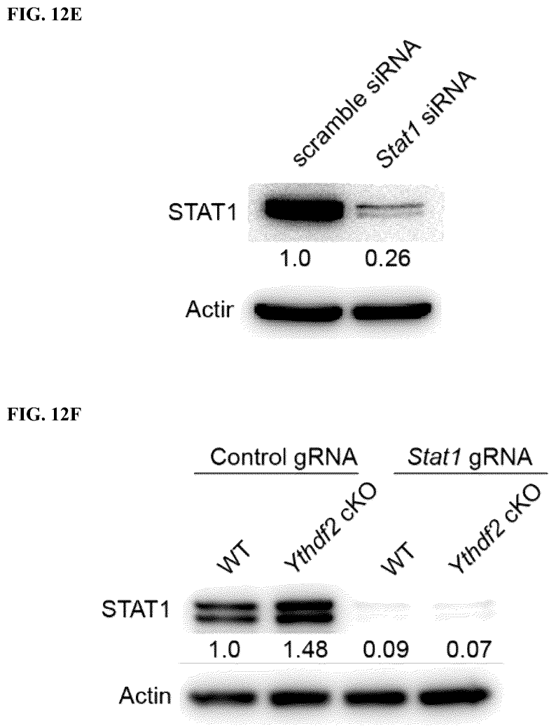

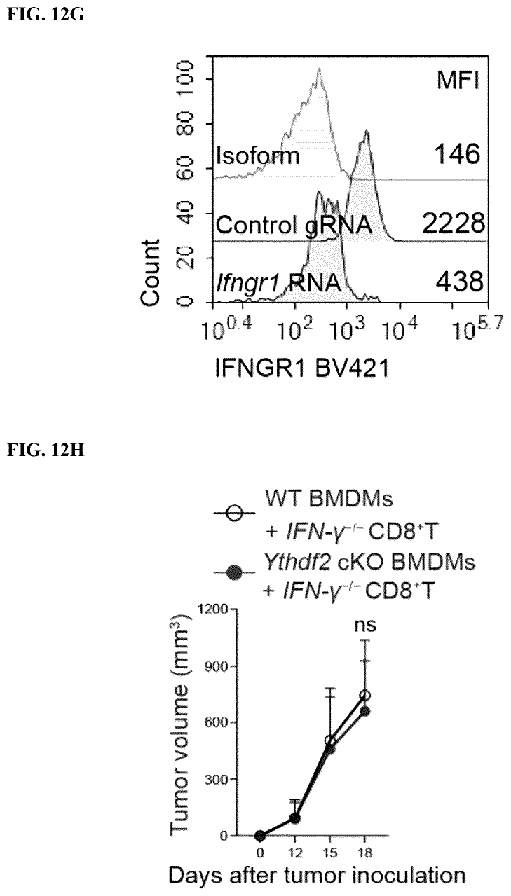

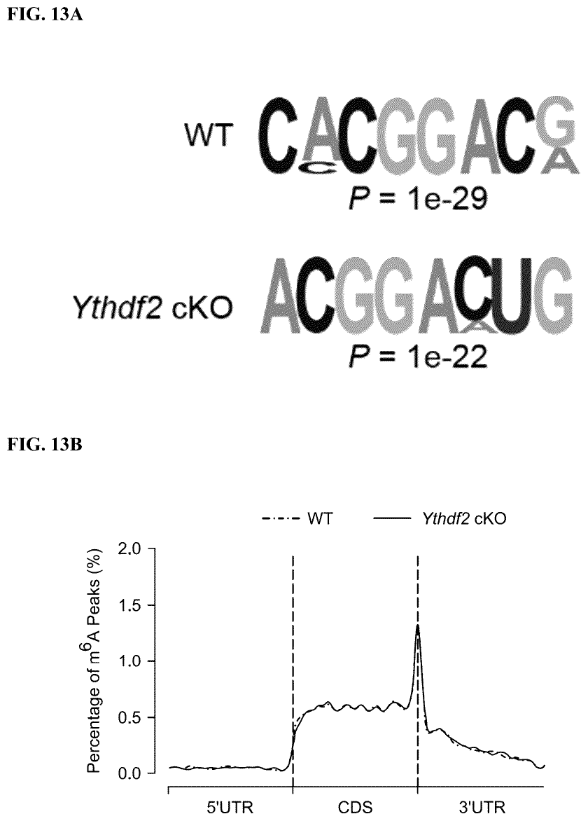

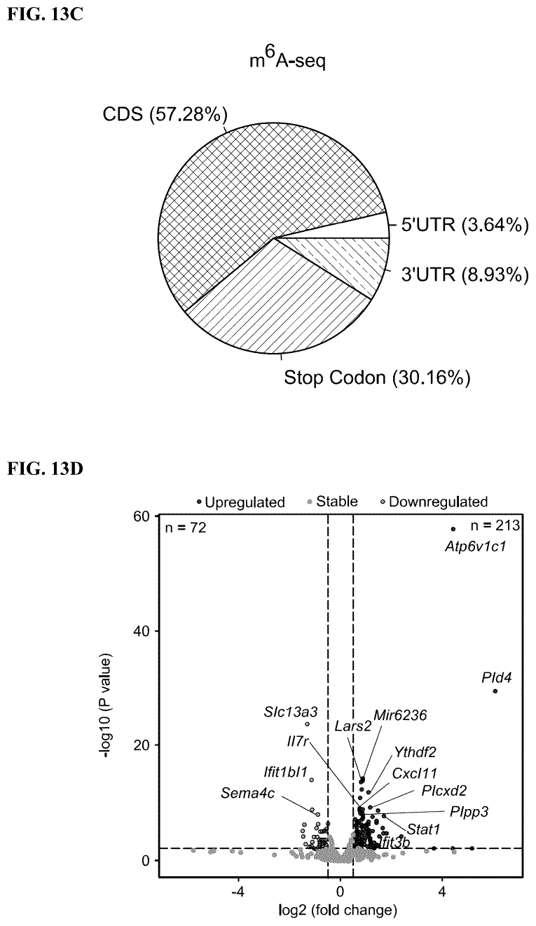

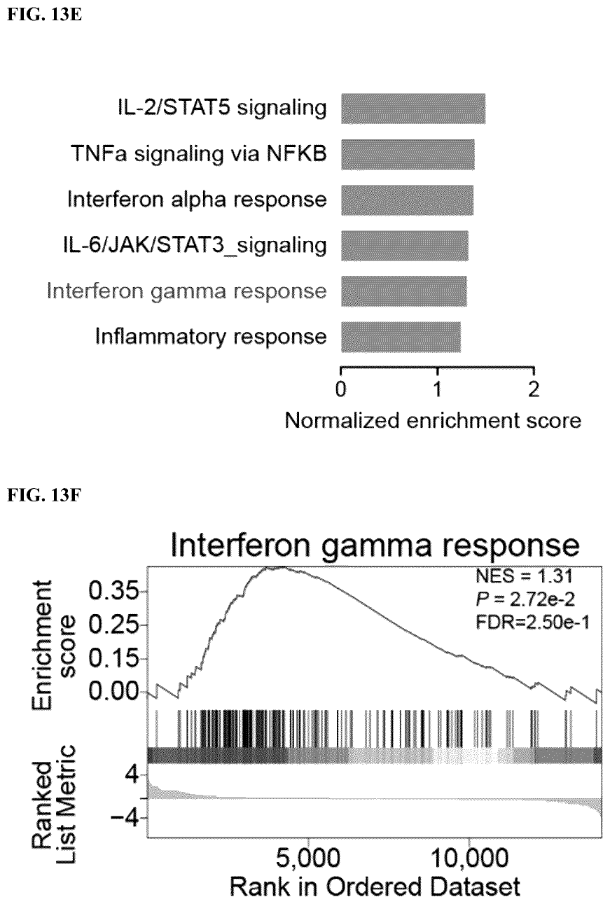

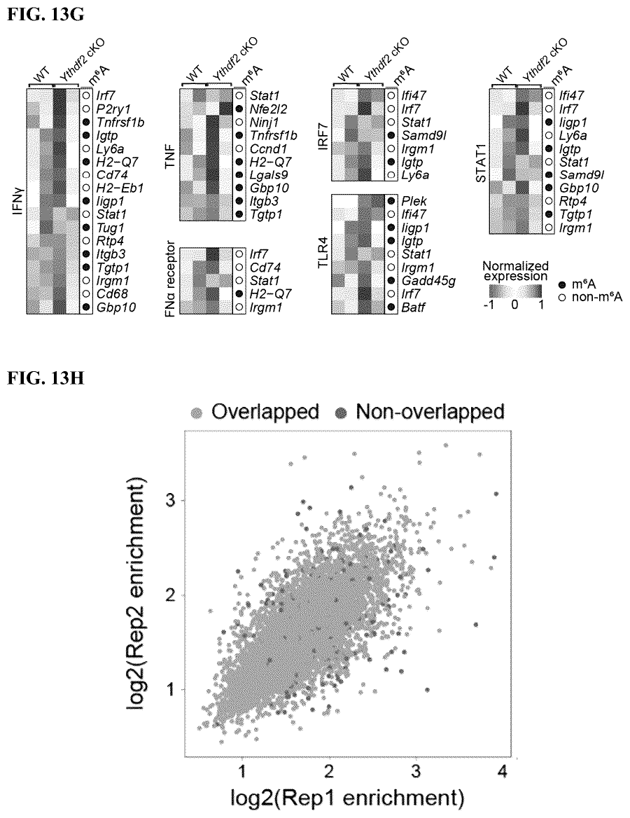

As YTHDF2 is known to induce the degradation of target mRNAs by reading m⁶A modification sites, we looked further into the molecular mechanism underlying reprogramming to M1 macrophages. (Ref 28). By m⁶A sequencing in M1 macrophages from WT and Ythdf2 cKO mice and then performing m⁶A peak calling and motif enrichment analysis, we identified the m⁶A consensus motif GGAC present in both strains of mice, indicating successful enrichment of m⁶A (FIG. 13A). The m⁶A modifications were mainly in protein-coding transcripts and were abundant in coding sequences (CDSs), 3′ untranslated regions (UTRs), and near stop codons (FIGS. 13B-13C). RNA-seq revealed 213 upregulated genes, including Stat1, and 72 downregulated genes in M1 macrophages from Ythdf2 cKO mice (FIG. 13D). GSEA showed significant enrichment of the IFN-γ response (FIGS. 13E-13F), which is consistent with our analysis from scRNA-seq.

A recent study demonstrated that YTHDF2-deficient hematopoietic stem cells (HSC) displayed activated proinflammatory pathways. (Ref 45). Given that proinflammatory signals are known to drive the activation and polarization of macrophages, we also evaluated the proinflammatory pathways regulated by YTHDF2 deficiency in macrophages. (Ref 46). YTHDF2^{-/-} deficient macrophages showed up-regulation of transcripts involved in multiple proinflammatory pathways, such as IFN-γ, TNF-α, IFN-α, IFN regulatory factor 7 (IRF7), STAT1, and Toll like receptor 4 (TLR4) (FIG. 13G). Moreover, a large proportion of these up-regulated transcripts were m⁶A modified (FIG. 13G). Our findings indicate that YTHDF2 is an essential inhibitor of inflammatory response exerted by macrophages.

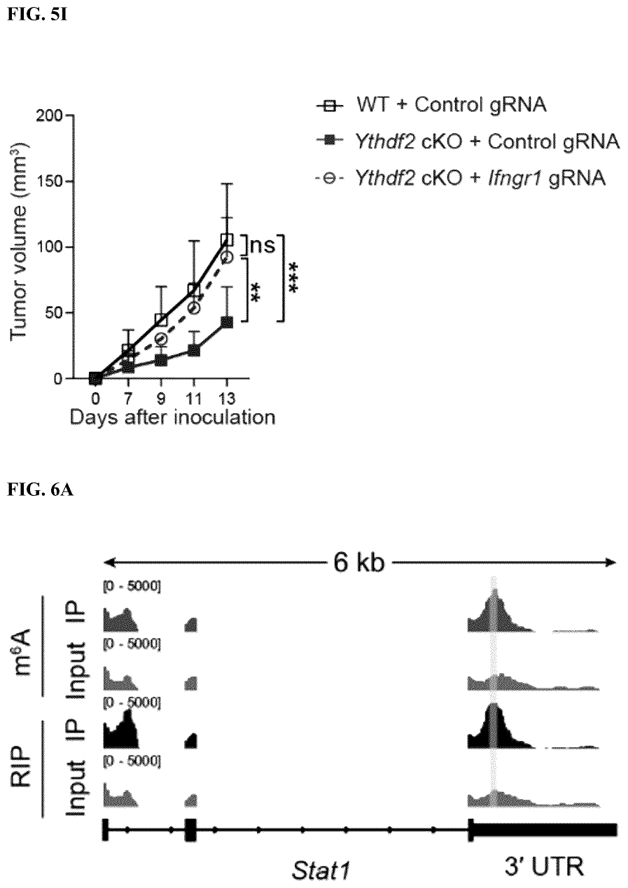

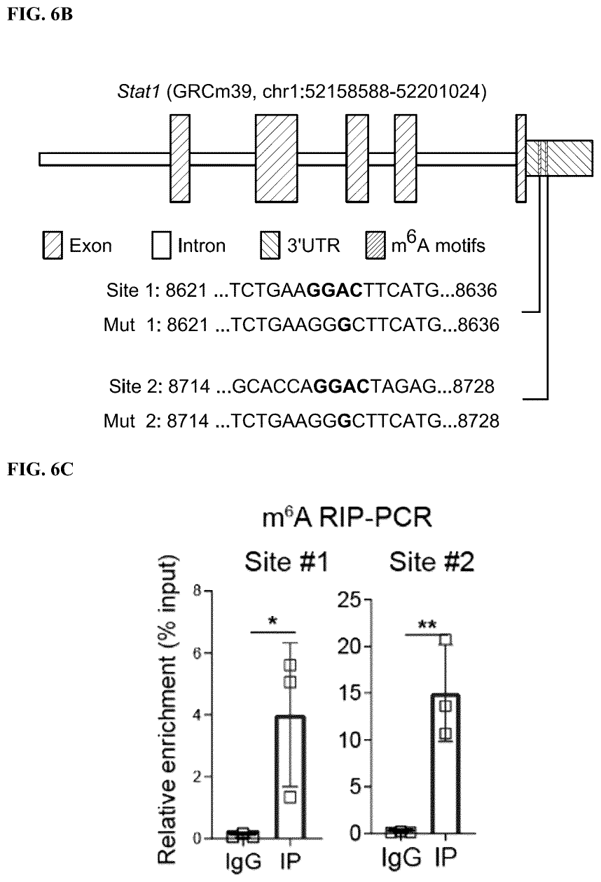

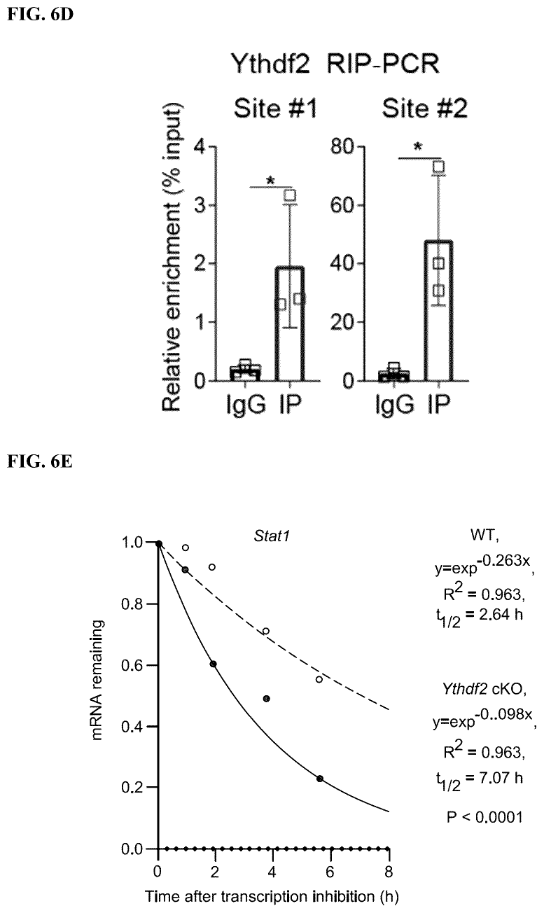

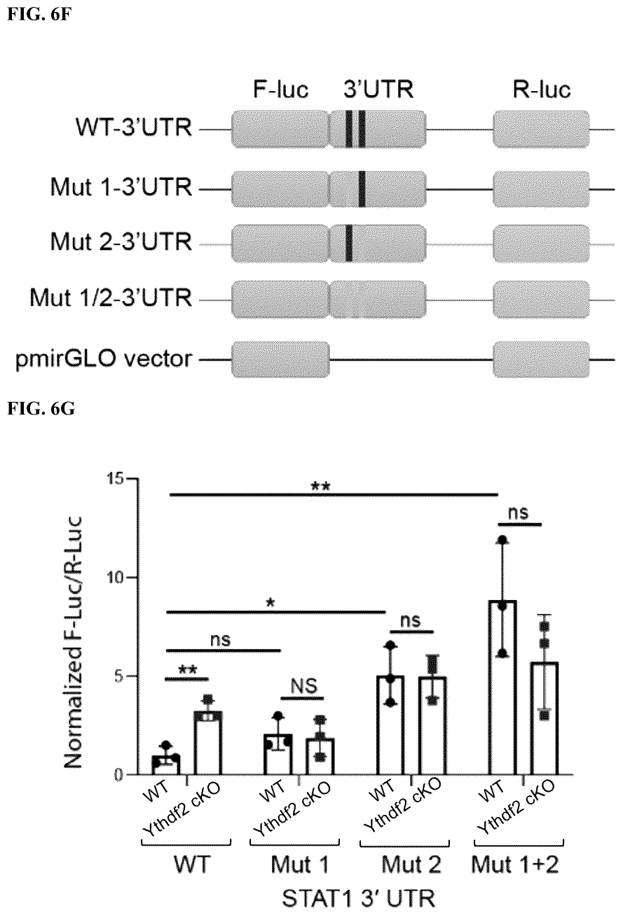

Using a YTHDF2 antibody to map direct target transcripts bound by YTHDF2 in M1 macrophages, we then performed RNA immunoprecipitation)-seq (RIP). The results were highly reproducible between two biological replicates (FIG. 13H). The YTHDF2-binding sites were predominantly in the coding region, the 3′UTR, and the stop codon (FIGS. 13I-13J). In line with our aforementioned functional characterization demonstrating that STAT1 is a potential target of YTHDF2, the Integrative Genomics Viewer revealed a good fit between the m⁶A peaks and the YTHDF2-binding sites in the 3′UTR of Stat1 (FIG. 6A). As there are two m⁶A modification sites within the 3′UTR of Stat1 (FIG. 6B), we performed qPCR and then RIP with either m⁶A or YTHDF2 antibody. This approach confirmed that two Stat1 sites in the 3′UTR region were indeed m⁶A methylated and were enriched, predominately by YTHDF2 in M1 macrophages (FIGS. 6C-6D). An mRNA stability assay showed that YTHDF2 deficiency significantly increased the half-life of Stat1 mRNA in M1 macrophages (FIG. 6E), indicating that STAT1 is directly regulated by YTHDF2 in M1 macrophages. To further explore the potential role of m⁶A methylation sites in Stat1expression, we constructed a firefly luciferase reporter construct containing WT Stat1m⁶A methylation sites, their mutants 1 or 2, or both (GGAC to GGGC for each site) at the 3′UTR (FIG. 6F). The 3′UTR-reporter dual-luciferase assay showed that the normalized luciferase activity in YTHDF2-deficient macrophages was significantly higher than that in WT macrophages (FIG. 6G). However, mutating either one or both m⁶A motifs abolished that difference (FIG. 6G), indicating that m⁶A methylation in the Stat1 3′UTR is responsible for the stability of Stat1 mRNA. Collectively, our findings indicate that YTHDF2 reprograms macrophages at least in part by reducing the stability of Stat1mRNA.

### YTHDF2 Expression is Regulated Through IL-10—STAT3 Signaling in TAMs

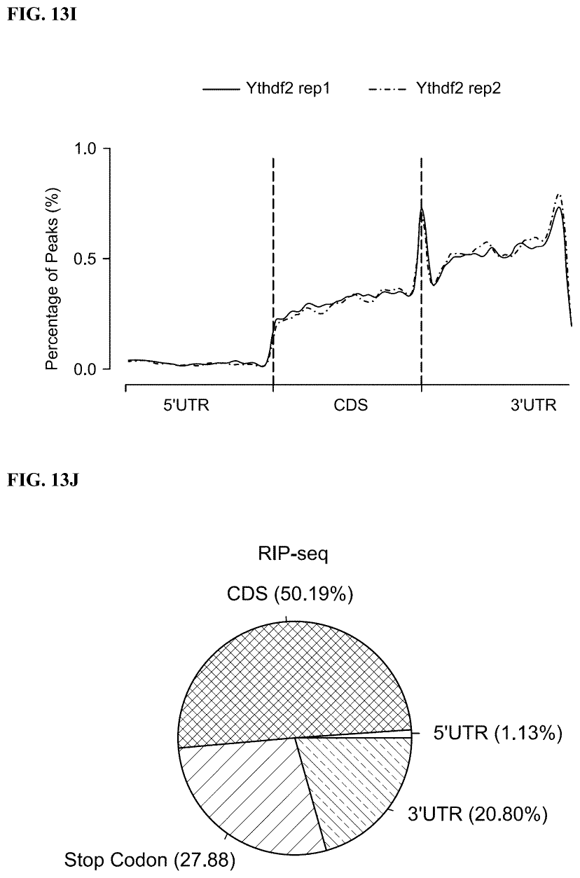

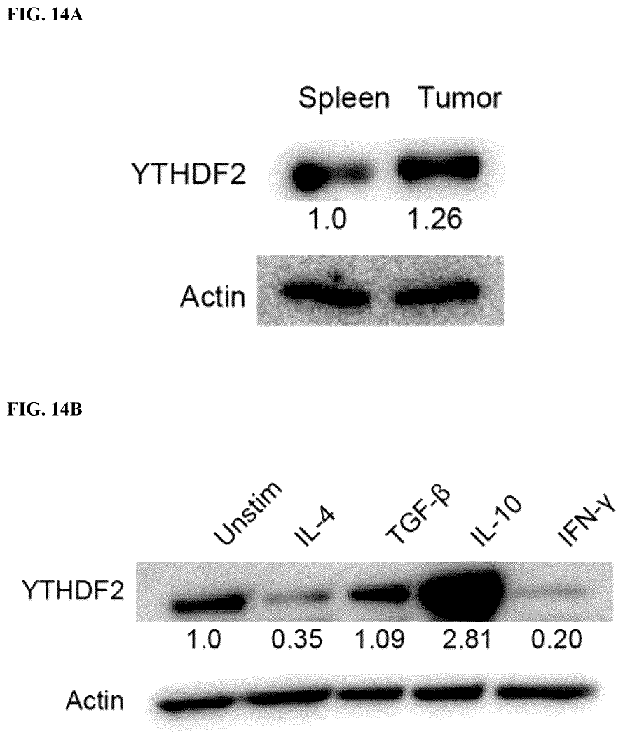

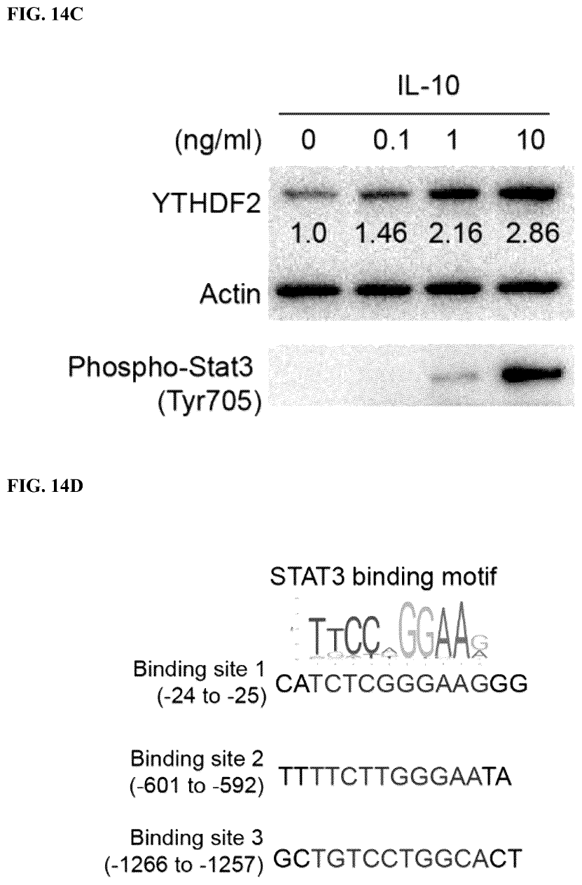

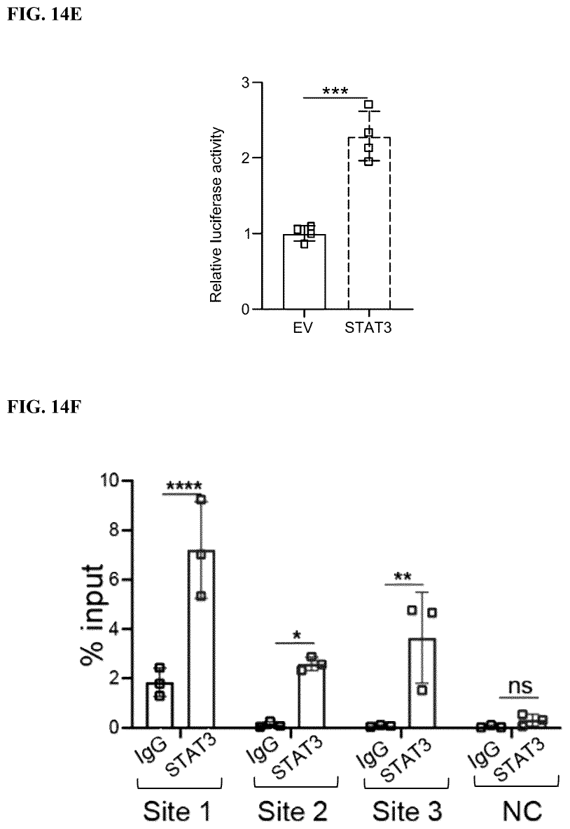

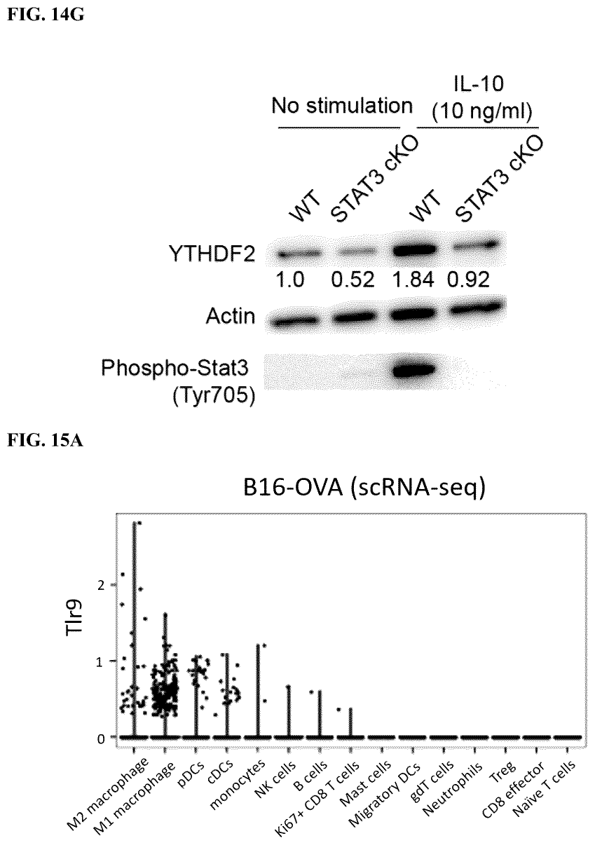

Above we show that YTHDF2 is a negative regulator of TAMs through IFN-γ—STAT1 signaling (FIGS. 5-6). However, the upstream mechanism by which TME regulates YTHDF2 expression in TAMs in the context of the TME remains undetermined. We found that macrophages isolated from MC38 TME had higher levels of YTHDF2 compared to those isolated from the spleen of tumor-bearing mice (FIG. 14A). The polarization process of TAMs is directly controlled by cytokines within the TME, including IFN-γ, IL-4, IL-10, and TGF-β¹². We then examined the protein levels of YTHDF2 in BMDMs upon stimulation with the aforementioned cytokines. The results showed that IL-10 had the strongest effect on the induction of YTHDF2 expression compared to other cytokines, such as IL-4, TGF-β, and IFN-γ (FIG. 14B). Moreover, IL-10 induced YTHDF2 expression in BMDMs in a dose-dependent manner (FIG. 14C). The binding of IL-10 to the IL-10 receptor results in the activation of the JAK1/STAT3 cascade⁴⁷. We thus analyzed the ENCODE chromatin immunoprecipitation sequencing (ChIP-seq) data in ChIPBase and the Animal Transcription Factor DataBase to identify the putative transcription factors (TFs) that directly regulate YTHDF2 expression (AnimalTFDB)^{48,49} Remarkably, the analysis of the two databases showed that STAT3 was 2^{nd} on the TF on the list of 24 overlapping common TFs that bind to YTHDF2 (FIG. 21). Of note, three binding sites for STAT3 in the promoter regions of the mouse Ythdf2 gene were predicted by JASPAR (http://jaspar.genereg.net) (FIG. 14D). A luciferase reporter assay showed that STAT3 directly activated Ythdf2 gene transcription in BMDMs (FIG. 14E). ChIP-qPCR showed that STAT3 has significant enrichment on three sites over normal IgG control (FIG. 14F), indicating STAT3 binds directly to the Ythdf2 gene promoter in IL-10-stimulated BMDMs. To further confirm that YTHDF2 is a downstream factor regulated by STAT3, we used Stat3^{fl/fl}/Lyz2^{Cre} mice (hereafter referred to as Stat3 cKO mice)⁵⁰. We treated BMDMs from WT and Stat3 cKO mice with IL-10 and found that YTHDF2 was significantly decreased in BMDMs from Stat3 cKO mice compared to those from WT mice (FIG. 14G). Collectively, our findings indicate that YTHDF2 expression in TAMs is regulated at least in part through IL-10—STAT3 signaling.

TLR9-Targeted YTHDF2 Silencing in TAMs Suppresses Tumor Growth and Synergizes with Anti-PD-L1 Blockade

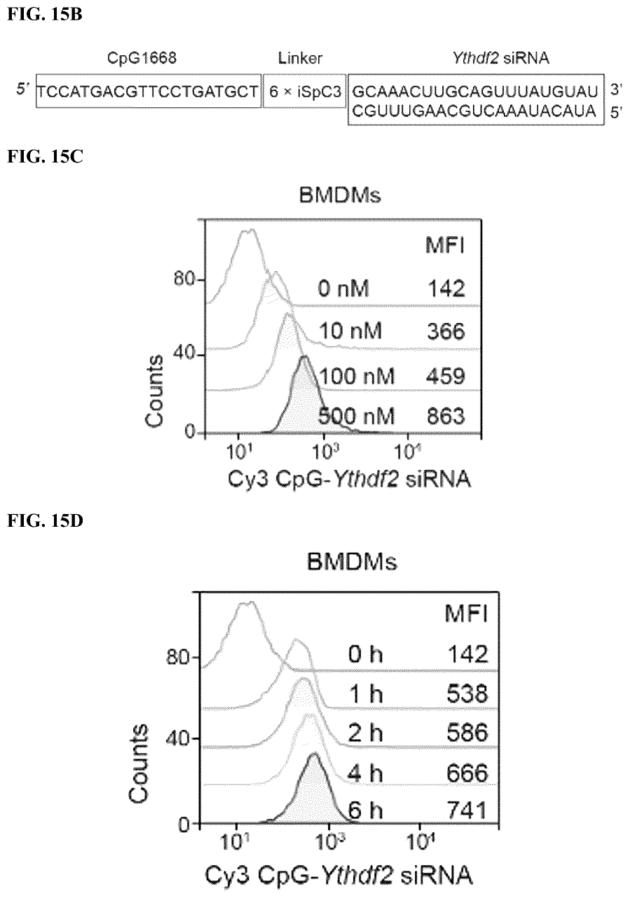

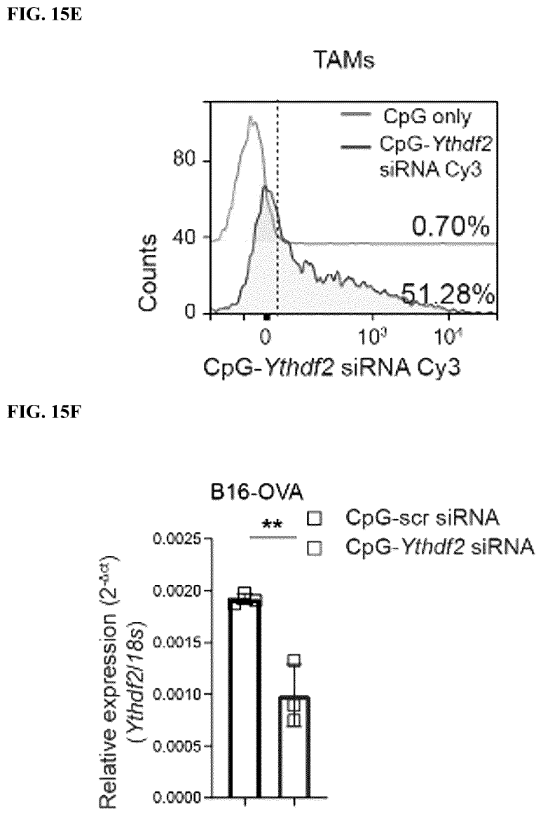

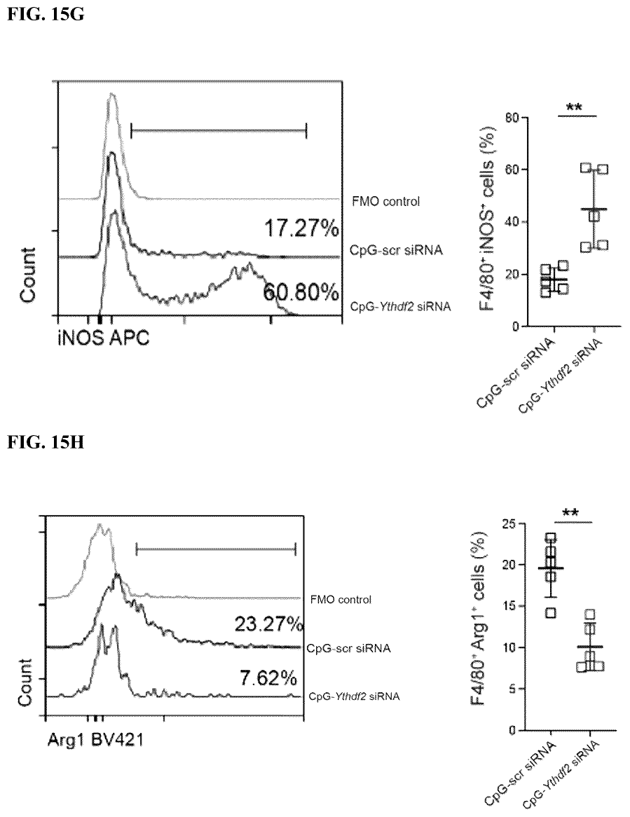

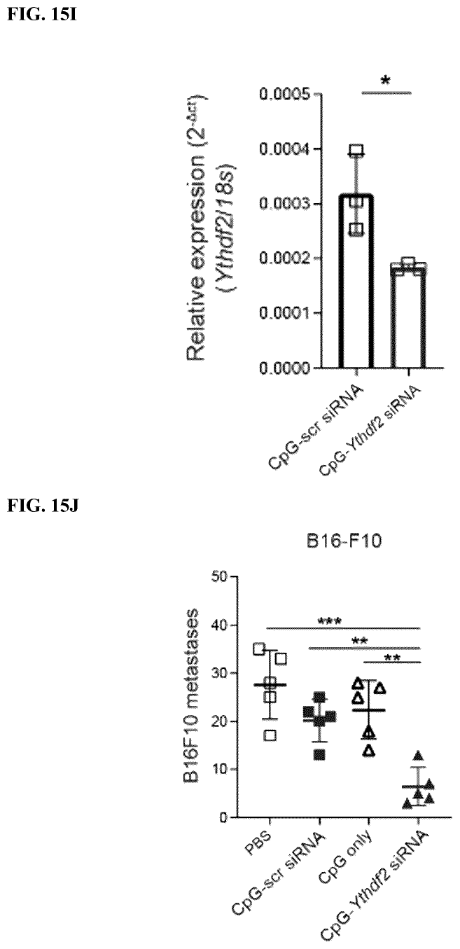

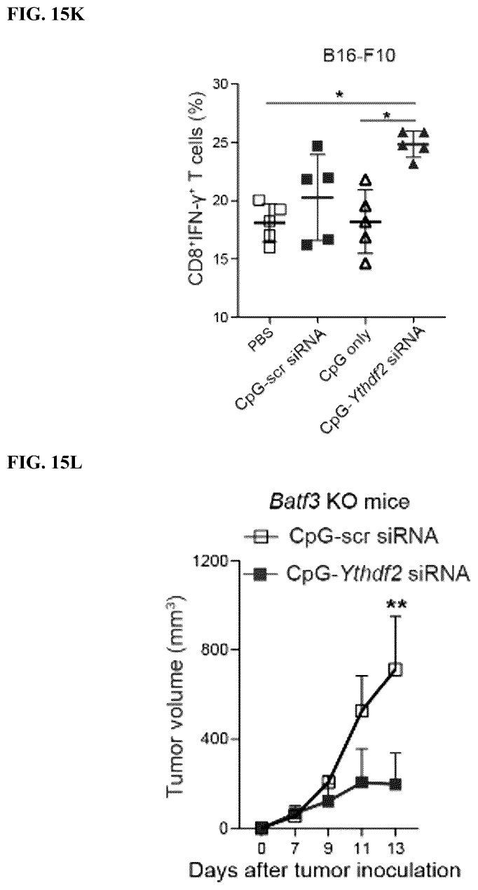

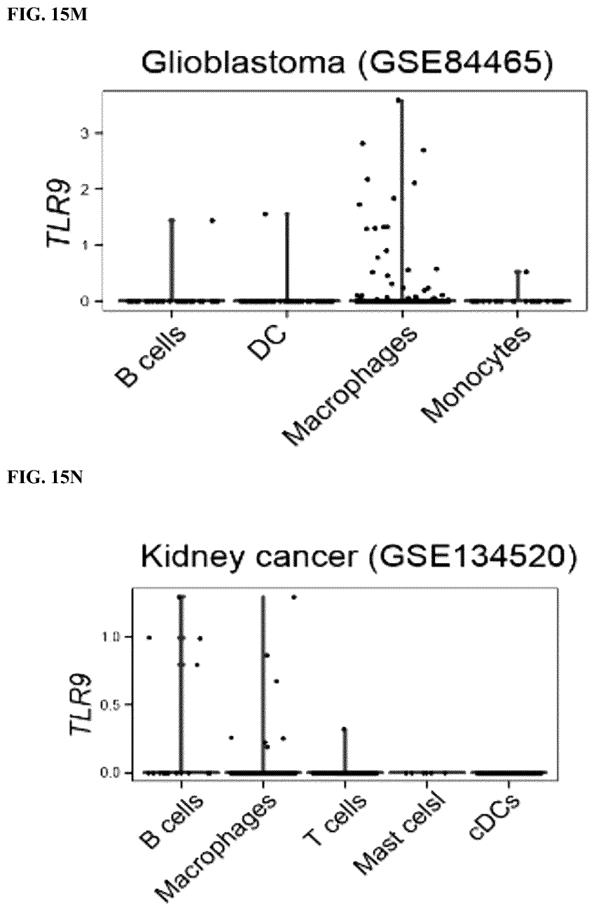

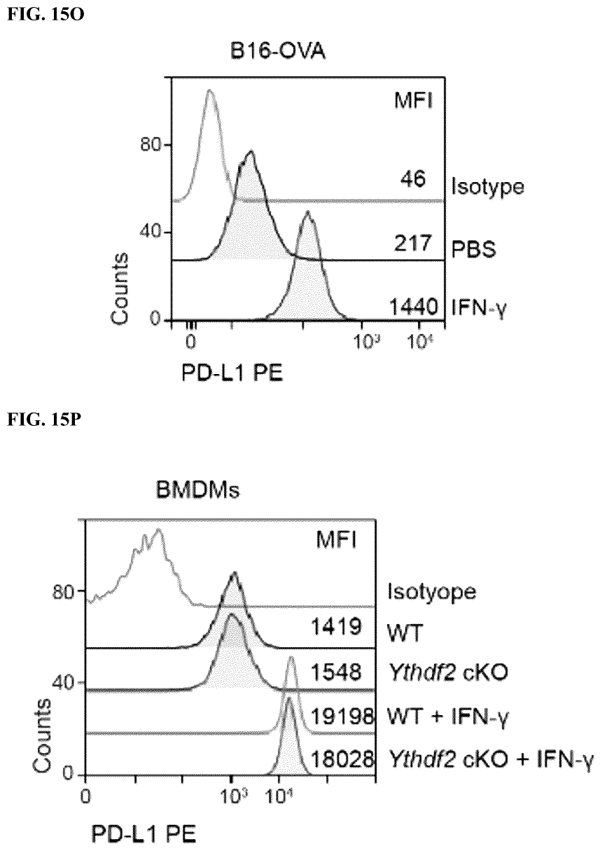

Our study demonstrates that YTHDF2 deficiency reprograms TAM polarization to the M1 phenotype, thereby strengthening anti-tumor immunity. This unique property provides a strong rationale for targeting YTHDF2 for cancer treatment or prevention of cancer recurrence. Although as of yet there are no small-molecule YTHDF2 inhibitors, gene silencing using oligonucleotide-based therapeutics (ONTs) such as siRNA, antisense oligonucleotides, or decoy ODN are showing promise against cancers and other diseases⁵¹. Our team previously generated oligonucleotide-based strategies for cell-selective STAT3 targeting in vivo⁵². By conjugating Stat3 siRNA to synthetic TLR9 agonists CpG ODN, we selectively delivered them into TLR9-positive cells, such as TAMs⁵². We have shown promising therapeutic effects in acute myeloid leukemia, B cell lymphoma, and certain solid tumors⁵³⁻⁶⁰ Therefore, we evaluated whether TLR9-targeted Ythdf2 silencing could reprogram TAMs, promote anti-tumor immunity, and suppress tumor growth. From our scRNA-seq data, we found that Tlr9 is mainly expressed by M2 macrophages within the TME (FIG. 15A). We then conjugated Ythdf2 siRNA to a TLR9 agonist (single-stranded CpG1668 ODN), using a synthetic carbon linker⁵² (FIG. 15B). In vitro and in vivo uptake assays showed that macrophages could efficiently take up Cy3-labeled-CpG-Ythdf2 siRNA (FIGS. 15C-15E). Moreover, qPCR analysis of sorted TAMs showed that mRNA levels of Ythdf2 were significantly lower in the CpG-Ythdf2 siRNA group than in the CpG-scrambled siRNA group (FIG. 15F). Intra-tumoral treatment with CpG-Ythdf2 siRNA significantly up-regulated the proportion of M1 TAMs and down-regulated that of M2 TAMs compared to treatment with CpG-scrambled siRNA (FIGS. 15G-15H).

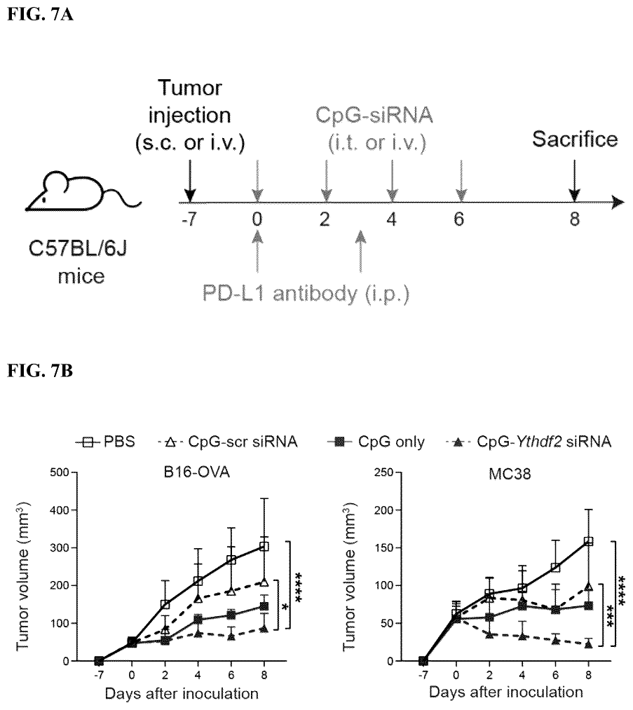

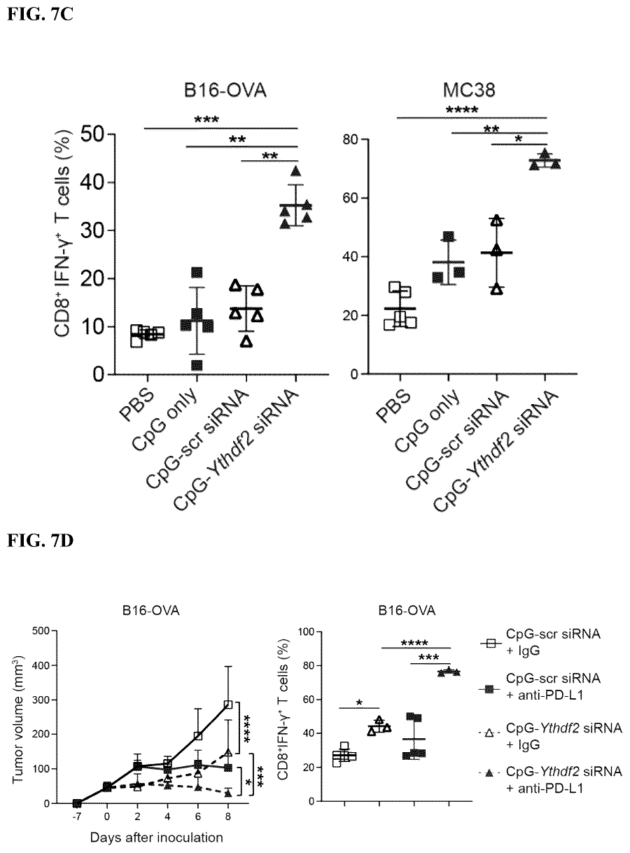

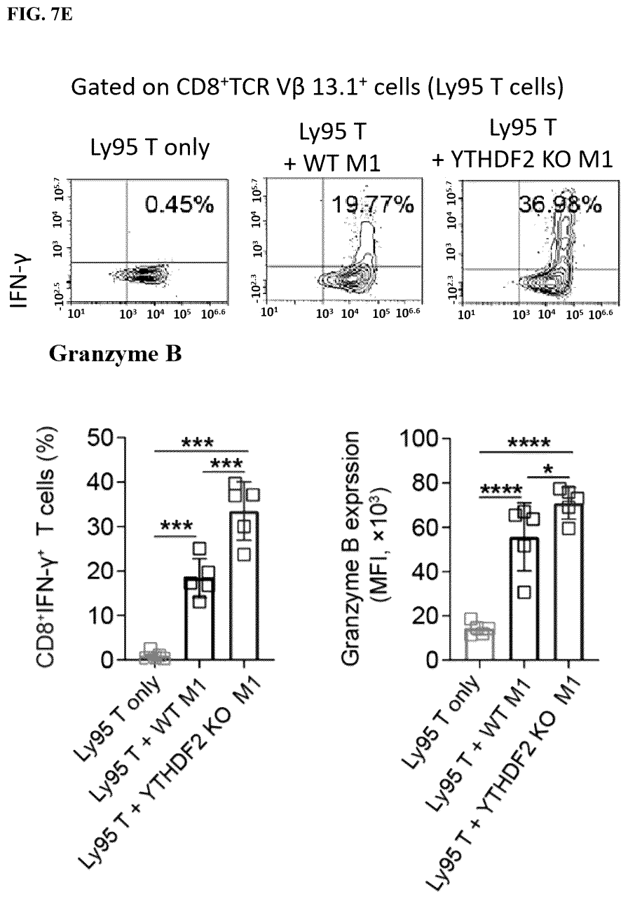

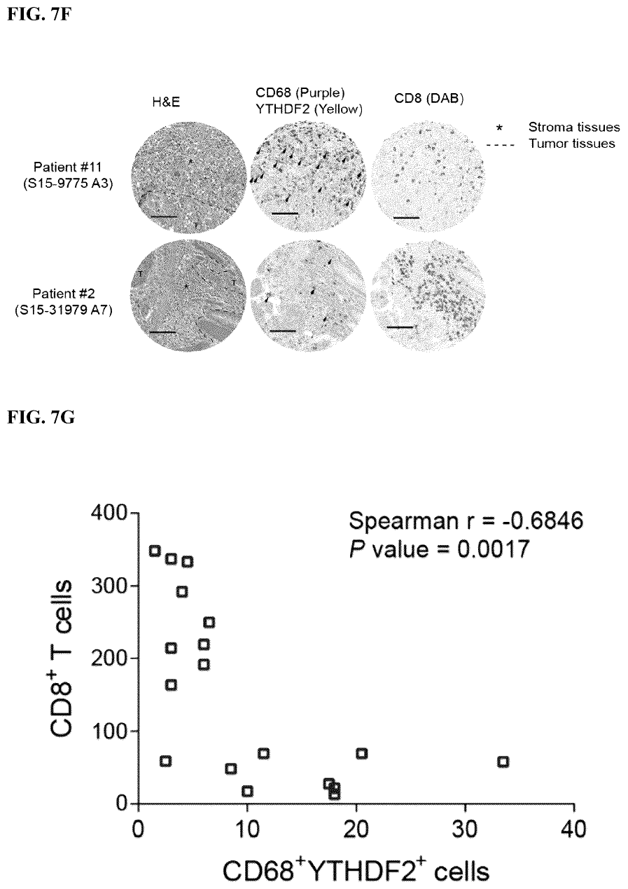

We then established B16-OVA and MC38 tumor models to determine how intra-tumoral delivery of CpG-Ythdf2 siRNA would affect tumor growth (FIG. 7A). As expected, it significantly inhibited tumor growth compared to intra-tumoral delivery of CpG-scrambled siRNA in two tumor models (FIG. 7B). We also investigated the possibility that systemic administration of CpG-Ythdf2 siRNA could have some anti-tumor effects. Intravenous injection of CpG-Ythdf2 siRNA inhibited tumor metastasis in an experimental B16F10 lung metastasis model when compared to systemic delivery of CpG-scrambled siRNA (FIGS. 15I-15J). In addition, there was a significantly higher percentage of IFN-γ-producing CD8⁺ T cells in CpG-Ythdf2 siRNA-treated mice compared to the CpG-scrambled siRNA group in all three tumor models (FIG. 7C and (FIG. 15K). DCs express TLR9 (FIG. 15A) and can be activated by CpG⁶¹. To determine whether the anti-tumor effect of CpG-Ythdf2 siRNA was dependent on DCs, we established the B16-OVA tumor model in the background of Batf3 KO mice, which lack cDC1 cells⁶². We found that CpG-Ythdf2 siRNA still inhibited tumor growth compared to CpG-scrambled siRNA without cDC1 cells (FIG. 15L), which excludes a contribution of the anti-tumor effect from DCs. Moreover, leveraging scRNA-seq data sets, we discovered a relatively high expression of TLR9 in TAMs in some cancers (FIGS. 15M-15N). Collectively, these results indicate that silencing YTHDF2 by targeted delivery of CpG-Ythdf2 siRNA to TAMs via TLR9 promotes anti-tumor immunity and inhibits tumor growth.

In recent years, immune checkpoint inhibitors (ICIs) have been widely used in tumor immunotherapy⁴. Our findings show that TAMs with deficient or silenced YTHDF2 can turn “cold” tumors into “hot” tumors by improving the infiltration of IFN-γ producing CD8⁺ T cells. Continuous IFN-γ stimulation can upregulate PD-L1 in cancer cells and macrophages, thereby inactivating anti-tumor T cell responses^{63, 64}. After confirming that PD-L1 expression was significantly upregulated in B16-OVA cells and BMDMs treated with IFN-γ (FIGS. 15O-15P), we treated B16-OVA tumor-bearing mice with CpG-Ythdf2 siRNA in combination with an anti-PD-L1 antibody. The combination therapy showed a better anti-tumor effect, as documented by reduced tumor growth and increased CD8⁺ cytotoxic T cell responses compared to CpG-Ythdf2 siRNA alone or anti-PD-L1 mAb alone (FIG. 7D). These data indicate that delivery of CpG-Ythdf2 siRNA in combination with a PD-L1 blockade might improve outcomes in patients who respond poorly to a checkpoint blockade.

### YTHDF2 Abrogation Improves Anti-Tumor CD8⁺ T Cell Responses in Human Macrophages

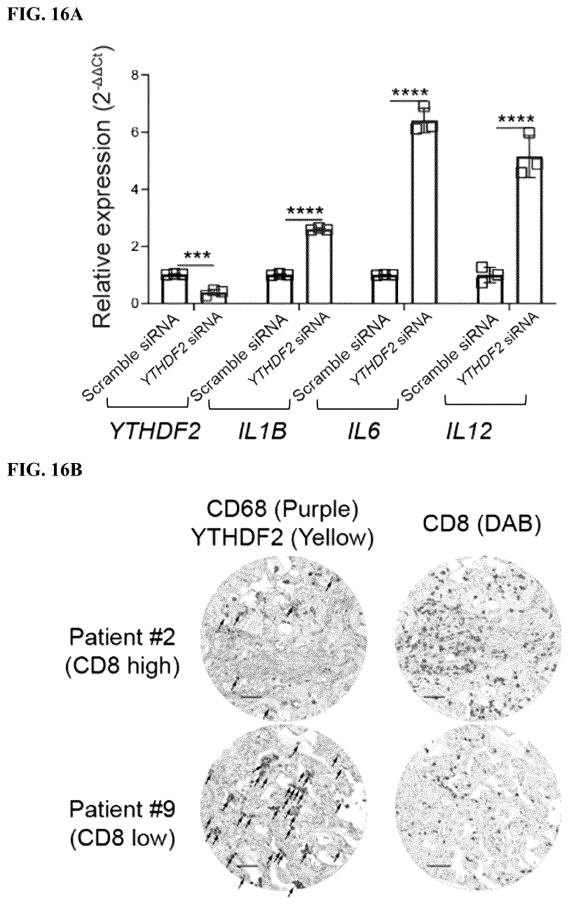

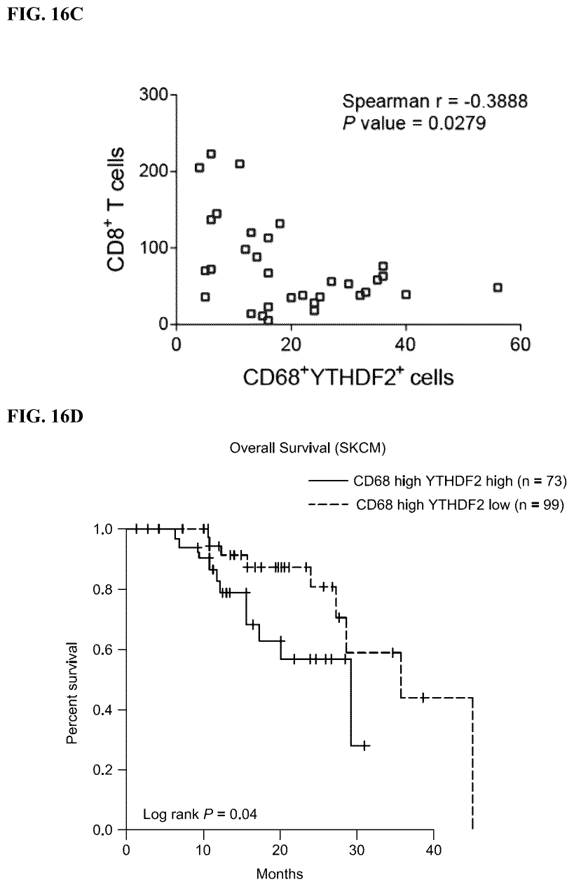

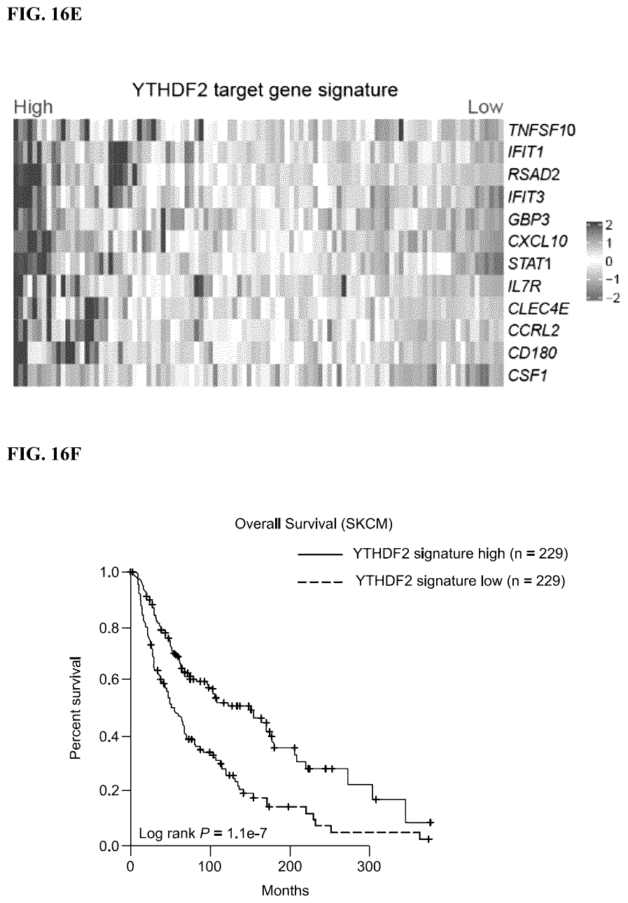

In agreement with our findings in mouse macrophages, knock-down of YTHDF2 using siRNA in human macrophages also enhances the expression of pro-inflammatory cytokines (FIG. 16A). To test the ability of YTHDF2-deficient human macrophages to stimulate tumor-specific T cell responses, we used a recently developed TCR-transduced human T cells (Ly95 T cells) recognizing the HLA-A*0201-restricted human testis cancer antigen New York esophageal squamous cell carcinoma 1 (NY-ESO-1) system^{65,66} Incubating Ly95 T cells with YTHDF2 knock-down macrophages preloaded with a NY-ESO₁₅₇₋₁₆₅ peptide had significantly increased IFN-γ and granzyme B production in Ly95 T cells compared to those co-cultured with peptide-preloaded WT macrophages (FIG. 7E). These data indicate that YTHDF2 exerts the same function in human macrophages as we found in mice.

Reduced YTHDF2 Expression in TAMs and Upregulated YTHDF2 Target Gene Signatures are Associated with Improved Overall Survival in Cancer Patients

To determine whether our experimental observations can translate to human patients with cancer, we examined the protein level of YTHDF2 through immunohistochemical staining of tumor tissues from patients with colon cancer or lung cancer. We found that the number of YTHDF2⁺CD68⁺ macrophages in the tumor stroma tissues inversely correlated to the number of tumor-infiltrated CD8⁺ T cells in these patients (FIGS. 7G-7G; FIGS. 16B-16C), indicating that reduced YTHDF2⁺CD68⁺ macrophages correlate with increased CD8⁺ T cell responses in the TME in human cancers. In agreement with this, the analysis of patient samples derived from SKCM from TCGA revealed an association of lower YTHDF2 expression in patient tumors with high CD68⁺ macrophage infiltration that correlates with improved overall survival (OS) in SKCM (FIG. 16D). Based on our sequencing data from the mouse, we generated a YTHDF2 target signature containing 12 genes (FIG. 16E). High YTHDF2 target gene signature correlated with better OS compared to a low YTHDF2 target gene signature in SKCM patients (FIG. 16F). Collectively, our findings demonstrate that YTHDF2 in human macrophages could be a therapeutic target for cancer immunotherapy.

### DISCUSSION

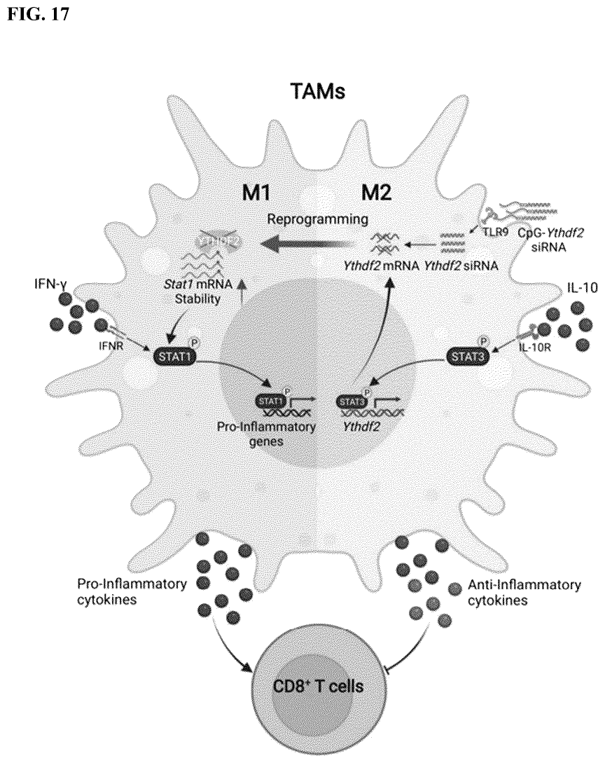

As TAMs have an important function in tumor immunity and their infiltration into the TME associates with poor prognosis in most cancers⁶⁷, there have been extensive attempts to target these innate immune cells for improvement of cancer immunotherapy. These efforts include inhibition of macrophage recruitment into the TME, reprogramming TAMs to an anti-tumor M1-like type, and eliciting macrophage-mediated phagocytosis⁶⁸. However, TAMs contribute to both anti- and pro-tumoral activities. Therefore, strategies to reprogram rather than ablate TAMs may harness their pro-tumoral properties⁶⁸. Indeed, depletion of both monocytes and macrophages by colony-stimulating factor 1 receptor inhibitors has shown substantial toxicity over time in the clinic⁶⁹ Currently, several strategies, including our team's, which aim to reprogram TAMs are in preclinical or clinical trials. Potential therapeutic agents include anti-CD47 antibodies, anti-CD40 antibodies, Toll-like receptor agonists, histone deacetylase inhibitors, and PI3Kγ inhibitors^{67, 70, 71}. Here we have identified YTHDF2 as a novel negative regulator of TAMs. Specifically, we demonstrated that genetic deletion of YTHDF2 induces M1-type polarization in macrophages, thereby enhancing CD8⁺ T cell-mediated anti-tumor immunity (FIG. 17). Based on our findings, we developed a strategy for targeting YTHDF2 specifically in TAMs by CpG-siRNA entering via TLR9. Our CpG-conjugated-Ythdf2 siRNA approach showed profound anti-tumor effects. Our study supports the development of a CpG-Ythdf2 siRNA clinical strategy alone for in situ immunotherapy against cancer or its combination with anti-PD-L1 therapy.

YTHDF2 is one of the most important m⁶A readers that acts by specifically recognizing and binding to m⁶A-containing RNAs and promoting degradation of target transcripts²⁸. We previously reported that YTHDF2 plays multifaceted roles in NK cell immunity³⁶. YTHDF2 positively regulates NK cell homeostasis, maturation, IL-15-mediated survival, as well as anti-tumor and anti-viral activity³⁶. However, the role of YTHDF2 in macrophages and how its deficiency in this one cell type impacts other immune cells have largely been unknown. Therefore, we generated three different mouse tumor models and used human cells to address these two issues. Surprisingly, we found that YTHDF2 deficiency in macrophages has effects opposite to its deficiency in NK cells, indicating that YTHDF2 has different functions in different types of immune cells. The differential roles of YTHDF2 in macrophages and NK cells may not affect the therapeutic role of CpG-conjugated-siRNA that we developed, as CpG-conjugated-siRNA can reach the cytoplasm of macrophages, while NK cells do not uptake it⁵². Knockdown of YTHDF2 in tumor cells identifies its intrinsic oncogenic role in tumor progression^{33,72-75}. However, how directly targeting tumor cells change immune responses in the TME and the role of YTHDF2 in other immune cells such as DCs and T cells are largely unknown. Our study provides proof-of-concept evidence that appropriately targeting YTHDF2 can reshape the TME to be more inflammatory for tumor eradication, while further studies of this protein in other cells in the TME can further optimize the therapeutic effect of targeting YTHDF2.

As a new regulatory layer of gene expression in post-transcriptional levels, RNA m⁶A methylation has been shown to affect multiple aspects of the mRNA metabolism, including but not limited to RNA structure, splicing, translation, and decay⁷⁶. Several studies have provided proof-of-concept evidence that small-molecule inhibitors targeting m⁶A modifiers, such as FTO, ALKBH5, and METTL3, represent a promising strategy for cancer therapy^{20, 21, 77-79}. However, inhibitors that target readers, including YTHDF2, have not been discovered. One possible reason is that the readers may share the same or overlapping binding sites of the target gene but exert opposite functions, which can result in beneficial and detrimental effects simultaneously. To overcome this challenge, we provided an alternative direction in this study by using cell-specific siRNA conjugates. This approach can specifically silence the m⁶A modified in a cell of interest in vivo in an efficient manner, which may be a promising therapeutic strategy for future cancer treatment.

ICT has revolutionized cancer treatment and improved the survival of patients in the clinic. However, patients can quickly develop resistance and such that only approximately 30% of patients benefit from ICT^{3, 64,80} Combinational therapies usually result in better outcomes than monotherapies, and this should be the case for checkpoint blockade-based cancer immunotherapy 9. Elucidation of a novel epigenetic mechanism, e.g., m⁶A modifications, in tumor cells and immune cells may help to better understand the immune resistance mechanisms of ICT and provide us with superior opportunities for combinational therapies²⁶. In the current study, we indeed observed that targeting the m⁶A reader YTHDF2 in TAMs enhances both innate and adaptive immune responses to tumor cells, which reverses the immunosuppressive TME. Further optimizing this combination therapy may overcome the resistance of ICT and increase the survival of patients treated with the inhibitors.

In summary, we identified YTHDF2 as a novel negative regulator of TAMs. We then developed an innovative reagent, CpG-conjugated-Ythdf2 siRNA, for targeting YTHDF2 specifically in TAMs via TLR9. Our approach produced profound anti-tumor effects. Taken together, our data uncover a previously unappreciated mechanism by which YTHDF2 in TAMs sabotages anti-tumor immunity. It also supports the translation of our CpG-Ythdf2 siRNA strategy into the clinic for treatment against cancer, especially in combination with anti-PD-L1 immunotherapy.

### Methods

Cell lines. The murine melanoma cell line B16F10 was provided by Professor Hua Yu (City of Hope National Medical Center). B16-OVA cells were provided by Professor Marcin Kortylewski (City of Hope National Medical Center). The murine colon adenocarcinoma cell line MC38 was provided by Professor Saul Priceman (City of Hope National Medical Center). All cell lines were cultured in Dulbecco's modified Eagle medium (DMEM, Gibco) supplemented with 10% fetal bovine serum (FBS, Gibco). Cells were incubated at 37° C. with 5% CO₂.

Tumor models and treatments. B16-OVA (2×10⁵) or MC38 cells (2×10⁵) were implanted subcutaneously (s.c.) into the flank of mice. The length (a) and width (b) of the tumors were measured starting on day 7 or 9 and every three days thereafter, and the tumor volume was calculated with the formula a×b²/2. For the lung metastatic melanoma model, B16F10 cells (1×10⁵) were injected intravenously (i.v.) into the mice. Fourteen days after injection, mice were euthanized for post-mortem analysis. Metastatic nodules in the lung were analyzed macroscopically and counted. For CD8⁺ T cell depletion, mice were injected intraperitoneally (i.p.) with 200 g of anti-CD8 antibody (clone 2.43, BioXcell) on days 0 and 7 after tumor inoculation. For macrophage depletion, mice were injected i.p. with 200 μL of liposomal clodronate (Encapsula NanoSciences) on days 0 and 7 after tumor inoculation. To neutralize IFN-γ in vivo, mice were injected i.p. with 200 μg of anti-IFN-γ antibody (clone XMG1.2, BioXcell) on days 0 and 7 after tumor inoculation. To co-transfer with macrophages, B16-OVA (2×10⁵) or MC38 cells (2×10⁵) were mixed with an equal number of bone marrow-derived macrophages (BMDMs) from WT or Ythdf2-cKO mice and implanted s.c. into the flank of WT mice, or Rag1^{-/-} mice injected i.v. without or with CD3⁺ T cells (5×10⁶). To knock down Stat1 using siRNA, BMDMs were transfected with siGENOME mouse Stat1 siRNA (Catalog: M-058881-02-0005, Horizon) at 100 nM using lipofectamine 3000 (Invitrogen). 48 h later, cells were collected and mixed with an equal number of B16-OVA cells (2×10⁵) and implanted s.c. into the flank of WT mice.

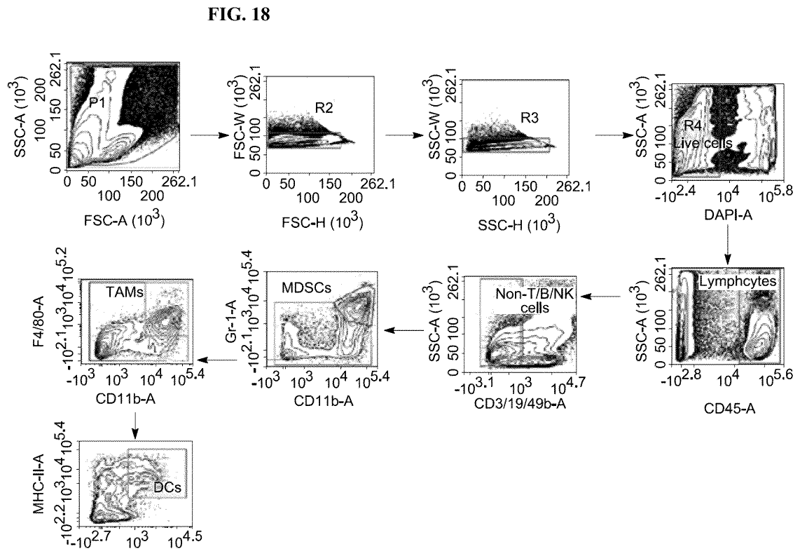

Flow cytometry. Single-cell suspensions were prepared from the spleen and tumor tissues of WT and Ythdf2 cKO mice as described previously³⁶. Flow cytometry analysis and cell sorting were performed on a BD LSRFortessa X-20 and FACSAria Fusion Flow Cytometer (BD Biosciences), respectively. Data were analyzed using NovoExpress software (Agilent Technologies). The following fluorescence dye-labeled antibodies purchased from BD Biosciences, Biolegend, or MBL were used in this study: CD3ε(145-2C11), CD4 (GK1.5), CD8 (53-6.7), CD8 (KT15), NK1.1 (PK136), CD11b (M1/70), CD11c (N418), CD45 (30-F11), CD45.1 (A20), CD45.2 (104), CD62L (MEL-14), CD44 (IM7), CD119 (GR20), Ly6C (HK1.4), Ly6G (IA8), F4/80 (BM8), MHC-II (M5/114.15.2), PD-L1 (MIH7), Gr-1 (RB6-8C5), IFN-γ (XMG1.2), iNOS (CXNFT), arginase 1 (AlexF5), anti-mouse H-2K^{b} bound to SIINFEKL (25-D1.16), and H-2K^{b} MuLV p15E Tetramer-KSPWFTTL. H-2K^{b} SIINFEKL-PE was kindly provided by the NIH Tetramer Core Facility. For examining IFN-γ production from T cells, cells were first stimulated with leukocyte activation cocktail (Cat. 550583, BD Biosciences) in the presence of the protein transport inhibitor brefeldin A for 6 h. Cells were stained with the indicated cell-surface markers and fixed/permeabilized using a Fixation/Permeabilization Kit (eBioscience). Cell pellets were resuspended in PBS with 2% FBS for FACS analysis. To isolate macrophages from spleen and tumor tissues from MC38-bearing mice, MC38 cells (2×10⁵) were implanted s.c. into the flank of WT mice. Fourteen days after tumor inoculation, single-cell suspensions were prepared from the spleen and pooled tumor tissues. Macrophages (CD11b+F4/80⁺) were sorted using FACSAria Fusion Flow Cytometer (BD Biosciences). The gating strategy for DCs, TAMs, and MDSCs from tumor tissues is shown in (FIG. 18).

Differentiation and polarization of bone marrow-derived macrophages (BMDMs). Bone marrow was extracted from the femurs and tibias of WT or Ythdf2 cKO mice. All bone marrow cells were flushed out and filtered through a 70 μm cell strainer. After centrifugation, red blood cells were lysed. The resultant bone marrow cells were resuspended in RPMI-1640 (Gibco) supplemented with 10% FBS (Gibco), 1% penicillin/streptomycin (Gibco), and 50 μM 2-mercaptoethanol (Sigma) in the presence of 20 ng/mL M-CSF (PeproTech) for 5-7 days. BMDMs were stimulated with 100 ng/ml LPS (Sigma) plus 20 ng/ml IFN-γ (PeproTech) or 20 ng/ml IL-4 (PeproTech) separately for 24 h to obtain M1- or M2-type macrophages, respectively. In some experiments, BMDMs were rested in RPMI-1640 with 10% FBS for 24 h before stimulation. Rested BMDMs were then treated with IL-4 (10 ng/ml), TGF-β (10 ng/ml, PeproTech), IL-10 (10 ng/ml, PeproTech), or IFN-γ (10 ng/ml), respectively for 24 h.

Antigen-presentation assay. BMDMs from WT or Ythdf2 cKO mice were harvested on day 6 and stimulated for 24 h with 100 ng/ml LPS plus 20 ng/ml IFN-γ for M1 polarization or 20 ng/ml IL-4 for M2 polarization, respectively. The M1 or M2 cells were then washed and co-cultured with the SIINFEKEL peptide (10 μg/ml, InvivoGen) or ovalbumin protein (0.2 mg/ml, InvivoGen) for 6 h. Cells were washed and co-cultured with naïve CD8⁺ T cells from OT-I mice for three days. Brefeldin A was added to the co-cultures 4 h before the end of incubation. The cross-priming capacity of macrophages was then evaluated by flow cytometry as IFN-γ production by CD8⁺ T cells.

IFN-γ enzyme-linked immunosorbent spot assay (ELISPOT). B16-OVA cells were injected subcutaneously into the right flank of C57BL/6J mice. Fourteen days later, 3×10⁵ lymphocytes were isolated from draining lymph nodes and re-stimulated with 10 μg/ml SIINFEKEL for 48 h. IFN-γ production was determined with an IFN-γ single-color ELISPOT kit (Cellular Technology) according to the manufacturer's protocol (Cellular Technology). The visualized spots were counted with a CTL-ImmunoSpot S6 Analyzer (Cellular Technology).

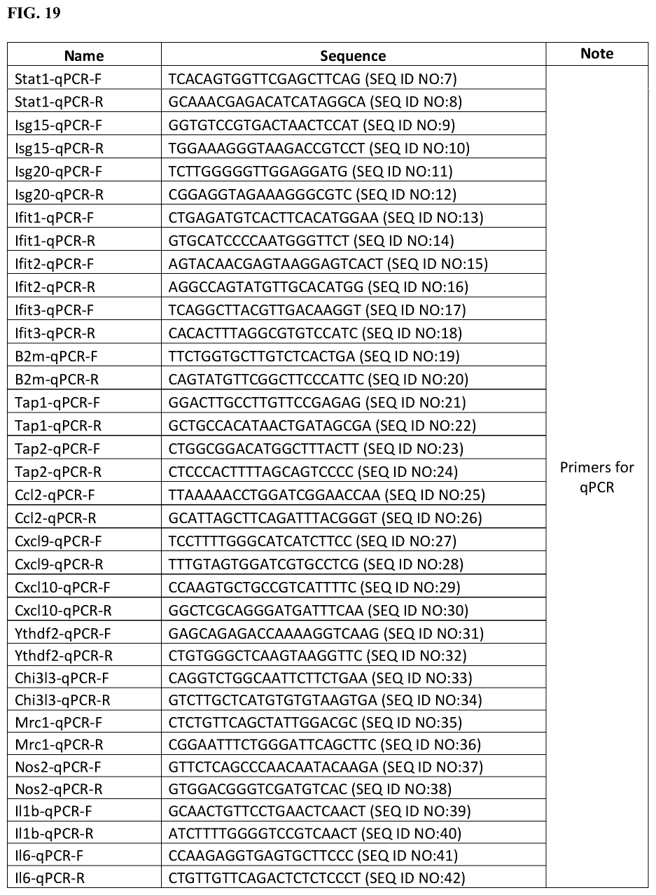

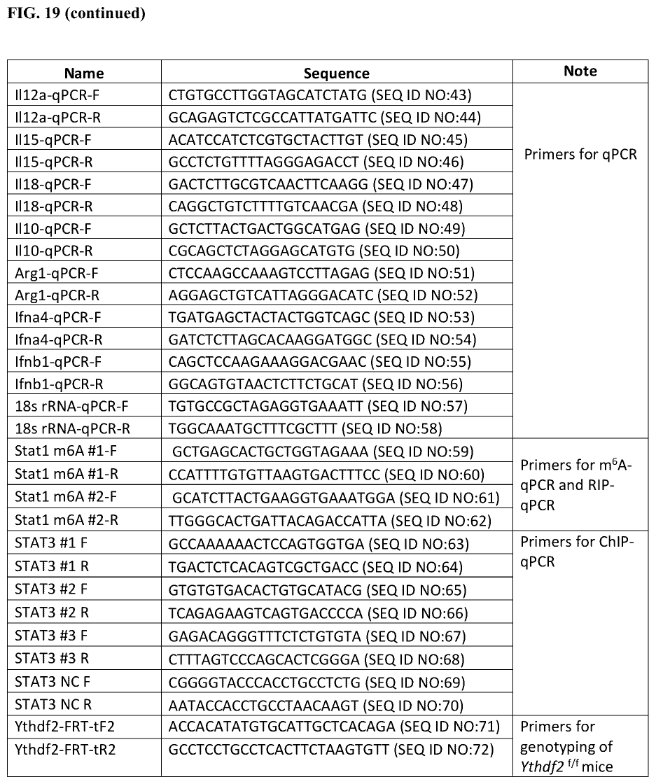

Quantitative real-time RT-PCR (qPCR). RNA was isolated using an RNeasy Mini Kit (QIAGEN) and then reverse transcribed to cDNA with PrimeScript RT Reagent Kit with gDNA Eraser (Takara Bio) following the manufacturer's instructions. mRNA expression levels were analyzed using SYBR Green PCR Master Mix and a QuantStudio 12K Flex Real-Time PCR System (both from Thermo Fisher Scientific). Primer sequences are listed in FIG. 19.

Immunoblotting was performed according to standard procedures, as previously described^{81, 82}. The following antibodies were used: YTHDF2 (MBL, RN123PW), iNOS (CST, 13120S), arginase-1 (CST, 93668S), STAT1 (CST, 9172), phospho-Stat1 (Tyr701) (CST, 9167), phospho-Stat3 (Tyr705) (CST, 9145), and beta-actin (Proteintech, 66009-1-Ig). The immunoblots were visualized with SuperSignal West Femto Maximum Sensitivity Substrate (Cat. 34096, Thermo Fisher Scientific). Densitometric analysis was performed to quantify the intensity of gel bands by ImageJ. Target protein levels were normalized to the expression of β-actin.

Single-cell RNA-sequencing (scRNA-seq). Tumors from WT or Ythdf2 cKO mice were extracted and digested with collagenase type IV (1 mg/ml) and DNase type I (30 U/ml) for 30 min at 37° C. The resulting cells were filtered through 70 m cell strainers, washed with phosphate-buffered saline (PBS), lysed in red blood cell buffer, and resuspended in PBS. Tumor-infiltrating immune cells (CD45⁺ cells) were sorted in a FACSAria Fusion Flow Cytometer (BD Biosciences). The cell suspension (300-600 living cells per microliter) was loaded onto a Chromium Single Cell Controller (10× Genomics) to generate single-cell gel beads in the emulsion according to the manufacturer's protocol. Libraries were subsequently prepared using a Single Cell 5′Library and Gel Bead Kit and sequenced on an Illumina NovaSeq 6000 instrument with a pair-end 100 bp (PE100) reading strategy.

scRNA-seq analysis. Cell Ranger (version 5.0.0) was used to process the raw data, demultiplex cellular barcodes, map reads to the mouse genome (mm10), and generate digital gene expression matrices. 14,404 single cells from WT mice and 11,398 from Ythdf2 cKO mice were detected. The data were then read into the Seurat R package (version 4.0.4) for further processing⁸³. Less prevalent genes (<3) and cells with less prevalent genes (<200) were removed. Low-quality cells were removed if the number of expressed genes was <200 or >5000. Cells were also removed if their proportion of mitochondrial gene expression was >7.5%. The remaining 19,530 single cells were used in downstream analyses. The top 2,500 variable genes identified by the “vst” method were used in principal component analysis; 1-30 principal components (PCs) were used in the FindNeighbors function. The FindClusters function was used to identify clusters (res=0.7), which were then visualized with 2D tSNE plots. To identify specific ligand-receptor pairs between M1 macrophages and effector CD8⁺ T cells, CellPhoneDB software (version 2.1.7)⁴² was used based on the scRNA-seq data. Receptors and ligands expressed in at least 10% of cells were analyzed. Gene ontology (GO) analysis of marker genes derived from scRNA-seq was performed using DAVID (https://david.ncifcrf.gov/). The marker genes were also used for Gene Set Enrichment Analysis (GSEA) using all Gene Ontology terms, with 1000 permutations⁸⁴. The networks and pathway analyses were carried out using Ingenuity Pathway Analysis (IPA QIAGEN Inc)⁸⁵.

m⁶A-seq. Total RNA was isolated by TRIzol reagent (Thermo Fisher Scientific) from 50 million M1-type BMDMs of WT and Ythdf2 cKO mice. m⁶A immunoprecipitation (m⁶A-IP) was performed according to standard procedures, as previously described 36. Sequencing was performed at the Translational Genomics Research Institute (TGen) on an Illumina NovaSeq 6000 platform with 100-bp paired-end reads (PE100). Sequencing reads were mapped to the mouse genome (mm10) using HISAT2 (Version: v2.1.0)⁸⁶. m⁶A enriched peaks from m⁶A-seq samples were identified by MACS2 peak-calling software (version 2.2.6) with the corresponding input sample serving as control⁸⁷. MACS2 was run with default options except for “-nomodel, -keepdup all” to turn off fragment size estimation and to keep all uniquely-mapping reads, respectively. m⁶A peaks were visualized using Integrative Genomics Viewer software (http://www.igv.org). The motifs enriched in m⁶A peaks were analyzed by HOMER (version 4.11)⁸⁸. Each peak was annotated based on Ensembl gene annotation information by applying BEDTools'intersectBed (version 2.30.0). HTSeq (version 0.11.3)⁸⁹ was used to calculate read counts in input samples. Then, expression levels for genes were determined by calculating FPKM (total exon fragments/mapped reads (millions)×exon length (kB))⁸⁹. The differentially expressed genes between WT and Ythdf2 cKO mice were detected by R package DEseq2 (version 1.34.0)⁹⁰ and genes with log 2 (fold change) >0.5 or log 2 (fold change)<−0.5 and a P-value <0.01 were considered as differentially expressed genes.

YTHDF2 RNA immunoprecipitation sequencing (RIP-Seq). Fifty million M1-type BMDMs from WT and Ythdf2 cKO mice were lysed with two volumes of lysis buffer [10 mM HEPES pH 7.6, 150 mM KCl, 2 mM EDTA, 0.5% NP-40, 0.5 mM DTT, 1:100 protease inhibitor cocktail (Thermo Fisher Scientific), and 400 U/mL SUPERase-In RNase Inhibitor (Thermo Fisher Scientific)]. YTHDF2 RIP was performed according to the protocol as previously described³⁶. A DNA library was generated with a KAPA RNA HyperPrep Kit (Roche) and sequenced on the Illumina NovaSeq 6000 platform. Sequencing reads were mapped to the mouse genome (mm10) using HISAT2 (Version: v2.1.0)⁸⁶. The target binding regions of YTHDF2 were identified using MACS2 software (version 2.2.6)⁸⁷. HOMER (version 4.11) was used to find motifs enriched in YTHDF2 binding regions⁸⁸. The target genes were annotated based on Ensembl gene annotation information by applying BEDTools'intersectBed (version 2.30.0).

mRNA stability assay. M1-type BMDMs were treated with actinomycin D (5 μg/ml, Sigma, Cat. A9415) for the indicated time. Untreated cells were used as 0 h. Cells were collected at the indicated time, and total RNA was extracted from the cells for qPCR. The mRNA half-life (t_{1/2}) was calculated using the method previously described³⁶. Primer sequences are listed in FIG. 19.

Dual-luciferase reporter assays. pmirGLO Dual-Luciferase miRNA Target Expression Vector was purchased from Promega. WT, mutant 1, mutant 2, or mutant 1 and 2 3′UTR of Stat1 gene fragments were synthesized by Twist Bioscience and cloned into the pmirGLO vector. WT or Ythdf2 cKO BMDMs were transfected with the pmirGLO reporter plasmids using a mouse macrophage nucleofector kit (Cat. VPA-1009, Lonza) with program Y-001 on a Nucleofector I Device (Amaxa). The cells were harvested for lysis 24 h after transfection. The mouse pGL4-Ythdf2 reporter plasmid was generated as previously described³⁶. Mouse Stat3 pcDNA3 plasmid was purchased from Addgene (#8706). BMDMs were co-transfected with the pGL4-Ythdf2 reporter plasmid and Stat3 overexpression plasmids or an empty vector, together with a pRL-TK Renilla reporter plasmid (Promega) for normalization of transfection efficiency. The cells were harvested for lysis 24 h after transfection. Luciferase activity was quantified fluorimetrically with the Dual-Luciferase System (Promega).

Chromatin immunoprecipitation (ChIP) assays. ChIP assays were performed using a Pierce Magnetic ChIP Kit (Cat. no. 26157; Thermo Fisher Scientific) according to the manufacturer's instructions. Briefly, BMDMs were pretreated with IL-10 (10 ng/ml) for 1 h. 5×10⁶ cells were cross-linked with 1% formaldehyde at 37° C. for 10 min and quenched with 0.125 M glycine. After cell lysis, cross-linked chromatin was sheared using Bioruptor Pico (Diagenode) with 30 s on/30 s off for 30 cycles. Sonicated chromatin was then used for immunoprecipitation with 10 L anti-phospho-Stat3 (Tyr705, Cat. 9145; Cell Signaling Technologies) or corresponding normal rabbit IgG (Cat. 2729; Cell Signaling Technologies) at 4° C. overnight. Following reversal of crosslinking, the DNA immunoprecipitated by the indicated antibody was tested by qPCR using primers listed in FIG. 19.

Oligonucleotide design, synthesis, and treatment. The sequences of mouse cell-specific CpG1668-siRNAs have been described previously⁵². The phosphothioated oligodeoxyribonucleotide (ODN) and sense strands (SS) of siRNAs were linked using 6 units of C3 carbon chain linker, (CH2)₃ (Glen Research, Sterling, VA). The resulting constructs were hybridized to complementary siRNA antisense sense (AS) strands to generate CpG-siRNA conjugates (asterisks indicate phosphothioation sites). Sequences of single-stranded constructs are listed below:

CpG-Ythdf2 siRNA (SS): 5′-(SEQ ID NO: 1)-linker-(SEQ ID NO: 3).
Ythdf2 siRNA (AS): SEQ ID NO: 2.
CpG-scrambled RNA (SS): 5′ (SEQ ID NO: 1)-linker-
C*U*UACGCUGAGUACUUCGAUU 3′ (SEQ ID NO: 4).
CpG-scrambled RNA (AS): A*A*UCGAAGUACUCAGCGUAAG*U*U (SEQ ID NO: 5).

For the uptake studies, CpG-Ythdf2 siRNA (SS) was labeled using fluorescein Cy3. In vivo delivery of CpG-Ythdf2 siRNA for treatment began when the largest diameter of the tumors reached 5-8 mm-typically 5-7 days after inoculation. Mice were injected intratumorally every other day with 5 mg/kg of CpG-Ythdf2 siRNA, CpG-scrambled siRNAs, CpG alone or PBS in a volume of 20 μL. Tumor growth was monitored every other day.

YTHDF2 siRNA Knock-Down in Human Monocyte-Derived Macrophages Using Nucleofection

HLA-A*02 positive human monocytes were pre-tested by flow cytometry using anti-HLA-A2 antibody (Biolegend, clone: bb7.2) and enriched from the peripheral blood mononuclear cells (PBMCs) using RosetteSep Human Monocyte Enrichment Cocktail (STEMCELL Technologies, Cat #15068) and Ficoll-Paque (GE Healthcare). 1×10⁶ freshly isolated monocytes were transfected with siGENOME Human YTHDF2 siRNA (300 nM, Cat. M-021009-01-0005, Horizon) or scramble siRNA in 20 μL/reaction of room-temperature P3 primary cell 4D-Nucleofector X solution (Lonza, V4XP-3032) with program EA-100 on a 4D-Nucleofector system (Lonza). Transfected monocytes were seeded in a 24-well plate and cultured in RPMI 1640 with 10% heat-inactivated FBS (Gibco) at 37° C. in a 5% CO2 humidified incubator in the presence of human M-CSF (25 ng/ml, Proteintech). Half of the culture media were removed and replaced with fresh media containing M-CSF on day 3. Cells were harvested on day 7 and the knock-down efficiency was determined b qPCR.

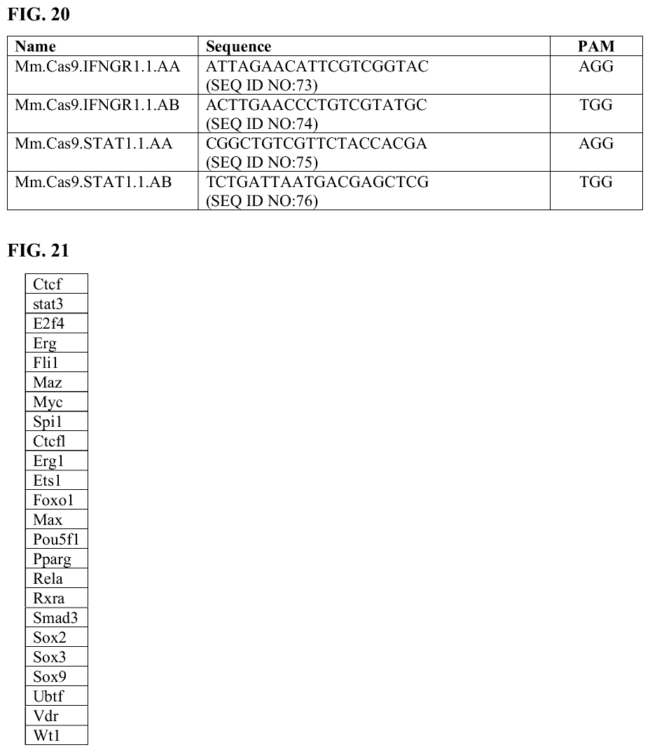

CRISPR-Cas9-mediated knock-out in BMDMs: Stat1 and Ifng1 were knock-out in BMDMs using Alt-R CRISPR System (IDT, USA). CRISPR RNAs (crRNAs) were predesigned and purchased from IDT and the sequences are listed in FIG. 20. To prepare the crRNA/tracrRNA duplex, the Alt-R crRNA and Alt-R-tracrRNA were reconstituted to 200 μM with Nuclease-Free Duplex Buffer (IDT) and mixed at equimolar concentrations in a sterile PCR tube to a final concentration of 100 μM. Oligos were annealed by heating at 95° C. for 5 min in a PCR thermocycler and allow to cool to room temperature for 15 min. To form Cas9-ribonucleoprotein (RNP), the annealed crRNA/tracrRNA duplexes were mixed with Cas9 (Alt-R S.p. Cas9 Nuclease V3) at a 1.2:1 molar ratio for each reaction in a sterile PCR tube, by incubation at room temperature for 10 min. To provide maximal knock out efficacy, two crRNA sequences were pooled when preparing the RNP⁹¹. BM cells were harvested and washed twice with 1×PBS. 1×10⁶ cells per reaction were resuspended in 20 μl of P3 primary nucleofection solution (Lonza). 5 μl RNP complex and 1 μl electroporation enhancer were added to 20 μl cell/P3 nucleofection solution and pipetted up and down three to five times gently to mix while avoiding bubbles. The cell/RNP mix was then immediately loaded into the supplied nucleofector cassette strip (Lonza). The strip was inserted into the Lonza 4D-Nucleofector and nucleofected with the buffer P3, program CM-137. The cells were cultured in the presence of M-CSF for 5-7 days. BMDMs were harvested for subsequent assays or mixed with an equal number of B16-OVA cells (2×10⁵) and implanted s.c. into the flank of WT mice. Knock-out efficiency was examined by immunoblotting or flow cytometry.

NY-ESO-1-specific Ly95 T cell response: Human T cells were isolated from PBMCs using RosetteSep Human T cell Enrichment Cocktail (STEMCELL Technologies, Cat #15061) and Ficoll-Paque (GE Healthcare). Enriched T cells were stimulated with a magnetic bead coated with anti-CD3/CD28 (11131D, Gibco) and IL-2 (50 U/ml) for 24 h. Activated T cells were transduced with a high titer lentiviral vector expressing NY-ESO-1-reactive Ly95 TCR⁶⁵. Transgenic TCR expression was determined by flow cytometric analysis using an anti-human V013.1 TCR chain antibody (Beckman Coulter: clone IMMU 222)^{65, 92}. HLA-A2 positive YTHDF2 knock-down and control monocytes derived macrophages were polarized to M1 type using IFN-γ (20 ng/ml) plus LPS (100 ng/ml) for 24 h, and then were incubated with HLA-A*02-restricted NY-ESO-1 (157-165, SLLMWITQC) peptide (MBL, SP1065) (2 μg/mL) for 2 h, washed three times with cell culture medium, and mixed with autologous Ly95 T cells at a ratio 1:1 in complete cell culture media. 18 h later, NY-ESO-specific activation of the Ly95 T cells was assessed by measuring intracellular IFN-γ and granzyme B in gated CD8⁺ TCRVβ13.1⁺ cells.

Immunohistochemistry of human biopsies and analysis: Tumor biopsies from 18 colorectal cancer patients were previously described and obtained from the University of Chicago with informed consent. (Ref 23). Tissue microarray with 32 lung cancer tissues was obtained from Pathology Cores & Biobanking Shared Resources at the City of Hope National Medical Center with informed consent. Single or double IHC was performed on Ventana Discovery Ultra IHC autostainer (Ventana Medical Systems, Roche Diagnostics, Indianapolis, USA) using the Discovery HQ-HRP-DAB detection system (Ventana). Briefly, samples were sectioned at a thickness of 5 m and put on positively charged glass slides. The slides were loaded on the machine, deparaffinization, rehydration, endogenous peroxidase activity inhibition, and antigen retrieval were first performed. For single IHC staining, the primary antibody was incubated with DISCOVERY anti-Rabbit HQ followed by DISCOVERY anti-HQ-HRP incubation. For double IHC staining, two antigens were sequentially detected and heat inactivation was used to prevent antibody cross-reactivity between the same species. Following each primary antibody incubation, DISCOVERY anti-Rabbit NP or DISCOVERY anti-Rabbit HQ and DISCOVERY anti-NP-AP or DISCOVERY anti-HQ-HRP were incubated. The stains were visualized with DISCOVERY ChromoMap DAB Kit, DISCOVERY Yellow Kit, or DISCOVERY Purple Kit accordingly and counterstained with hematoxylin (Ventana) and coverslipped. After staining, the whole slide images were acquired with NanoZoomer S360 Digital Slide Scanner (Hamamatsu) and viewed by NDP.view image viewer software. The following primary antibodies were used in this study. YTHDF2 (Cat #: ab246514, Abcam, 1:400), CD68 (Cat #: 790-2931, Ventana, read to use), and CD8 (Cat #: 790-4460, Ventana, read to use). To count the cells in the stroma tissues, two random circular regions of interest (ROIs) with size 150 mm² at magnification×20 were analyzed. YTHDF2⁺CD68⁺ cells and CD8⁺ cells were counted and reviewed by an independent pathologist. Correlations between the average infiltration of CD8⁺ T cells and average numbers of YTHDF2⁺CD68⁺ macrophages were evaluated using Spearman correlation method. Blinded staining and blinded analysis were performed for IHC experiments.

Analysis of cancer patient data from TCGA: Gene expression and clinical data for TCGA skin cutaneous melanoma (SKCM) were downloaded from UCSC Xena (http://xena.ucsc.edu/). Samples without survival information were excluded. Patients were classified as either CD68^{high} or CD68^{low} based on the expression level using a 50% cutoff. CD68^{high} patients were further stratified into either YTHDF2^{high} or YTHDF2^{low} based on expression level by testing cutoffs using the percentile whose association with overall survival of the whole cohort had the lowest P-value. Survival analyses were performed using the “survival” R package (version 3.2-13) and Kaplan-Meier methods with a Log-rank test. For the YTHDF2 target gene signature, we generated the YTHDF2 target gene set by overlapping the genes from scRNA-seq, m⁶A-seq, and RIP-seq. For each target gene, patient-wise Z-scores were calculated for gene signature heatmaps, which were plotted using the “ComplexHeatmap” R package (version 2.11.1). Twelve target genes were selected for survival analysis using the Gene Expression Profiling Interactive Analysis 2 (GEPIA2) webserver (http://gepia2.cancer-pku.cn) with a 50% cutoff value. P-value was calculated using the Kaplan-Meier methods with the Log-rank test. (Ref 93).

Analysis of public scRNA-seq data: All public database scRNA-seq data were downloaded from the GEO database (glioblastoma: GSE84465; colorectal cancer: GSE146771, GSE178341; breast cancer: GSE114725, kidney cancer, GSE134520). For each set of data, the steps of quality control, dimensionality reduction, clustering, and cell annotation were strictly carried out according to the parameters provided in the corresponding studies. Myeloid cells were then extracted, and expression of YTHDF2 in myeloid cells was compared between the normal and the tumor groups.

Statistical analysis: Data are presented by descriptive statistics such as mean±s.d. For continuous endpoints such as flow cytometry data or RT-PCR data, a Student's t-test or paired t-test was used to compare two independent or matched conditions/groups, and one-way ANOVA models were used to compare three or more independent conditions/groups. linear mixed models (i.e. two-way ANOVA with mixed-effects models in PRISM) were used to account for the variance-covariance structure due to repeated measures such as tumor volume. Mouse survival functions were estimated by the Kaplan-Meier method and compared by log-rank tests. All tests were two sided. P values were adjusted for multiple comparisons by Holm-Šídík procedure. A P value of <0.05 was considered statistically significant. *P<0.05, **P<0.01, ***P<0.001, ****P<0.0001. Prism software v.9.0 (GraphPad) and SAS v.9.4 (SAS Institute) were used to perform statistical analyses.

### Example 2

In this study, we identified that YTHDF2, an RNA epitranscriptome regulator, plays a central role in regulating both tumor immune evasion and immunoediting. In tumor cells, YTHDF2 restricts the expression of the chemokine CX3CL1 and thus limits macrophage recruitment and polarization. Ythdf2-deficient tumor cells also recruit more macrophages via CX3CL1, which are then polarized into anti-tumoral macrophages in the presence of CD8⁺ T cell-derived IFN-γ. These anti-tumoral macrophages in turn activate CD8⁺ T cells through antigen cross-presentation. In addition, Ythdf2-deficient tumor cells show higher sensitivity to CD8⁺ T cell mediated cytotoxicity by dampening tumor glycolysis metabolism. Interestingly, CD8⁺ T cells and their associated IFN-γ can edit tumor cells by downregulating YTHDF2 expression via autophagy-mediated protein degradation. We have identified a small molecule compound that specifically targets and degrades YTHDF2, but not YTHDF1 and YTHDF3 in tumor cells. This compound not only exhibits a potent antitumor effect but displays enhanced anti-tumor efficacy when combined with PD-L1 blockade. Our findings indicate that YTHDF2 is a tumor intrinsic regulator that orchestrates immune evasion and editing.

YTHDF2 Regulates Tumor Escape from CD8⁺ T Cell-Mediated Immune Surveillance

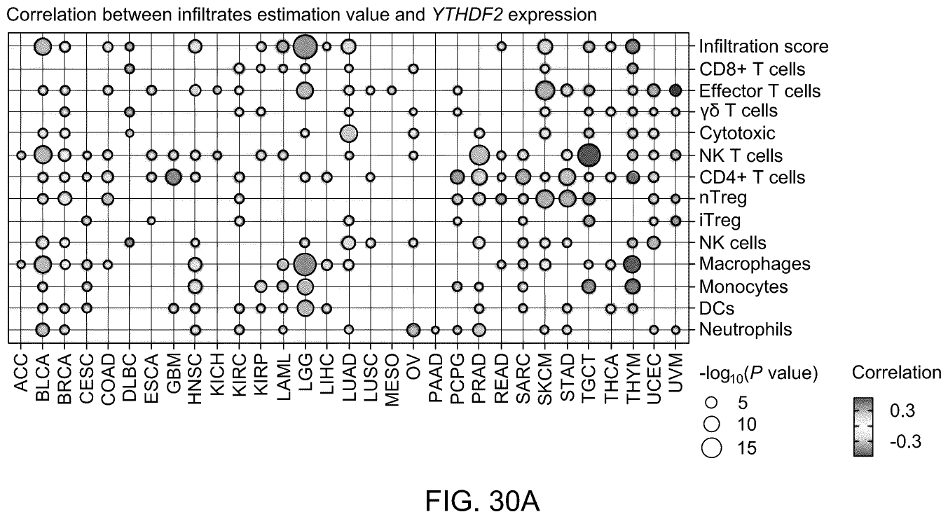

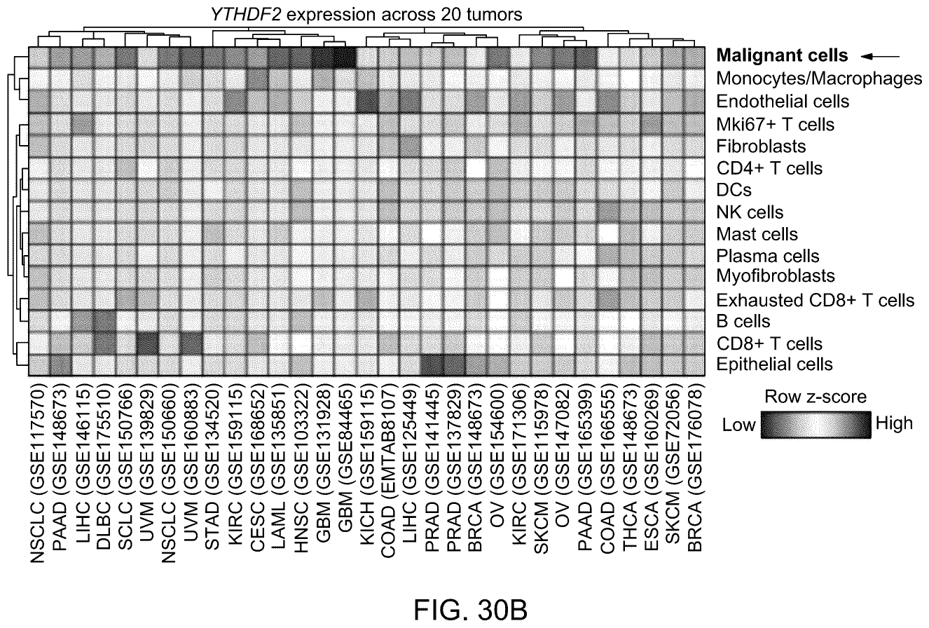

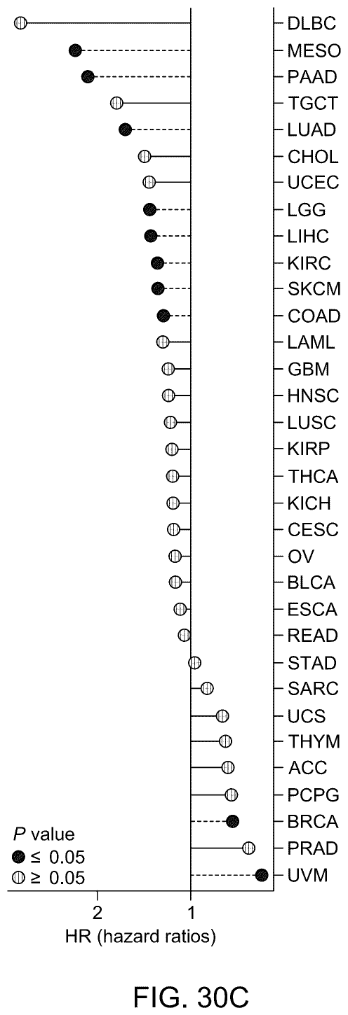

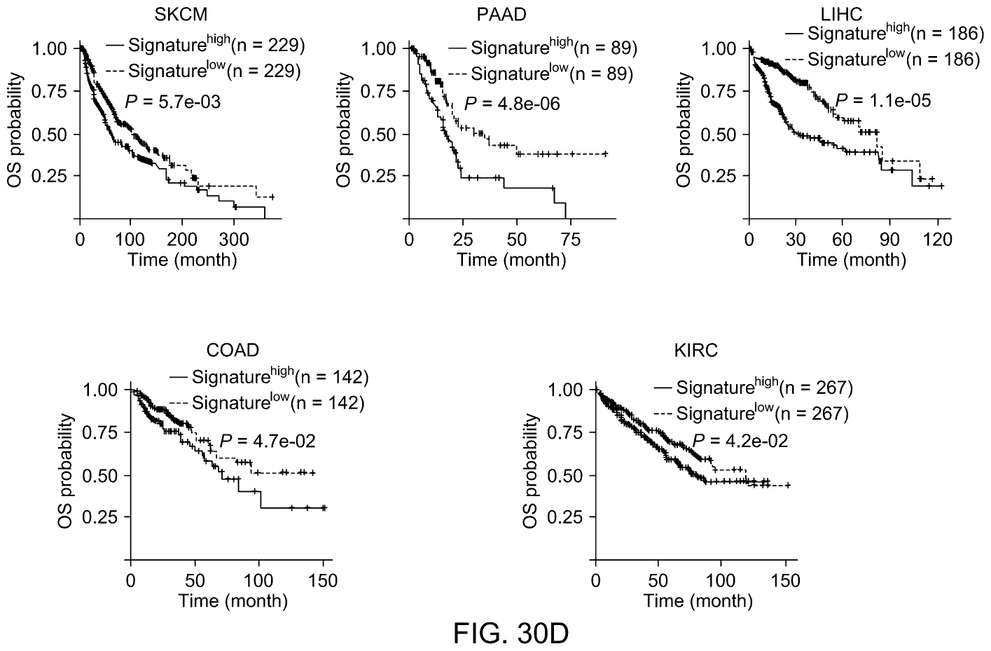

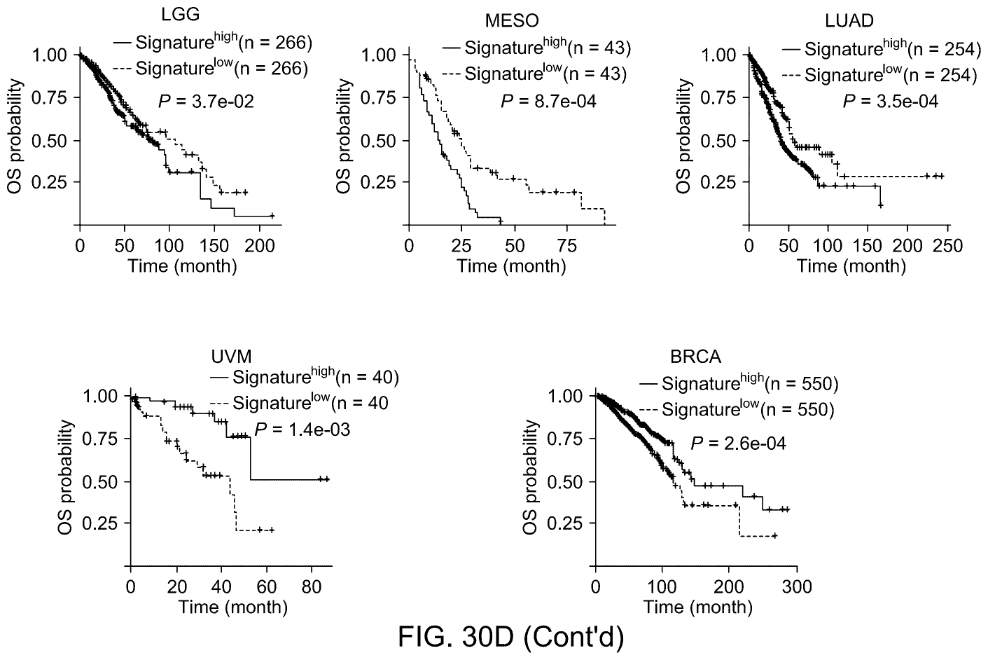

We previously reported that the expression of YTHDF2 mRNA was significantly upregulated in 14 types of tumors, such as colon adenocarcinoma (COAD), skin cutaneous melanoma (SKCM), and pancreatic ductal adenocarcinoma (PAAD) (Ma et al., 2023b). By further analyzing The Cancer Genome Altas (TCGA) datasets, we observed a negative correlation between the expression of YTHDF2 mRNA and immune cells infiltrate estimation value in COAD, SKCM, PAAD, lung adenocarcinoma (LUAD), and liver hepatocellular carcinoma (LIHC) (FIG. 30A). Analysis of public single-cell RNA-seq (scRNA-seq) data indicated that expression of YTHDF2 was higher in 20 tumors but lower in immune cells (FIG. 30B). In addition, high expression of the YTHDF2 was correlated with poor prognosis (FIG. 30C) and shorter overall survival in multiple cancers such as COAD, SKCM, PAAD, and LIHC patients (FIG. 30D). These data indicate that YTHDF2 may play a role in tumor progression and limiting immune infiltration.

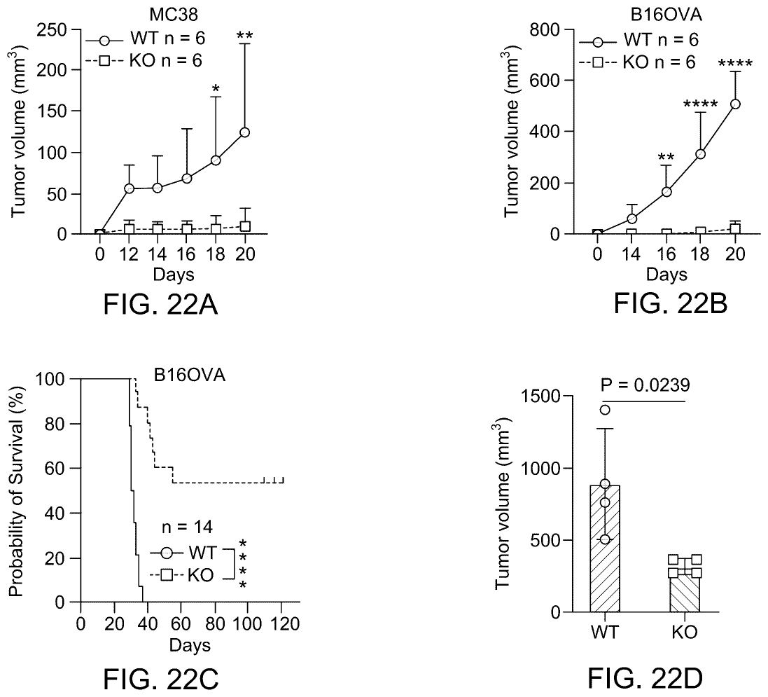

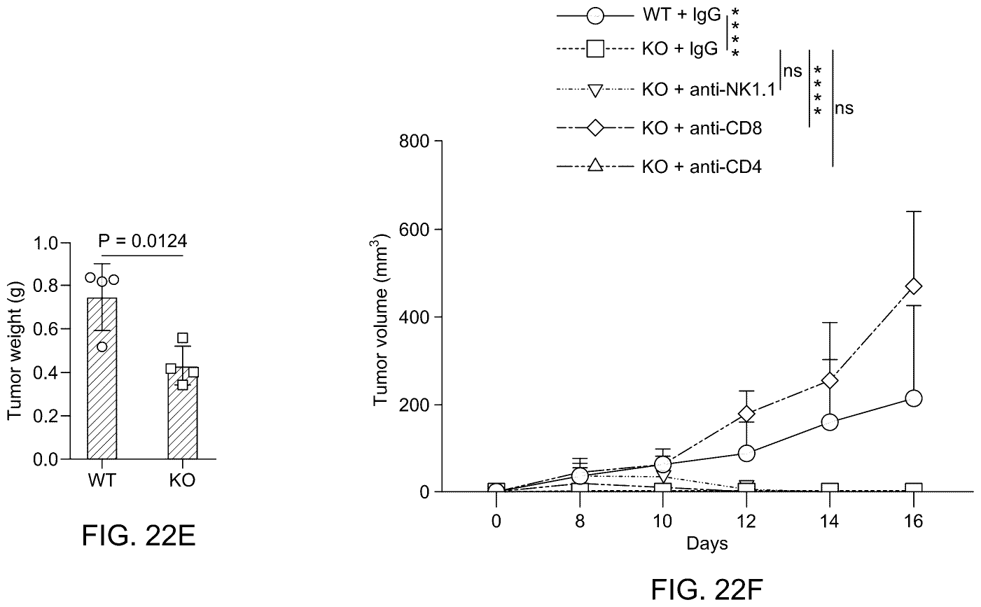

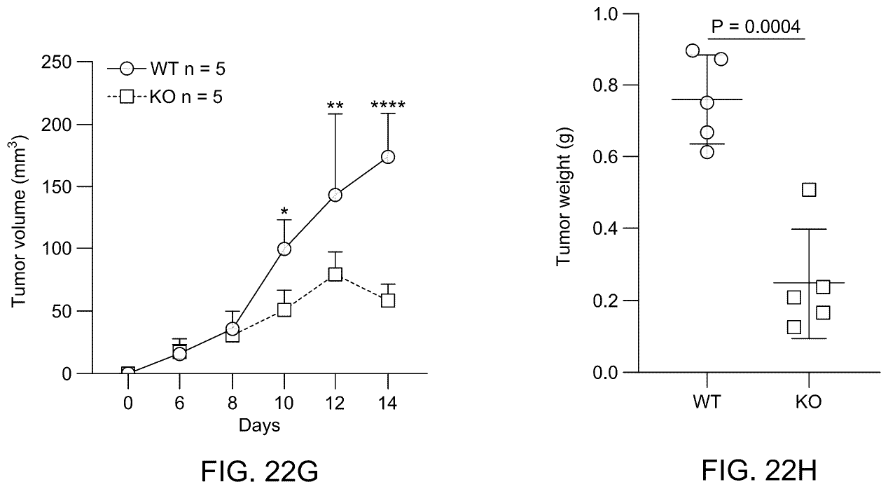

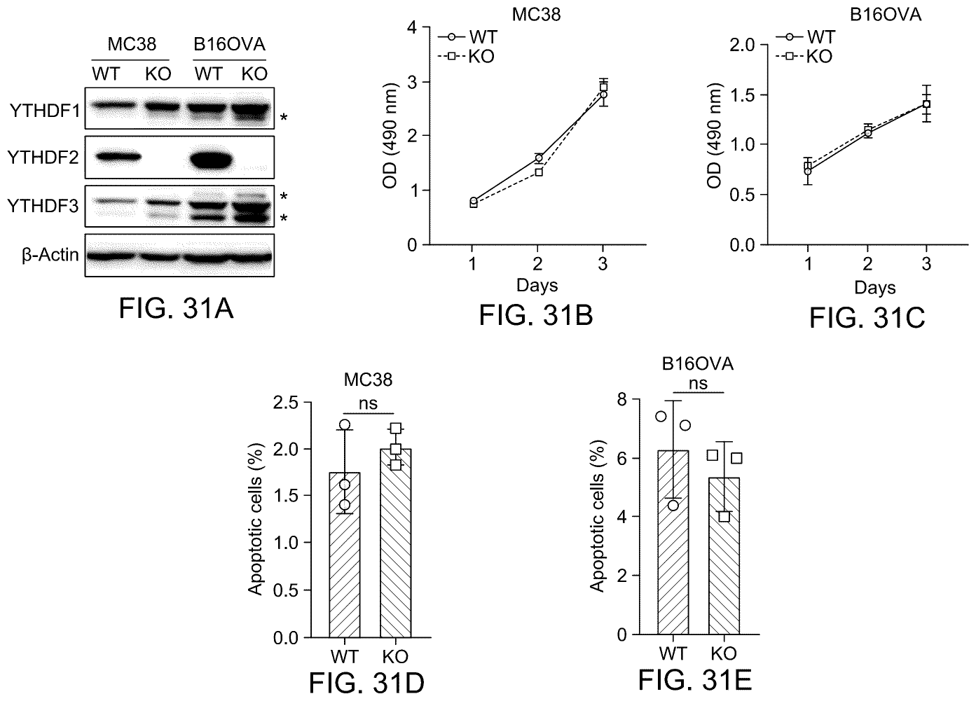

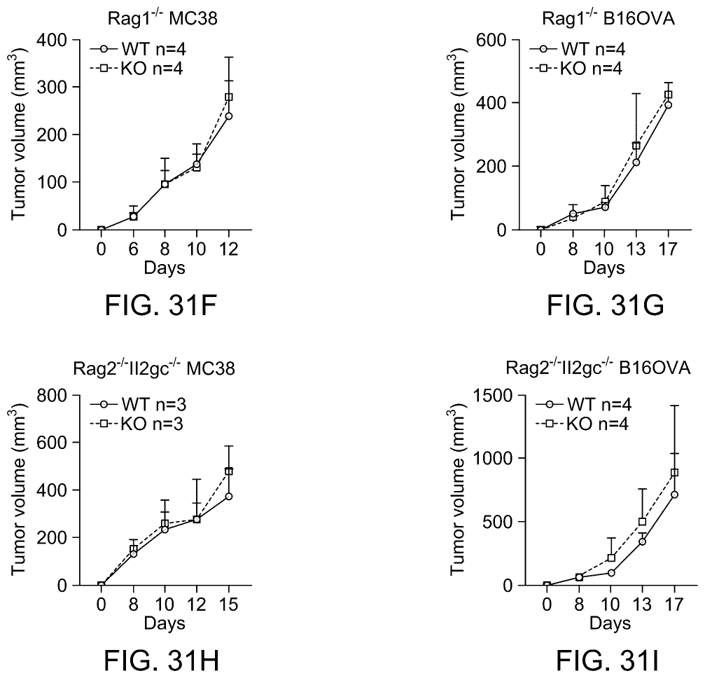

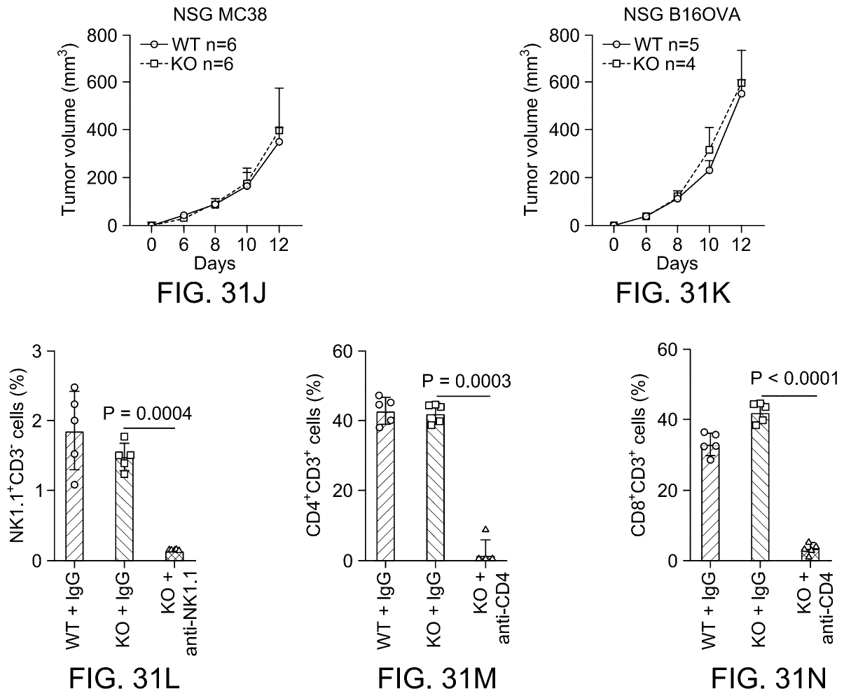

To elucidate the role of tumor-intrinsic YTHDF2 in tumorigenesis and progression, we used CRISPR-Cas9 technology to genetically delete the Ythdf2 gene in two murine tumor cell lines: the MC38 colon cancer cell line and B16-OV melanoma cell line. Immunoblotting showed that the YTHDF2 protein was completely depleted in two cell lines without affecting YTHDF1 or YTHDF3 expression (FIG. 31A). Surprisingly, Ythdf2-KO had no discernable effect on proliferative or apoptosis phenotypes of B16-OVA and MC38 cells in vitro (FIGS. 31B-31E), or tumor growth in immunodeficient Rag1^{-/-}, Rag2^{-/-}Il2gc^{-/-}, and NSG hosts (FIGS. 31F-31K). By contrast, Ythdf2-KO B16-OVA and MC38 tumors did not grow in immunocompetent C57BL/6 hosts (FIGS. 22A-22B). Moreover, Ythdf2-KO B16-OVA tumor bearing mice had longer survival compared to WT tumor bearing mice (FIG. 22C). These data indicate that YTHDF2 regulates tumor progression in immunocompetent mice. To further validate the role of YTHDF2 in tumor growth, we performed tumor transplantation assay by inoculating WT or Ythdf2-KO B16-OVA tumors that were derived from immunodeficient Rag1^{-/-} mice into immunocompetent C57BL/6 mice. The results showed that Ythdf2-KO B16-OVA tumors grew slower than WT tumors when re-transplanted into immunocompetent mice (FIGS. 22D-22E). These data indicate that tumor intrinsic YTHDF2 promotes tumor growth through inducing tumor immune evasion. To determine which cell type contributes to the tumor immunosurveillance, we depleted NK cells, CD4⁺ cells, or CD8⁺ cells using neutralizing antibodies in B16-OVA tumor model (FIGS. 31L-31N). The results showed that depletion of CD8⁺ T cells, but not CD4⁺ T cells or NK cells abolished tumor growth between WT and Ythdf2-KO B16-OVA tumors (FIG. 22F). Furthermore, when Rag1^{-/-} mice were implanted with WT or Ythdf2-KO MC38-OVA tumors and then adoptively transferred with OT1 CD8⁺ T cells, Ythdf2-KO tumors were significantly smaller than WT tumors (FIGS. 22G-22H). Collectively, these results indicate that YTHDF2 regulates tumor escape from CD8⁺ T cell-mediated immune surveillance.

### YTHDF2 Deficiency Remodels the Immunosuppressive TME

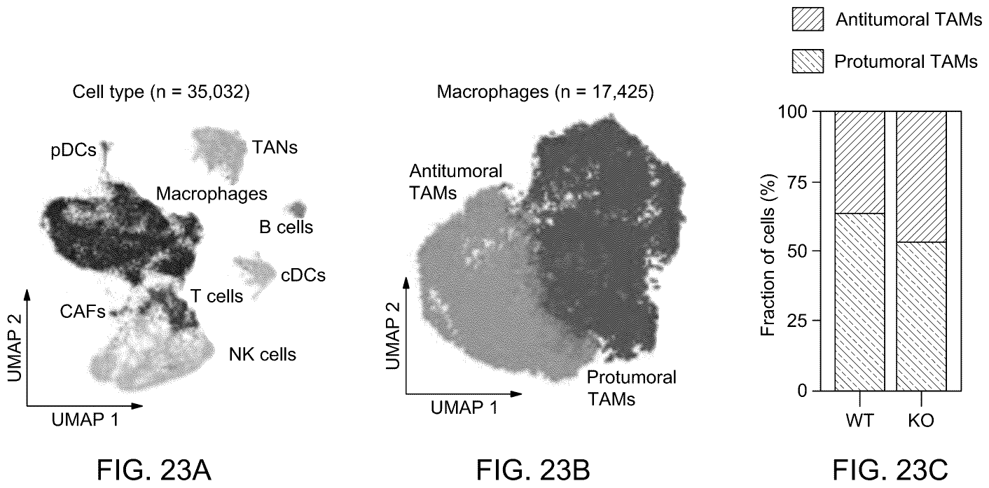

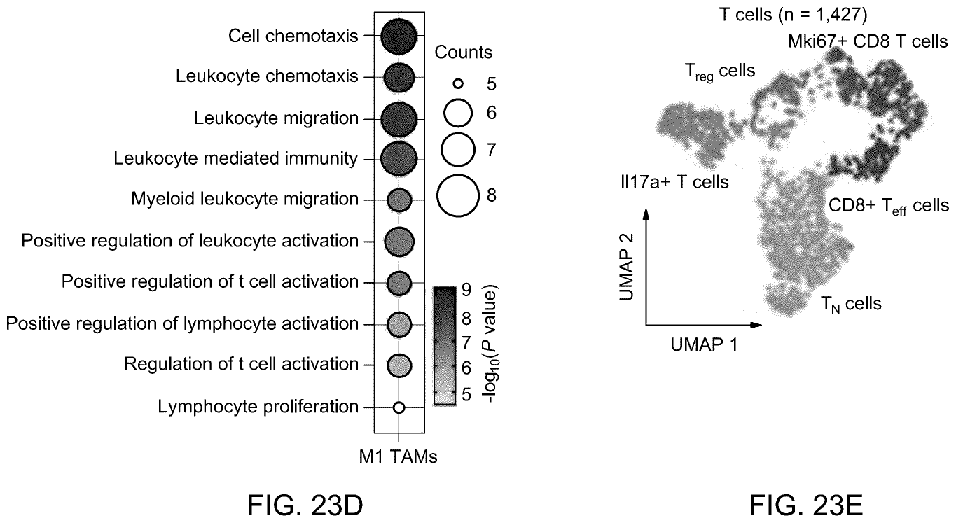

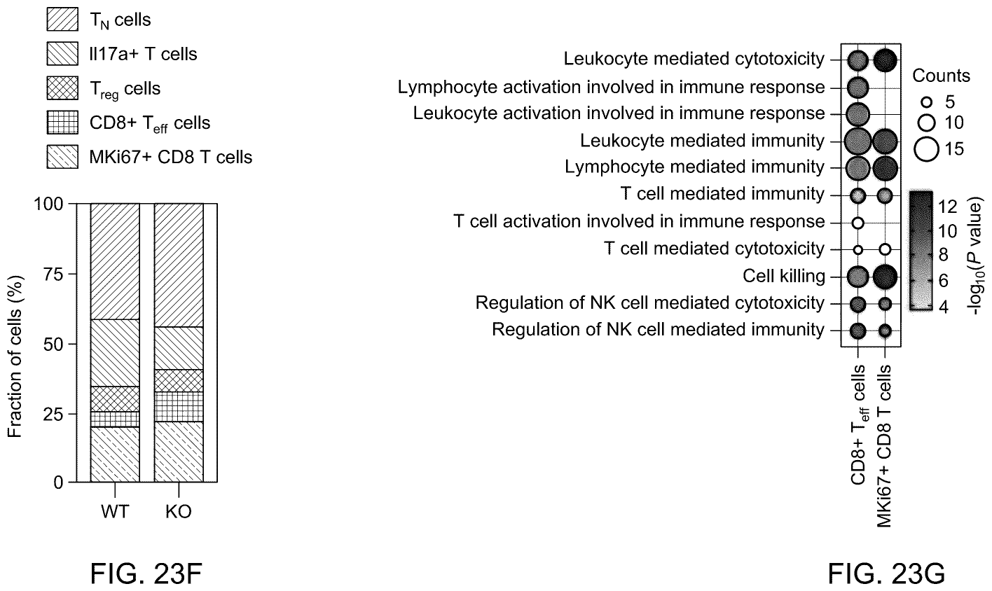

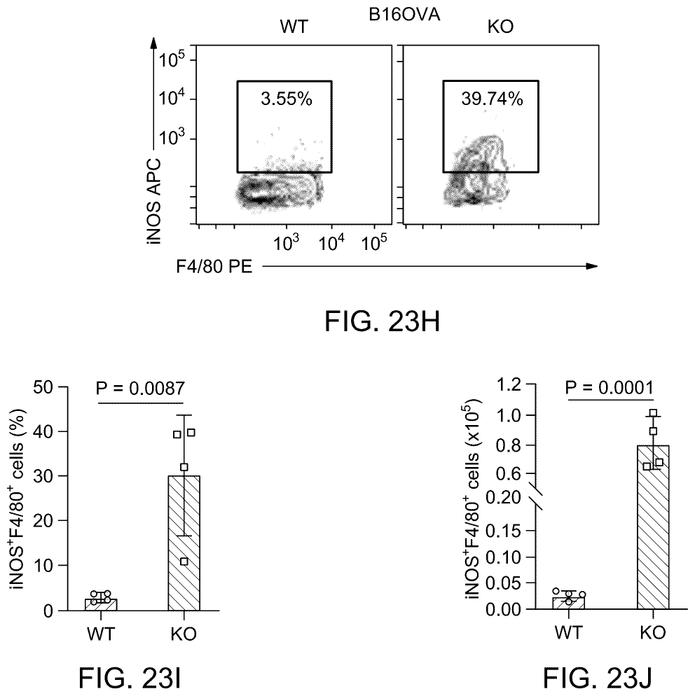

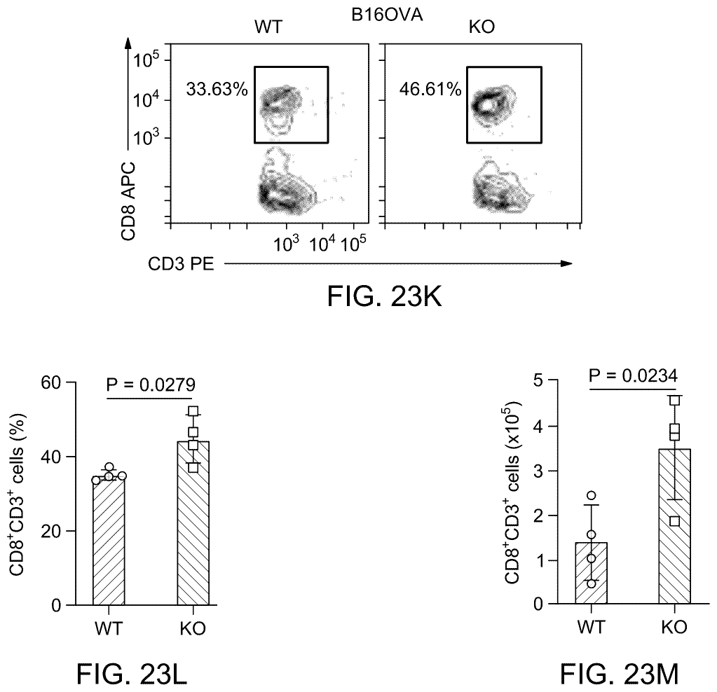

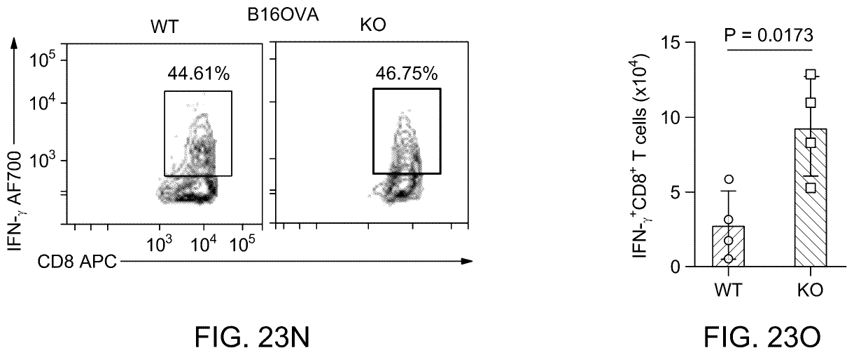

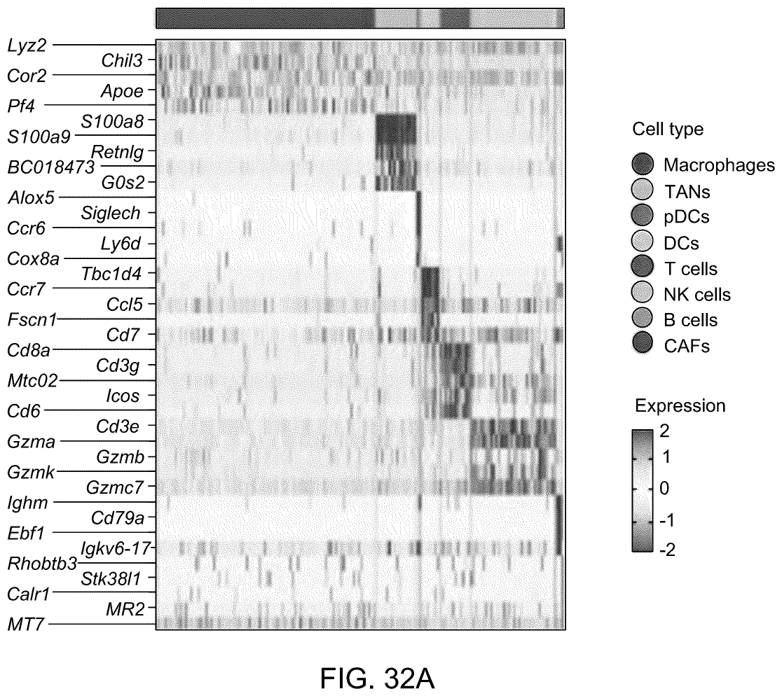

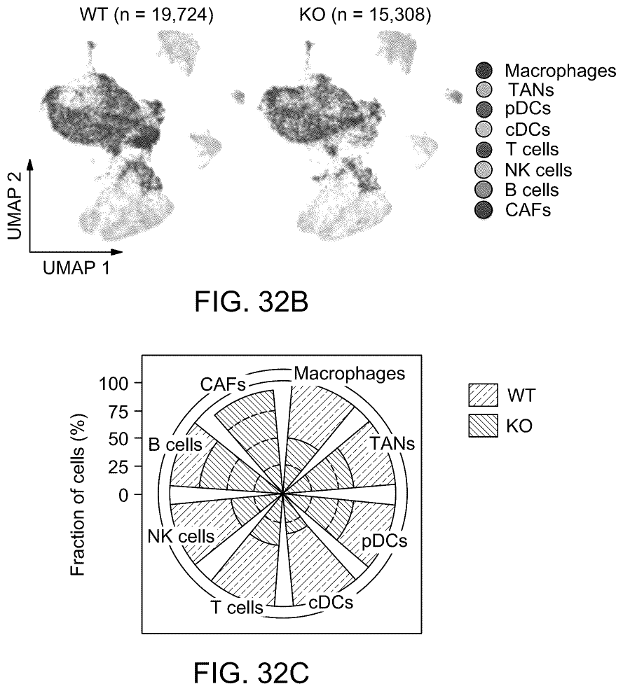

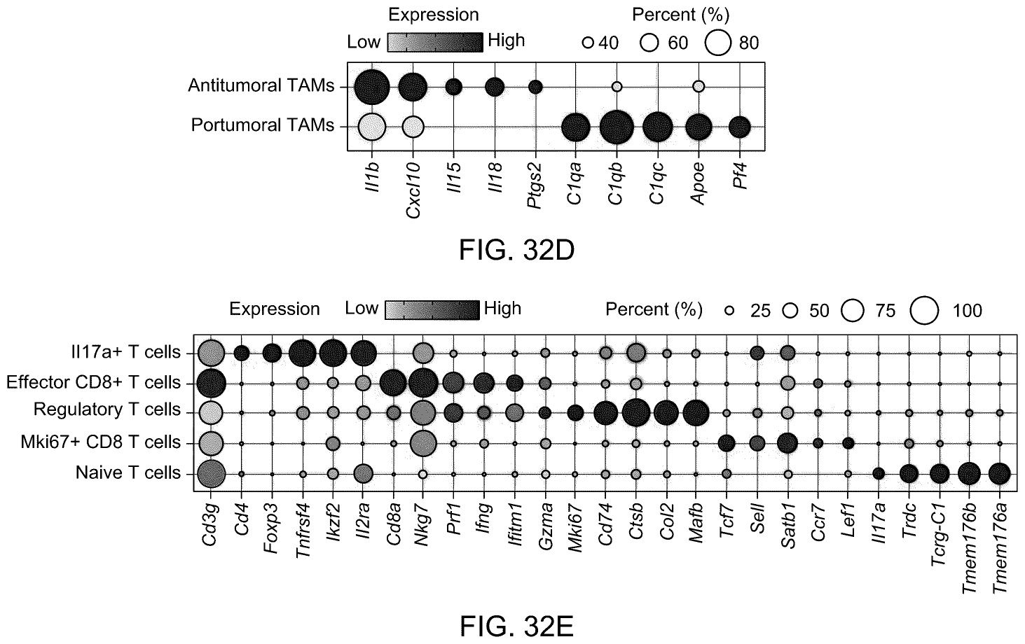

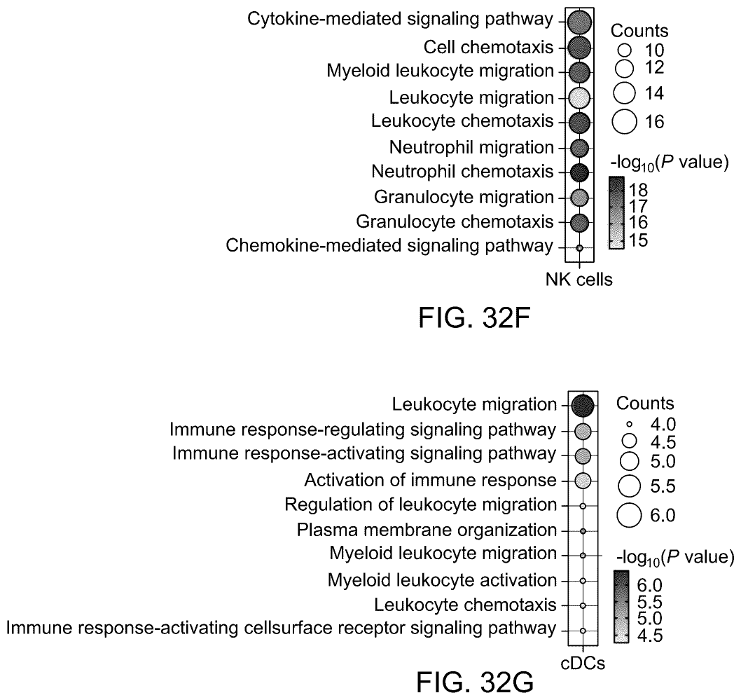

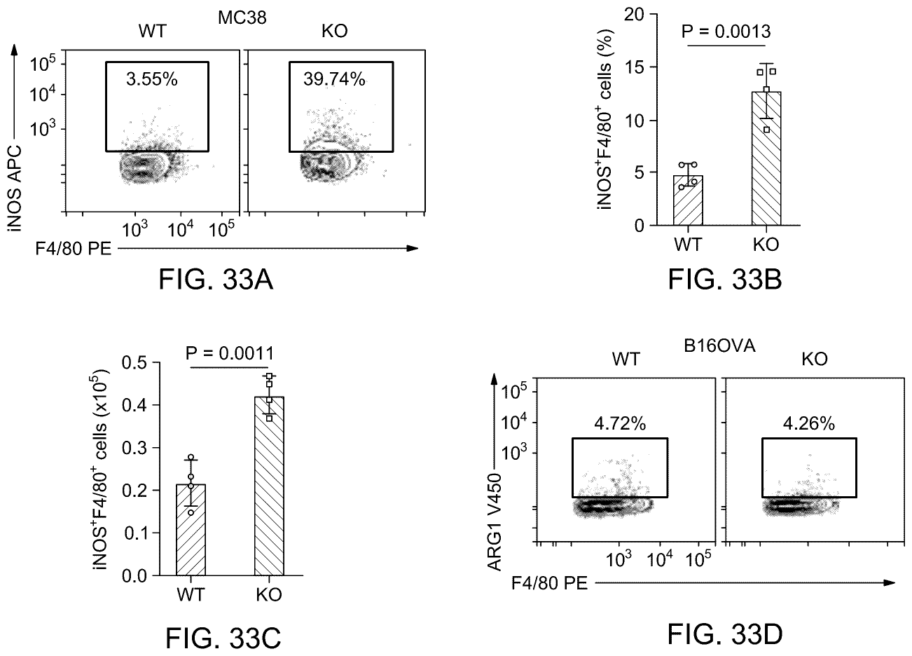

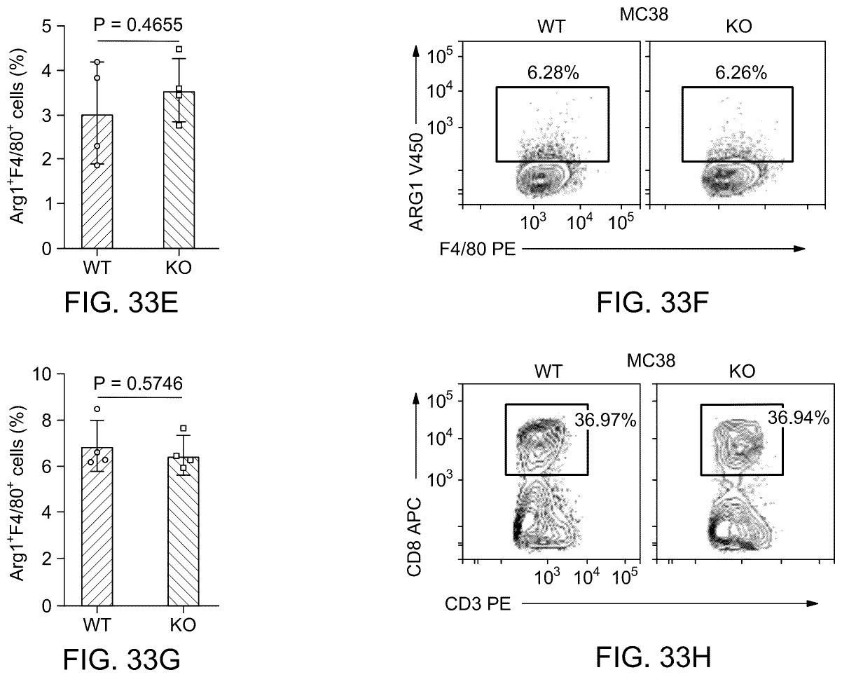

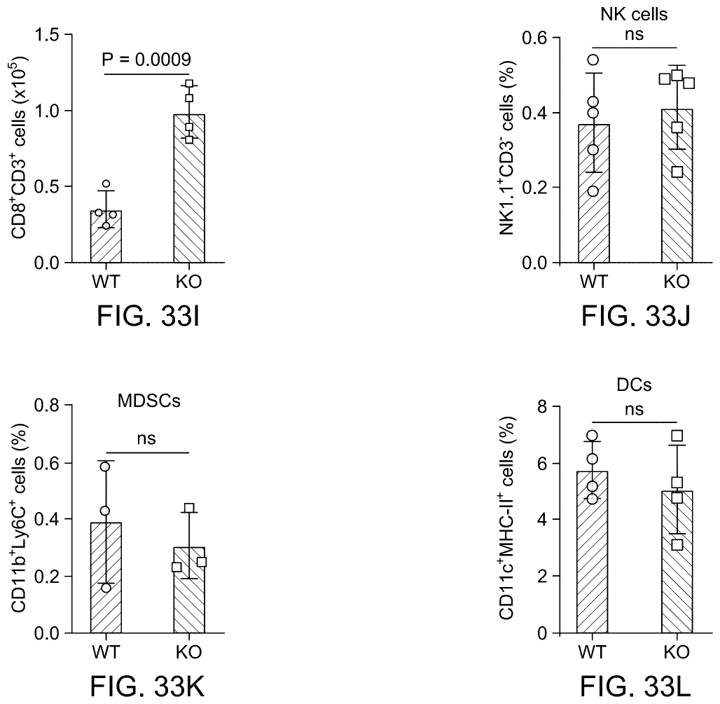

Tumors can evade the immune system through various mechanisms, such as creating an immunosuppressive tumor microenvironment (TME) (Kim and Cho, 2022). To explore whether loss of YTHDF2 in tumor cells affects the TME, we performed single-cell transcriptome analysis of tumor infiltrating CD45⁺ immune cells from WT and Ythdf2-KO B16-OVA tumors. Using published cell type-specific gene markers (Zhang et al., 2019), cell types with similar expression patterns were identified (FIG. 32A). UMAP analysis of total immune cell population identified 8 cell clusters, including macrophages, TANs (tumor associated neutrophils), pDCs, cDCs, T cells, NK cells, B cells, and CAFs (cancer associated fibroblasts) (FIG. 23A). Overall, the proportion of each cell cluster was similar between Ythdf2-KO and WT tumors (FIGS. 32B-32C). To gain deeper insight into the heterogeneity of the TME, we performed subcluster analyses of these infiltrated immune cells. We identified two subclusters of macrophages: anti-tumoral macrophages (expressing Il1b, Il15, Il18, Ptgs2, and Cxcl10) and pro-tumoral macrophages (expressing C1qa, C1qb, C1qc, Apoe, and Pf4) (FIG. 32D). Of note, the proportion of anti-tumoral macrophages was higher in Ythdf2-KO tumors, whereas the proportion of pro-tumoral macrophages was lower in Ythdf2-KO tumors compared to those in WT tumors (FIGS. 23B-23C), indicating a switch from pro-tumoral to anti-tumoral macrophages in Ythdf2-KO tumors. Gene ontology (GO) analysis showed that up-regulated DEGs in anti-tumoral macrophages were mostly enriched in proinflammatory pathways, such as cell chemotaxis, leukocyte migration, leukocyte mediated immunity, positive regulation of T cell activation, etc. (FIG. 23D). Flow cytometry analysis revealed a significant increase in the percentage of iNOS⁺F4/80⁺ anti-tumoral macrophages (FIG. 23H-23J and 33A-33C). However, the percentage of Arg1⁺F4/80⁺ pro-tumoral macrophages remained unchanged in Ythdf2-KO B16-OVA or MC38 tumors compared to WT tumors (FIG. 33D-33G). These results indicate that Ythdf2-KO promotes the recruitment or polarization of anti-tumoral macrophages in the TME.

In the T cell cluster, we identified the effector CD8⁺ T cell subpopulation (expressing Cd3g, Cd8a, Ifitm1, Nkg7, Ifng, Gzma, Prf1), Mki67⁺CD8⁺ T cell subpopulation (expressing Cd3g, Cd8a, Mki67, Cd74, Ctsb, Ccl2, and Mafb), Treg cell subpopulation (expressing Cd3g, Foxp3, Ikzf2, Il2ra, and Tnfrsf4), IL-17-producing γδT cell subpopulation (expressing Cd3g, Cd4, IL-17a, Trdc, and Tcrg-C1), and naïve T cell subpopulation (expressing Satb1, Tcf7, Sell, Ccr7, and Lef1) (FIGS. 2E, 23E). By comparing the abundance of each subpopulation, we found a larger proportion of effector CD8⁺ T cells and Mki67⁺CD8⁺ T cells in Ythdf2-KO tumors compared to WT tumors (FIG. 23F). In contrast, we found that Treg cell and IL-17-producing γδT cell subpopulations, which are known as tumor promotion cells (McAllister et al., 2014; Vignali et al., 2008), in lower proportions among Ythdf2-KO tumors compared to WT tumors (FIG. 23F). GO analysis showed that up-regulated DEGs in effector CD8⁺ T cell and Mki67⁺CD8⁺ T cell subpopulations were mostly enriched in tumor-killing pathways, such as leukocyte mediated cytotoxicity, T cell-mediated cytotoxicity, and cell killing (FIG. 23G). Moreover, the absolute number of CD8⁺ T cells, as well as IFN-γ CD8⁺ T cells were significantly increased in both types of tumors as shown by flow cytometry (FIGS. 23K-23O, 33H-33I). These data indicate that CD8⁺ T cells accumulate in the Ythdf2-KO TME. Although GO analysis showed that up-regulated DEGs in NK cell and cDC clusters were also enriched in antitumor immune pathway (FIG. 32F-32G), our flow cytometry data showed that tumor-infiltrated NK cells, MDSCs, and DCs were comparable between WT and Ythdf2-KO tumors (FIGS. 33J-33L). Taken together, these results indicate that tumor YTHDF2^{-/-} deficiency reshapes the immunosuppressive TME by increasing anti-tumoral macrophages and effector CD8⁺ T cells.

### YTHDF2 Deficiency Inhibits Tumor Growth Through Recruitment of CX3CR1⁺ Macrophages Via CX3CL1

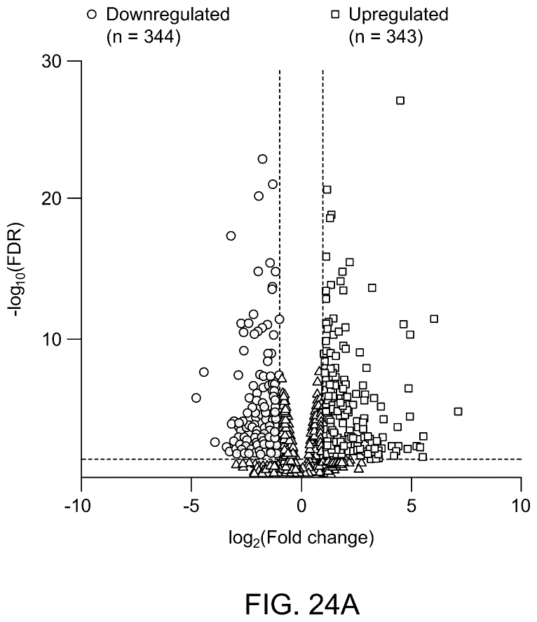

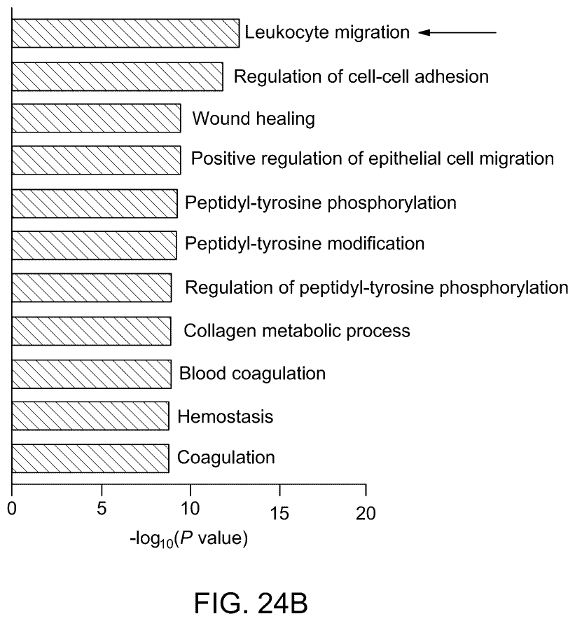

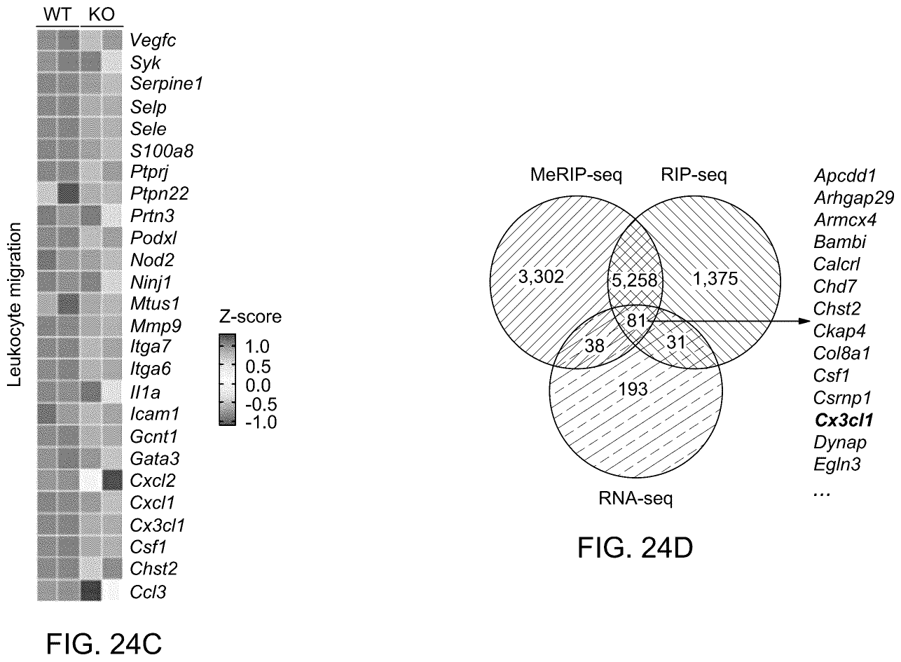

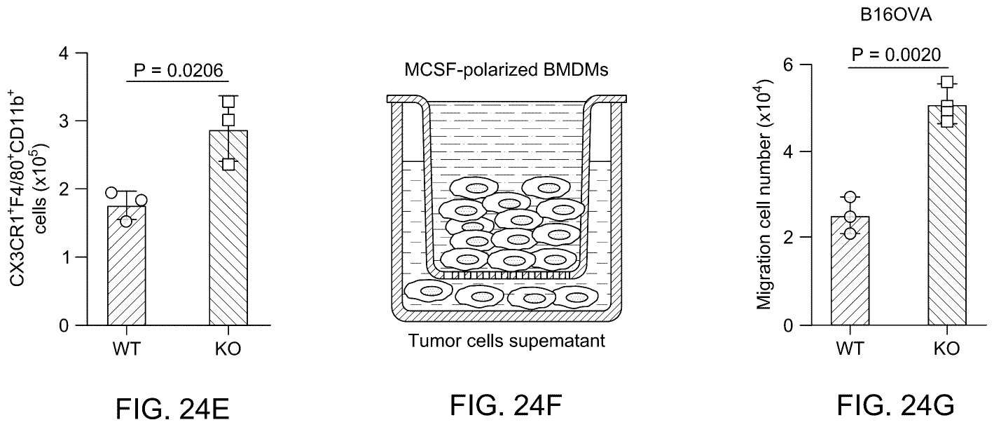

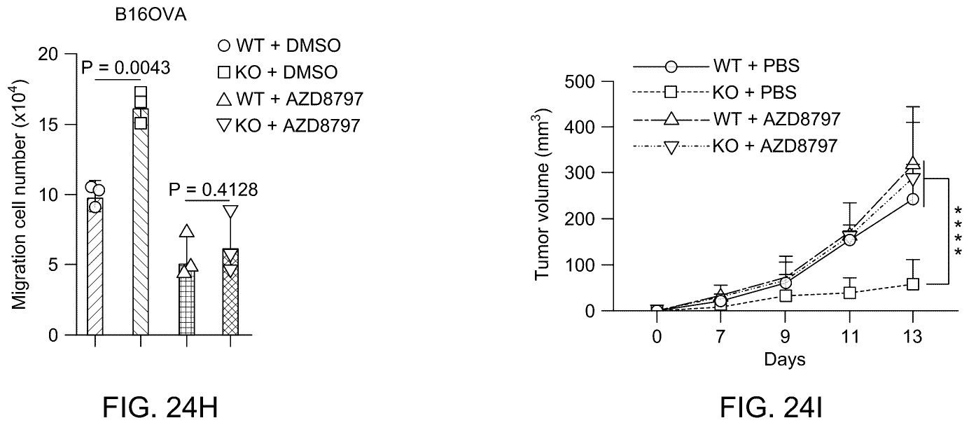

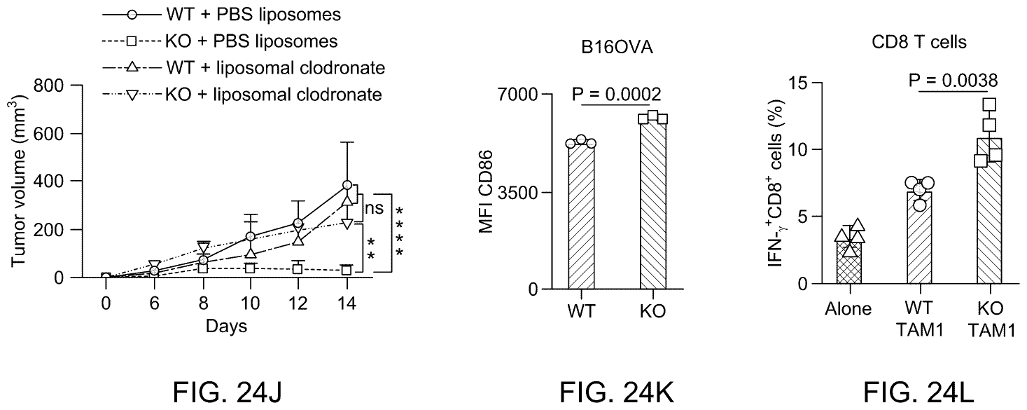

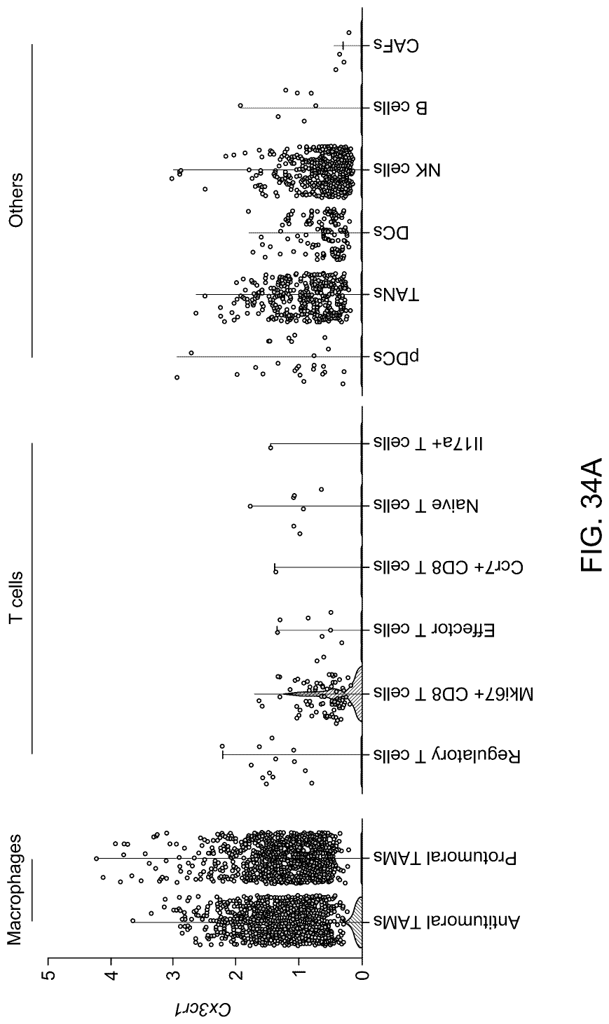

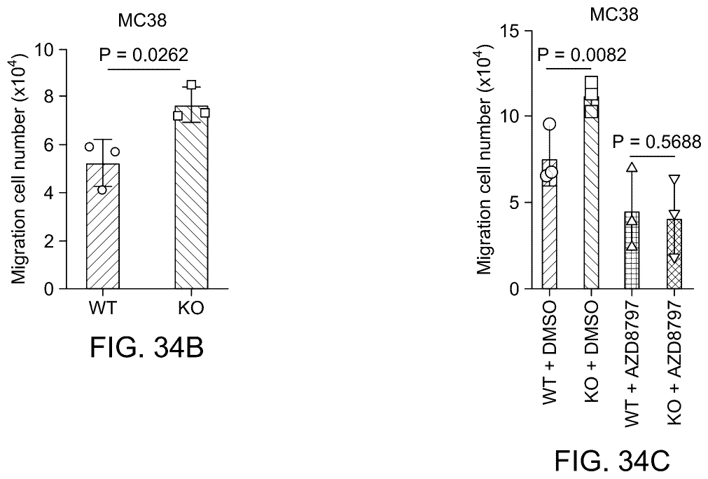

To elucidate the mechanism by which tumor-intrinsic Ythdf2-deficient remodels the TME, we performed bulk RNA-seq of WT and Ythdf2-KO MC38 tumor cells. DEGs analysis showed that 343 genes were significantly up-regulated while 344 were downregulated in Ythdf2-KO tumor cells (FIG. 24A). GO analysis showed that up-regulated DEGs were mostly enriched in leukocyte migration pathways, such as leukocyte migration, regulation of leukocyte migration, myeloid leukocyte migration, leukocyte chemotaxis, cell chemotaxis, macrophage chemotaxis, macrophage migration, etc. (FIG. 24B), indicating that YTHDF2 regulates immune cell chemotaxis. In the leukocyte migration pathway, we observed significant upregulation of Ccl3, Cxcl1, Cxcl2, and Cx3cl1 gene expression in Ythdf2-KO tumor cells (FIG. 24C). These changes may be involved in macrophage recruitment (Cook et al., 1995; Kaur et al., 2015; Vries et al., 2015; Zhang et al., 2023). To gain further mechanistic insight, we performed RNA immunoprecipitation sequencing (RIP-seq) and m⁶A-seq in both MC38 WT and Ythdf2-KO tumor cells. We then identified potential targets by overlapping transcripts from RNA-seq up-regulated DEGs, m⁶A-seq, and RIP-seq. A set of 81 transcripts bound by YTHDF2 and marked with m⁶A in both MC38 WT and Ythdf2-KO tumor cells were differentially expressed between MC38 WT and Ythdf2-KO tumor cells. Among them, cx3cl1 was a potential candidate (FIG. 24D). Of note, our scRNA-seq results showed that Cx3cr1 (the ligand of Cx3cl1 (Imai et al., 1997)) was highly expressed in anti-tumoral macrophages (FIG. 34A). Flow cytometry showed increased infiltration of CX3CR1⁺CD86⁺ anti-tumoral macrophages in Ythdf2-KO tumors compared to WT tumors (FIG. 24E), indicating that Cx3cl1 may be involved in macrophage migration into TME.

To this end, a transwell assay was established (FIG. 24F) to assess the effect of WT versus Ythdf2-KO tumor cell-derived CX3CL1 on the migration of macrophages. Ythdf2-KO tumor supernatant induced more macrophage migration to the bottom of the well compared to WT tumor supernatant (FIGS. 24G, 34B). After blocking CX3CR1 signaling using a specific inhibitor (AZD8797), differences in macrophage migration between WT and Ythdf2 KO groups disappeared (FIGS. 24H, 34C). Furthermore, AZD8797 treatment abolished the difference of tumor growth between WT and Ythdf2-KO tumor (FIG. 24I). Finally, depleting macrophages using liposomal clodronate also eliminated the difference in tumor growth between WT and Ythdf2-KO tumor (FIG. 24J). Together, these results indicate that loss of YTHDF2 upregulates the expression of CX3CL1 in tumor cells, which induces CX3CR1⁺ macrophage recruitment.

YTHDF2 Regulates the Stability of Cx3cl1 mRNA in Tumor Cells

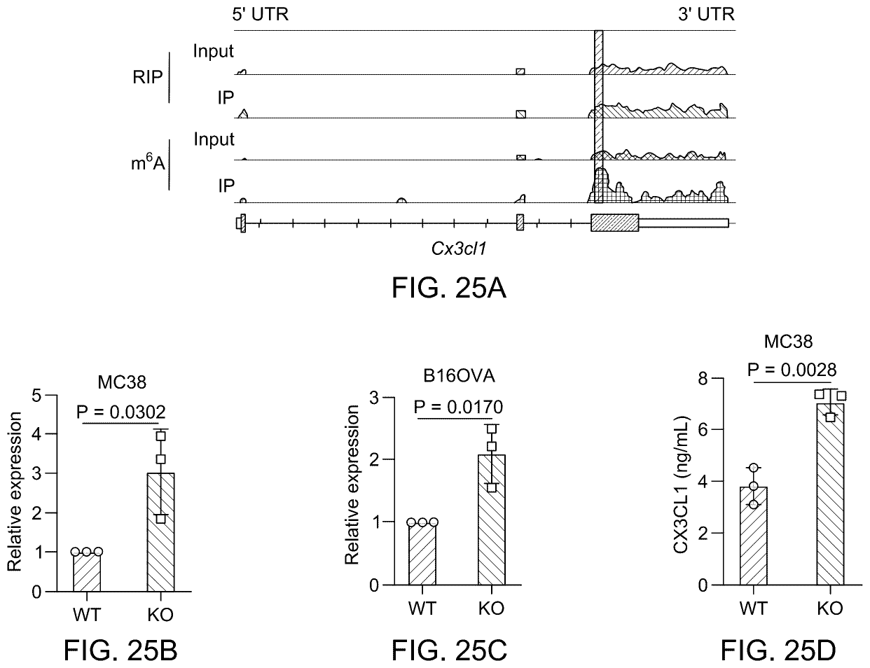

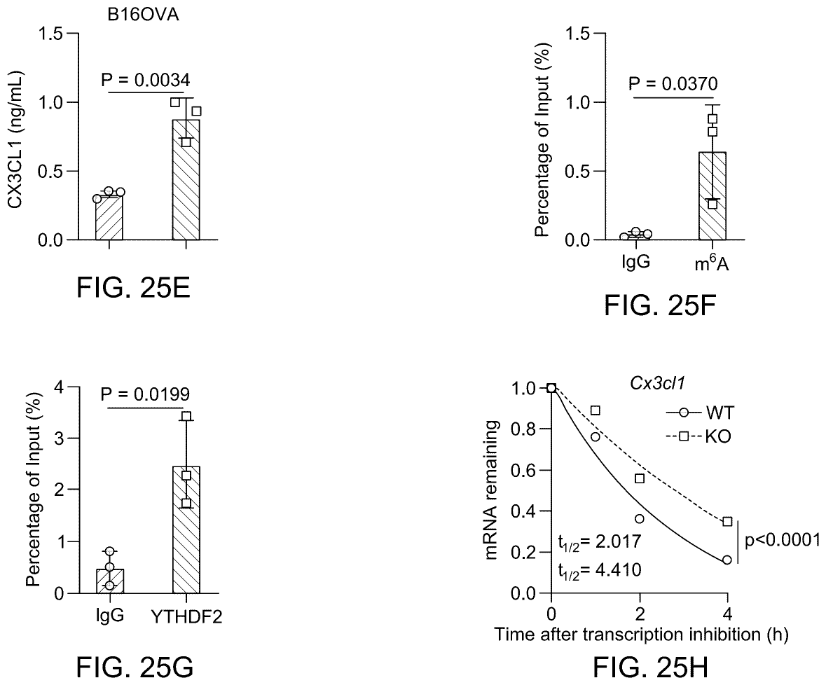

We next elucidated the molecular mechanism by which YTHDF2 regulates CX3CL1 expression. The m⁶A peaks fit well with the YTHDF2-binding site at the 3′-UTR of Cx3cl1, as shown by the Integrative Genomics Viewer (FIG. 25A). Quantitative PCR (qPCR) analysis validated that the mRNA expression of Cx3cl1 was significantly up-regulated in Ythdf2-KO tumors compared to WT tumors (FIGS. 25B-25C). Moreover, Ythdf2-KO tumor cells produced more CX3CL1 protein compared to WT tumor cells in the supernatant (FIGS. 25D-25E). RIP using either m⁶A or YTHDF2 antibody following qPCR confirmed that Cx3cl1 was indeed m⁶A methylated and enriched predominately by YTHDF2 in MC38 tumor cells (FIGS. 25F-25G). mRNA stability assay showed that Cx3cl1 had longer half-lives in Ythdf2-KO tumor cells compared to those in WT tumor cells (FIG. 25), indicating that Cx3cl1 is directly regulated by YTHDF2 in tumor cells. These observations indicate that YTHDF2 regulates the stability of Cx3cl1 mRNA in tumor cells.

### Ythdf2-Deficiency Promotes the Polarization and Antigen Presentation of Anti-Tumoral Macrophages

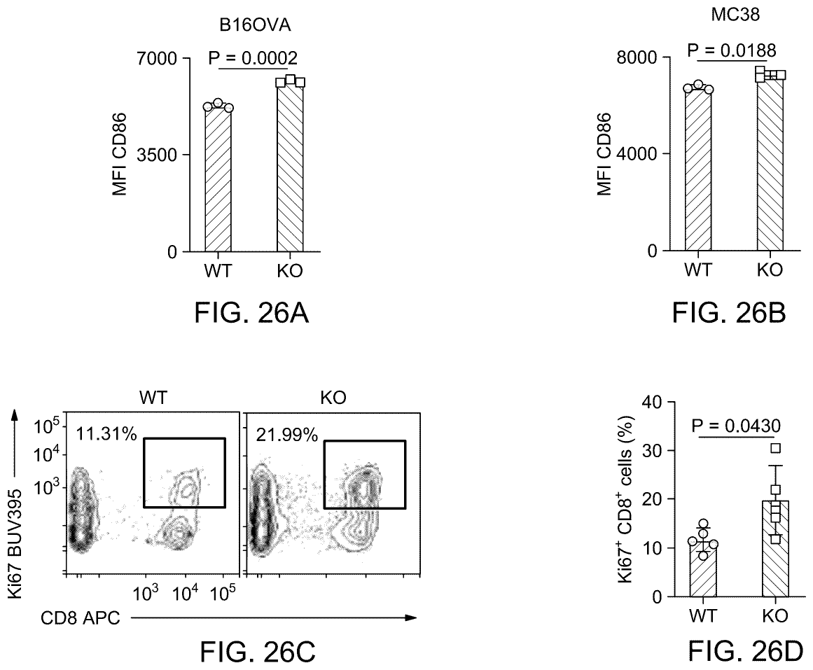

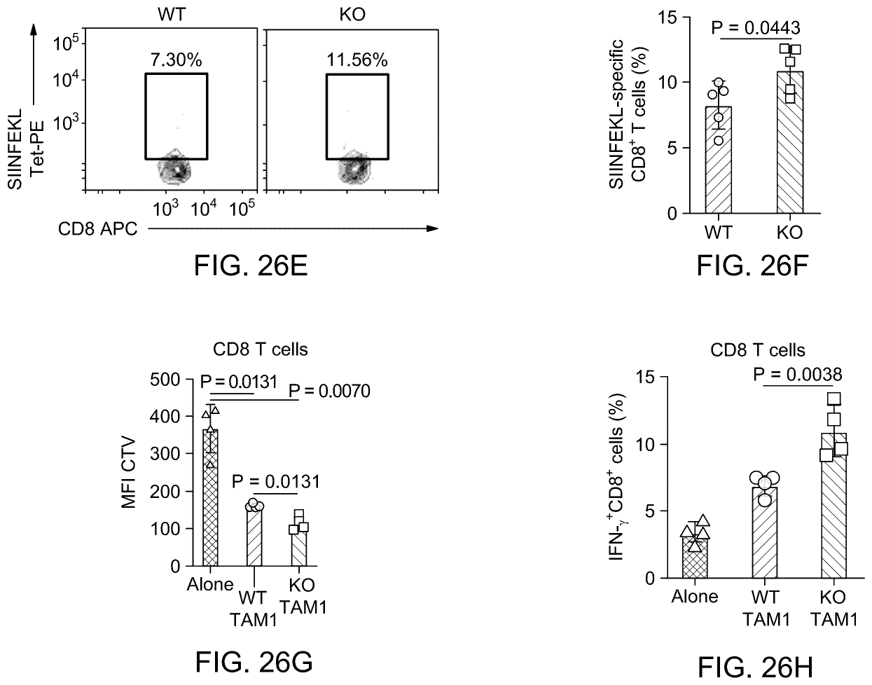

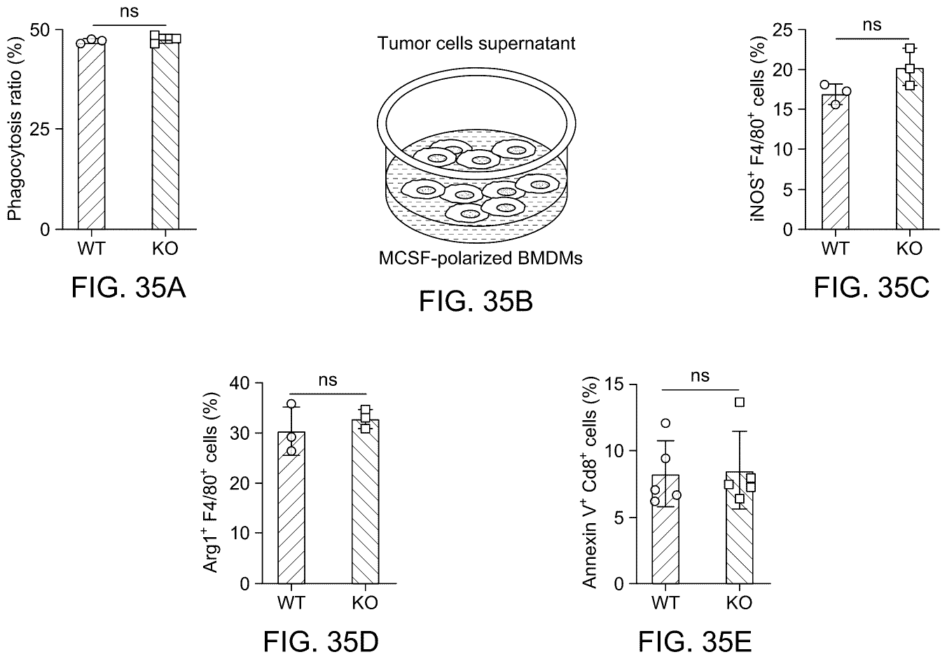

Having established that Ythdf2 deficiency inhibits tumor growth by recruiting macrophages, we further assessed whether YTHDF2 could regulate macrophage phagocytosis or polarization. First, we investigated the impact of Ythdf2-KO tumors on the phagocytic activity of macrophages. CTV-labeled WT or Ythdf2-KO MC38 tumor cells were cocultured with BMDMs for 4 hours. Flow cytometry revealed that there were similar levels of phagocytized tumor cells (FIG. 35A). Next, we performed an in vitro polarization assay by exposing macrophages to tumor supernatant (FIG. 35B). Compared to WT tumor supernatant, Ythdf2-KO tumor supernatant showed no significant changes in either anti-tumoral macrophages (expressing iNOS) or pro-tumoral macrophages (expressing ARG1) (FIGS. 35C-35D), indicating that Ythdf2-KO tumor supernatant alone has no impact on macrophage polarization. However, anti-tumoral macrophages could be polarized by pro-inflammatory stimuli such as IFN-γ (Martinez and Gordon, 2014). Since our previous results indicated the presence of increased production of IFN-γ within the Ythdf2-KO TME (FIGS. 23N-23O), Ythdf2 deficiency might promote anti-tumoral macrophages polarization in the presence of IFN-γ. Indeed, Ythdf2-KO tumor supernatant significantly up-regulated the expression of the anti-tumoral marker CD86 compared to WT tumor cells in the presence of IFN-γ (FIGS. 26A-26B). These data indicate that YTHDF2 may enhance anti-tumoral macrophages polarization in the presence of IFN-γ.

The viability of tumor infiltrating CD8⁺ T cells was similar between WT and Ythdf2-KO mice, as shown by annexin V staining (FIG. 35E). However, the percentage of Ki67⁺CD8⁺ T cells was higher in Ythdf2-KO tumors compared to those in WT tumors (FIGS. 26C-26D). In addition, the frequency of tumor infiltrating SIINFEKL-specific CD8⁺ T cells was significantly higher in Ythdf2-KO B16-OVA tumors compared to those in WT tumors (FIGS. 26E-26F). These results indicate that Ythdf2-deficiency in tumor cells promotes both CD8⁺ T cell proliferation and effector function. It has been observed that macrophages can promote T cell proliferation and effector function by cross-presenting tumor antigens (DeNardo and Ruffell, 2019). To explore whether anti-tumoral TAMs from Ythdf2 KO tumors have an increased antigen presentation activity, we isolated tumor infiltrating CD86⁺ TAMs from both WT and Ythdf2-deficient B16-OVA tumor bearing mice and co-cultured these TAMs with naïve OT1 CD8⁺ T cells in the presence of OT1 peptide SIINFEKEL for 2 days. CD8⁺ T cells had increased proliferation and produced more IFN-γ when co-cultured with TAMs isolated from Ythdf2-KO B16-OVA tumor bearing mice compared to those isolated from WT tumors (FIGS. 26G-26H). Collectively, these results indicate that Ythdf2-deficiency promotes anti-tumoral macrophage polarization and enhances antigen cross-presentation.

Ythdf2 deficiency dampens tumor glycolysis and subsequently enhances CD8⁺ T cell mitochondrial respiration and effector function.

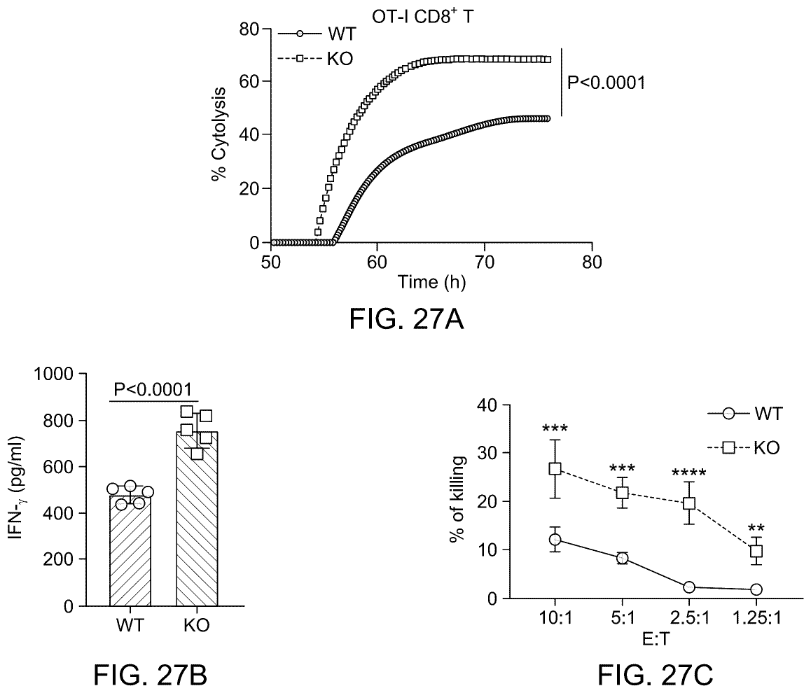

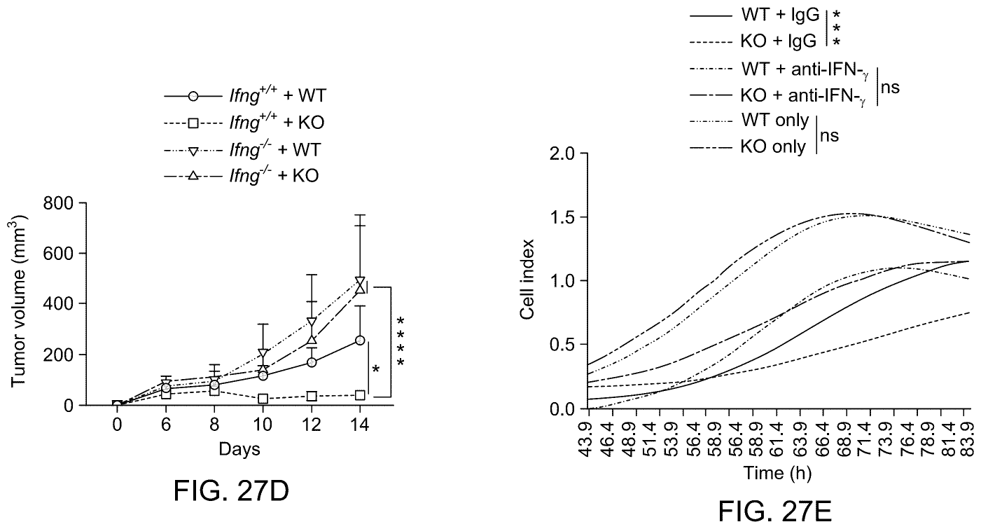

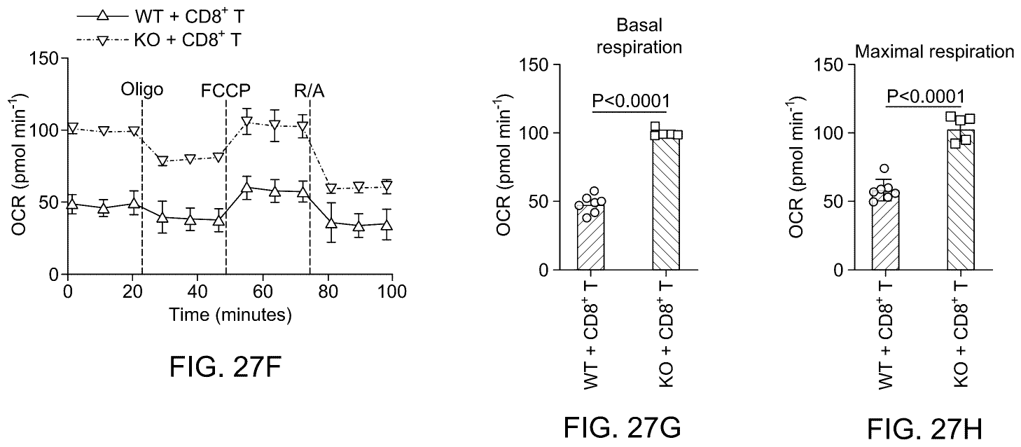

Although anti-tumoral TAMs can prime CD8⁺ T cell activation via antigen cross presentation, we wanted to assess whether Ythdf2-deficient tumor cells could directly regulate the function of CD8⁺ T cells. We measured antigen-specific killing activity by CD8⁺ T cells in a classical cytotoxic T lymphocytes assay (Zeng et al., 2022), using OVA-specific OT1 CD8⁺ T cells cocultured with either WT or Ythdf2-KO B16-OVA tumor cells. The cytotoxic activity by real time cell analysis results showed that Ythdf2-deficient tumor cells were more susceptible to OT1 CD8⁺ T cell killing in vitro (FIG. 27A). In addition, OT1 CD8⁺ T cells produced more IFN-γ when cocultured with Ythdf2-KO B16-OVA tumor cells compared to those cocultured with WT B16-OVA tumor cells (FIG. 27B). Similar results were found using MC38-OVA cells (FIG. 27C). Furthermore, we observed that Ythdf2-KO B16-OVA tumors grew similarly compared to WT tumors when implanted into Ifng-KO hosts (FIG. 27D). Meanwhile, Ythdf2-deficient tumor cells have similar susceptibility to OT1 CD8⁺ T cell killing in vitro when compared to WT tumor cells in the absence of IFN-γ signaling (FIG. 27E). Taken together, these findings indicate that YTHDF2 regulates tumor cell sensitivity to CD8⁺ T cell cytotoxicity in an IFN-γ-dependent manner.

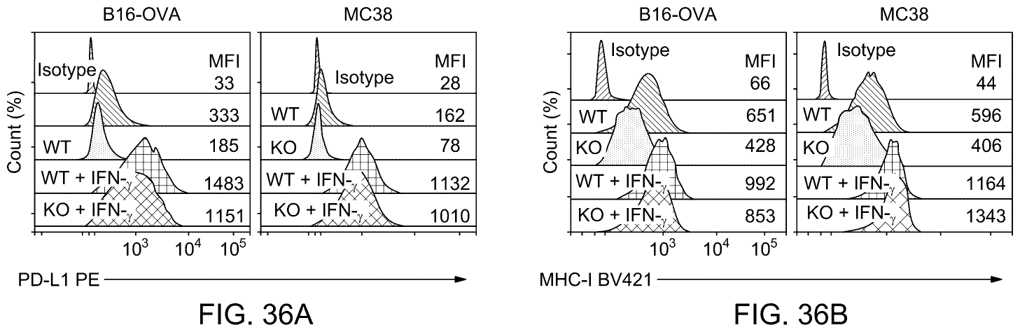

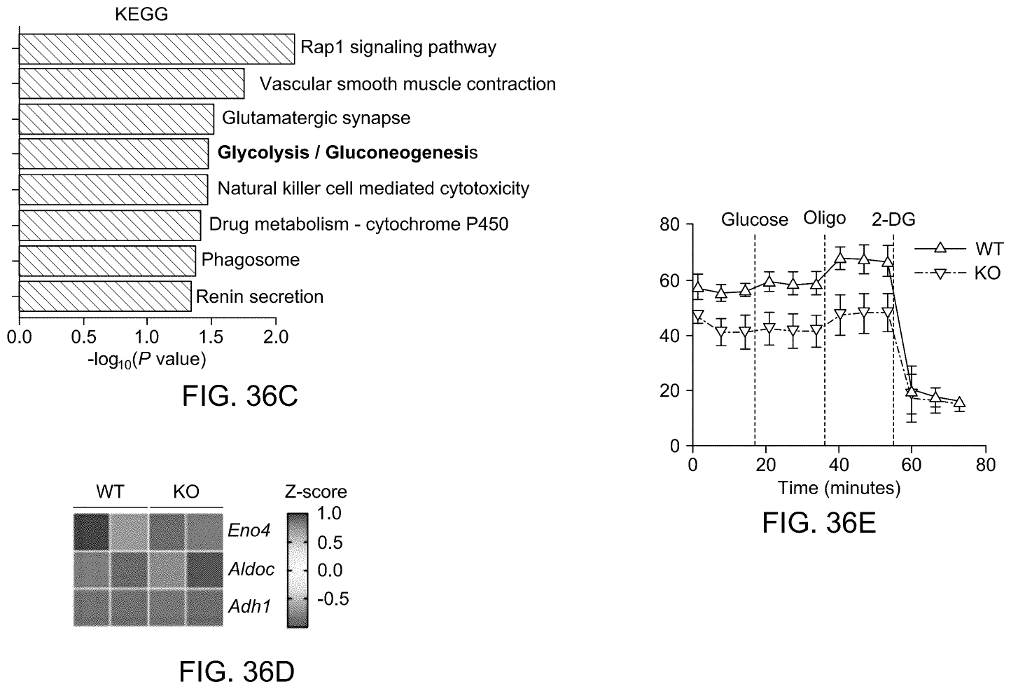

Next, we investigated the mechanism by which Ythdf2 deficiency renders tumor cells more sensitive to CD8⁺ T cell cytotoxicity. PD-L1 and MHC-I are the two most critical molecules that regulate tumor evasion (Diskin et al., 2020; Garcia-Lora et al., 2003). However, the expression levels of both PD-L1 and MHC-I were comparable between WT and Ythdf2-KO tumor cells (FIG. 36A-36B), indicating other factors are involved. The high consumption of glucose by tumors is known to restrict T cell metabolism and effector function by creating an acidic and hypoxic TME (Chang et al., 2015; Renner et al., 2019). Our Kyoto Encyclopedia of Genes and Genomes (KEGG) analysis showed that the glycolysis pathway was enriched in Ythdf2-KO tumor cells (FIG. 36C). Additionally, we observed a downregulation of several glycolysis-related genes, such as Eno4, Aldoc, and Adn1 in Ythdf2-KO tumor cells (FIG. 36D). These findings indicate that Ythdf2-KO tumors modify glycolysis metabolism and thus create a nonacidic and hyperoxia TME that lifts restrictions on CD8⁺ T cells. Using a Seahorse XF bioanalyzer, we confirmed that Ythdf2-KO tumor cells had significantly decreased glycolytic capacity compared to WT tumor cells (FIG. 36E). Moreover, CD8⁺ T cells co-cultured with Ythdf2-KO tumor cells exhibited a higher basal and maximum OCR compared to those co-cultured with WT tumor cells (FIGS. 27F-27H). These data indicate that Ythdf2-KO tumor cells stimulate CD8⁺ T cell effector function by dampening tumor glycolysis and subsequently enhancing CD8⁺ T cell mitochondrial respiration.

### IFN-γ Downregulates YTHDF2 Expression Via Autophagy-Mediated Protein Degradation in Tumor Cells

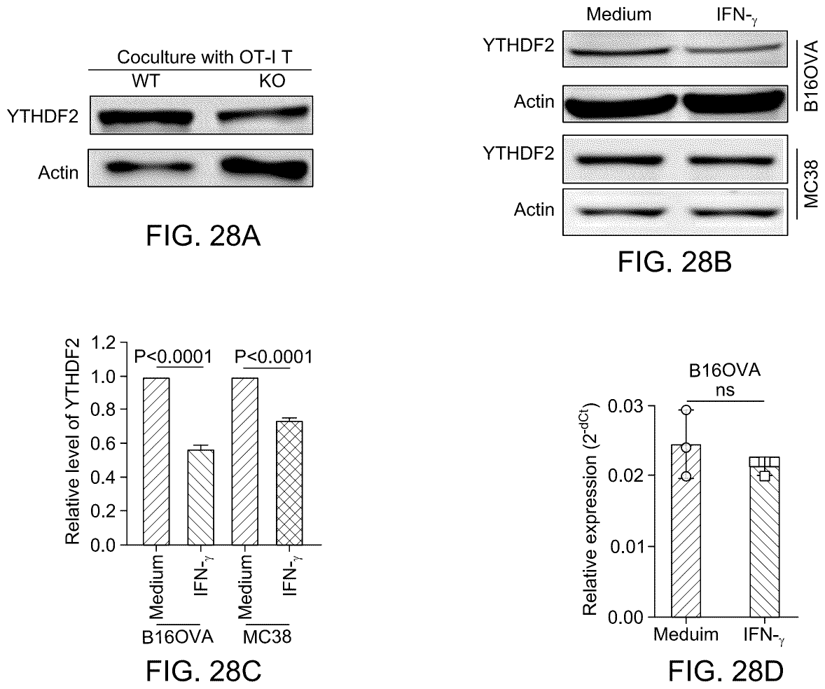

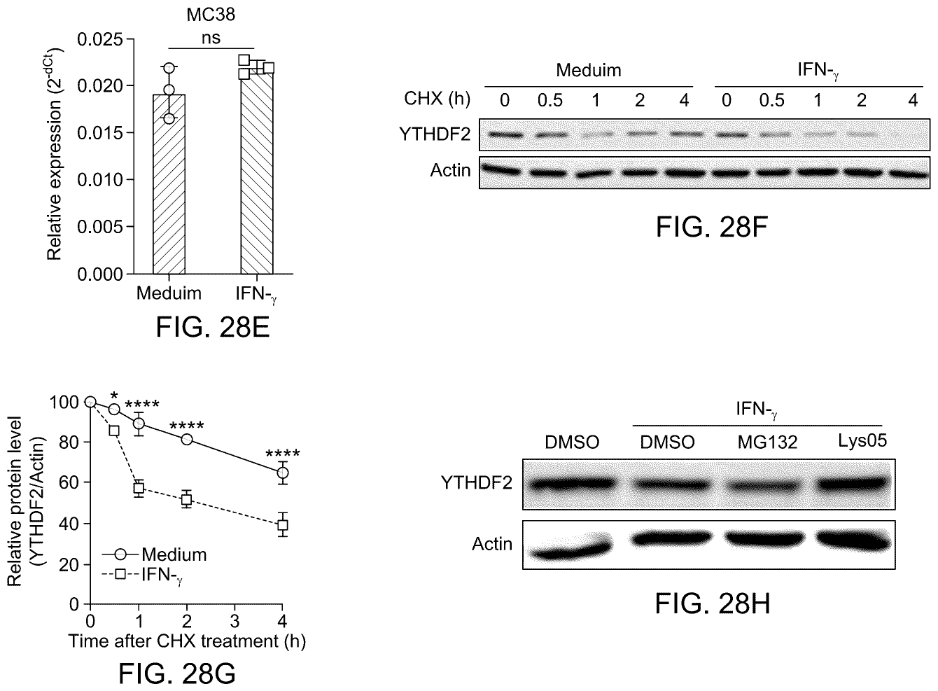

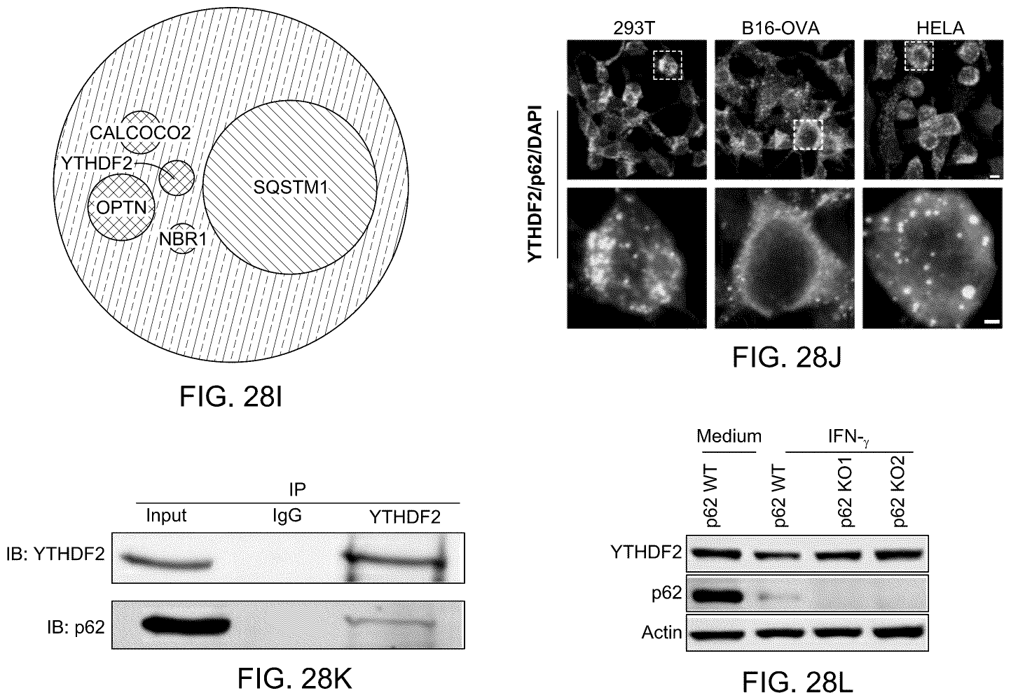

Immune cells, such as CD8⁺ T cells, usually edit tumor cells and trigger them to evade immune surveillance (Kim et al., 2007). We found that tumor cells that were cocultured with CD8⁺ T cells had a lower expression level of YTHDF2 compared to that were not cocultured (FIG. 28A). IFN-γ is the master effector cytokine produced by CD8⁺ T cells, and stimulation of tumor cells with IFN-γ had the same effect in decreasing YTHDF2 expression (FIGS. 28B-28C), indicating that CD8⁺ T cells and their associated production of IFN-γ could decrease the expression of YTHDF2 in tumor cells. To determine the mechanism by which IFN-γ downregulates YTHDF2, we first assessed whether IFN-γ affects Ythdf2 expression at mRNA levels. Our results showed that Ythdf2 mRNA had no change following treatment with IFN-γ (FIGS. 28D-28E), indicating that IFN-γ regulates YTHDF2 at the post-transcriptional level. Next, we pretreated tumor cells with cycloheximide (CHX), which prevents de novo protein synthesis, and then stimulated with IFN-γ. The results showed that YTHDF2 protein degraded quicker in the presence of IFN-γ compared to untreated cells (FIGS. 28F-28G), indicating that IFN-γ regulates YTHDF2 protein stability. To determine which protein degradation pathway is involved in this process, we used various inhibitors to block either the proteasome or the autophagic-lysosomal pathways (Klionsky, 2007; Mizushima et al., 2008). The results showed that treatment with the lysosome inhibitor Lys05, but not proteasome inhibitor MG132, could reverse the YTHDF2 degradation induced by IFN-γ in tumor cells (FIG. 28H), indicating that IFN-γ regulates YTHDF2 protein stability via the autophagy pathway.

Different adapter proteins can be recruited to interact with the cargo to promote protein degradation (He and Klionsky, 2009; Johansen and Lamark, 2011). We therefore assumed that YTHDF2 may interact with the adapter protein of the autophagy pathway to mediate its degradation. Integrated interactions database (IID) (Kotlyar et al., 2016) was used to predict protein-protein interactions (PPI) for YTHDF2 and autophagy adapters (SQSTM1, NBR1, NDP52, and OPTN). This analysis showed that YTHDF2 may preferably bind to p62 (encoding by Sqstml) (FIG. 28I), which transports the cargo to autophagosomes for degradation (Liu et al., 2016). It has also been reported that p62 can mediate the degradation of some other m⁶A proteins (Cui et al., 2021; Yang et al., 2022), and IFN-γ facilitates the recruitment of p62 to a cargo protein (Lee et al., 2015). Our immunofluorescence results showed the colocalization of YTHDF2 and p62 in several types of tumor cells (FIG. 28J). A co-immunoprecipitation assay showed that YTHDF2 physically bound to p62 in B16-OVA cells (FIG. 28K). Notably, p62 deletion markedly abolished the YTHDF2 protein degradation in IFN-γ treated tumor cells (FIG. 28L). Take together, these findings indicate that p62 binds to YTHDF2 and promotes its autophagic degradation in tumor cells.

### Development of a Small Molecule Degrader Targeting YTHDF2 for Cancer Treatment in the Absence or Presence of a PD-L1 Blockade

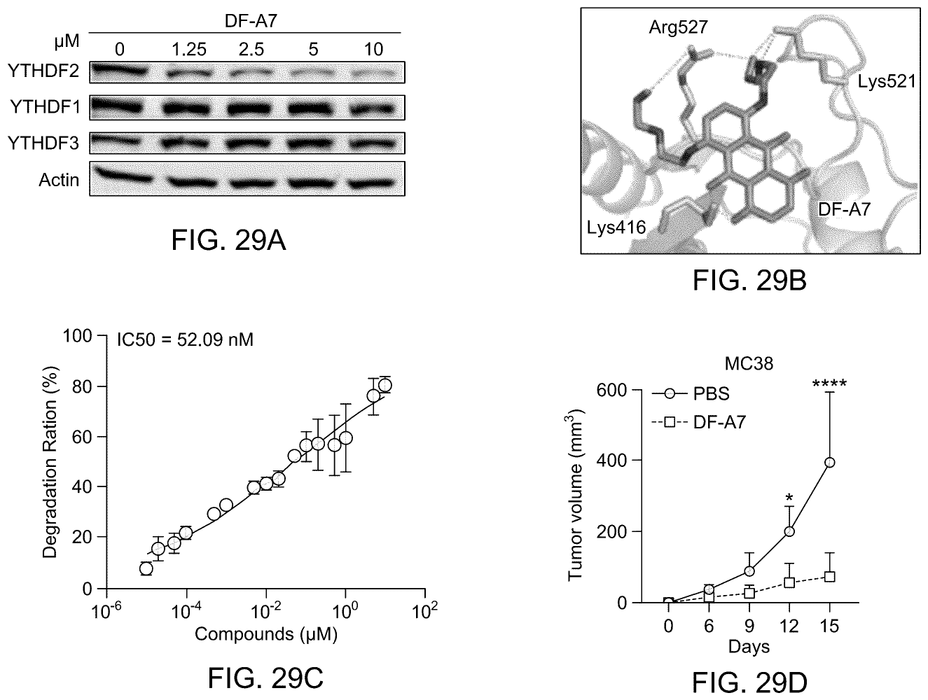

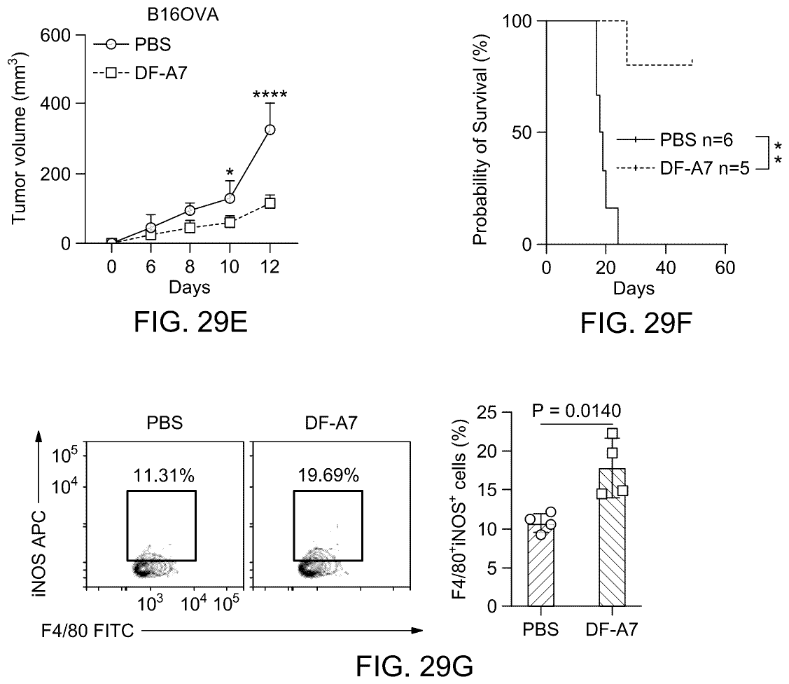

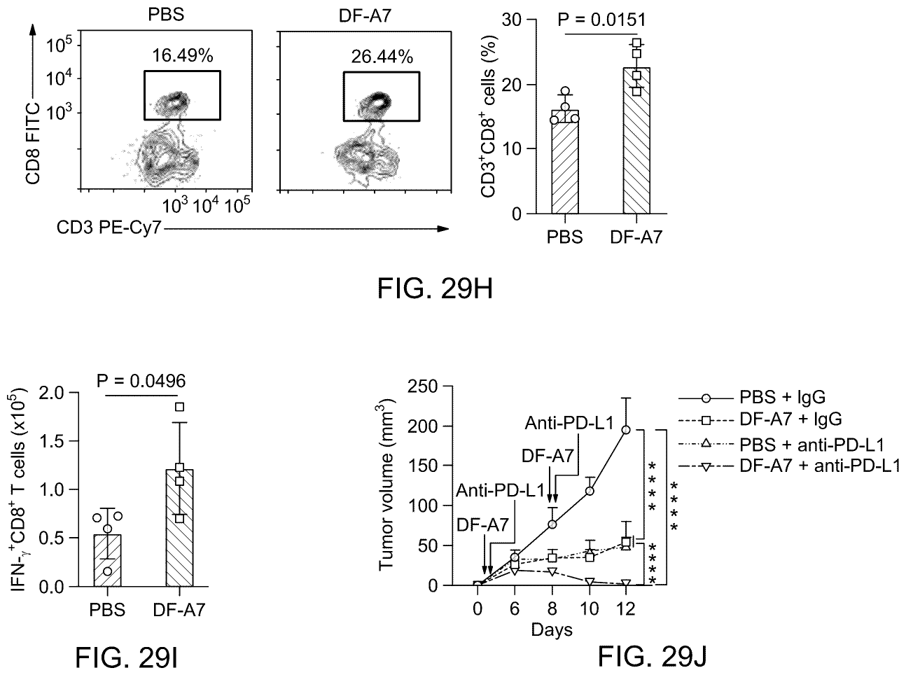

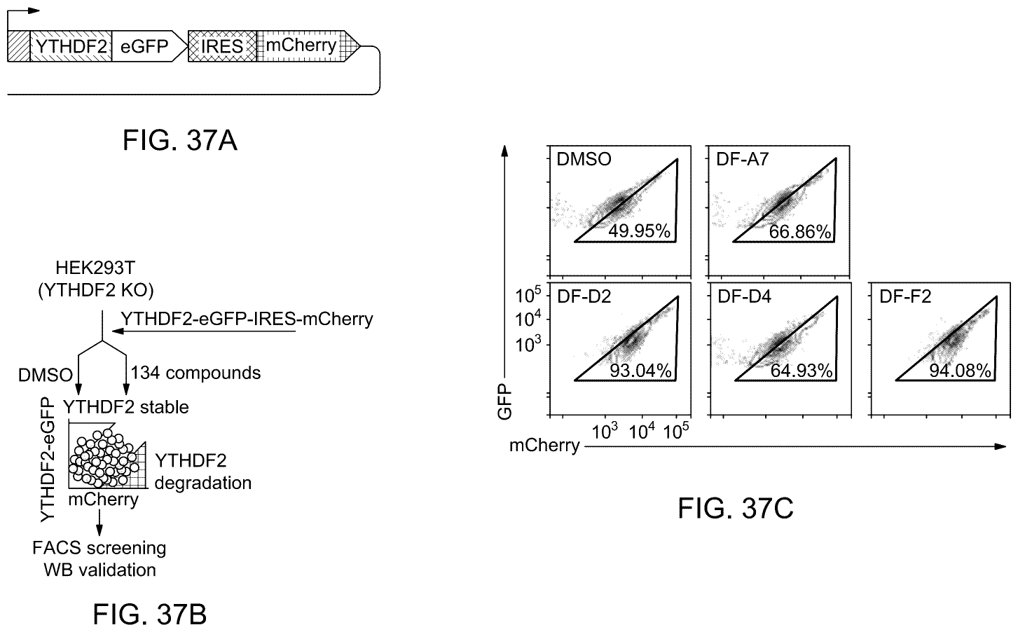

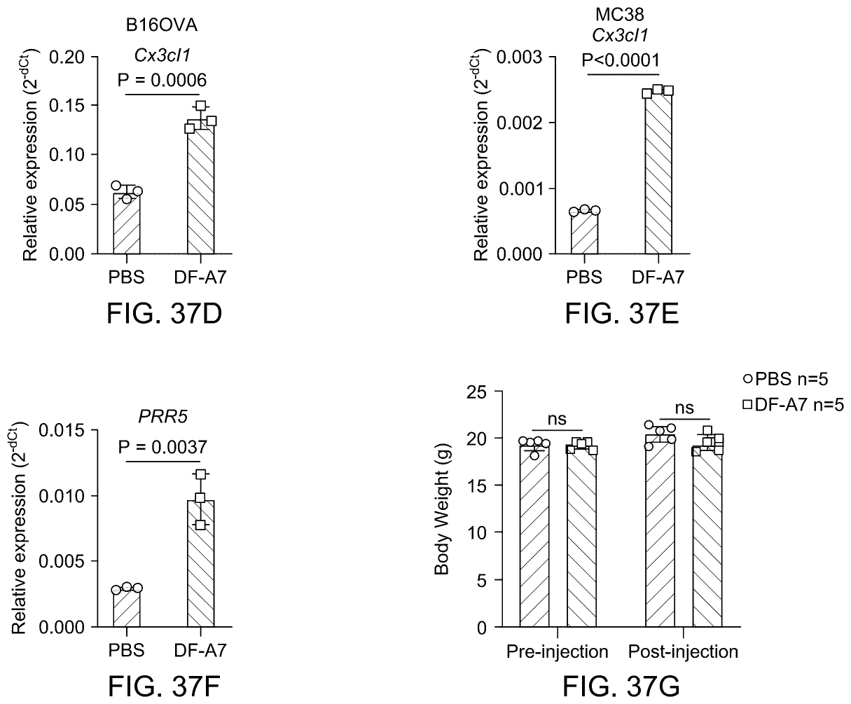

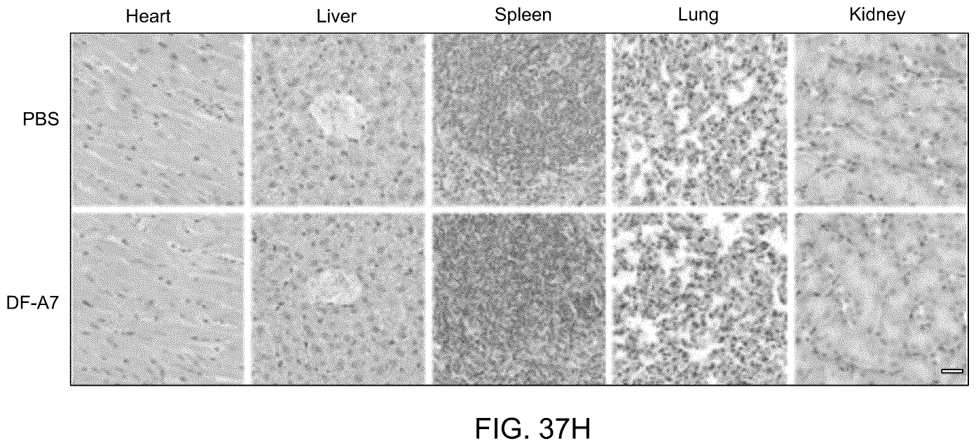

Our findings indicate that tumor intrinsic YTHDF2 is a promising target for cancer immunotherapy. Given that m⁶A reader proteins share the same or overlapping binding sites on target genes yet have contrasting functions (Zaccara and Jaffrey, 2020), it presents a challenge to screen for inhibitors that selectively target specific reader proteins. Alternatively, targeted protein degradation is a promising approach to remove select proteins through small molecules that can induce the degradation of target proteins through the protein degradation system (Dale et al., 2021; Raina and Crews, 2017). Therefore, we first conducted a structure-based virtual screening to identify potential small molecules that can bind with YTHDF2 from a library containing approximately 100K compounds. A total of 134 candidate compounds with high docking scores were preliminarily selected. Next, to further screen for a small molecule that could potentially serve as a small molecule degrader inducing YTHDF2 protein degradation, we generated a fluorescent reporter system in HEK293T cells. In this system, the gene encoding the full-length YTHDF2 protein was fused with eGFP, followed by an internal ribosome entry site (IRES), and mCherry (FIG. 37A) (Slabicki et al., 2020b). The extent of degradation can be determined by measuring the levels of YTHDF2_{eGFP} normalized to mCherry (as the ratio of eGFP/mCherry) expression. Specifically, following treatment with the small molecule candidate compounds, a 1:1 ratio of eGFP/mCherry indicates that its protein stability is unaffected, while a ratio greater than 1 indicates increased stability and less than 1 indicates degradation (FIG. 37B) (Slabicki et al., 2020a). Among 134 compounds, four compounds facilitated the degradation of YTHDF2_{eGFP} (FIG. 37C). We then performed immunoblotting to verify the degradation of YTHDF2_{eGFP} and validated that one compound, named DF-A7, mediated the degradation of YTHDF2 without affecting the degradation of YTHDF1 and YTHDF3 (FIG. 29A), indicating that DF-A7 specifically targets and degrades YTHDF2. To validate the direct binding of DF-A7 to YTHDF2, we generated the binding structure of DF-A7 with YTHDF2 protein using flexible docking. The complex showed that DF-A7 was mainly bound with YTHDF2 protein at K416, K521, and R527 sites (FIG. 29B). Immunoblotting showed that DF-A7 degraded YTHDF2 with an IC50 around 50 nM (FIG. 29C). Using the mRNA level of Cx3cl1 and PRR5 as readouts (two direct targets of YTHDF2) (Liu et al., 2018), we further validated that DF-A7 increased the mRNA level of Cx3cl1 and PRR5 after degrading YTHDF2 (FIGS. 37D-37F).

We then evaluated the anti-tumor efficacy of DF-A7. B16-OVA and MC38 tumor-bearing mice received i.p. injections of DF-A7 on day 1 and day 8 post tumor implantation with PBS used as vehicle control. We observed that DF-A7 significantly inhibited tumor growth compared to vehicle control in both tumor models (FIGS. 29D-29E). Moreover, DF-A7 treatment significantly prolonged the survival of mice compared to animals treated with vehicle control (FIG. 29F). Flow cytometry of cells isolated from tumor bearing mice showed both increased tumor infiltrating anti-tumoral macrophages and IFN-γ-producing CD8⁺ T cells within DF-A7 treated tumors compared to vehicle controls (FIGS. 29G-29I). Furthermore, we observed no differences in mouse body weight (FIG. 37G) or in the morphology of the heart, liver, spleen, lung, and kidney (FIG. 37H) between the DF-A7 treated group and vehicle group. More importantly, we also investigated whether a combination treatment of DF-A7 and a PD-L1 blocking agent could further improve therapeutic outcomes. Compared to the groups receiving monotherapy, mice that were administered the combination therapy demonstrated the slowest tumor growth (FIG. 29J). Together, our results indicate that targeting with DF-A7 can inhibit tumor growth and also displays improved anti-tumor efficacy when used in combination with PD-L1 blockade therapy.

### DISCUSSION

In this study, we identified a central role for YTHDF2 in tumor immune evasion and immunoediting. Tumor intrinsic YTHDF2 not only inhibits macrophage recruitment and anti-tumoral polarization, but also suppresses CD8⁺ T cell antitumor effector function. This eventually establishes a permissive immunosuppressive tumor microenvironment that ultimately promotes tumor progression. YTHDF2 also contributes to CD8⁺ T cell-mediated immunoediting, which renders tumor cells more resistant to the cytotoxicity of CD8⁺ T cells and aids in maintaining the metabolic fitness of tumor cells. Finally, we developed a specific and potent YTHDF2 degrader, which shows strong anti-tumor efficacy either alone or in combination with anti-PD-L1 therapy.

TAMs are potent regulators of tumor-associated immune suppression in the TME (Anderson and Simon, 2020). Our group recently reported that YTHDF2 plays a critical role in shaping the TME through reprogramming TAMs (Ma et al., 2023b). Ablation of YTHDF2 in macrophages reprogrammed TAMs towards the tumor suppressive anti-tumoral type (Ma et al., 2023b). Myeloid-derived suppressor cells (MDSCs) are another crucial negative regulator of antitumor immune responses. More recently, Wang et al. reported that loss of YTHDF2 in MDSCs inhibits their infiltration and suppressive function, thereby augmenting antitumor immunity and overcoming tumor radio resistance (Wang et al., 2023). Our current study shows that tumor intrinsic YTHDF2 also negatively regulates the tumor TME and aids in progression. Given the detrimental roles of YTHDF2 in TAMs, MDSCs, and tumor cells, a strategy of dual-targeting YTHDF2 in both tumor cells and suppressive immune cells presents a promising strategy for tumor immunotherapy.

Within recent years, continuous efforts are being made to identify highly effective and safe lead compounds for targeting m⁶A modification (Cully, 2019; Gu et al., 2020; Yankova et al., 2021). The demethylase FTO inhibitors, such as rhein (Chen et al., 2012), meclofenamic acid (Huang et al., 2015), FB23/FB23-2 (Huang et al., 2019), and Dac51 (Liu et al., 2021) have all demonstrated potent antitumor capacity. Additionally, the METTL3 inhibitors STM2457 (Yankova et al., 2021) and UZH1a (Moroz-Omori et al., 2021) show antitumor activity in acute myeloid leukemia. However, a safe, effective, and druggable inhibitor targeting m⁶A readers has not yet been identified. A recent study reported a potential YTHDF2 inhibitor, DC-Y13-27, that promotes antitumor immune responses by targeting YTHDF2 in MDSCs (Wang et al., 2023). Similar to the mechanism of most m⁶A inhibitors, DC-Y13-27 inhibits tumor progression by preventing the binding of YTHDF2 with m⁶A sites. However, whether DC-Y13-27 could also inhibit the binding of YTHDF1/YTHDF3 with m⁶A sites has not been validated. Moreover, with an IC50 of 22 μM, it appears unlikely that DC-Y13-27 will be a viable drug candidate. Here, leveraging structure-based virtual screening of a library with over 100,000 small compounds, we identified the small molecule DF-A7 that binds YTHDF2 and subsequently promotes its degradation. Interestingly, DF-A7 specifically promotes the degradation of YTHDF2 while having no effect on YTHDF1 or YTHDF3. Of note, DF-A7 degrades YTHDF2 in an IC50 of 50 nM, which is 500-fold lower than that of DC-Y13-27. This indicates that DF-A7 will enhance antitumor function via targeting m⁶A modification in tumor cells at achievable dose levels.

In conclusion, our work reveals the tumor intrinsic and central role of the m⁶A reader YTHDF2 in tumor immune evasion and editing through CD8⁺ T cells. Our previous work and current study highlight that YTHDF2 is an excellent target not only within immune cells but also in tumor cells. Finally, we have identified DF-A7 as the first safe, effective, and druggable small compound inhibitor of YTHDF2 for this specific targeting purpose.

### Methods

Mice. Rag1^{-/-} mice were kindly provided by Z. Sun (City of Hope National Medical Center). Wild-type C57BL/6, OT1 (003831), NOD scid gamma (NSG) (005557), and Ifng knockout (KO) (002287) mice were purchased from The Jackson Laboratory. Rag2^{-/-}Il2rg^{-/-} mice were provided by the animal facility at City of Hope. All experimental mice were bred and maintained under specific pathogen-free conditions. 6- to 12-week-old mice of both sexes were used for experiments. Mice were housed in the City of Hope Animal Facility with a 12-hours light/12-hours dark cycle and temperatures of ˜18-23° C. with 40-60% air humidity. All animal experiments were approved by the City of Hope Institutional Animal Care and Use Committee.

Cell lines. The mouse colon adenocarcinoma cell line MC38 was provided by S. Priceman (City of Hope National Medical Center). Ovalbumin (OVA)-expressing B16 (B16-OVA) cells were provided by M. Kortylewski (City of Hope National Medical Center). Ovalbumin (OVA)-expressing MC38 (MC38-OVA) cells were provided by Z. Sun (City of Hope National Medical Center). HEK293T and HELA cells were purchased from ATCC. All cell lines were cultured in DMEM complete medium (Dulbecco's modified Eagle medium (Gibco) supplemented with 10% fetal bovine serum (FBS; Gibco)). Cells were incubated at 37° C. with 5% CO2.

Generation of CRISPR-edited Ythdf2-deficiency tumor cell lines. To generate MC38, MC38-OVA, and B16-OVA Ythdf2-KO cell lines, we used crisprRNA (crRNA) (5-CGAACCTTACTTGAGCCCAC (SEQ ID NO:6)) targeting exon 2 of the Ythdf2 gene. In brief, pre-transcribed Alt-R® CRISPR-Cas9 crRNAs and Alt-R® CRISPR-Cas9 tracrRNA (Catalog #1072532) were purchased from IDT (Integrated DNA Technologies, Inc., Coralville, Iowa). Guide RNA (gRNA) was prepared by incubating 200 μM each of crRNA and tracrRNA together in a total volume of 5 μl in Nuclease-Free IDTE, pH 7.5 (1×TE solution, Catalog #11-01-02-02) at 95° C. for 5 minutes. The Cas9/RNP complex was formed by incubating 1.7 μl of Alt-R® S.p. HiFi Cas9 Nuclease V3 protein (105 pmol) (Catalog #1081060), 1.2 μl of gRNA (120 pmol), and 2.1 μl of PBS in a total volume of 5 μl for 10 minutes at room temperature. MC38, MC38-OVA and B16-OVA tumor cells were resuspended in 20 μl of P3 Primary Cell 4D-Nucleofector™ X Solution and 5 μl of Cas9/RNP complex and 1 μl of 100 μM of Alt-R® Cas9 Electroporation Enhancer (Catalog #1075915), and electroporated using Lonza 4D-Nucleofector system with pulse DP-113 (MC38, MC38-OVA) and DJ-110 (B16-OVA). After electroporation, tumor cells were rested for 3 days in DMEM complete medium before assessing the efficiency of CRISPR modification using immunoblotting.

Tumor models and treatment. MC38 or B16-OVA cells were implanted subcutaneously (s.c.) into the flanks of 6- to 12-week-old female and male mice. The length (a) and width (b) of the tumors were measured starting on day 6 (tumor implanted as day 0) and either every other day or every three days thereafter. The tumor volume was calculated with the formula a×b²/2. The maximal tumor size was limited to 15 mm in diameter, as specified in our approved animal protocol. For experiments with CD8⁺ T cell, CD4⁺ T cell, or NK cell depletion, mice were injected intraperitoneally (i.p.) with 200 μg of anti-CD8 (clone 2.43, BioXcell), anti-CD4 (clone GK1.5, BioXcell), or anti-NK1.1 (clone PK136, BioXcell), respectively, on day 0 and 7 after tumor inoculation. For macrophage depletion, mice were injected i.p. with 200 μl of liposomal clodronate (Encapsula NanoSciences) on days 0 and 7 after tumor inoculation. For tumor transplantation from immunodeficient to immunocompetent mice, tumor cells were initially inoculated into Rag1^{-/-} mice. When the tumor volume reached over 200 mm³, tumor fragments were transplanted into wild-type C57BL/6 mice. For CX3CR1 blocking, mice were injected i.p. with 1 mg/kg of AZD8797 (HY13848, MCE, USA) once per day after tumor inoculation.

Flow cytometry. Single-cell suspensions were prepared from the tumor tissue of MC38 or B16-OVA implantation mice, as described previously (Ma et al., 2023b). Flow cytometry analysis and cell sorting were performed on BD LSRFortessa X-20 and FACSAria Fusion flow cytometers (BD Biosciences), respectively. Data were analyzed using NovoExpress software (Agilent Technologies). The following fluorescent dye-labeled antibodies purchased from Biolegend, Biosciences were used in this study: CD3 (17A2), CD4 (GK1.5), CD8 (53-6.7), CD8 (KT15), NK1.1 (PK136), CD11b (M1/70), CD11c (N418), CD45 (30-F11), Ly6C (HK1.4), Ly6G (IA8), F4/80 (BM8), MHC class II (M5/114.15.2), IFNγ (XMG1.2), iNOS (CXNFT), Arg1 (AlexF5), CD86 (GL1), CX3CR1 (SA011F11), Ki67 (B56), PD-L1 (10F.9G2), MHC-I (28-8-6) and anti-mouse H-2K^{b} bound to SIINFEKL (25-D1.16). H-2Kb SIINFEKL-PE was kindly provided by the NIH Tetramer Core Facility. For examining IFN-γ production from T cells, cells were first stimulated with a leukocyte activation cocktail (50583, BD Biosciences) in the presence of the protein transport inhibitor brefeldin A for 4 hours. Cells were then stained with the indicated cell surface markers and fixed/permeabilized using a fixation/permeabilization kit (eBioscinece). Cell pellets were resuspended in PBS with 2% FBS for flow cytometry analysis.

Generation of bone-marrow-derived macrophages (BMDMs). Bone marrow was extracted from the femurs and tibias of C57BL/6 mice. All bone marrow cells were flushed out and filtered through a 100-μm cell strainer. After centrifugation, red blood cells were lysed. The resultant bone marrow cells were resuspended in RPMI1640 complete medium (RPMI1640 (Gibco) supplement with 10% FBS (Gibco), 1% penicillin/streptomycin (Gibco) and 50 μM 2-mercaptoethanol (Sigma)) in the presence of 20 ng/mL macrophage colony-stimulating factor (M-CSF; Peprotech) for 5-7 days. After stimulating, the BMDMs are ready to use.

Cell migration assay. Supernatant from cultured MC38 WT or Ythdf2-KO tumor cells was collected and centrifuged to remove dead cells. Tumor culture supernatant was used as conditioned medium. Differentiated BMDMs were seeded at 3×10⁵ cells in 300 μl RPMI1640 complete medium into a 3-μm insert before migration experiments. Inserts containing BMDMs were transferred to a 24-well plate with either 400 μl WT or Ythdf2-KO conditioned media and 400 μl RPMI1640 complete medium. Cells migrating into the plate were harvested and counted after 24 hours co-culture. For experiments involving CX3CR1 blocking, BMDMs in the insert were pretreated overnight with 10 μM AZD8797 (MedChemExpress, HY-13848).

BMDMs polarization assay. BMDMs were cultured with 50% WT or Ythdf2-KO conditioned media in the absence or presence of mouse IFN-γ (1 ng/mL) for 24 hours. BMDM polarization was examined by flow cytometry. CD86 and Arg1 were used as markers for pro-tumoral and anti-tumoral macrophages, respectively.

Ex vivo T cell coculture assay. CD8⁺ T cells were isolated from splenic single cell suspension following the EasySep Mouse CD8 isolation kit (StemCell Technologies, 19853). Cells were then activated with mouse anti-CD3/CD28 antibodies (Biolegend, 100302 and 102102 respectively) for 2 days supplemented with human IL-2 (50 U/mL). Activated-T cells were then stained with Cell Tracer Violet (CTV, Thermo Fisher Scientific, C34571) at 1 μM for 20 min at 37° C. with 5% CO2. CTV-labeled T cells were plated at 2×10⁵ cells per well in a 96-well plate. Tumor cells were then added at a ratio of 10:1. For the antigen presentation assay, WT or Ythdf2-KO B16-OVA cells were collected on day 14 and processed as described in the flow cytometry section. CD86⁺ TAMs were isolated from the WT and Ythdf2-KO tumors following cell sorting. After isolation, cells were primed with SIINFEKEL peptide (10 μg/mL, InvivoGen) for 2 hours. Primed CD86⁺ TAMs were washed and cocultured with CTV labeled naïve CD8⁺ T cells isolated from OT1 mice for 3 days. Brefeldin A was added to the cocultures for 4 hours prior to the end of incubation. The cross-priming capacity of TAMs was then evaluated by flow cytometry as was the IFN-γ production by CD8⁺ T cells and the proportion of CTV-low CD8⁺ T cells.

Seahorse XF96 respirometry. WT or Ythdf2-KO B16-OVA cells were seeded at 2×10⁴ per well in a XF96 plate and stabilized overnight. The extracellular acidification rate (ECAR) was measured by the XF96 extracellular flux analyzer with glucose stress fuel flex test kits (Agilent). Measurements of ECAR were performed according to the manufacturer's instructions. 1.5×10⁵ per well CD8⁺ T cells were co-cultured with WT or Ythdf2-KO B16-OVA cells for 24 hours and subsequently seeded in the XF96 plate and stabilized for 2 hours. The oxygen consumption rate (OCR) was measured by the XF96 extracellular flux analyzer with Mito Stress test kits (Agilent). Measurements of OCR were performed according to the manufacturer's instructions. All the results were analyzed using Wave software (Seahorse/Agilent).

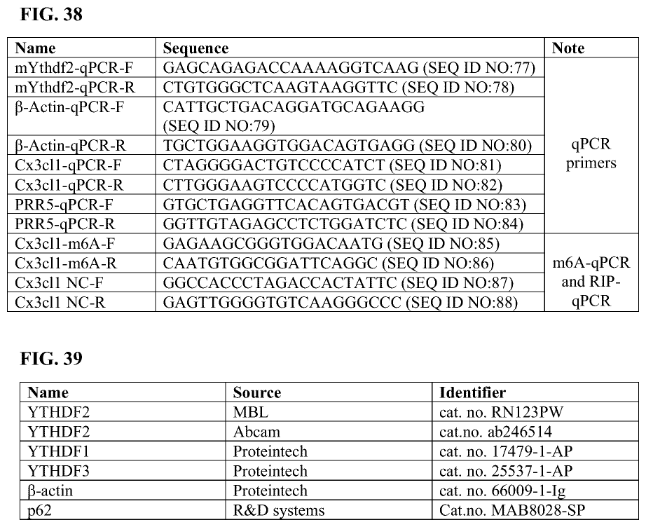

Reverse transcription polymerase chain reaction (RT-PCR). RNA was isolated using a RNeasy mini kit (QIAGEN) and then reverse transcribed to cDNA with a PrimeScript RT reagent kit with gDNA Eraser (Takara Bio) following the manufacturer's instructions. mRNA expression levels were analyzed using SYBR Green PCR master mix and a QuantStudio 12K Flex real-time PCR system (both from Thermo Fisher Scientific). Primer sequences are listed in FIG. 38.

RNA stability assay. Tumor cells were seeded in 24-well plates at 2×10⁵ cells/mL. 5 g/mL of Actinomycin D (Sigma-Aldrich, A9415) was added. After 0, 1, 2, and 4 hours of incubation, cells were collected, and total RNA was purified using RNeasy mini kit (QIAGEN). RNA stability was determined using RT-qPCR analysis as described previously (Ma et al., 2023b).

Immunoblotting. Immunoblotting was performed according to standard procedures, as previously described (Ma et al., 2023a). In brief, cells were lysed in RIPA Lysis and Extraction Buffer (Thermo Fisher Scientific, 89900). Protein concentration was assessed using the BCA Protein Assay Kit (Thermo Fisher Scientific, 23225). Samples were resolved using SDS-PAGE gels and transferred to PVDF membranes. Membranes were blocked with Intercept® Blocking Buffer (LI-COR, 927-60001) for 30 min at room temperature and incubated overnight at 4° C. with primary antibodies. Immunoblots were visualized with Odyssey CLx Imager (LI-COR). Immunoblotting antibodies are listed in FIG. 39.

Protein degradation assay. B16-OVA tumor cells were treated with mouse IFN-γ (100 ng/mL, Peprotech) for 24 hours. Treated cells were then supplied with the translation inhibitor cycloheximide (CHX, 50 ng/mL, Sigma-Aldrich). Cells were collected at 0.5, 1, 2, and 4 hours after CHX treatment. Cells without inhibitor were dubbed 0 hour. Protein levels of YTHDF2 were determined by immunoblotting and subsequently quantified with Image Studio™ Acquisition Software.

Coimmunoprecipitation. Cells were lysed in lysis buffer (10% glycerol, 50 mM Hepes-KOH (pH 7.5), 100 mM KCl, 2 mM EDTA, 0.1% NP-40, 10 mM NaF, 0.25 mM Na3VO4, 50 mM β-glycerophosphate, 2 mM dithiothreitol, and 1× protease and phosphatase inhibitor cocktail) and were incubated on ice for 15 min. Cell lysates were centrifuged at 14,000 g for 15 min at 4° C., and supernatant were collected. Cell lysates were incubated with YTHDF2 antibody (5 g per sample) overnight at 4° C. with rotation. Dybeads Protein A (20 μL) (10001D, Invitrogen) was added to each tube, followed by rotating at 4° C. for 2 hours. The beads were separated out with a magnet and washed five times with cold lysis buffer. The remaining beads were resuspended in 4×LDS (Lithium dodecyl sulfate) Sample buffer (Invitrogen) and incubated at 70° C. for 10 min. The supernatants were then collected and used for immunoblotting.

scRNA-seq. Implanted B16-OVA WT or Ythdf2-KO tumors from C57BL/6 mice were extracted and digested with collagenase type IV (1 mg/mL) and DNase type I (30 U/mL) for 30 min at 37° C. The resulting cells were filtered through 100-μm cell strainers, washed in PBS, lysed in red blood cell buffer, and resuspended in PBS. Tumor-infiltrating immune cells (CD45⁺ cells) were sorted in a FACSAria Fusion flow cytometry (BD Biosciences). The cell suspension (300-600 living cells per mL) was loaded onto a Chromium Single Cell Controller (10× Genomics) to generate single-cell gel beads in the emulsion according to the manufacture's protocol. Libraries were subsequently prepared using a Single Cell 5′ Library and Gel Bead kit and sequenced on an Illumina NovaSeq 6000 instrument with a paired-end 100-base pair reading strategy.

scRNA-seq analysis. Raw data were processed with CellRanger (version 7.1.0), demultiplex cellular barcodes, map reads to the mouse genome (mm10). Downstream analyses were performed in R (version 4.3.0) using Seurat (version 4.9.9). Less prevalent genes (<3) and cells with less prevalent genes (<200) were removed. Low-quality cells were removed if the number of expressed gene was <200 or >5000. Cells were also removed if the proportion of mitochondrial gene expression was >7.5%. The top 3000 genes identified by the ‘vst’ method were used in principle-component analysis; 1 to 30 principal components (PCs) were used in the FindNeighbors function. The FindClusters function was used to identify clusters (res=0.2), which were then visualized with Uniform manifold approximation and projection (UMPA) plots. Marker genes and differentially expressed genes (DEGs) were identified using the FindMarkers function, and only genes with adjusted P values of <0.05 and log fold changes of >1.5 (determined by two-sided Wilcoxon rank-sum test and adjusted using Bonferroni correction) were considered.

m⁶A-seq. Total RNA was isolated with TRIzoll reagent (Thermo Fisher Scientific) from 50 million MC38 WT or Ythdf2-KO tumor cells. m⁶A immunoprecipitation was performed according to standard procedures, as previously reported (Ma et al., 2021). Sequencing was performed at the Translational Genomics Research Institute (TGen) on an Illumina NovaSeq 6000 platform with 100-base pair paired-end reads (PE100). Sequencing reads were mapped to the mouse genome (mm10) using HISAT2 (version 2.2.1) (Kim et al., 2015). m⁶A-enriched peaks from m⁶A-seq samples as control. The MACS2 software was run with default options except for ‘-nomodel, -keepdup all’ to turn off fragment size estimation and to keep all uniquely mapped reads, respectively. m⁶A peaks were visualized using Integrative Genomics Viewer software (version 2.8.13). The motifs enriched in m⁶A peaks were analyzed by HOMER (version 4.11). Each peak was annotated based on Ensembl gene annotation information by applying BEDTools'intersectBed (version 2.30.0). FeatureCounts (version 2.0.6) was used to calculate read counts in input samples. The DEGs between MC38 WT and Ythdf2-KO tumor cells were detected with the R package DEseq2 (version 1.38.3), and genes with a log 2 (fold change) of >1 or <−1 and False Discovery Rate (FDR) of <0.05 were considered as DEGs.

YTHDF2 RIP-seq. 50 million MC38 WT or Ythdf2-KO tumor cells were lysed with two volumes of lysis buffer (10 mM HEPES pH7.6, 150 mM KCl, 2 mM EDTA, 0.5% NP-40, 0.5 mM DTT, 100× protease inhibitor cocktail (Thermo Fisher Scientific) and 400 U/mL SUPERase-InRNase inhibitor (Thermo Fisher Scientific)). YTHDF2 RIP was performed according to the protocol as previously reported (Ma et al., 2021). A DNA library was generated with a KAPA RNA HyperPrep kit (Roche) and sequenced on the Illumina NovaSeq 6000 platform. Sequencing reads were mapped to the mouse genome (mm10) using HISAT2 (version 2.2.1). The target binding peaks of YTHDF2 were identified using the MACS2 software (version 2.2.6). The HOMER software (version 4.11) was used to find motifs enriched in YTHDF2-binding peaks. The target genes were annotated based on Ensembl gene annotation information by applying BEDTools'intersectBed (version 2.30.0).

Analysis of data from TCGA. We acquired The Cancer Genome Atlas (TCGA) gene expression matrices and associated clinical data from UCSC Xena (http://xena.ucsc.edu/). The study did not consider samples that lacked survival information. To define a YTHDF2^{-/-} associated signature in the malignant cells of each tumor, we integrated and analyzed pan-cancer single-cell RNA sequencing (scRNA-seq) data, obtained from the studies by Avishai Gavish et al. (Gavish et al., 2023) and Gabriela S. Kinker et al. (Kinker et al., 2020). Malignant epithelial cells of each tumor were grouped according to whether YTHDF2 was expressed. Subsequently, we conducted a differentially expressed genes (DEGs) analysis employing the FindMarker function, utilizing its default parameters. DEGs were deemed the YTHDF2^{-/-} related signature within the malignant cells of each tumor. For each tumor, we evaluated signature employing the default parameters of the GSVA R package (version 1.48.2) (Hänzelmann et al., 2013). We used a 50% cutoff for the signature score to dichotomize each tumor into groups designated as signature^{high} and signature^{low}. Finally, we performed survival analysis using the survival R package (version 3.5-5) and Kaplan-Meier method with a log-rank test.

Virtual screening. To discover the candidates of small molecule compound targeting YTHDF2, the K416/R527-containing pocket of YTHDF2, which is responsible for recognizing m⁶A-RNA (Zhu et al., 2014), was selected to conducted virtual screening by using Discovery Studio 2020 software (BIOVIA). The crystal structure of YTHDF2's YTH domain was downloaded from the Research Collaboration for Structural Bioinformatics Protein Data Bank (RCSB PDB) (PDB ID: 4WQN). The compound library (126,633 compounds) was provided by ChemDiv. The LibDock program was utilized to perform molecular docking calculation (Input Site Sphere=5.95962, 73.2763, −13.3272, 12; Number of Hotspots=100; Docking Tolerance=0.25; Docking Preferences=High Quality; Conformation Method=FAST). The obtained poses were then ranked by the values of LibDockScore. The compounds with LibDockScore over 130 were selected for subsequent biological evaluation.

Flexible docking. DF-A7 and the K416/R527-containing pocket of YTHDF2 (PDB ID: 4WQN) were utilized to conduct molecular docking via the flexible docking program in Discovery Studio 2020 software (BIOVIA). 3D conformations of DF-A7 were generated via the Prepare Ligands program of Discovery Studio 2020 software. These conformations were then submitted to flexible docking (Input Site Sphere=5.95962, 73.2763, −13.3272, 12; Maximum Number=100; Number of Hotspots=100; Max Hits to Save=10; Tolerance=0.25; RMSD Filter=2.0; Conformation Method=FAST; Energy Threshold=20.0; Simulated Annealing=True), in which the K416/R527-containing pocket of YTHDF2 and DF-A7 were endowed with CHARM force field. After in silico calculation, the obtained poses were ranked by their values of CDOCKER_INTERACTION_ENERGY. Top-10 poses were chosen to analyze the key residues mediating the DF-A7-YTHDF2 interaction. The graphic images of flexible docking were processed by PyMoL v1.8 software.

Statistical analysis. To estimate the statistical significance of differences between two groups, a paired or un-paired Student's t-tests were used to calculate P values. One-way analysis of variance or two-way analysis of variance with multiple comparisons test were performed when more than two groups were compared. Survival analysis was performed using Kaplan-Meier curves and evaluated with log-rank Mantel-Cox tests. Error bars indicate the standard deviation (SD). P values are labeled in the figures. P values were denoted as follows: *P<0.05, **P<0.01, ***P<0.001, ****P<0.0001. Statistical analyses were performed by using GraphPad Prism (version 9.0).

### Example 3

CD4⁺ T cells are key regulators of the adaptive immune system. They can differentiate into various subsets, including T helper 1 (Th1), Th2, Th9, Th17, and regulatory T (Treg) cells, which are crucial for immune responses and genesis of disease. However, it remains unclear whether and how RNA N⁶-methyladenosine (m⁶A) modification, which plays a vital role in controlling mRNA stability and translation, is involved in T helper cell differentiation and function. Here, we demonstrate that YTHDF2, a key reader protein known to destabilize m⁶A-modified mRNA, is a negative regulator of Th9 cell differentiation and effector function. Ablation of Ythdf2 in both mouse and human naïve CD4⁺ T cells significantly promotes the differentiation of naïve CD4⁺ T cells into Th9 cells. Mechanistically, Ythdf2 depletion stabilizes Gata3 mRNA in CD4⁺ T cells, thus enhancing Th9 cell differentiation under IL-4 signaling. Moreover, Ythdf2-deficient Th9 cells secrete higher levels of effector cytokines, IL-9 and IL-21, leading to increased tumor infiltration and cytotoxic activity of CD8⁺ T cells and NK cells, thereby showing higher anti-tumor activity than wild-type (WT) Th9 cells in vivo. Additionally, depletion of YTHDF2 in human chimeric antigen receptor (CAR) Th9 cells enhances the expression of immune activation markers and reduces the expression of terminal differentiation genes, resulting in superior antitumor efficacy in vivo compared to WT CAR-Th9 cells. In conclusion, our findings establish that YTHDF2 acts to limit Th9 differentiation and function, while loss of YTHDF2 enhances Th9 cell differentiation and antitumor immunity. Targeting YTHDF2 represents an excellent strategy for enhancing Th9 and CAR-Th9 cell-based immunotherapies against cancer.

### YTHDF2 Deficiency Enhances Th9 Cell Differentiation in Mice

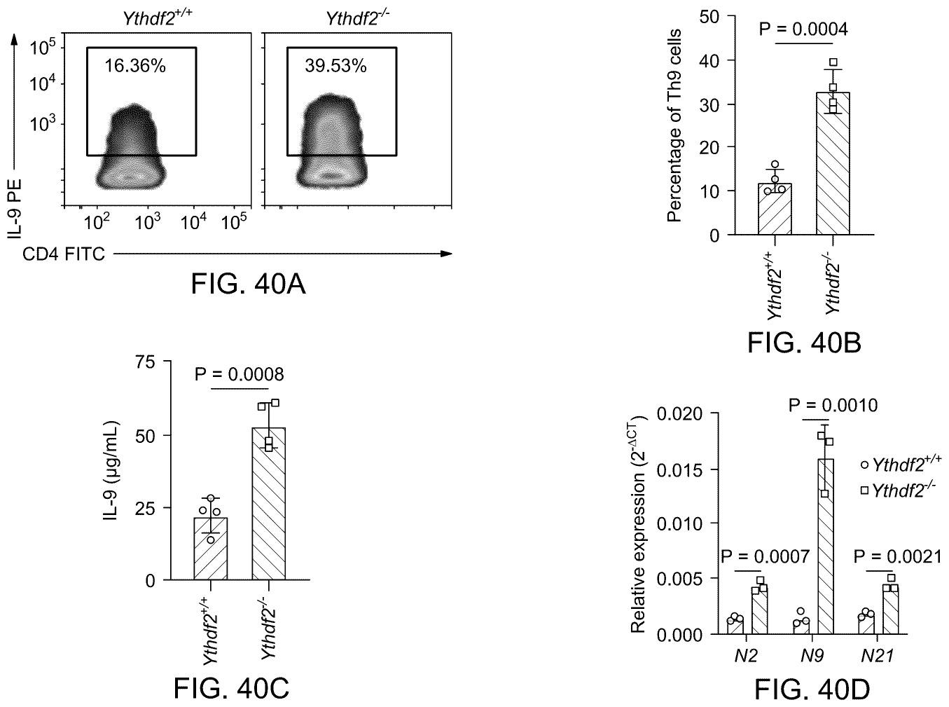

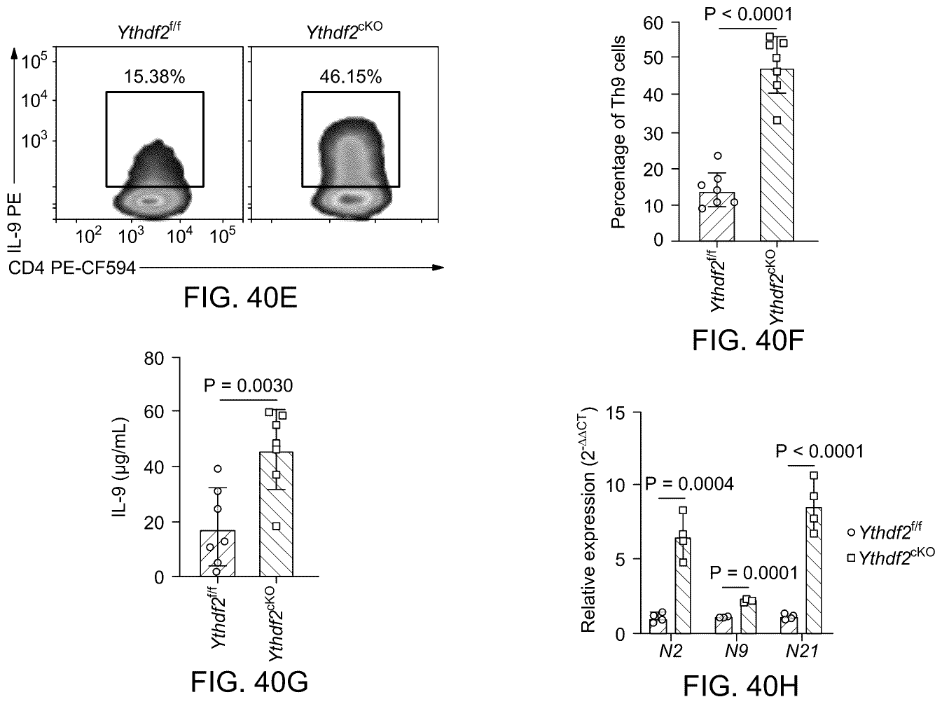

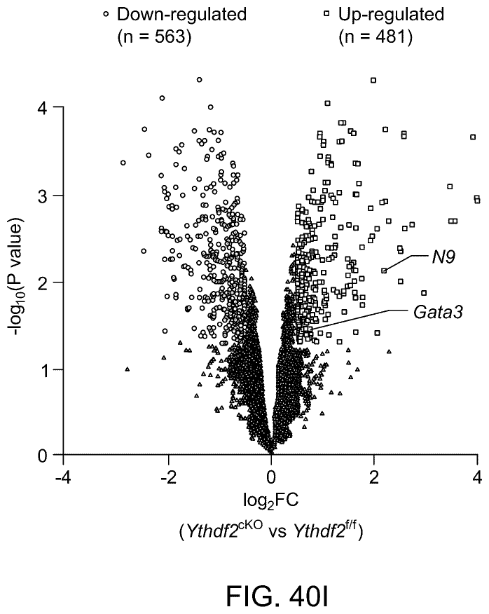

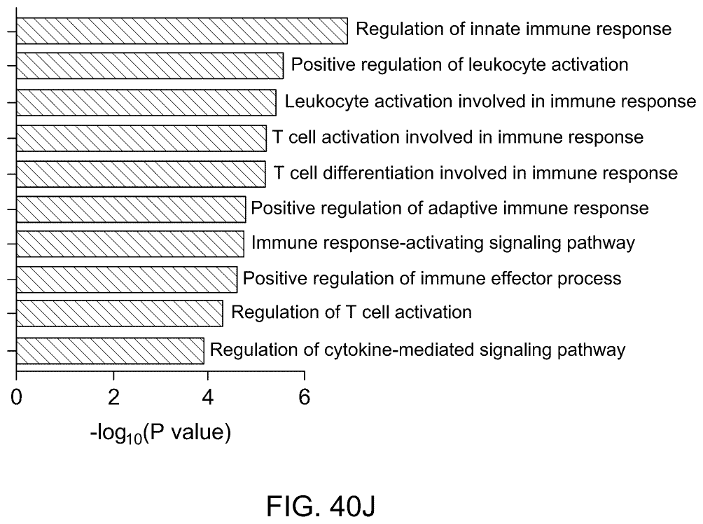

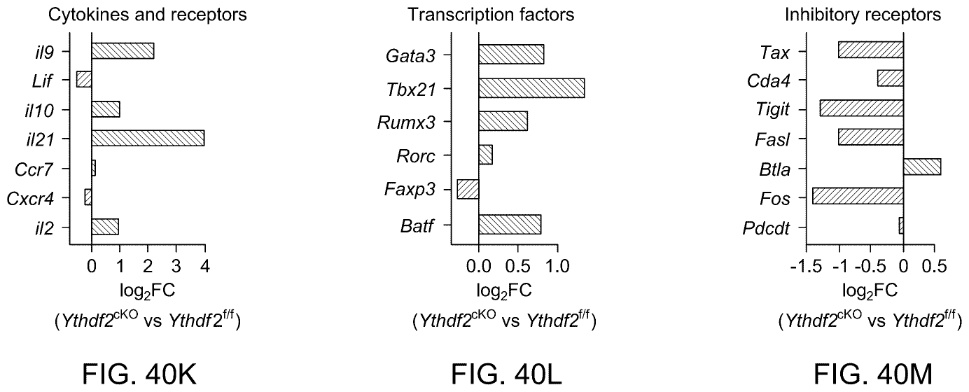

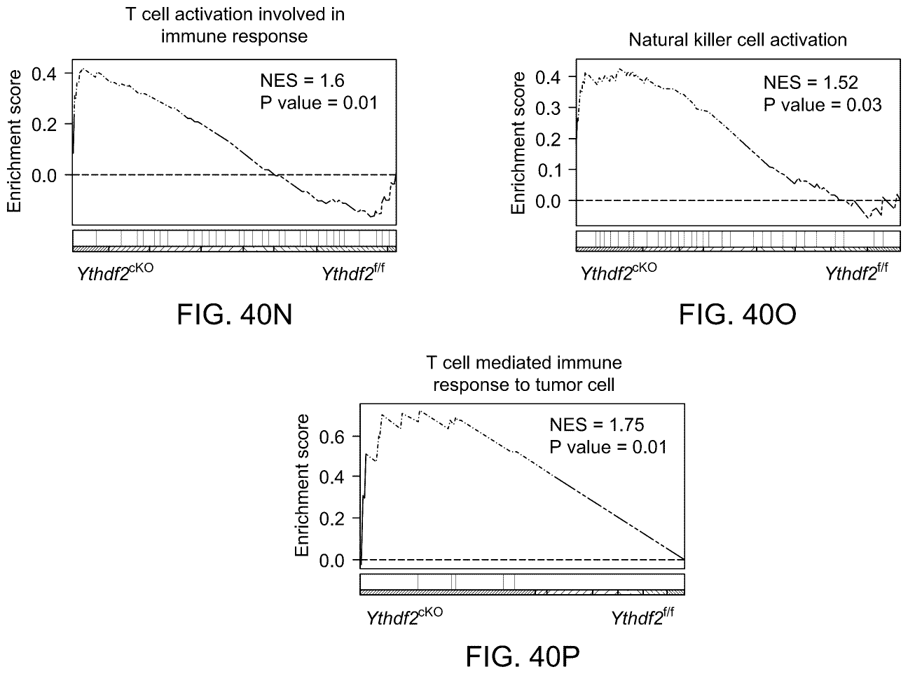

To determine the role of the YTHDF2 in CD4⁺ T cell differentiation, we used the CRISPR-Cas9 method to deplete the Ythdf2 gene (Ythdf2^{-/-}) in mouse naïve CD4⁺ T cells. The knockout (KO) efficacy was validated by immunoblotting. After that, we cultured and differentiated the naïve CD4⁺ T cells into different T cell subsets in vitro. We observed that the deletion of Ythdf2 did not affect the differentiation of Th1, Th2, and Th17 as shown by the relevant cytokine production, or of Treg cells as shown by the induction of Foxp3. However, ablation of Ythdf2 dramatically enhanced Th9 cell differentiation. Ythdf2^{-/-}Th9 cells produced 2.5-fold more IL-9 than Ythdf2^{+/+}Th9 cells when measuring intracellular staining by flow cytometry and the cultured supernatant by ELISA (FIGS. 40A-40C). Ythdf2^{-/-} Th9 cells also showed a significant increase in Th9 cell signature cytokines, including Il2, Il9, and Il21 at mRNA levels, compared to Ythdf2^{+/+} Th9 cells (FIG. 40D). These results indicate that loss of YTHDF2 specifically enhances Th9 cell differentiation in mice.

To further validate our observations, we generated Ythdf2^{f/f} CD4^{Cre} mice by crossing Ythdf2^{f/f} mice²⁴ with CD4-Cre mice (hereafter Ythdf2^{f/f} CD4^{Cre} denoted as Ythdf2^{cKO}). The deletion of Ythdf2 in CD4⁺ T cells was verified by immunoblotting. These mice exhibit a normal distribution of T cells in the thymus, spleen, and lymph nodes. Then, we isolated the naïve CD4⁺ T cells from either Ythdf2^{f/f} or Ythdf2^{cKO} mice and differentiated them into different CD4⁺ T cell subsets as shown earlier. Similarly, we observed that the deficiency of YTHDF2 in mouse CD4⁺ T cells did not affect the differentiation of Th1, Th2, Th17, and Treg cells as shown by flow cytometry. However, consistent with the results in Ythdf2^{-/-} naïve CD4⁺ T cells, naïve CD4⁺ T cells from Ythdf2^{cKO} mice differentiate into more Th9 cells compared to those from Ythdf2^{f/f} mice (FIGS. 40E-40G). Additionally, Ythdf2-deficient Th9 cells expressed higher mRNA levels of Il2, Il9, and Il21 compared to wild-type (WT) Th9 cells (FIG. 1H). Collectively, these results confirm that YTHDF2 limits the differentiation of Th9 cells, and its loss enhances Th9 cell differentiation in mice.

### Ythdf2-Deficient Th9 Cells Display a Distinct Gene Signature

To further understand the difference between WT and Ythdf2-deficient Th9 cells, we characterized the global transcriptional profile of WT and Ythdf2-deficient Th9 cells using bulk RNA-sequencing (RNA-seq). Differentially expressed genes (DEG) analysis showed that 481 genes were significantly upregulated while 563 genes were downregulated in Ythdf2-deficient Th9 cells compared to WT Th9 cells (FIG. 40I). Gene ontology (GO) analysis showed that upregulated DEGs were mostly enriched in innate or adoptive immune-related activation signaling pathways (FIG. 40J). We further characterized the transcript profiles of Th9 cells from Ythdf2^{f/f} and Ythdf2^{cKO} mice. Ythdf2-deficient Th9 cells displayed increased effector cytokines such as Il2, Il9, and Il21 (FIG. 40K). Ythdf2-deficient Th9 cells highly expressed Gata3, and Tbx21, important TFs that control Th9 cell differentiation (39,51) (FIG. 40L). Ythdf2-deficient Th9 cells also upregulated Batf (52) (FIG. 40L), which has been reported to cooperate with other TFs to promote IL-9 production (53). Particularly, Ythdf2-deficient Th9 cells expressed lower levels of inhibitory receptors, including Ctla4, Fas1, Fos, and Tigit, and TFs that induce T cell exhaustion, such as Tox (54, 55) (FIG. 40M), indicating that Ythdf2-deficient Th9 cells may display enhanced activation when compared to WT Th9 cells. Additionally, Gene Set Enrichment Analysis (GSEA) revealed significantly enriched pathways in Ythdf2-deficient Th9 cells related to “T cell activation involved in immune response”, “natural killer cell activation”, and “T cell-mediated immune response to tumor cell” (FIGS. 40N-40P). Collectively, these findings indicate that YTHDF2 deficiency in Th9 cells induces substantial changes in pathways associated with enhanced adaptive and innate immunity, thereby providing evidence that YTHDF2-deficient Th9 cells may exhibit more potent effector function compared to WT Th9 cells.

### Loss of YTHDF2 Promotes Th9 Cell Differentiation Through the IL-4-GATA3 Signaling Pathway

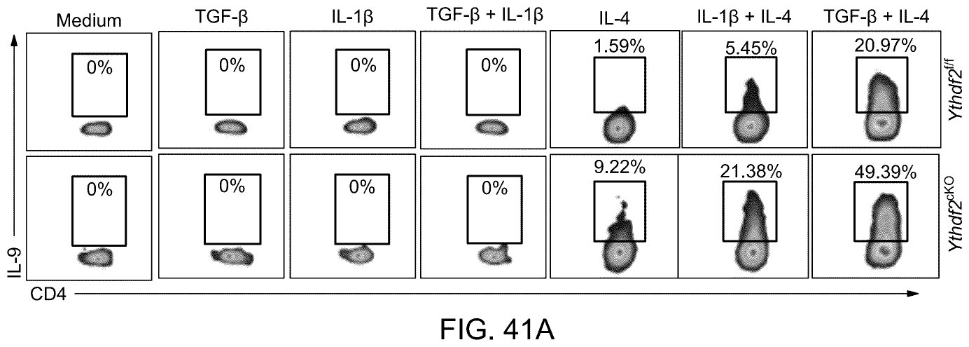

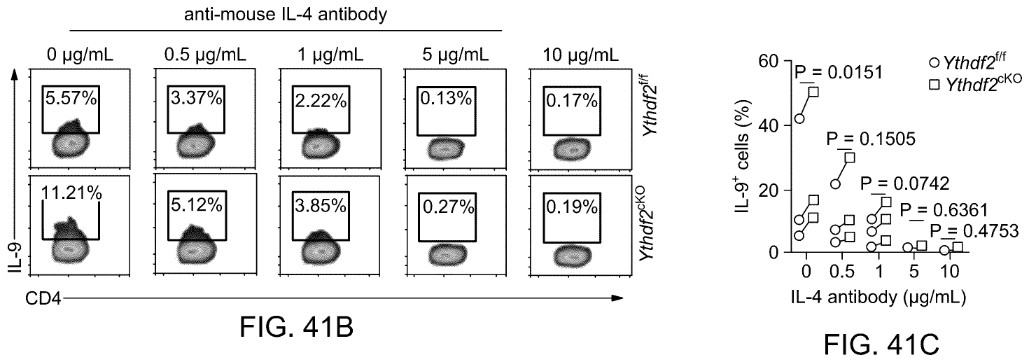

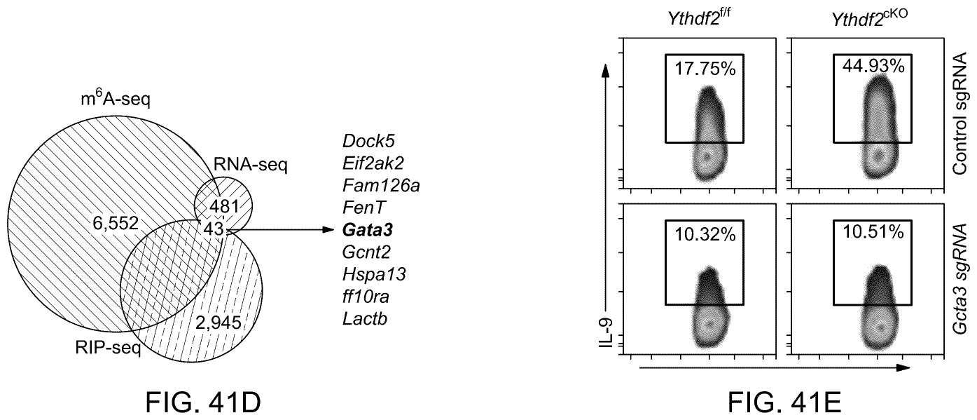

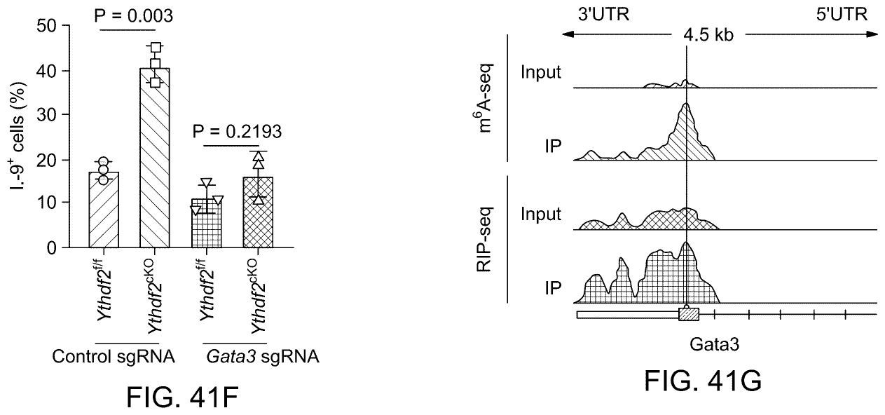

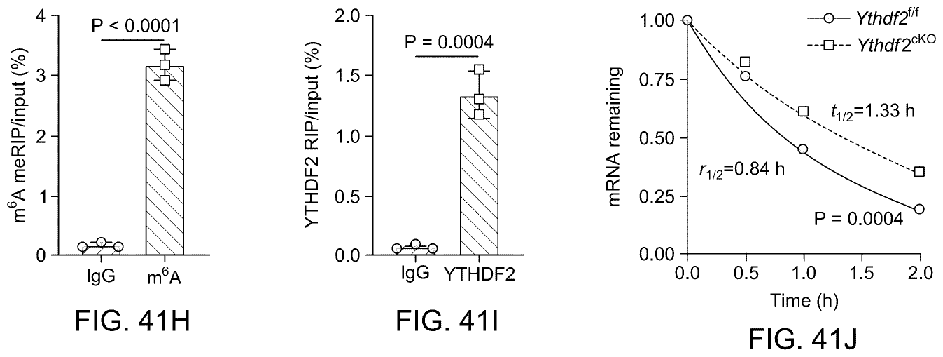

Th9 cells are generated from naïve CD4⁺ T cells in the presence of TL-4 and TGF-β or IL-4 and IL-1β but minimally in the presence of IL-4 alone (39-41). We compared naïve CD4⁺ T cells from Ythdf2^{f/f} mice and Ythdf2^{cKO} mice and found that neither could produce IL-9 after being cultured with either TGF-β or IL-1β alone or their combination (FIG. 41A). However, unlike naïve CD4⁺ T cells from Ythdf2^{f/f} mice, naïve CD4⁺ T cells from Ythdf2^{cKO} mice could produce a substantial amount of IL-9 in response to IL-4 alone and could produce substantially more IL-9 than naïve CD4⁺ T cells from Ythdf2^{f/f} mice when cultured in TGF-β plus IL-4, or IL-1β plus TL-4 (FIG. 41A). These data indicate that TL-4 signaling is critical for naïve CD4⁺ T cell differentiation to Th9 cells in the absence of YTHDF2. To further consolidate the role of IL-4 in Th9 cell differentiation from naïve Ythdf2^{cKO} CD4⁺ T cells, we induced Th9 cell differentiation with IL-4 and TGF-β in the presence of different doses of IL-4-blocking antibodies. As expected, the percentage of Th9 cells significantly declined with the increased dose of IL-4-blocking antibody in Ythdf2^{cKO} mice (FIGS. 41B-41C), supporting the observation that IL-4 signaling is critical for naïve CD4⁺ T cell differentiation to Th9 cells in the absence of YTHDF2.

To gain further mechanistic insight, we performed m⁶A-seq using an m⁶A antibody in both WT and Ythdf2-deficient Th9 cells and RNA immunoprecipitation antibody sequencing (RIP-seq) using a YTHDF2 antibody in WT Th9 cells. m⁶A sequencing, m⁶A peak calling and motif enrichment analysis identified the m⁶A consensus motif GGACU in both Ythdf2^{f} and Ythdf2^{cKO} Th9 cells, indicating successful enrichment of m⁶A. The m⁶A modifications were mainly in protein-coding transcripts and were abundant in coding sequences and 3′ untranslated regions (UTRs). GO enrichment analysis of genes with m⁶A peaks revealed that most m⁶A-marked transcripts in Th9 cells were enriched in pathways involved in RNA splicing, mitotic cell cycle, and DNA replication. From the YTHDF2 RIP-seq results, the YTHDF2-binding peaks were predominantly in the coding region, the 3′UTR, and the stop codon in murine Th9 cells. GO enrichment analysis showed that the YTHDF2 target transcripts were enriched in lymphocyte differentiation, regulation of development growth, and T-cell differentiation. We then identified potential targets by overlapping up-regulated DEGs from RNA-seq data, m⁶A-modified transcripts from m⁶A-seq, and YTHDF2^{-/-} bound transcripts from YTHDF2 RIP-seq. A set of 43 transcripts bound by YTHDF2 and marked with m⁶A in WT and Ythdf2-deficient Th9 cells were selected (FIG. 41D). Among them, we found that Gata3, the main TF downstream of IL-4 and also a key TF that controls Th9 cell differentiation by directly binding in the 11-9 promoter and inducing its transcription (39, 56), was upregulated in Ythdf2-deficient Th9 cells and marked by m⁶A modification (FIG. 41D). qPCR, immunoblotting, and flow cytometry results showed that Ythdf2-deficient Th9 cells had higher mRNA and protein levels of GATA3 compared to WT Th9 cells. Moreover, knockdown of Gata3 by siRNA or KO of Gata3 using CRISPR-Cas9 in Ythdf2-deficient naïve CD4⁺ T cells abolished the difference in the IL-9 production between WT and Ythdf2-deficient Th9 cells (FIGS. 41E-41F). Collectively, these results show that loss of YTHDF2 promotes the Th9 cell differentiation by enabling the IL-4-GATA3 signaling pathway.

YTHDF2 Regulates the Stability of Gata3 mRNA in Th9 Cells

Since YTHDF2 mainly controls the stability of m⁶A-modified mRNA and induces their degradation (13), we hypothesized that YTHDF2 may directly regulate the mRNA stability of Gata3 mRNA in Th9 cells. Integrative Genomics Viewer indicated a good fit between the m⁶A peaks and the YTHDF2-binding peaks in the 3′ UTR of Gata3 (FIG. 41G). RIP assay using either m⁶A or YTHDF2 antibody following qPCR confirmed the m⁶A methylation of the Gata3 site in the 3′ UTR (FIG. 41H). Additionally, the enrichment of YTHDF2 binding at this site was observed in Th9 cells (FIG. 41I). mRNA stability assays showed that the half-life of Gata3 mRNA was significantly increased in Ythdf2-deficient Th9 cells compared to WT Th9 cells (FIG. 41J). These results indicate that loss of YTHDF2 promotes the Th9 cell differentiation at least in part by enhancing the Gata3 mRNA stability in Th9 cells.

### Antigen-Specific Ythdf2-Deficient Th9 Cells Elicit Higher Anti-Tumor Activity Compared to WT Th9 Cells

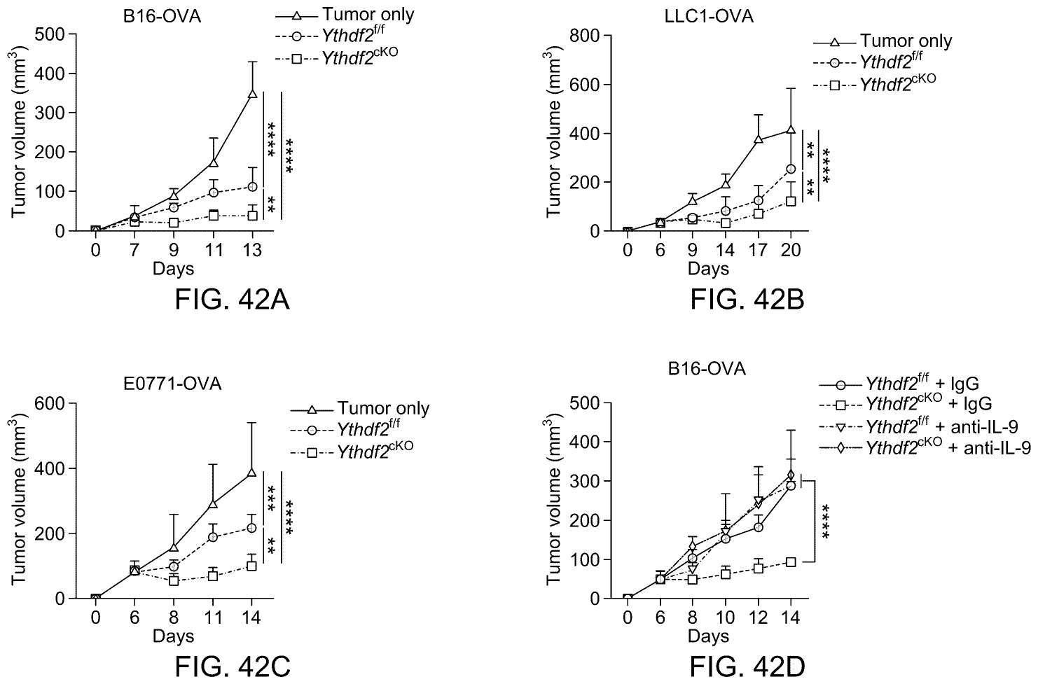

Having elucidated the role and mechanism of action of YTHDF2 in Th9 cell differentiation, we next investigated the role of YTHDF2 in Th9 cell-mediated antitumor activity in vivo. We employed ovalbumin (OVA)-expressing B16 melanoma (B16-OVA), LLC1 lung cancer (LLC1-OVA), and E0771 breast cancer (E0771-OVA) cell lines, and generated OVA-specific Th9 cells from OT-II-Ythdf2^{f/f} or OT-II-Ythdf2^{cKO} mice, which have a TCR specific for the OVA peptide (323-339) presented by MHC class II molecules on CD4⁺ T cells. We intravenously (i.v.) injected OVA-specific Th9 cells into C57BL/6 mice on the same day when the three cell lines were subcutaneously (s.c.) implanted in separate mice. Mice treated with OVA-specific WT Th9 cells showed significantly reduced tumor growth compared to mice receiving phosphate-buffered saline (PBS) (FIGS. 42A-42C), which was consistent with prior studies (45, 47). However, the adoptive transfer of OVA-specific Ythdf2-deficient Th9 cells significantly inhibited tumor growth compared to mice receiving OVA-specific WT Th9 cells (FIG. 42A-42C). Importantly, neutralizing IL-9 using an anti-mouse IL-9 antibody eliminated the difference in tumor growth between mice that received OVA-specific WT Th9 cells and those that received OVA-specific Ythdf2-deficient Th9 cells (FIG. 42D). Thus, compared to antigen-specific WT Th9 cells, antigen-specific Ythdf2-deficient Th9 cells can elicit a significantly stronger anti-tumor activity in vivo that is dependent on IL-9.

### Antigen-Specific Ythdf2-Deficient Th9 Cells Increase the Infiltration of DCs, Cytotoxic CD8⁺ T Cells, and NK Cells

Antigen-specific Th9 cells exert anti-tumor function through several mechanisms: (1) by secreting granzyme-B to directly kill the tumor cell; (2) by promoting mast cell-mediated anti-tumor activity; (3) by enhancing NK cell anti-tumor effects via IL-21; and (4) by provoking CD8⁺ T cell-mediated antitumor immunity. We found that WT and Ythdf2-deficient Th9 cells expressed similar levels of granzyme-B. In addition, we adoptively transferred OVA-specific Th9 cells from OT-II-Ythdf2^{f/f} or OT-II-Ythdf2^{cKO} mice into NSG mice, which show serious defects in both innate and adaptive immune systems, or Rag2^{-/-}γc^{-/-} mice, which lack mature T, B, and NK cells, that were inoculated with B16-OVA tumor cells. The results showed that mice that received OVA-specific WT Th9 cells had tumor growth comparable to those that received OVA-specific Ythdf2-deficient Th9 cells. These results indicate that the stronger anti-tumor activity of the Ythdf2-deficient Th9 cells is not cell-intrinsic. See Refs 42, 45, 48, 57-61.

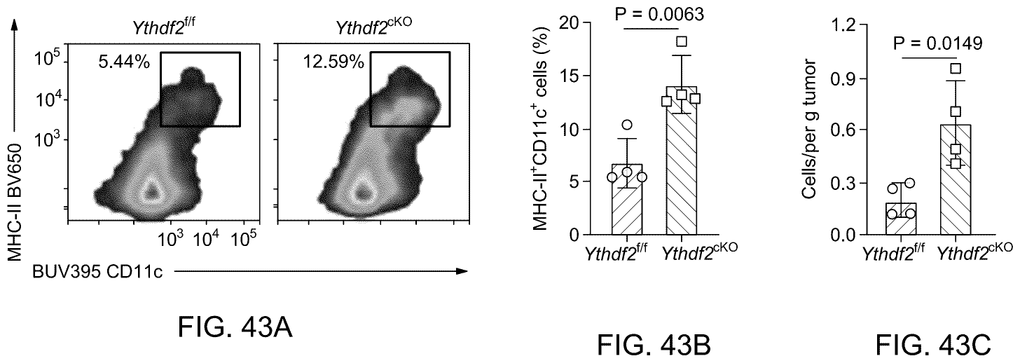

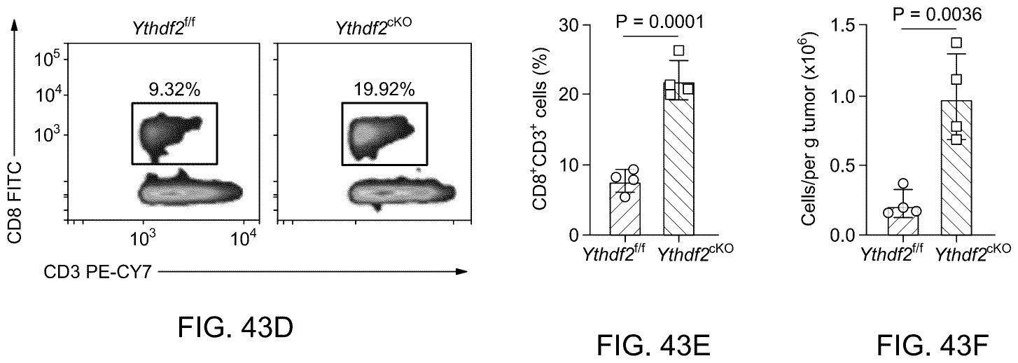

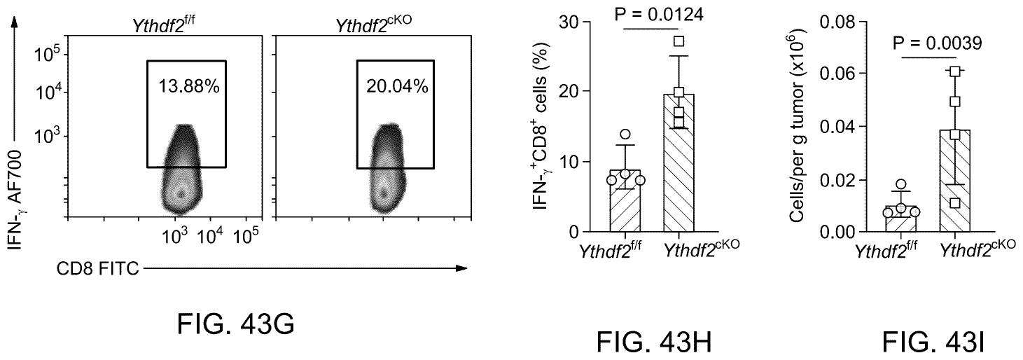

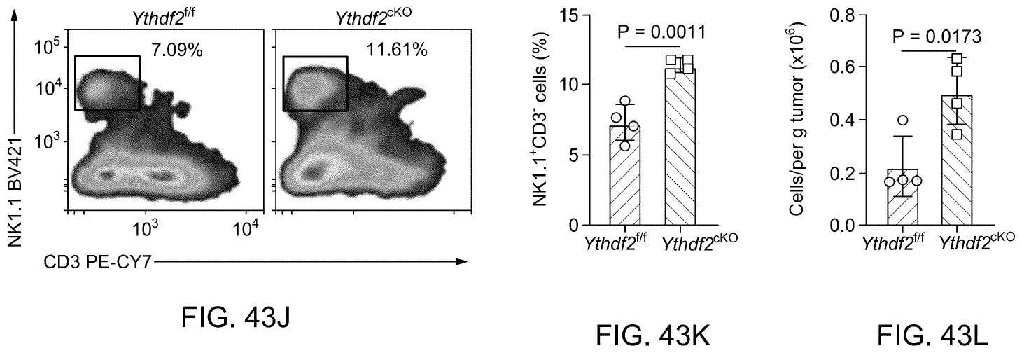

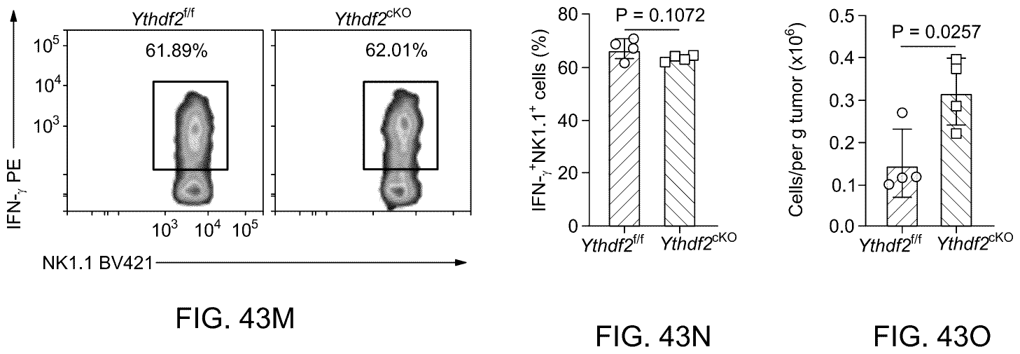

Analysis of the tumor-infiltrated cells in B16-OVA tumor models revealed that the percentages and absolute numbers of mast cells (c-Kit⁺FcεR1⁺), macrophages (F4/80⁺CD11 b⁺), and MDSCs (Gr1⁺CD11b⁺) in tumor tissues were comparable between tumor-bearing mice that were received OVA-specific Th9 cells from OT-II-Ythdf2^{f/f} and OT-II-Ythdf2^{cKO} mice, indicating that these innate immune cells may not be involved in the stronger tumor inhibition by Ythdf2-deficient Th9 cells. In this model, however, we found that the percentages and absolute numbers of DCs (CD11c⁺MHC-II⁺) (FIGS. 43A-43C), the percentages and absolute numbers of CD8⁺ T (CD3⁺CD8⁺) (FIGS. 43D-43F), and the percentages and absolute numbers of IFN-γ-producing CD8⁺ T cells (FIGS. 43G-43I) were significantly increased in tumor tissues from mice that received OVA-specific Ythdf2-deficient Th9 cells compared to those received OVA-specific WT Th9 cells. We also observed a significant increase in the percentages and absolute numbers of NK cells (CD3⁻NK1.1⁺) in tumor tissues from mice that received OVA-specific Ythdf2-deficient Th9 cells compared to those received OVA-specific WT Th9 cells (FIGS. 43J-43L). Moreover, mice that received OVA-specific Ythdf2-deficient Th9 cells had increased absolute numbers of IFN-γ-producing NK cells compared to those that received OVA-specific WT Th9 cells, although the percentages showed no difference (FIGS. 43M-43O). We also validated these findings in another two tumor models (data not shown). Taken together, these results demonstrated that antigen-specific Ythdf2-deficient Th9 cells increase the infiltration of DCs, cytotoxic CD8⁺ T cells, and NK cells, indicating that these cells are indirectly involved in the stronger tumor inhibition by Ythdf2-deficient Th9 cells.

### YTHDF2 Deficiency Promotes Th9 Cell Anti-Tumor Activity by Enhancing Both Innate and Adaptive Anti-Tumor Immune Responses

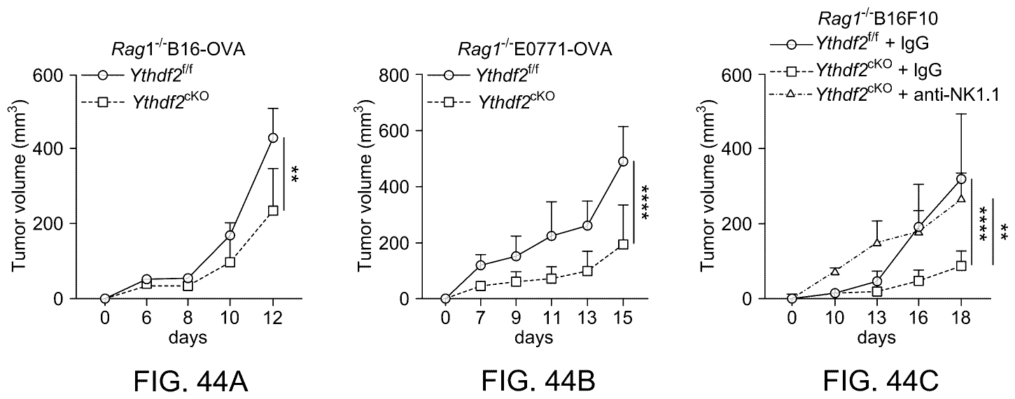

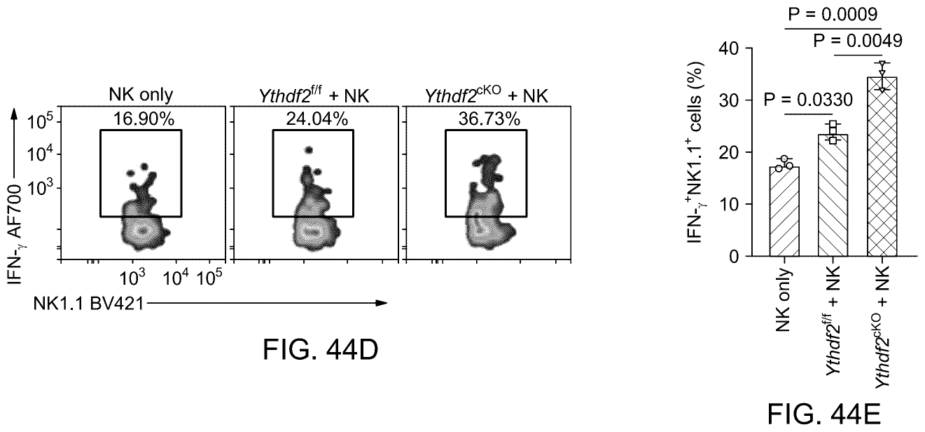

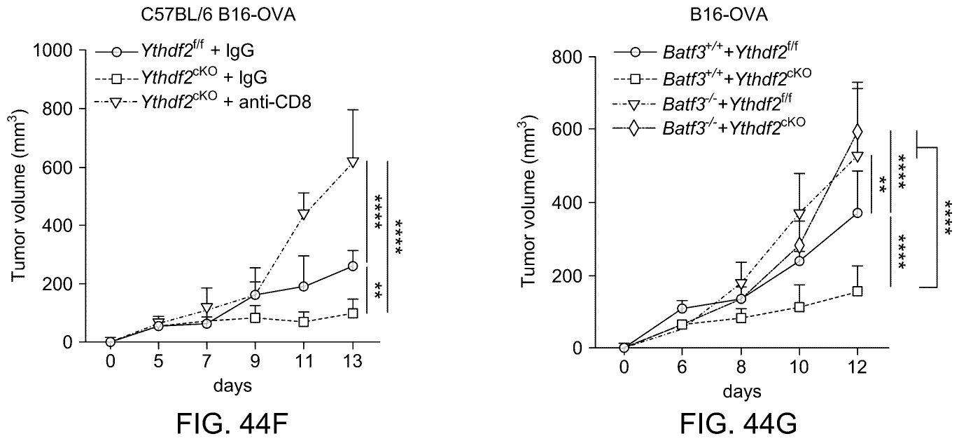

We further investigated whether the anti-tumor effect of Ythdf2-deficient Th9 cells was dependent on NK cells. To exclude the effect of adaptive immunity, we adoptively transferred OVA-specific Th9 cells from OT-II-Ythdf2^{f/f} or OT-II-Ythdf2^{cKO} mice into Rag1^{-/-} mice that were inoculated with B16-OVA or E0771-OVA tumor cells. Rag1^{-/-} mice lack mature T and B cells but have normal NK cells (62). We found that Rag1^{-/-} mice that received OVA-specific Ythdf2-deficient Th9 cells showed slower tumor growth compared to those that received OVA-specific WT Th9 cells (FIGS. 44A-44B). Moreover, depleting NK cells abolished the difference in tumor growth in Rag1^{-/-} mice (FIG. 44C). We observed that the percentages and the absolute numbers of NK cells, as well as the IFN-γ NK cells, were significantly increased in tumor tissues from Rag1^{-/-} mice that received OVA-specific Ythdf2-deficient Th9 cells compared to those that received OVA-specific WT Th9 cells. We also measured the cytotoxicity of NK cells against NK cell-sensitive B16F10 tumor cells in the presence of either WT or Ythdf2-deficient Th9 cells. The real-time cell analysis (RTCA) demonstrated that NK cells showed a trend toward higher cytotoxicity against B16F10 tumor cells cocultured with Ythdf2-deficient Th9 cells compared to those cocultured with WT Th9 cells. NK cells produced significantly more IFN-γ when cocultured with B16F10 tumor cells in the presence of Ythdf2-deficient Th9 cells compared to those with WT Th9 cells (FIGS. 44D-44E). Taken together, these findings indicate that Ythdf2-deficient Th9 cells promote in vivo tumor killing indirectly via enhanced activation of NK cell cytotoxicity and IFN-γ production.

We then explored the role of CD8⁺ T cells in the anti-tumor function of Ythdf2-deficient Th9 cells. We found that when the population of OVA-specific Ythdf2-deficient Th9 cells were adoptively transferred into B16-OVA tumor-bearing C57BL/6 mice that were depleted of CD8⁺ T cells, the difference in tumor growth was abolished (FIG. 44F). These results indicate that CD8⁺ T cells are critical in mediating the anti-tumor function of Ythdf2-deficient Th9 cells.

DCs are critical to cross-present tumor antigens and to induce anti-tumoral CD8⁺ T cell responses. Th9 cells can promote tumor-specific CD8⁺ response through CCL20/CCR6-dependent recruitment of DCs. Batf3^{-/-} mice lack conventional type 1 DCs. The observed decrease in tumor growth with OVA-specific Ythdf2-deficient Th9 cells was lost when OVA-specific WT Th9 cells mice or OVA-specific Ythdf2-deficient Th9 cells were adoptively transferred into B16-OVA bearing Batf3^{-/-} mice (FIG. 5G). These results indicate that Ythdf2-deficient Th9 cells elicit the strong anti-tumor CD8⁺ T cell response via the requisite participation of DCs. See Refs 45, 63, 64.

Collectively, these findings demonstrate that YTHDF2 deficiency promotes Th9 cell anti-tumor activity by enhancing both innate and adaptive anti-tumor immune responses.

### YTHDF2 Deficiency Promotes Th9 Cell Differentiation in Humans

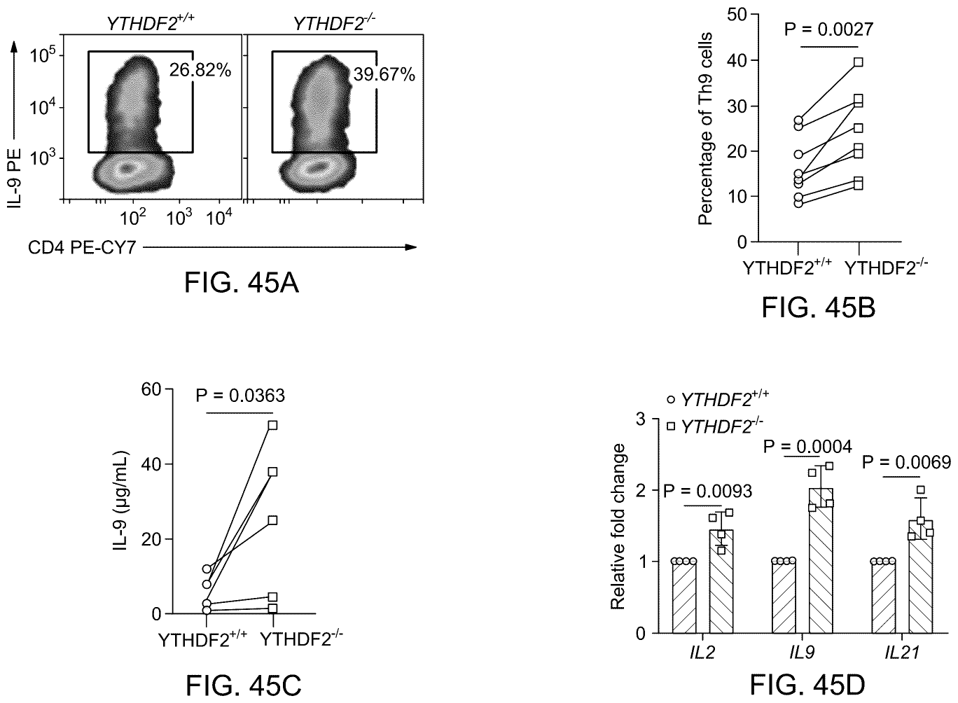

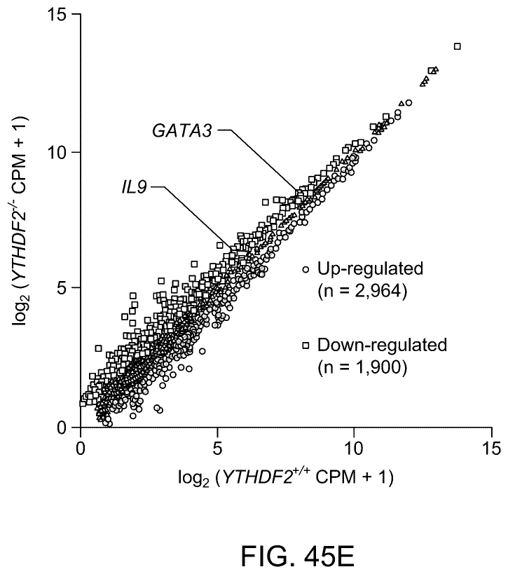

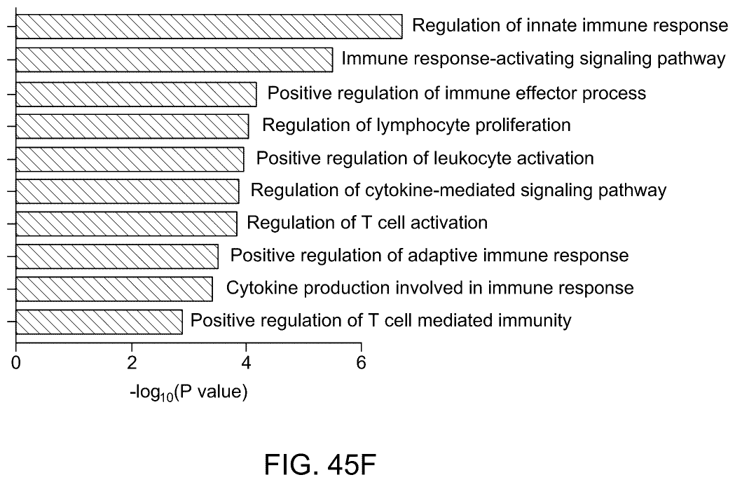

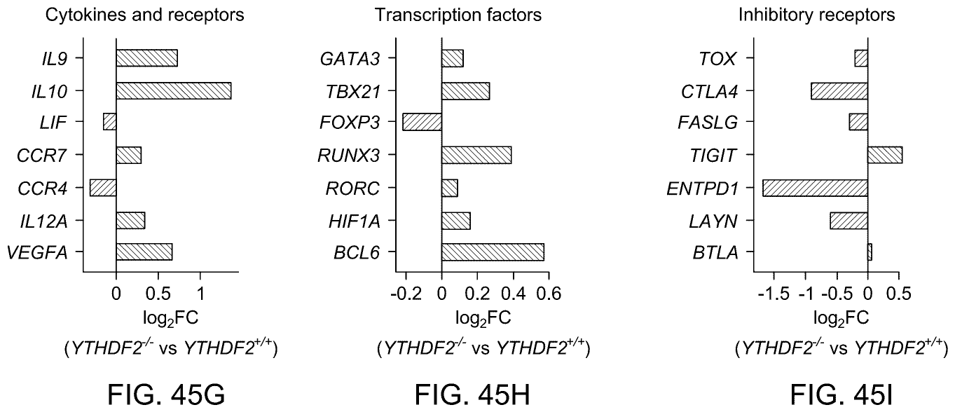

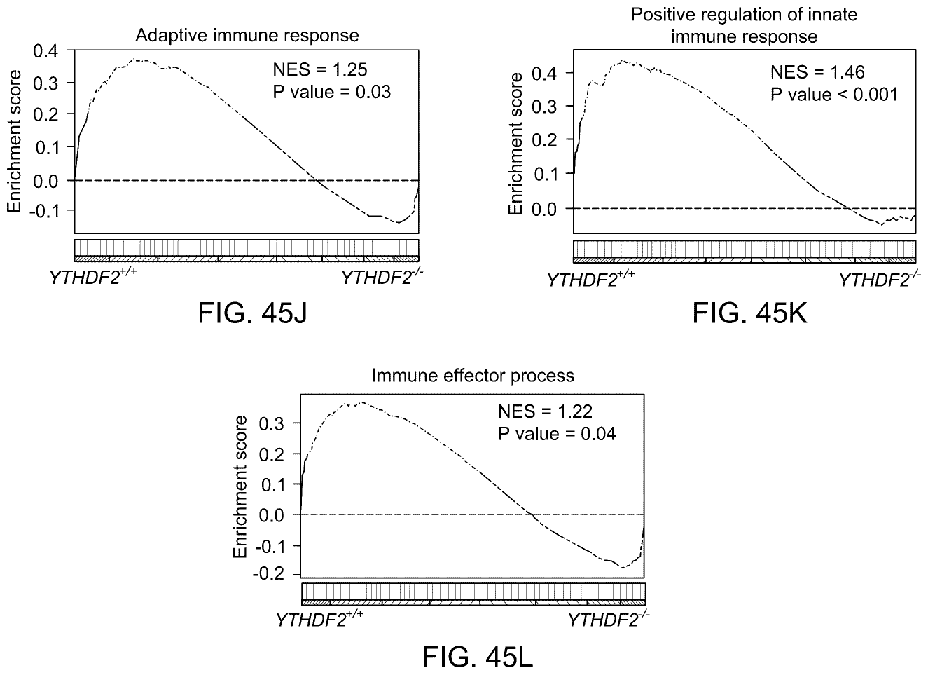

After gaining knowledge from mouse studies, we determined whether YTHDF2 also regulates the differentiation of human Th9 cells. For this purpose, we used CRISPR/Cas9 method to KO YTHDF2 (YTHDF2^{-/-}) in human naïve CD4⁺ T cells. YTHDF2^{+/+} and YTHDF2^{-/-} cells were subsequently polarized under the Th9-culture condition. Deletion of YTHDF2 significantly enhanced human Th9 cell differentiation as detected by flow cytometry and ELISA (FIGS. 45A-45C). YTHDF2^{-/-} Th9 cells expressed higher mRNA levels of signature cytokines, such as IL2, IL9, and IL21, compared to YTHDF2^{+/+} Th9 cells (FIG. 45D). These results indicate that the negative role of YTHDF2 in Th9 cell differentiation is conserved between humans and mice. We then characterize the transcriptional profile of YTHDF2^{+/+} and YTHDF2^{-/-} by bulk RNA-seq analysis. The volcano plot showed a differential expression of 4864 genes, including 2964 upregulated genes and 1900 downregulated genes in YTHDF2^{-/-} Th9 cells compared to YTHDF2^{+/+} Th9 cells (FIG. 45E). GO analysis showed that upregulated DEGs were mostly enriched in multiple immune response activation signaling pathways including innate immune response activation and T cell activation (FIG. 45F). In addition, we observed similar expression patterns of cytokines (FIG. 45G), TFs (FIG. 45H), and inhibitory receptors (FIG. 45I) as we found in mouse Th9 cells, including IL9, GATA3, and TOX. Additionally, the GSEA results also showed similar upregulated pathways involved in adaptive immune and innate immune activation (FIGS. 45J-45L). Taken together, these results indicate that YTHDF2 deficiency promotes Th9 cell differentiation in humans.

### Human YTHDF2 Deficient-CAR-Th9 Cells Display Enhanced Anti-Tumor Activity In Vivo

Human IL-9-producing CAR Th9 (CAR-Th9) cells have shown potent anti-tumor activity against established tumors in vivo (49). Since YTHDF2 exerts a conserved role in Th9 cell differentiation between humans and mice and YTHDF2 deficiency promotes Th9 cell anti-tumor activity by enhancing both innate and adaptive anti-tumor immune responses in mice, we hypothesized that deletion of YTHDF2 could improve CAR-Th9 cell antitumor activity. To this end, we first established and optimized a method for YTHDF2^{-/-} CAR-Th9 cell polarization and expansion (see Methods). We transduced the human Th9 cells with our epidermal growth factor receptor (EGFR) CAR construct or our prostate stem cell antigen (PSCA) CAR construct. See Refs. 65-66.

Flow cytometry showed that both YTHDF2^{+/+} and YTHDF2^{-/-} Th9 cells showed comparable surface density expression of CAR constructs. Both types of YTHDF2^{-/-} EGFR CAR-Th9 cells and YTHDF2^{-/-} PSCA CAR-Th9 cells produced more IL-9 than their respective YTHDF2^{+/+} CAR-Th9 cell counterparts during the in vitro differentiation. Both YTHDF2^{-/-} EGFR CAR-Th9 cells and YTHDF2^{-/-} PSCA CAR-Th9 cells exhibited similar cytolytic activity against their respective target tumor cells compared to their YTHDF2^{+/+} CAR-Th9 cells counterparts. We then evaluated the anti-tumor activity of CAR-Th9 cells in vivo.

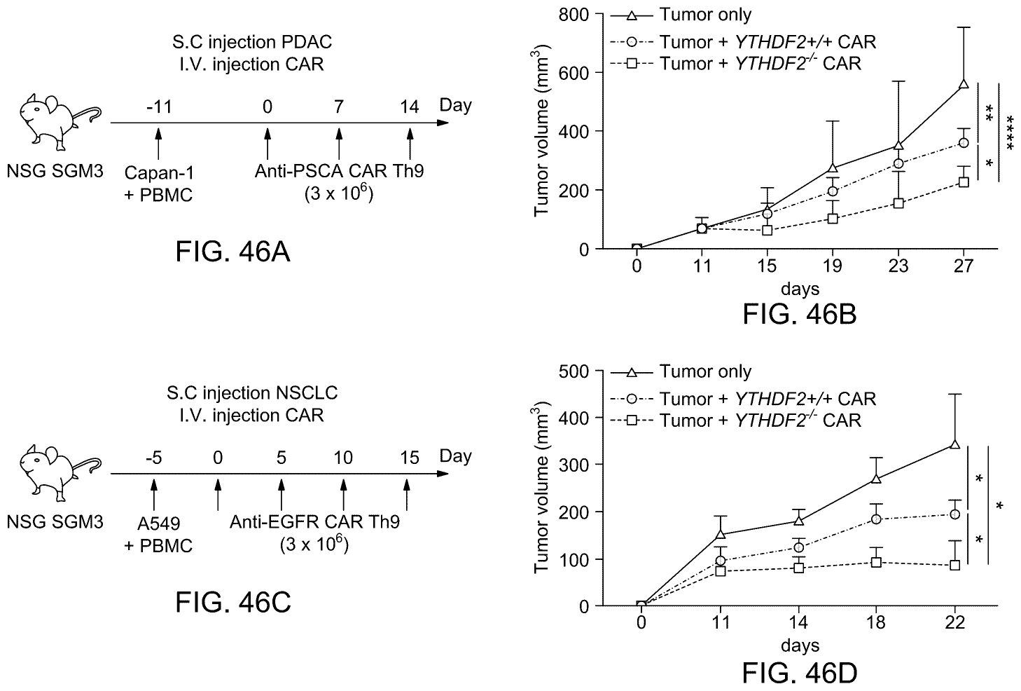

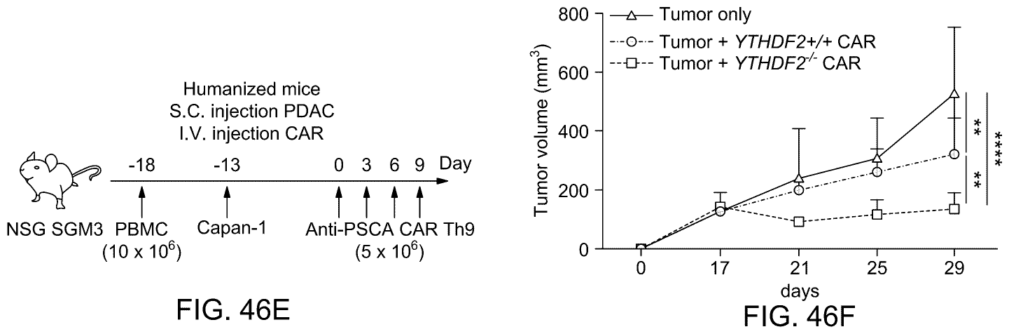

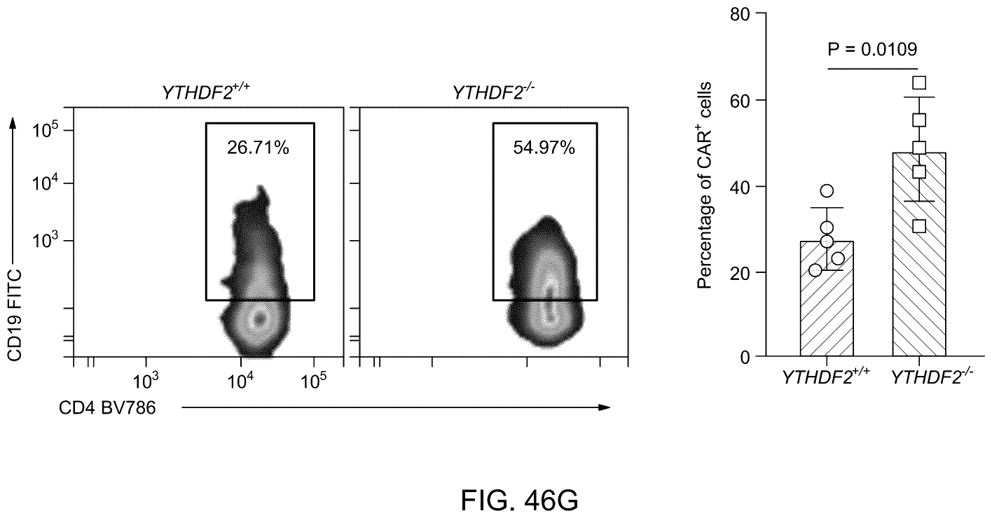

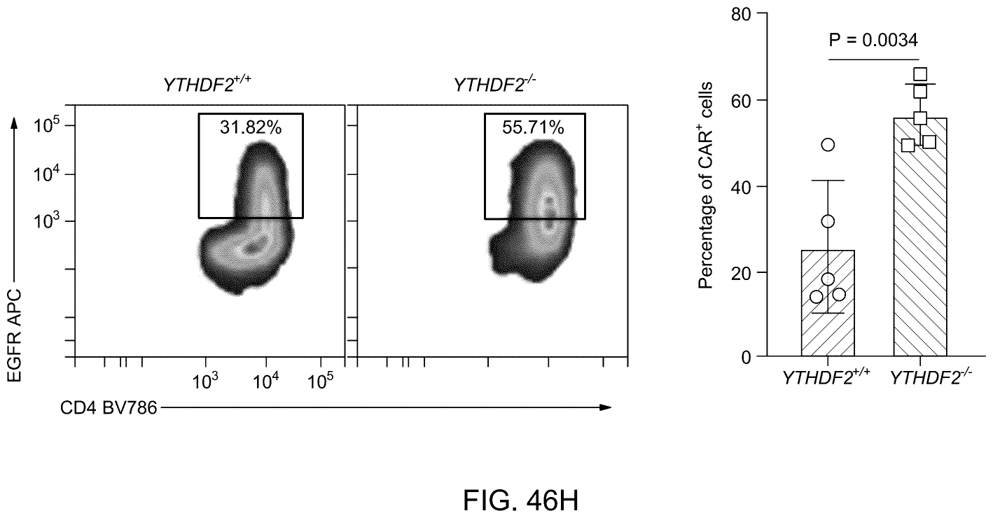

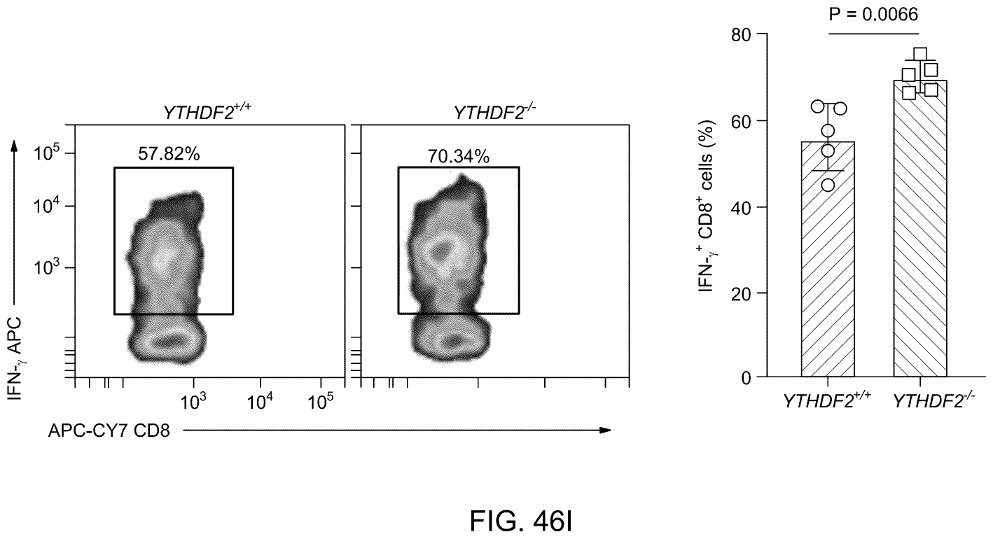

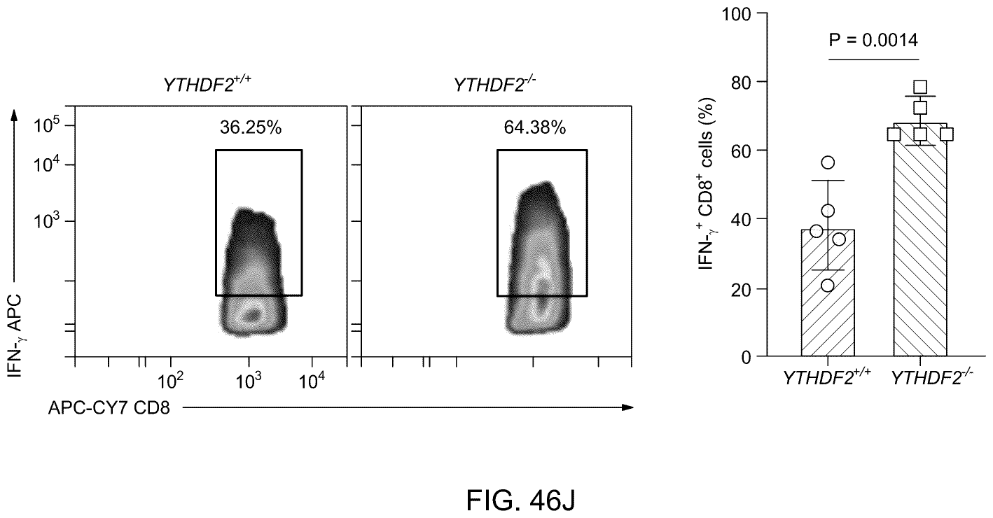

The human lung cancer cell line A549, which expresses EGFR, and the human pancreatic cancer cell line Capan-1, which expresses PSCA, were s.c. injected into separate NSG mice and once tumors were established, mice with the EGFR⁺ A549 tumors received 3×10⁶ either YTHDF2^{+/+} EGFR CAR-Th9 cells or YTHDF2^{-/-} EGFR CAR-Th9 cells i.v. via the tail vein. The results showed that mice receiving YTHDF2^{-/-} EGFR CAR-Th9 cells had a similar tumor growth rate compared to those receiving YTHDF2^{+/+} EGFR CAR-Th9 cells. The same results were observed with the YTHDF2^{+/+} PSCA CAR-Th9 cells or YTHDF2^{-/-} PSCA CAR-Th9 cells when i.v. injected into the NSG mice inoculated with the PSCA⁺ Capan-1 tumors. These results are expected because we have shown that mouse Ythdf2-deficient Th9 cells need both endogenous innate and adaptive immune systems to fulfill their anti-tumor effects. We therefore evaluated the anti-tumor activity of human YTHDF2^{-/-} CAR-Th9 cells in the presence of human peripheral blood mononuclear cells (PBMCs). To this end, firstly, we s.c. inoculated 3×10⁶ Capan-1 tumor cells that were premixed with 1×10⁶ PBMCs into NSG-SGM3 mice, which express physiological levels of three human cytokines IL-3, GM-CSF, and c-KITL to support the engraftment of human immune cells. These mice were then received i.v. injection of 3×10⁶ either YTHDF2^{+/+} PSCA CAR-Th9 cells or YTHDF2^{-/-} PSCA CAR-Th9 cells (FIG. 46A). Infusion of YTHDF2^{-/-} PSCA CAR-Th9 cells showed a significantly stronger anti-tumor effect compared to YTHDF2^{+/+} PSCA CAR-Th9 cells (FIG. 46B). The same results were observed with the YTHDF2^{+/+} EGFR CAR-Th9 cells or YTHDF2^{-/-} EGFR CAR-Th9 cells when i.v. injected into the NSG mice inoculated with the A549 tumors (FIGS. 46C-46D).

To further validate these results, we generated a humanized mouse model in which NSG-SGM3 mice were i.v. infused with 10×10⁶ PBMCs. We s.c. implanted Capan-1 tumor cells into these humanized mice 7 days after immune cells were reconstituted. After the tumors were established, 5×10⁶ either YTHDF2^{+/+} PSCA CAR-Th9 cells or YTHDF2^{-/-} PSCA CAR-Th9 cells were i.v. infused into these tumor-bearing mice (FIG. 46E). The results showed that mice treated with YTHDF2^{-/-} PSCA CAR-Th9 cells had significantly reduced tumor growth compared to those receiving YTHDF2^{+/+} PSCA CAR-Th9 cells (FIG. 46F). Collectively, these findings indicate that in the presence of PBMCs, YTHDF2^{-/-} CAR-Th9 cells display enhanced anti-tumor activity in vivo when compared to YTHDF2^{+/+} CAR-Th9 cells. Lastly, analysis of the tumor-infiltrated cells revealed that compared to the infusion of YTHDF2^{+/+} EGFR or PSCA CAR-Th9 cells, the administration of YTHDF2^{-/-} EGFR or PSCA CAR-Th9 cells resulted in significantly increased infiltration of EGFR CAR⁺ or PSCA CAR⁺ cells (FIGS. 46G-46H) and IFN-γ⁺CD8⁺ T cells (FIGS. 46I-46J), indicating that human YTHDF2^{-/-} CAR-Th9 cells exert their anti-tumor activity by enhancing adaptive anti-tumor immune responses.

### DISCUSSION

The experiments demonstrated that loss of YTHDF2 enables Th9 cell differentiation from naïve CD4⁺ T cells and enhances Th9 cell-mediated anti-tumor function in both mice and humans by increasing NK cell, DC, and CD8⁺ T cell infiltration and anti-tumor function. Moreover, engineered YTHDF2^{-/-} human CAR-Th9 cells exhibited improved tumor control in xenografted mice in the presence of human PBMCs. IL-4 signaling is required for Th9 cell differentiation from naïve CD4⁺ T cells and here we show it is the absence of YTHDF2 that enhances the IL-4-mediated stabilization of GATA3 so it can in turn further drive Th9 cell differentiation.

IL-9 and IL-21 are pivotal cytokines secreted by Th9 cells, each exerting distinct functions and governed by unique regulatory mechanisms in the immune response. IL-9 exerts its effects by binding to the IL-9 receptor (IL-9R), a heterodimer composed of the IL-9R alpha chain and a common gamma chain (γc). The interaction of IL-9 with its receptor activates the downstream signaling cascades, primarily through the JAK/STAT pathway, with significant activation of STAT1 and STAT3. Moreover, a recent study demonstrated that NK cells show a robust response to IL-9 stimulation, suggesting that IL-9 may serve as a direct regulator in enhancing NK cell functionality. However, the precise regulatory mechanisms underlying how IL-9 promotes NK cell responses remain to be fully elucidated, warranting further investigation to clarify the pathways and molecular interactions involved. IL-21 plays a direct role in augmenting the functional capacity of NK cells and CD8⁺ T cells through its interaction with the IL-21 receptor expressed in these cells. In this study, our findings indicate that loss of YTHDF2 in Th9 cells leads to the substantially increased production of IL-21 compared to WT Th9 cells, which is likely to directly contribute to the enhancement of NK cell-mediated cytotoxicity and CD8⁺ T cell survival and activation consistent with previous reports. Our study also showed that Th9 cells enhanced CD8⁺ T cell-mediated cytotoxicity through means other than IL-21, in that in the absence of DCs, the enhanced CD8⁺ T cell-mediated cytotoxicity in Ythdf2-deficient Th9 cells was lost. Th9 cells are known to secrete several chemokines, such as CCL17 and CCL20, which play a role in recruiting and activating dendritic cells (DCs). As essential antigen-presenting cells, DCs can further stimulate the proliferation and anti-tumor activity of CD8⁺ T cells. In our experimental model, we observed no significant difference in tumor growth in the DC-deficient mice treated with either WT or Ythdf2-deficient Th9 cells, indicating that Ythdf2-deficient Th9 cells enhance CD8⁺ T cell function through the recruitment and activation of DCs. See Refs. 37, 45, 58, 67-69, 71-80.

CAR-T cell therapy, an important type of cancer immunotherapy, has demonstrated effectiveness across various types of lymphoid malignancies. However, durable long-term remission remains rare, with most patients eventually experiencing a relapse. This highlights an urgent need to enhance the therapeutic efficacy of CAR-T cell treatments. Recent studies have shown that CAR-Th9 cells exhibit superior anticancer activity, particularly against solid tumors, suggesting a promising avenue for improving CAR-T cell-based therapies. Compared to CAR-Th1 cells, CAR-Th9 cells exhibit a central memory phenotype, reduced exhaustion, increased proliferative capacity, and enhanced longevity in vivo, underscoring their promising potential for cancer immunotherapy. YTHDF2 plays context-dependent roles in immune regulation, exerting different effects depending on the specific cell type. As immunobiology emerges as a critical field, the role of the m⁶A machinery in fine-tuning tumor immunity has gained significant attention. YTHDF2 has been observed to regulate immunosuppressive myeloid cells in both natural and therapy-induced cancer scenarios. Additionally, YTHDF2 controls anti-tumor immunity by regulating Treg cells. YTHDF2 deficiency in Treg cells leads to increased apoptosis and impaired suppressive function of Treg cells which reduces tumor growth. See Refs. 33, 34, 49, 81-83

In our current study, YTHDF2-deficient CAR-Th9 cell-treated tumor-bearing mice displayed a superior anti-tumor response, accompanied by an increased infiltration of antitumor immune cells, including CD8⁺ T cells, within the TME. Notably, our findings indicate that YTHDF2-deficient CAR-Th9 cells exhibit higher expression of activation markers associated with T-cell effector functions. This indicates that engineered YTHDF2^{-/-} CAR-Th9 cells will offer a more effective therapeutic strategy for treating patients with solid tumors. The observed lower expression of inhibitory genes such as Tox, Ctla4, and Fas1 indicates that YTHDF2 is involved in regulating the expression of these genes.

In conclusion, our findings indicate that YTHDF2 limits Th9 cell differentiation from WT naïve CD4⁺ T cells. Specifically, YTHDF2 restrains Th9-mediated tumor control by inhibiting the production of IL-9 and IL-21, which are crucial for activating both innate and adaptive immune responses against tumors. Deletion of YTHDF2 enhances the anti-tumor activity of Th9 cells, which depends on their collaboration with other immune cells. These results highlight a effective strategy for improving tumor-infiltrating lymphocyte-based cancer therapies. Modulating YTHDF2 to boost antitumor immune responses will provide new opportunities for advancing immunotherapeutic approaches in cancer treatment.

### Materials and Methods

Mice. Ythdf2^{f/f} mice, generated as previously described (24) were crossed with CD4^{Cre} mice (The Jackson Laboratory) to generate Ythdf2^{cKO} mice. Rag1^{-/-} mice were kindly provided by Z. Sun (City of Hope National Medical Center). Batf3^{-/-} mice were kindly provided by M. Kortylewski (City of Hope National Medical Center). Wild-type C57BL/6 (000664), NOD scid gamma (NSG) (005557), and NSG-SGM3 (013062) were purchased from The Jackson Laboratory. Rag2^{-/-}Il2rg^{-/-} mice were provided by the animal facility at City of Hope. All experimental mice were bred and maintained under specific pathogen-free conditions. 6- to 12-week-old mice of both sexes were used for experiments. Mice were housed in the City of Hope Animal Facility with a 12-hour light/12-hour dark cycle and temperatures of ˜18-23° C. with 40-60% air humidity. All animal experiments were approved by the City of Hope Institutional Animal Care and Use Committee.

Cell lines. Ovalbumin (OVA)-expressing B16 (B16-OVA) cells were provided by M. Kortylewski (City of Hope National Medical Center). LLC1 cells were provided by Edwin Manuel (City of Hope National Medical Center). E0771 cells were provided by Zhen Bouman Chen (City of Hope National Medical Center). The HEK293T, A549, and Capan-1 cell lines were purchased from ATCC. These above cell lines were cultured in DMEM complete medium (Dulbecco's modified Eagle medium (Gibco) supplemented with 10% fetal bovine serum (FBS; Gibco)). Cells were incubated at 37° C. with 5% CO2.

Human samples. Peripheral blood samples from deidentified healthy donors who consented to an Institutional Review Board-approved protocol (IRB 18238) were obtained from the Michael Amini Transfusion Medicine Center of City of Hope National Medical Center.

Generation of CRISPR-edited Ythdf2-deficient mouse and human naïve CD4⁺ T cells. To generate mouse Ythdf2-KO and human YTHDF2^{-/-} KO naïve CD4⁺ T cells, we used CRISPR RNA (crRNA) (5-CGAACCTTACTTGAGCCCAC) (SEQ ID NO:6) targeting the mouse Ythdf2 gene and crRNA (5-TGAAGCTGCTTGGTCTACGG) (SEQ ID NO:89) targeting the human YTHDF2 gene. In brief, pre-transcribed Alt-R® CRISPR-Cas9 crRNAs and Alt-R® CRISPR-Cas9 tracrRNA (Catalog #1072532) were purchased from IDT (Integrated DNA Technologies, Inc., Coralville, Iowa). Guide RNA (gRNA) was prepared by incubating 200 μM each of crRNA and tracrRNA together in a total volume of 5 μl in Nuclease-Free 1× Tris-EDTA solution (IDT, Catalog #11-01-02-02) at 95° C. for 5 minutes. The Cas9/RNP complex was formed by incubating 1.2 μl of Alt-R® S.p. HiFi Cas9 Nuclease V3 protein (105 pmol) (Catalog #1081060), 1.2 μl of gRNA (120 pmol), and 2.1 μl of PBS in a total volume of 5 μl for 10-20 minutes at room temperature. Purified naïve CD4⁺ T cells were resuspended in 20 μl of P3 Primary Cell 4D-Nucleofector™ X Solution and 5 μl of Cas9/RNP complex and 1 μl of 100 μM Alt-R® Cas9 Electroporation Enhancer (Catalog #1075915), and electroporated using Lonza 4D-Nucleofector system with pulse DN-100 (for mouse T cells) and EO-115 (for human T cells). After electroporation, naïve CD4⁺ T cells were stimulated and polarized into Th9 cells. The efficiency of CRISPR modification was assessed using immunoblotting. To KO mouse Gata3 in mouse CD4⁺ T cells, the crRNA 5-GACTTACATCCGAACCCGGT (SEQ ID NO:90) is used for gRNA generation.

Gene knockdown using nucleofection. For gene knockdown in mouse CD4⁺ T cells, activated CD4⁺ T cells were transfected with siRNA or scrambled siRNA in 20 μl per reaction of room temperature P3 primary cell 4D-Nucleofector X solution (Lonza, V4XP-3032) with program DN-100 on a 4D-Nucleofector system (Lonza). Transfected CD4⁺ T cells were seeded in a 24-well plate and cultured in RPMI 1640 with 10% heat-inactivated FBS (Gibco) at 37° C. in a 5% CO₂ humidified incubator, differentiated into Th9 cells as described in this study. The culture medium was removed and replaced with a fresh medium every two days. Cells were collected on day 5 for immunoblotting and flow cytometric analysis. The Gata3 and negative control DsiRNA were purchased from IDT (mm. Ri. Gata3.13).

In vitro Th-cell differentiation. Mouse naïve CD4⁺ T cells were isolated from spleens and lymph nodes of Ythdf2^{f/f}, Ythdf2^{cKO}, OT-II-Ythdf2^{f/f}, OT-II-Ythdf2_{cKO}, or Wild-type C57BL/6 mice using STEMCELL mouse naïve CD4⁺ T cells isolation kit according to the manufacturer's instructions. The isolated CD4⁺ T cells were then stimulated with plate-coated anti-mouse CD3 (5 μg/ml) and anti-CD28 (2 μg/ml) antibodies and differentiated into CD4⁺ T cell subsets with: (a) Th9-polarized medium supplemented with IL-2 (100 IU/ml), IL-4 (30 ng/ml), TGF-β (2 ng/ml), and anti-IFN-γ monoclonal antibodies (mAbs; 10 μg/ml); (b) Th1-polarized medium supplemented with IL-2 (100 IU/ml) and IL-12 (10 ng/ml); (c) Th2-polarized medium supplemented with IL-4 (20 ng/ml) and anti-IFN-γ mAbs (10 μg/ml); (d) Th17-polarized medium supplemented with IL-6 (30 ng/ml), TGF-β (2 ng/ml), and anti-IFN-γ mAbs (10 μg/ml); (f) T-reg-polarized medium supplemented with IL-2 (100 IU/ml) and TGF-β (2 ng/ml). After culturing for a total of 5 days, differentiated Th cells were harvested and cytokine expression was detected by flow cytometry, ELISA, or RT PCR.

For in vitro human Th9 cell differentiation, human T cells were isolated from the peripheral blood of healthy donors with the human T cell enrichment cocktail (STEMCELL) according to the manufacturer's instructions, followed by human naïve CD4⁺ T cells isolation kit (STEMCELL) to enrich human naïve CD4⁺ T cells further. In some experiments, naïve CD4⁺ T cells were isolated by flow cytometry from the peripheral blood mononuclear cells of healthy donors with the flow antibodies panel: anti-human CD3, anti-human CD4, anti-human CD45RA, and anti-human CD45RO. The purified human naïve CD4⁺ T cells were performed by the CRISPR/Cas9 gene editing system to KO YTHDF2 and then stimulated with the human CD3/CD28 T cell activator (STEMCELL, 10991) for two days in the Th9-polarized condition (human IL-4 (30 ng/ml), human TGF-β (2 ng/ml), and anti-human IFN-γ mAbs (10 μg/ml)). After the initial two days of culture, cells were provided with human IL-2 (100 IU/ml). After culturing for a total of 7 days, differentiated Th9 cells were harvested, and cytokine expression was detected by flow cytometry, ELISA, or RT PCR.

CAR construction, production, and transduction into human CD4⁺ T cells. The EGFR-CAR cassette sequentially includes a signal peptide, the light chain of an anti-EGFR antibody, a linker, the heavy chain of an anti-EGFR antibody, a hinge, the CD28 costimulatory domain, and CD3ζ, as previously described. See Ma, R. et al. An Oncolytic Virus Expressing IL15/IL15Rα Combined with Off-the-Shelf EGFR-CAR NK Cells Targets Glioblastoma. Cancer research 81, 3635-3648 (2021), incorporated by reference herein. The PSCA CAR was designed to sequentially consist of a signal peptide, an anti-PSCA single-chain fragment variable, a hinge, the transmembrane domain, CD28, and CD3ζ, as reported by our previous study. See Teng, K.-Y. et al. Off-the-Shelf Prostate Stem Cell Antigen-Directed Chimeric Antigen Receptor Natural Killer Cell Therapy to Treat Pancreatic Cancer. Gastroenterology 162, 1319-1333 (2022), incorporated by reference herein. To produce retrovirus, GP2-HEK293T cells with a confluency of 70%-80% were transfected with a pCIR retrovirus vector expressing an anti-EGFR CAR or anti-PSCA CAR cassette plasmid using Lipofectamine 3000 Reagent (ThermoFisher), as previously described. See Teng et al. Gastroenterology 162, 1319-1333 (2022). To generate CAR-Th9 cells, YTHDF2^{-/-} WT and YTHDF2^{-/-} KO naïve CD4⁺ T cells were stimulated with human T-activator CD3/CD28 (STEMCELL) at a concentration of 20 μl per 0.5×10⁶ T cells. Forty-eight hours after activation, T cells were mixed with the retrovirus and 8 μg/ml polybrene (Millipore), followed by centrifugation for 2 hours at 1,800 g at 32° C. After that, the cells were cultured with retrovirus for 4-6 hours, and then the cells were washed and resuspended with Th9-polarized media for further culturing. Human CD4⁺ T cells were then cultured in the presence of IL-4 (30 ng/mL), TGF-β (2 ng/mL), and anti-IFN-γ antibody (10 μg/mL) for the initial two days, while IL-2 was extra added after retrovirus infection. Since initial isolation, CAR-Th9 cells were harvested and used for in vitro or in vivo experiments at days 12-14.

xCELLigence killing for tumor assays. Tumor cell lines, including A549 and Capan-1, were seeded onto 96-well RTCA E-plates (Agilent, Santa Clara, CA) and incubated for 24 h to facilitate attachment and proliferation. Th9 and/or NK cells (For combination killing, Th9:NK=1:1) were added at 5:1 Effector-to-Target (E:T) ratios, while control wells contained only tumor cells in the media. Tumor growth was assessed and reported as a normalized cell index. Impedance measurements were conducted at 15-minute intervals over several using the xCELLigence machine (Agilent). The results were presented as cell index, using the RTCA immunotherapy module software (Agilent).

### Tumor Models and Adoptive Transfer

For subcutaneous mouse models, 1×10⁶ B16-OVA tumor cells, E0771-OVA tumor cells, or LLC1-OVA tumor cells were subcutaneously (s.c.) implanted into the flanks of 6- to 12-week-old female and male C57BL/6 mice. 5 days after tumor injection, mice were treated with adoptive transfer of 3×10⁶ Ythdf2^{f/f} or Ythdf2^{cKO} Th9 cells. Cyclophosphamide (CTX, MCE) was administered intraperitoneally as a single dose at 200 mg/kg 1 day before Th9-cell transfer. The length (a) and width (b) of the tumors were measured starting on the indicated day (tumor implanted on day 0) and either every other day or every three days thereafter. Maximal tumor sizes were limited to 15 mm in diameter, as specified in our approved animal protocol. Mice were euthanized at indicated days for further experiments.

For human tumor models, 3×10⁶ Capan-1 tumor cells or 3×10⁶ A549 tumor cells were mixed with/without 1×10⁶ PBMCs. The mixture was resuspended with PBS and Matrigel (1:1) and then inoculated into the 6- to 12-week-old female and male NSG-SGM3 mice, s.c. in the flank. After the tumor implantation, mice were treated with adoptive transfer of autologous 3×10⁶ YTHDF2^{-/-} WT anti-PSCA and anti-EGFR CAR-Th9 cells, as well as YTHDF2^{-/-} KO anti-PSCA and anti-EGFR CAR-Th9 cells. CAR-Th9 cells were adoptively transferred into the mice on the indicated days. Tumor sizes were measured at indicated days after tumor inoculation.

For the humanized mice models, 6- to 12-week-old female and male NSG-SGM3 mice received intravenous injections of 10×10⁶ PBMCs. The NSG-SGM3 mice that successfully generated human immune cells were grouped randomly and then implanted with 3×10⁶ Capan-1 tumor cells. After tumor inoculation, mice were treated with adoptive transfer of autologous 5×10⁶ YTHDF2^{-/-} WT or YTHDF2^{-/-} KO anti-PSCA CAR-Th9 cells. CAR-Th9 cells were adoptively transferred into the mice every two days for a total of four times. Tumor burdens were evaluated at indicated days by measuring the tumor size.

Flow cytometry. Single-cell suspensions were prepared from the tumor tissue of tumor implantation mice, as described previously (91). Flow cytometry analysis and cell sorting were performed on BD LSRFortessa X-20 and FACSAria Fusion flow cytometers (BD Biosciences), respectively. Data were analyzed using NovoExpress software (Agilent Technologies). The following fluorescent dye-labeled antibodies purchased from Biolegend or BD Biosciences were used in this study: For mouse: CD3 (OKT3), CD4 (GK1.5), CD8 (53-6.7), NK1.1 (PK136), CD11b (M1/70), CD11c (N418), CD45 (30-F11), F4/80 (BM8), MHC class II (M5/114.15.2), Gr1 (RB6-8C5), IL-9 (RM9A4), GATA3 (16E10A23), IL-13 (W17010B), IL-17A (TC11-18H10.1), FOXP3 (QA20A67), CD44 (IM7), CD62L (MEL-14), c-Kit (ACK2), FcεRI (MAR-1), IFγ (XMG1.2); For human: CD3 (OKT3), CD4 (SK3), IL-9 (MH9A4), CD45 (2D1), CD45RA (5H9), CD45RO (UCHL1), CD19 (HIB19), EGFR (AY13), CD8 (SK1), IFNγ (B27). For examining intracellular cytokines production, cells were first stimulated with a leukocyte activation cocktail (50583, BD Biosciences) in the presence of the protein transport inhibitor brefeldin A for 4 hours. Cells were then stained with the indicated cell surface markers and fixed/permeabilized using a fixation/permeabilization kit (eBioscinece). Cell pellets were resuspended in PBS with 2% FBS for flow cytometry analysis.

Reverse transcription polymerase chain reaction (RT-PCR). RNA was isolated from Th9 cells using an RNeasy mini kit (QIAGEN) and then reverse transcribed to cDNA with a PrimeScript RT reagent kit with gDNA Eraser (Takara Bio) following the manufacturer's instructions. mRNA expression was analyzed using an SYBR Green PCR master mix and a QuantStudio 12K Flex real-time PCR system (both from Thermo Fisher Scientific).

RNA stability assay. Ythdf2^{f/f} or Ythdf2^{cKO} Th9 cells were seeded in 24-well plates at 2×10⁵ cells/mL. 5 μg/mL of Actinomycin D (Sigma-Aldrich, A9415) was added. After indicated hours of incubation, cells were collected, and total RNA was purified using an RNeasy Mini kit (QIAGEN). RNA stability was determined using RT-qPCR analysis as described previously (33).

Immunoblotting. Immunoblotting was performed according to standard procedures, as previously described (92). In brief, cells were lysed in RIPA Lysis and Extraction Buffer (ThermoFisher Scientific, 89900). Protein concentration was assessed using the BCA Protein Assay Kit (ThermoFisher Scientific, 23225). Samples were resolved using SDS-PAGE gels and transferred to PVDF membranes. Membranes were blocked with Intercept® Blocking Buffer (LI-COR, 927-60001) for 30 min at room temperature and incubated overnight at 4° C. with primary antibodies. Immunoblots were visualized with Odyssey CLx Imager (LI-COR). [0383]m⁶A-seq and bulk RNA-seq analysis. Total RNA was isolated with TRIzoll reagent (Thermo Fisher Scientific) from 50 million Ythdf2^{f/f} or Ythdf2^{cKO} Th9 cells. m⁶A immunoprecipitation was performed according to standard procedures, as previously reported (24). Sequencing of mouse samples was performed at the Translational Genomics Research Institute (TGen) on an Illumina NovaSeq 6000 platform with 100-base pair single-end reads, while sequencing of human samples was performed at the Translational Genomics Research Institute (TGen) on an Illumina NovaSeq 6000 platform with 100-base pair paired-end reads. Sequencing reads were mapped to the mouse genome (mm10) using STAR (version 2.7.4) (93). For m⁶A-seq, exomePeak2 (version 1.16.2) was run with default options. m⁶A peaks were visualized using Integrative Genomics Viewer software (version 2.8.13). The motifs enriched in m⁶A peaks were analyzed by HOMER (version 4.11). Each peak was annotated based on Ensembl gene annotation information by bedtools (version 2.30.0). For bulk RNA-seq, featureCounts (version 2.0.6) was used to calculate read counts. The DEGs between WT and Ythdf2-KO Th9 cells were detected with R package edgeR (version v. 3.28.1).

YTHDF2 RIP-seq. 50 million Ythdf2^{f/f} or Ythdf2^{cKO} Th9 cells were lysed with two volumes of lysis buffer (10 mM HEPES pH7.6, 150 mM KCl, 2 mM EDTA, 0.5% NP-40, 0.5 mM DTT, 100× protease inhibitor cocktail (Thermo Fisher Scientific) and 400 U/mL SUPERase-InRNase inhibitor (Thermo Fisher Scientific)). YTHDF2 RIP was performed according to the protocol as previously reported (24). A DNA library was generated with a KAPA RNA HyperPrep kit (Roche) and sequenced on the Illumina NovaSeq 6000 platform. Sequencing reads were mapped to the mouse genome (mm10) using STAR (version 2.7.4). The binding peaks of YTHDF2 were identified using exomePeak2 (version 1.16.2) with default options. The HOMER (version 4.11) was used to find motifs enriched in YTHDF2-binding peaks. The target genes were annotated based on Ensembl gene annotation information by bedtools (version 2.30.0).

Statistical analysis. Data were summarized by descriptive statistics (means, SD, etc). Continuous endpoints will be compared by Student's t-test or one-way ANOVA between 2 or more independent groups. Paired t-test or linear mixed models (equivalently one- or two-way ANOVA with repeated measures per GraphPad) will be used to compare two or more matched groups or groups with over-time repeated measures. Survival data were assessed by Kaplan-Meier curves and log-rank tests. All tests are two-sided. P values were adjusted for multiple comparisons by the Holm-Šídík method. A P value less than or equal to 0.05 is considered statistically significant. In each figure, error bars indicate the standard deviation (SD). P values were denoted as follows: *P<0.05, **P<0.01, ***P<0.001, ****P<0.0001. Statistical analyses were performed by using GraphPad Prism (version 10).

Although embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following embodiments define the scope of the invention and that methods and structures within the scope of these embodiments and their equivalents be covered thereby. All patent, patent publications, and non-patent literature cited herein is incorporated by reference herein in its entirety for all purposes.

### REFERENCES FOR BACKGROUND AND EXAMPLE 1

1. Hodi, F. S. et al. N Engl J Med 363, 711-723 (2010).
2. Garon, E. B. et al. N Engl J Med 372, 2018-2028 (2015).
3. Powles, T. et al. Nature 515, 558-562 (2014).
4. Ribas et al, Science 359, 1350-1355 (2018).
5. Gibney, et al, Lancet Oncol 17, e542-e551 (2016).
6. Samstein, R. M. et al. Nat Genet 51, 202-206 (2019).
7. Le, D. T. et al. Science 357, 409-413 (2017).
8. Tang, T. et al. Signal Transduct Target Ther 6, 72 (2021).
9. Murciano-Goroff, Y. R., Warner, A. B. & Wolchok, J. D. Cell Res 30, 507-519 (2020).
10. Schupp, J. et al. Cell Immunol 343, 103713 (2019).
11. Engblom, C., Pfirschke, C. & Pittet, M. J. Nat Rev Cancer 16, 447-462 (2016).
12. Noy, R. & Pollard, J. W. Immunity 41, 49-61 (2014).
13. Ruffell, B. et al. Cancer Cell 26, 623-637 (2014).
14. Qian, B. Z. & Pollard, J. W. Cell 141, 39-51 (2010).
15. Broz, M. L. et al. Cancer Cell 26, 638-652 (2014).
16. Gu, Y et al. The evolving landscape of N(6)-methyladenosine modification in the tumor microenvironment. Mol Ther 29, 1703-1715 (2021).
17. Wang, L. et al. m(6) ARNA methyltransferases METTL3/14 regulate immune responses to anti-PD-1 therapy. EMBO J 139, e104514 (2020).
18. Yang, S. et al. m(6)A mRNA demethylase FTO regulates melanoma tumorigenicity and response to anti-PD-1 blockade. Nat Commun 10, 2782 (2019).
19. Liu, Y et al. Cell Metab (2021).
20. Li, N. et al. Proceedings of the National Academy of Sciences of the United States of America 117, 20159-20170 (2020).
21. Su, R. et al. Targeting FTO Suppresses Cancer Stem Cell Maintenance and Immune Evasion. Cancer Cell 38, 79-96 e11 (2020).
22. Yin, H. et al. RNA m6A methylation orchestrates cancer growth and metastasis via macrophage reprogramming. Nat Commun 12, 1394 (2021).
23. Han, D. et al. Anti-tumour immunity controlled through mRNA m(6)A methylation and YTHDF1 in dendritic cells. Nature 566, 270-274 (2019).
24. Dong, L. et al. The loss of RNA N(6)-adenosine methyltransferase Mettl14 in tumor-associated macrophages promotes CD8(+) T cell dysfunction and tumor growth. Cancer Cell (2021).
25. Xiong, J. et al. Lactylation-driven METTL3-mediated RNA m(6)A modification promotes immunosuppression of tumor-infiltrating myeloid cells. Mol Cell (2022).
26. Li, X., Ma, S., Deng, Y, Yi, P. & Yu, J. Targeting the RNA m(6)A modification for cancer immunotherapy. Mol Cancer 21, 76 (2022).
27. Wang, X. et al. N(6)-methyladenosine Modulates Messenger RNA Translation Efficiency. Cell 161, 1388-1399 (2015).
28. Wang, X. et al. N6-methyladenosine-dependent regulation of messenger RNA stability. Nature 505, 117-120 (2014).
29. Li, A. et al. Cytoplasmic m(6)A reader YTHDF3 promotes mRNA translation. Cell Res 27, 444-447 (2017).
30. Shi, H. et al. YTHDF3 facilitates translation and decay of N(6)-methyladenosine-modified RNA. Cell Res 27, 315-328 (2017).
31. Dixit, D. et al. The RNA m6A Reader YTHDF2 Maintains Oncogene Expression and Is a Targetable Dependency in Glioblastoma Stem Cells. Cancer Discov 11, 480-499 (2021).
32. Li, W. et al. Comprehensive analysis of N6-methylandenosine regulators and m6A-related RNAs as prognosis factors in colorectal cancer. Mol Ther Nucleic Acids 27, 598-610 (2022).
33. Einstein, J. M. et al. Inhibition of YTHDF2 triggers proteotoxic cell death in MYC-driven breast cancer. Mol Cell 81, 3048-3064 e3049 (2021).
34. Feng, Z. Y., Wang, T., Su, X. & Guo, S. Identification of the m(6)A RNA Methylation Regulators WTAP as a Novel Prognostic Biomarker and Genomic Alterations in Cutaneous Melanoma. Front Mol Biosci 8, 665222 (2021).
35. Yoshihara, K. et al. Inferring tumour purity and stromal and immune cell admixture from expression data. Nat Commun 4, 2612 (2013).
36. Ma, S. et al. The RNA m6A reader YTHDF2 controls NK cell antitumor and antiviral immunity. J Exp Med 218 (2021).
37. Clausen, B. E., Burkhardt, C., Reith, W., Renkawitz, R. & Forster, I. Conditional gene targeting in macrophages and granulocytes using LysMcre mice. Transgenic Res 8, 265-277 (1999).
38. den Haan, J. M., Lehar, S. M. & Bevan, M. J. CD8(+) but not CD8(−) dendritic cells cross-prime cytotoxic T cells in vivo. J Exp Med 192, 1685-1696 (2000).
39. Gholamin, S. et al. Disrupting the CD47-SIRPalpha anti-phagocytic axis by a humanized anti-CD47 antibody is an efficacious treatment for malignant pediatric brain tumors. Sci Transl Med 9 (2017).
40. Muntjewerff, E. M., Meesters, L. D. & van den Bogaart, G. Antigen Cross-Presentation by Macrophages. Front Immunol 11, 1276 (2020).
41. Dixon, K. O. et al. TIM-3 restrains anti-tumour immunity by regulating inflammasome activation. Nature (2021).
42. Efremova, M., Vento-Tormo, M., Teichmann, S. A. & Vento-Tormo, R. CellPhoneDB: inferring cell-cell communication from combined expression of multi-subunit ligand-receptor complexes. Nat Protoc 15, 1484-1506 (2020).
43. Le Bon, A. et al. Cross-priming of CD8+ T cells stimulated by virus-induced type I interferon. Nat Immunol 4, 1009-1015 (2003).
44. Lawrence, T. & Natoli, G. Transcriptional regulation of macrophage polarization: enabling diversity with identity. Nat Rev Immunol 11, 750-761 (2011).
45. Mapperley, C. et al. The mRNA m6A reader YTHDF2 suppresses proinflammatory pathways and sustains hematopoietic stem cell function. J Exp Med 218 (2021).
46. Murray, P. J. et al. Macrophage activation and polarization: nomenclature and experimental guidelines. Immunity 41, 14-20 (2014).
47. Murray, P. J. Understanding and exploiting the endogenous interleukin-10/STAT3-mediated anti-inflammatory response. Curr Opin Pharmacol 6, 379-386 (2006).
48. Zhou, K. R. et al. ChIPBase v2.0: decoding transcriptional regulatory networks of non-coding RNAs and protein-coding genes from ChIP-seq data. Nucleic Acids Res 45, D43-D50 (2017).
49. Hu, H. et al. AnimalTFDB 3.0: a comprehensive resource for annotation and prediction of animal transcription factors. Nucleic Acids Res 47, D33-D38 (2019).
50. Zhang, C. et al. STAT3 Activation-Induced Fatty Acid Oxidation in CD8(+) T Effector Cells Is Critical for Obesity-Promoted Breast Tumor Growth. Cell Metab 31, 148-161 e145 (2020).
51. Roberts, T. C., Langer, R. & Wood, M. J. A. Advances in oligonucleotide drug delivery. Nat Rev Drug Discov 19, 673-694 (2020).
52. Kortylewski, M. et al. In vivo delivery of siRNA to immune cells by conjugation to a TLR9 agonist enhances antitumor immune responses. Nat Biotechnol 27, 925-932 (2009).
53. Zhang, Q. et al. TLR9-mediated siRNA delivery for targeting of normal and malignant human hematopoietic cells in vivo. Blood 121, 1304-1315 (2013).
54. Gao, C. et al. TLR9 signaling in the tumor microenvironment initiates cancer recurrence after radiotherapy. Cancer Res 73, 7211-7221 (2013).
55. Hossain, D. M. et al. Leukemia cell-targeted STAT3 silencing and TLR9 triggering generate systemic antitumor immunity. Blood 123, 15-25 (2014).
56. Hossain, D. M. et al. TLR9-Targeted STAT3 Silencing Abrogates Immunosuppressive Activity of Myeloid-Derived Suppressor Cells from Prostate Cancer Patients. Clin Cancer Res 21, 3771-3782 (2015).
57. Zhang, Q. et al. Serum-resistant CpG-STAT3 decoy for targeting survival and immune checkpoint signaling in acute myeloid leukemia. Blood 127, 1687-1700 (2016).
58. Zhao, X. et al. B Cell Lymphoma Immunotherapy Using TLR9-Targeted Oligonucleotide STAT3 Inhibitors. Mol Ther 26, 695-707 (2018).
59. Moreira, D. et al. STAT3 Inhibition Combined with CpG Immunostimulation Activates Antitumor Immunity to Eradicate Genetically Distinct Castration-Resistant Prostate Cancers. Clin Cancer Res 24, 5948-5962 (2018).
60. Moreira, D. et al. Myeloid cell-targeted STAT3 inhibition sensitizes head and neck cancers to radiotherapy and T cell-mediated immunity. J Clin Invest 131 (2021).
61. Maurer, T. et al. CpG-DNA aided cross-presentation of soluble antigens by dendritic cells. Eur J Immunol 32, 2356-2364 (2002).
62. Edelson, B. T. et al. Peripheral CD103+ dendritic cells form a unified subset developmentally related to CD8alpha+ conventional dendritic cells. J Exp Med 207, 823-836 (2010).
63. Dong, H., Zhu, G., Tamada, K. & Chen, L. B7-H1, a third member of the B7 family, co-stimulates T-cell proliferation and interleukin-10 secretion. Nat Med 5, 1365-1369 (1999).
64. Sharma, P., Hu-Lieskovan, S., Wargo, J. A. & Ribas, A. Primary, Adaptive, and Acquired Resistance to Cancer Immunotherapy. Cell 168, 707-723 (2017).
65. Moon, E. K. et al. Blockade of Programmed Death 1 Augments the Ability of Human T Cells Engineered to TargetNY-ESO-1 to Control Tumor Growth after Adoptive Transfer. Clin Cancer Res 22, 436-447 (2016).
66. Robbins, P. F. et al. Single and dual amino acid substitutions in TCR CDRs can enhance antigen-specific T cell functions. J Immunol 180, 6116-6131 (2008).
67. Cassetta, L. & Pollard, J. W. Targeting macrophages: therapeutic approaches in cancer. Nat Rev Drug Discov 17, 887-904 (2018).
68. Mantovani, A., Marchesi, F., Malesci, A., Laghi, L. & Allavena, P. Tumour-associated macrophages as treatment targets in oncology. Nat Rev Clin Oncol 14, 399-416 (2017).
69. Cannarile, M. A. et al. Colony-stimulating factor 1 receptor (CSF1R) inhibitors in cancer therapy. J Immunother Cancer 5, 53 (2017).
70. Xu, B. et al. An oncolytic virus expressing a full-length antibody enhances antitumor innate immune response to glioblastoma. Nat Commun 12, 5908 (2021).
71. Tian, L. et al. Targeting Fc receptor-mediated effects and the “don't eat me” signal with an oncolytic virus expressing an anti-CD47 antibody to treat metastatic ovarian cancer. Clin Cancer Res (2021).
72. Zhang, C. et al. YTHDF2 promotes the liver cancer stem cell phenotype and cancer metastasis by regulating OCT4 expression via m6A RNA methylation. Oncogene 39, 4507-4518 (2020).
73. Li, J. et al. YTHDF2 mediates the mRNA degradation of the tumor suppressors to induce AKT phosphorylation in N6-methyladenosine-dependent way in prostate cancer. Mol Cancer 19, 152 (2020).
74. Dixit, D. et al. The RNA m6A reader YTHDF2 maintains oncogene expression and is a targetable dependency in glioblastoma stem cells. Cancer Discov (2020).
75. Paris, J. et al. Targeting the RNA m(6)A Reader YTHDF2 Selectively Compromises Cancer Stem Cells in Acute Myeloid Leukemia. Cell Stem Cell 25, 137-148 e136 (2019).
76. Shi, H., Wei, J. & He, C. Where, When, and How: Context-Dependent Functions of RNA Methylation Writers, Readers, and Erasers. Mol Cell 74, 640-650 (2019).
77. Su, R. et al. R-2HG Exhibits Anti-tumor Activity by Targeting FTO/m(6)A/MYC/CEBPA Signaling. Cell 172, 90-105 e123 (2018).
78. Huang, Y et al. Small-Molecule Targeting of Oncogenic FTO Demethylase in Acute Myeloid Leukemia. Cancer Cell 35, 677-691 e610 (2019).
79. Yankova, E. et al. Small-molecule inhibition of METTL3 as a strategy against myeloid leukaemia. Nature 593, 597-601 (2021).
80. Herbst, R. S. et al. Predictive correlates of response to the anti-PD-L1 antibody MPDL3280A in cancer patients. Nature 515, 563-567 (2014).
81. Wang, F. et al. Crosstalks between mTORC1 and mTORC2 variagate cytokine signaling to control NK maturation and effector function. Nat Commun 9, 4874 (2018).
82. Wang, Y et al. The IL-15-AKT-XBP1s signaling pathway contributes to effector functions and survival in human NK cells. Nat Immunol 20, 10-17 (2019).
83. Butler, A., Hoffman, P., Smibert, P., Papalexi, E. & Satija, R. Integrating single-cell transcriptomic data across different conditions, technologies, and species. Nat Biotechnol 36, 411-420 (2018).
84. Subramanian, A. et al. Gene set enrichment analysis: a knowledge-based approach for interpreting genome-wide expression profiles. Proceedings of the National Academy of Sciences of the United States of America 102, 15545-15550 (2005).
85. Kramer, A., Green, J., Pollard, J., Jr. & Tugendreich, S. Causal analysis approaches in Ingenuity Pathway Analysis. Bioinformatics 30, 523-530 (2014).
86. Kim, D., Langmead, B. & Salzberg, S. L. HISAT: a fast spliced aligner with low memory requirements. Nat Methods 12, 357-360 (2015).
87. Zhang, Y et al. Model-based analysis of ChIP-Seq (MACS). Genome Biol 9, R137 (2008).
88. Heinz, S. et al. Simple combinations of lineage-determining transcription factors prime cis-regulatory elements required for macrophage and B cell identities. Mol Cell 38, 576-589 (2010).
89. Anders, S., Pyl, P. T. & Huber, W. HTSeq—a Python framework to work with high-throughput sequencing data. Bioinformatics 31, 166-169 (2015).
90. Love, M. I., Huber, W. & Anders, S. Moderated estimation of fold change and dispersion for RNA-seq data with DESeq2. Genome Biol 15, 550 (2014).
91. Freund, E. C. et al. Efficient gene knockout in primary human and murine myeloid cells by non-viral delivery of CRISPR-Cas9. J Exp Med 217 (2020).
92. Singhal, S. et al. Human tumor-associated monocytes/macrophages and their regulation of T cell responses in early-stage lung cancer. Sci Transl Med 11 (2019).
93. Tang, Z., Kang, B., Li, C., Chen, T. & Zhang, Z. GEPIA2: an enhanced web server for large-scale expression profiling and interactive analysis. Nucleic Acids Res 47, W556-W560 (2019).

### REFERENCES FOR EXAMPLE 2

Anderson, N. M., and Simon, M. C. (2020). The tumor microenvironment. Current biology: CB 30, R921-r925.
Beatty, G. L., and Gladney, W. L. (2015). Immune escape mechanisms as a guide for cancer immunotherapy. Clinical cancer research: an official journal of the American Association for Cancer Research 21, 687-692.
Burr, M. L., Sparbier, C. E., Chan, K. L., Chan, Y. C., Kersbergen, A., Lam, E. Y. N., Azidis-Yates, E., Vassiliadis, D., Bell, C. C., Gilan, O., et al. (2019). An Evolutionarily Conserved Function of Polycomb Silences the MHC Class I Antigen Presentation Pathway and Enables Immune Evasion in Cancer. Cancer cell 36, 385-401.e388.
Chang, C. H., Qiu, J., O'Sullivan, D., Buck, M. D., Noguchi, T., Curtis, J. D., Chen, Q., Gindin, M., Gubin, M. M., van der Windt, G. J., et al. (2015). Metabolic Competition in the Tumor Microenvironment Is a Driver of Cancer Progression. Cell 162, 1229-1241.
Chen, B., Ye, F., Yu, L., Jia, G., Huang, X., Zhang, X., Peng, S., Chen, K., Wang, M., Gong, S., et al. (2012). Development of cell-active N6-methyladenosine RNA demethylase FTO inhibitor. Journal of the American Chemical Society 134, 17963-17971.
Chiappinelli, K. B., Strissel, P. L., Desrichard, A., Li, H., Henke, C., Akman, B., Hein, A., Rote, N. S., Cope, L. M., Snyder, A., et al. (2015). Inhibiting DNA Methylation Causes an Interferon Response in Cancer via dsRNA Including Endogenous Retroviruses. Cell 162, 974-986.
Cook, D. N., Beck, M. A., Coffman, T. M., Kirby, S. L., Sheridan, J. F., Pragnell, I. B., and Smithies, O. (1995). Requirement of MIP-1 alpha for an inflammatory response to viral infection. Science (New York, NY) 269, 1583-1585.
Cui, Y. H., Yang, S., Wei, J., Shea, C. R., Zhong, W., Wang, F., Shah, P., Kibriya, M. G., Cui, X., Ahsan, H., et al. (2021). Autophagy of the m(6)A mRNA demethylase FTO is impaired by low-level arsenic exposure to promote tumorigenesis. Nature communications 12, 2183.
Cully, M. (2019). Chemical inhibitors make their RNA epigenetic mark. Nature reviews Drug discovery 18, 892-894.
Dale, B., Cheng, M., Park, K. S., Kaniskan, H., Xiong, Y, and Jin, J. (2021). Advancing targeted protein degradation for cancer therapy. Nature reviews Cancer 21, 638-654.
DeNardo, D. G., and Ruffell, B. (2019). Macrophages as regulators of tumour immunity and immunotherapy. Nature reviews Immunology 19, 369-382.
Diskin, B., Adam, S., Cassini, M. F., Sanchez, G., Liria, M., Aykut, B., Buttar, C., Li, E., Sundberg, B., Salas, R. D., et al. (2020). PD-L1 engagement on T cells promotes self-tolerance and suppression of neighboring macrophages and effector T cells in cancer. Nature immunology 21, 442-454.
Falahat, R., Berglund, A., Perez-Villarroel, P., Putney, R. M., Hamaidi, I., Kim, S., Pilon-Thomas, S., Barber, G. N., and Mulé, J. J. (2023). Epigenetic state determines the in vivo efficacy of STING agonist therapy. Nature communications 14, 1573.
Garcia-Lora, A., Algarra, I., and Garrido, F. (2003). MHC class I antigens, immune surveillance, and tumor immune escape. Journal of cellular physiology 195, 346-355.
Gavish, A., Tyler, M., Greenwald, A. C., Hoefflin, R., Simkin, D., Tschernichovsky, R., Galili Darnell, N., Somech, E., Barbolin, C., Antman, T., et al. (2023). Hallmarks of transcriptional intratumour heterogeneity across a thousand tumours. Nature 618, 598-606.
Ghoneim, H. E., Fan, Y, Moustaki, A., Abdelsamed, H. A., Dash, P., Dogra, P., Carter, R., Awad, W., Neale, G., Thomas, P. G., et al. (2017). De Novo Epigenetic Programs Inhibit PD-1 Blockade-Mediated T Cell Rejuvenation. Cell 170, 142-157 e119.
Gu, J., Xu, J., You, Q., and Guo, X. (2020). Recent developments of small molecules targeting RNA m(6)A modulators. European journal of medicinal chemistry 196, 112325.
Hanahan, D. (2022). Hallmarks of Cancer: New Dimensions. Cancer discovery 12, 31-46. Hänzelmann, S., Castelo, R., and Guinney, J. (2013). GSVA: gene set variation analysis for microarray and RNA-seq data. BMC bioinformatics 14, 7.
He, C., and Klionsky, D. J. (2009). Regulation mechanisms and signaling pathways of autophagy. Annual review of genetics 43, 67-93.
Huang, Y, Su, R., Sheng, Y, Dong, L., Dong, Z., Xu, H., Ni, T., Zhang, Z. S., Zhang, T., Li, C., et al. (2019). Small-Molecule Targeting of Oncogenic FTO Demethylase in Acute Myeloid Leukemia. Cancer cell 35, 677-691.e610.
Huang, Y, Yan, J., Li, Q., Li, J., Gong, S., Zhou, H., Gan, J., Jiang, H., Jia, G. F., Luo, C., et al. (2015). Meclofenamic acid selectively inhibits FTO demethylation of m6A over ALKBH5. Nucleic acids research 43, 373-384.
Imai, T., Hieshima, K., Haskell, C., Baba, M., Nagira, M., Nishimura, M., Kakizaki, M., Takagi, S., Nomiyama, H., Schall, T. J., et al. (1997). Identification and molecular characterization of fractalkine receptor CX3CR1, which mediates both leukocyte migration and adhesion. Cell 91, 521-530.
Johansen, T., and Lamark, T. (2011). Selective autophagy mediated by autophagic adapter proteins. Autophagy 7, 279-296.
Kaur, T., Zamani, D., Tong, L., Rubel, E. W., Ohlemiller, K. K., Hirose, K., and Warchol, M. E. (2015). Fractalkine Signaling Regulates Macrophage Recruitment into the Cochlea and Promotes the Survival of Spiral Ganglion Neurons after Selective Hair Cell Lesion. The Journal of neuroscience: the official journal of the Society for Neuroscience 35, 15050-15061.
Kim, D., Langmead, B., and Salzberg, S. L. (2015). HISAT: a fast spliced aligner with low memory requirements. Nature Methods 12, 357-360.
Kim, R., Emi, M., and Tanabe, K. (2007). Cancer immunoediting from immune surveillance to immune escape. Immunology 121, 1-14.
Kim, S. K., and Cho, S. W. (2022). The Evasion Mechanisms of Cancer Immunity and Drug Intervention in the Tumor Microenvironment. Frontiers in pharmacology 13, 868695.
Kinker, G. S., Greenwald, A. C., Tal, R., Orlova, Z., Cuoco, M. S., McFarland, J. M., Warren, A., Rodman, C., Roth, J. A., Bender, S. A., et al. (2020). Pan-cancer single-cell RNA-seq identifies recurring programs of cellular heterogeneity. Nature genetics 52, 1208-1218.
Klionsky, D. J. (2007). Autophagy: from phenomenology to molecular understanding in less than a decade. Nature reviews Molecular cell biology 8, 931-937.
Kotlyar, M., Pastrello, C., Sheahan, N., and Jurisica, I. (2016). Integrated interactions database: tissue-specific view of the human and model organism interactomes. Nucleic acids research 44, D536-541.
Lee, Y, Sasai, M., Ma, Ji S., Sakaguchi, N., Ohshima, J., Bando, H., Saitoh, T., Akira, S., and Yamamoto, M. (2015). p62 Plays a Specific Role in Interferon-γ-Induced Presentation of a Toxoplasma Vacuolar Antigen. Cell reports 13, 223-233.
Liu, J., Eckert, M. A., Harada, B. T., Liu, S. M., Lu, Z., Yu, K., Tienda, S. M., Chryplewicz, A., Zhu, A. C., Yang, Y, et al. (2018). m(6)A mRNA methylation regulates AKT activity to promote the proliferation and tumorigenicity of endometrial cancer. Nature cell biology 20, 1074-1083.
Liu, W. J., Ye, L., Huang, W. F., Guo, L. J., Xu, Z. G., Wu, H. L., Yang, C., and Liu, H. F. (2016). p62 links the autophagy pathway and the ubiqutin-proteasome system upon ubiquitinated protein degradation. Cellular & Molecular Biology Letters 21, 29.
Liu, Y, Liang, G., Xu, H., Dong, W., Dong, Z., Qiu, Z., Zhang, Z., Li, F., Huang, Y, Li, Y, et al. (2021). Tumors exploit FTO-mediated regulation of glycolytic metabolism to evade immune surveillance. Cell metabolism 33, 1221-1233.e1211.
Liu, Z., Li, X., Gao, Y, Liu, J., Feng, Y, Liu, Y, Wang, J., Wang, C., Wang, D., He, J., et al. (2023). Epigenetic reprogramming of Runx3 reinforces CD8⁺ T-cell function and improves the clinical response to immunotherapy. Molecular cancer 22, 84.
Ma, S., Han, J., Li, Z., Xiao, S., Zhang, J., Yan, J., Tang, T., Barr, T., Kraft, A. S., Caligiuri, M. A., et al. (2023a). An XBP1s-PIM-2 positive feedback loop controls IL-15-mediated survival of natural killer cells. Science immunology 8, eabn7993.
Ma, S., Sun, B., Duan, S., Han, J., Barr, T., Zhang, J., Bissonnette, M. B., Kortylewski, M., He, C., Chen, J., et al. (2023b). YTHDF2 orchestrates tumor-associated macrophage reprogramming and controls antitumor immunity through CD8⁺ T cells. Nature immunology 24, 255-266.
Ma, S., Yan, J., Barr, T., Zhang, J., Chen, Z., Wang, L. S., Sun, J. C., Chen, J., Caligiuri, M. A., and Yu, J. (2021). The RNA m6A reader YTHDF2 controls NK cell antitumor and antiviral immunity. The Journal of experimental medicine 218.
Martinez, F. O., and Gordon, S. (2014). The M1 and M2 paradigm of macrophage activation: time for reassessment. F1000prime reports 6, 13.
McAllister, F., Bailey, J. M., Alsina, J., Nirschl, C. J., Sharma, R., Fan, H., Rattigan, Y, Roeser, J. C., Lankapalli, R. H., Zhang, H., et al. (2014). Oncogenic Kras activates a hematopoietic-to-epithelial IL-17 signaling axis in preinvasive pancreatic neoplasia. Cancer cell 25, 621-637.
Mittal, D., Gubin, M. M., Schreiber, R. D., and Smyth, M. J. (2014). New insights into cancer immunoediting and its three component phases—elimination, equilibrium and escape. Curr Opin Immunol 27, 16-25.
Mizushima, N., Levine, B., Cuervo, A. M., and Klionsky, D. J. (2008). Autophagy fights disease through cellular self-digestion. Nature 451, 1069-1075.
Moroz-Omori, E. V, Huang, D., Kumar Bedi, R., Cheriyamkunnel, S. J., Bochenkova, E., Dolbois, A., Rzeczkowski, M. D., Li, Y, Wiedmer, L., and Caflisch, A. (2021). METTL3 Inhibitors for Epitranscriptomic Modulation of Cellular Processes. ChemMedChem 16, 3035-3043.
Raina, K., and Crews, C. M. (2017). Targeted protein knockdown using small molecule degraders. Current opinion in chemical biology 39, 46-53.
Renner, K., Bruss, C., Schnell, A., Koehl, G., Becker, H. M., Fante, M., Menevse, A. N., Kauer, N., Blazquez, R., Hacker, L., et al. (2019). Restricting Glycolysis Preserves T Cell Effector Functions and Augments Checkpoint Therapy. Cell Rep 29, 135-150 e139.
Said, S. S., and Ibrahim, W. N. (2023). Cancer Resistance to Immunotherapy: Comprehensive Insights with Future Perspectives. Pharmaceutics 15.
Sanmamed, M. F., and Chen, L. (2018). A Paradigm Shift in Cancer Immunotherapy: From Enhancement to Normalization. Cell 175, 313-326.
Slabicki, M., Kozicka, Z., Petzold, G., Li, Y, D., Manojkumar, M., Bunker, R. D., Donovan, K. A., Sievers, Q. L., Koeppel, J., Suchyta, D., et al. (2020a). The CDK inhibitor CR8 acts as a molecular glue degrader that depletes cyclin K. Nature 585, 293-297.
Slabicki, M., Yoon, H., Koeppel, J., Nitsch, L., Roy Burman, S. S., Di Genua, C., Donovan, K. A., Sperling, A. S., Hunkeler, M., Tsai, J. M., et al. (2020b). Small-molecule-induced polymerization triggers degradation of BCL6. Nature 588, 164-168.
Topper, M. J., Vaz, M., Chiappinelli, K. B., DeStefano Shields, C. E., Niknafs, N., Yen, R. C., Wenzel, A., Hicks, J., Ballew, M., Stone, M., et al. (2017). Epigenetic Therapy Ties MYC Depletion to Reversing Immune Evasion and Treating Lung Cancer. Cell 171, 1284-1300 e1221.
Tormoen, G. W., Crittenden, M. R., and Gough, M. J. (2018). Role of the immunosuppressive microenvironment in immunotherapy. Advances in radiation oncology 3, 520-526.
Vignali, D. A. A., Collison, L. W., and Workman, C. J. (2008). How regulatory T cells work. Nature Reviews Immunology 8, 523-532.
Vries, M. H., Wagenaar, A., Verbruggen, S. E., Molin, D. G., Dijkgraaf, I., Hackeng, T. H., and Post, M. J. (2015). CXCL1 promotes arteriogenesis through enhanced monocyte recruitment into the peri-collateral space. Angiogenesis 18, 163-171.
Wang, L., Dou, X., Chen, S., Yu, X., Huang, X., Zhang, L., Chen, Y, Wang, J., Yang, K., Bugno, J., et al. (2023). YTHDF2 inhibition potentiates radiotherapy antitumor efficacy. Cancer cell 41, 1294-1308.e1298.
Yang, Y, Li, Q., Ling, Y, Leng, L., Ma, Y, Xue, L., Lu, G., Ding, Y, Li, J., and Tao, S. (2022). m6A eraser FTO modulates autophagy by targeting SQSTM1/P62 in the prevention of canagliflozin against renal fibrosis. Frontiers in immunology 13, 1094556.
Yankova, E., Blackaby, W., Albertella, M., Rak, J., De Braekeleer, E., Tsagkogeorga, G., Pilka, E. S., Aspris, D., Leggate, D., Hendrick, A. G., et al. (2021). Small-molecule inhibition of METTL3 as a strategy against myeloid leukaemia. Nature 593, 597-601.
Zaccara, S., and Jaffrey, S. R. (2020). A Unified Model for the Function of YTHDF Proteins in Regulating m(6)A-Modified mRNA. Cell 181, 1582-1595.e1518.
Zeng, Q., Saghafinia, S., Chryplewicz, A., Fournier, N., Christe, L., Xie, Y. Q., Guillot, J., Yucel, S.,
Li, P., Galván, J. A., et al. (2022). Aberrant hyperexpression of the RNA binding protein FMRP in tumors mediates immune evasion. Science (New York, NY) 378, eab17207.
Zhang, X., Lan, Y, Xu, J., Quan, F., Zhao, E., Deng, C., Luo, T., Xu, L., Liao, G., Yan, M., et al.
(2019). CellMarker: a manually curated resource of cell markers in human and mouse. Nucleic acids research 47, D721-d728.
Zhang, Y, Sang, R., Bao, J., Jiang, Z., Qian, D., Zhou, Y, Su, W., Wei, J., Zhao, L., Wei, Z., et al. (2023). Schwann cell-derived CXCL2 contributes to cancer pain by modulating macrophage infiltration in a mouse breast cancer model. Brain, behavior, and immunity 109, 308-320.
Zhu, T., Roundtree, I. A., Wang, P., Wang, X., Wang, L., Sun, C., Tian, Y, Li, J., He, C., and Xu, Y. (2014). Crystal structure of the YTH domain of YTHDF2 reveals mechanism for recognition of N6-methyladenosine. Cell research 24, 1493-1496.

### REFERENCES FOR EXAMPLE 3

1 Zhu, X. & Zhu, J. Int J Mol Sci 21, doi:10.3390/ijms21218011 (2020).
2 Zhu, J. & Paul, W. E. Blood 112, 1557-1569, (2008).
3 O'Shea, J. J. & Paul, W. E. Science (New York, N. Y) 327, 1098-1102, (2010).
4 Murphy, K. & Weaver, C. Janeway's Immunobiology. (W. W. Norton, 2016).
5 Swain et al Nature Reviews Immunology 12, 136-148, (2012).
6 Luckheeram, R. V., Zhou, R., Verma, A. D. & Xia, B. CD4⁺ T cells: differentiation and functions. Clinical & developmental immunology 2012, 925135, doi:10.1155/2012/925135 (2012).
7 Kim, H.-J. & Cantor, H. CD4 T-cell Subsets and Tumor Immunity: The Helpful and the Not-so-Helpful. Cancer immunology research 2, 91-98, doi:10.1158/2326-6066.CIR-13-0216 % J Cancer Immunology Research (2014).
8 Miggelbrink, A. M. et al. CD4 T-Cell Exhaustion: Does It Exist and What Are Its Roles in Cancer?Clinical Cancer Research 27, 5742-5752, doi:10.1158/1078-0432.CCR-21-0206% J Clinical Cancer Research (2021).
9 Liu, Y. et al. N6-methyladenosine-mediated gene regulation and therapeutic implications. Trends in molecular medicine 29, 454-467, doi:10.1016/j.molmed.2023.03.005 (2023).
10 Li, X., Ma, S., Deng, Y, Yi, P. & Yu, J. Targeting the RNA m6A modification for cancer immunotherapy. Molecular cancer 21, 76, doi:10.1186/si2943-022-01558-0 (2022).
11 Liu, Y. et al. N6-methyladenosine-mediated gene regulation and therapeutic implications. Trends in molecular medicine 29, 454-467, doi:10.1016/j.molmed.2023.03.005 (2023).
12 Li, X., Ma, S., Deng, Y, Yi, P. & Yu, J. Targeting the RNA m(6)A modification for cancer immunotherapy. Molecular cancer 21, 76, doi:10.1186/si2943-022-01558-0 (2022).
13 Wang, X. et al. N6-methyladenosine-dependent regulation of messenger RNA stability. Nature 505, 117-120, doi:10.1038/nature12730 (2014).
14 Zhang, H. et al. YTHDF2 upregulation and subcellular localization dictate CD8 T cell polyfunctionality in anti-tumor immunity. Nature communications 15, 9559, doi:10.1038/s41467-024-53997-6 (2024).
15 Jing, H. et al. METTL3 governs thymocyte development and thymic involution by regulating ferroptosis. Nature aging, doi:10.1038/s43587-024-00724-x (2024).
16 Guo, W. et al. Mettl3-dependent m(6)A modification is essential for effector differentiation and memory formation of CD8(+) T cells. Science bulletin 69, 82-96, doi:10.1016/j.scib.2023.11.029 (2024).
17 Ito-Kureha, T. et al. The function of Wtap in N(6)-adenosine methylation of mRNAs controls T cell receptor signaling and survival of T cells. Nature immunology 23, 1208-1221, doi:10.1038/s41590-022-01268-1 (2022).
18 Zhou, J. et al. m(6)A demethylase ALKBH5 controls CD4(+) T cell pathogenicity and promotes autoimmunity. Science advances 7, doi:10.1126/sciadv.abg0470 (2021).
19 Yao, Y et al. METTL3-dependent m(6)A modification programs T follicular helper cell differentiation. Nature communications 12, 1333, doi:10.1038/s41467-021-21594-6 (2021).
20 Grenov, A., Hezroni, H., Lasman, L., Hanna, J. H. & Shulman, Z. YTHDF2 suppresses the plasmablast genetic program and promotes germinal center formation. Cell reports 39, 110778, doi:10.1016/j.celrep.2022.110778 (2022).
21 Huang, H. et al. Mettl14-Mediated m6A Modification Is Essential for Germinal Center B Cell Response. Journal of immunology (Baltimore, Md. 1950) 208, 1924-1936, doi:10.4049/jimmunol.2101071 (2022).
22 Grenov, A. C. et al. The germinal center reaction depends on RNA methylation and divergent functions of specific methyl readers. The Journal of experimental medicine 218, doi:10.1084/jem.20210360 (2021).
23 Song, H. et al. METTL3-mediated m(6)A RNA methylation promotes the anti-tumour immunity of natural killer cells. Nature communications 12, 5522, doi:10.1038/s41467-021-25803-0 (2021).
24 Ma, S. et al. The RNA m6A reader YTHDF2 controls NK cell antitumor and antiviral immunity. The Journal of experimental medicine 218, doi:10.1084/jem.20210279 (2021).
25 Han, D. et al. Anti-tumour immunity controlled through mRNA m(6)A methylation and YTHDF1 in dendritic cells. Nature 566, 270-274, doi:10.1038/s41586-019-0916-x (2019).
26 Wen, C. et al. YTHDF1 loss in dendritic cells potentiates radiation-induced antitumor immunity via STING-dependent type I IFN production. The Journal of clinical investigation, doi:10.1172/jci181612 (2024).
27 Wang, H. et al. Mettl3-mediated mRNA m(6)A methylation promotes dendritic cell activation. Nature communications 10, 1898, doi:10.1038/s41467-019-09903-6 (2019).
28 Dong, L. et al. The loss of RNA N(6)-adenosine methyltransferase Mettl14 in tumor-associated macrophages promotes CD8(+) T cell dysfunction and tumor growth. Cancer cell 39, 945-957.e910, doi:10.1016/j.ccell.2021.04.016 (2021).
29 Yin, H. et al. RNA m6A methylation orchestrates cancer growth and metastasis via macrophage reprogramming. Nature communications 12, 1394, doi:10.1038/s41467-021-21514-8 (2021).
30 Zhuang, T. et al. ALKBH5-mediated m6A modification of IL-11 drives macrophage-to-myofibroblast transition and pathological cardiac fibrosis in mice. Nature communications 15, 1995, doi:10.1038/s41467-024-46357-x (2024).
31 Wang, X. et al. The m6A Reader IGF2BP2 Regulates Macrophage Phenotypic Activation and Inflammatory Diseases by Stabilizing TSC1 and PPARγ. Advanced science (Weinheim, Baden-Wurttemberg, Germany) 8, 2100209, doi:10.1002/advs.202100209 (2021).
32 Ge, X. et al. The Loss of YTHDC1 in Gut Macrophages Exacerbates Inflammatory Bowel Disease. Advanced science (Weinheim, Baden-Wurttemberg, Germany) 10, e2205620, doi:10.1002/advs.202205620 (2023).
33 Ma, S. et al. YTHDF2 orchestrates tumor-associated macrophage reprogramming and controls antitumor immunity through CD8⁺ T cells. Nature immunology 24, 255-266, doi:10.1038/s41590-022-01398-6 (2023).
34 Wang, L. et al. YTHDF2 inhibition potentiates radiotherapy antitumor efficacy. Cancer cell 41, 1294-1308.e1298, doi:10.1016/j.ccell.2023.04.019 (2023).
35 Lyu, Z. et al. Suppression of YTIDF2 attenuates autoimmune hepatitis by expansion of myeloid-derived suppressor cells. Journal of autoimmunity 135, 102993, doi:10.1016/j.jaut.2023.102993 (2023).
36 Gerlach, K. et al. TH9 cells that express the transcription factor PU.1 drive T cell-mediated colitis via IL-9 receptor signaling in intestinal epithelial cells. Nature immunology 15, 676-686, doi:10.1038/ni.2920 (2014).
37 Végran, F. et al. The transcription factor IRF1 dictates the IL-21-dependent anticancer functions of TH9 cells. Nature immunology 15, 758-766, doi:10.1038/ni.2925 (2014).
38 Xue, G. et al. Adoptive cell therapy with tumor-specific Th9 cells induces viral mimicry to eliminate antigen-loss-variant tumor cells. Cancer cell 39, 1610-1622.e1619, doi:10.1016/j.ccell.2021.09.011 (2021).
39 Dardalhon, V. et al. IL-4 inhibits TGF-beta-induced Foxp3⁺ T cells and, together with TGF-beta, generates IL-9⁺IL-10⁺ Foxp3(−) effector T cells. Nat Immunol 9, 1347-1355, doi:10.1038/ni.1677 (2008).
40 Veldhoen, M. et al. Transforming growth factor-beta ‘reprograms’ the differentiation of T helper 2 cells and promotes an interleukin 9-producing subset. Nat Immunol 9, 1341-1346, doi:10.1038/ni.1659 (2008).
41 Xue, G., Jin, G., Fang, J. & Lu, Y IL-4 together with IL-1beta induces antitumor Th9 cell differentiation in the absence of TGF-beta signaling. Nat Commun 10, 1376, doi:10.1038/s41467-019-09401-9 (2019).
42 Végran, F. et al. The transcription factor TRF1 dictates the IL-21-dependent anticancer functions of TH9 cells. Nature immunology 15, 758-766, doi:10.1038/ni.2925 (2014).
43 Chang, H. C. et al. The transcription factor PU.1 is required for the development of IL-9-producing T cells and allergic inflammation. Nat Immunol 11, 527-534, doi:10.1038/ni.1867 (2010).
44 Staudt, V. et al. Interferon-regulatory factor 4 is essential for the developmental program of T helper 9 cells. Immunity 33, 192-202, doi:10.1016/j.immuni.2010.07.014 (2010).
45 Lu, Y et al. Th9 cells promote antitumor immune responses in vivo. The Journal of clinical investigation 122, 4160-4171, doi:10.1172/jci65459 (2012).
46 Lu, Y et al. Tumor-specific IL-9-producing CD8⁺ Tc9 cells are superior effector than type-I cytotoxic Tc1 cells for adoptive immunotherapy of cancers. Proceedings of the National Academy of Sciences of the United States of America 111, 2265-2270, doi:10.1073/pnas.1317431111 (2014).
47 Lu, Y et al. Th9 Cells Represent a Unique Subset of CD4(+) T Cells Endowed with the Ability to Eradicate Advanced Tumors. Cancer cell 33, 1048-1060.e1047, doi:10.1016/j.ccell.2018.05.004 (2018).
48 Purwar, R. et al. Robust tumor immunity to melanoma mediated by interleukin-9-producing T cells. Nature medicine 18, 1248-1253, doi:10.1038/nm.2856 (2012).
49 Liu, L. et al. Enhanced CAR-T activity against established tumors by polarizing human T cells to secrete interleukin-9. Nature communications 11, 5902, doi:10.1038/s41467-020-19672-2 (2020).
50 Ma, X. et al. Cholesterol negatively regulates IL-9-producing CD8(+) T cell differentiation and antitumor activity. J Exp Med 215, 1555-1569, doi:10.1084/jem.20171576 (2018).
51 Xue, G., Jin, G., Fang, J. & Lu, Y J. N. c. IL-4 together with IL-1β induces antitumor Th9 cell differentiation in the absence of TGF-β signaling. 10, 1376 (2019).
52 Jabeen, R. et al. Th9 cell development requires a BATF-regulated transcriptional network. The Journal of clinical investigation 123, 4641-4653, doi:10.1172/jci69489 (2013).
53 Bassil, R. et al. BCL6 controls Th9 cell development by repressing 119 transcription. J Immunol 193, 198-207, doi:10.4049/jimmunol.1303184 (2014).
54 Khan, O. et al. TOX transcriptionally and epigenetically programs CD8(+) T cell exhaustion. Nature 571, 211-218, doi:10.1038/s41586-019-1325-x (2019).
55 Sekine, T. et al. TOX is expressed by exhausted and polyfunctional human effector memory CD8(+) T cells. Sci Immunol 5, doi:10.1126/sciimmunol.aba7918 (2020).
56 Nakatsukasa, H. et al. The DNA-binding inhibitor Id3 regulates IL-9 production in CD4(+) T cells. Nat Immunol 16, 1077-1084, doi:10.1038/ni.3252 (2015).
57 Abdul-Wahid, A. et al. Induction of antigen-specific TH 9 immunity accompanied by mast cell activation blocks tumor cell engraftment. Int J Cancer 139, 841-853, doi:10.1002/ijc.30121 (2016).
58 You, F. P. et al. Th9 cells promote antitumor immunity via IL-9 and IL-21 and demonstrate atypical cytokine expression in breast cancer. International immunopharmacology 52, 163-167, doi:10.1016/j.intimp.2017.08.031 (2017).
59 Chen, J. et al. Dectin-1-activated dendritic cells: A potent Th9 cell inducer for tumor immunotherapy. Oncoimmunology 5, e1238558, doi:10.1080/2162402X.2016.1238558 (2016).
60 Shultz, L. D. et al. Human lymphoid and myeloid cell development in NOD/LtSz-scid IL2R gamma null mice engrafted with mobilized human hemopoietic stem cells. Journal of immunology (Baltimore, Md.: 1950) 174, 6477-6489, doi:10.4049/jimmunol.174.10.6477 (2005).
61 Song, J. et al. A mouse model for the human pathogen Salmonella typhi. Cell host & microbe 8, 369-376, doi:10.1016/j.chom.2010.09.003 (2010).
62 Mombaerts, P. et al. RAG-1-deficient mice have no mature B and T lymphocytes. Cell 68, 869-877, doi:10.1016/0092-8674(92)90030-g (1992).
63 Spranger, S., Dai, D., Horton, B. & Gajewski, T. F. Tumor-Residing Batf3 Dendritic Cells Are Required for Effector T Cell Trafficking and Adoptive T Cell Therapy. Cancer Cell 31, 711-723 e714, doi:10.1016/j.ccell.2017.04.003 (2017).
64 Hildner, K. et al. Batf3 deficiency reveals a critical role for CD8alpha+ dendritic cells in cytotoxic T cell immunity. Science (New York, N. Y) 322, 1097-1100, doi:10.1126/science.1164206 (2008).
65 Ma, R. et al. An Oncolytic Virus Expressing IL15/IL15Rα Combined with Off-the-Shelf EGFR-CAR NK Cells Targets Glioblastoma. Cancer research 81, 3635-3648, doi:10.1158/0008-5472.CAN-21-0035% J Cancer Research (2021).
66 Teng, K.-Y et al. Off-the-Shelf Prostate Stem Cell Antigen-Directed Chimeric Antigen Receptor Natural Killer Cell Therapy to Treat Pancreatic Cancer. Gastroenterology 162, 1319-1333, doi:https://doi.org/10.1053/j.gastro.2021.12.281 (2022).
67 Xue, G., Jin, G., Fang, J. & Lu, Y IL-4 together with IL-1β induces antitumor Th9 cell differentiation in the absence of TGF-β signaling. Nature communications 10, 1376, doi:10.1038/s41467-019-09401-9 (2019).
68 Goswami, R. & Kaplan, M. H. A brief history of IL-9. Journal of immunology (Baltimore, Md. 1950) 186, 3283-3288, doi:10.4049/jimmunol.1003049 (2011).
69 Noelle, R. J. & Nowak, E. C. Cellular sources and immune functions of interleukin-9. Nature Reviews Immunology 10, 683-687, doi:10.1038/nri2848 (2010).
70 Cui, A. et al. Dictionary of immune responses to cytokines at single-cell resolution. Nature 625, 377-384, doi:10.1038/s41586-023-06816-9 (2024).
71 Skak, K., Frederiksen, K. S. & Lundsgaard, D. Interleukin-21 activates human natural killer cells and modulates their surface receptor expression. Immunology 123, 575-583, doi:10.1111/j.1365-2567.2007.02730.x (2008).
72 Shanley, M. et al. Interleukin-21 engineering enhances NK cell activity against glioblastoma via CEBPD. Cancer cell 42, 1450-1466.e1411, doi:10.1016/j.ccell.2024.07.007 (2024).
73 Parrish-Novak, J., Foster, D. C., Holly, R. D. & Clegg, C. H. Interleukin-21 and the IL-21 receptor: novel effectors of NK and T cell responses. Journal of leukocyte biology 72, 856-863 (2002).
74 Tian, Y & Zajac, A. J. IL-21 and T Cell Differentiation: Consider the Context. Trends in immunology 37, 557-568, doi:10.1016/j.it.2016.06.001 (2016).
75 Vivier, E. et al. Innate or adaptive immunity? The example of natural killer cells. Science (New York, N. Y) 331, 44-49, doi:10.1126/science.1198687 (2011).
76 Huntington, N. D., Vosshenrich, C. A. J. & Di Santo, J. P. Developmental pathways that generate natural-killer-cell diversity in mice and humans. Nature Reviews Immunology 7, 703-714, doi:10.1038/nri2154 (2007).
77 Stassen, M., Schmitt, E. & Bopp, T. From interleukin-9 to T helper 9 cells. 1247, 56-68, doi:https://doi.org/10.1111/j.1749-6632.2011.06351.x (2012).
78 He, S., Wang, L., Wu, Y, Li, D. & Zhang, Y CCL3 and CCL20-recruited dendritic cells modified by melanoma antigen gene-1 induce anti-tumor immunity against gastric cancer ex vivo and in vivo. Journal of experimental & clinical cancer research: CR 29, 37, doi:10.1186/1756-9966-29-37 (2010).
79 Stutte, S. et al. Proceedings of the National Academy of Sciences of the United States of America 107, 8736-8741, (2010).
80 Weber, C. et al. The Journal of clinical investigation 121, 2898-2910, (2011).
81 Cappell, K. M. & Kochenderfer, J. N. Long-term outcomes following CAR T cell therapy: what we know so far. Nature Reviews Clinical Oncology 20, 359-371, doi:10.1038/s41571-023-00754-1 (2023).
82 Shulman, Z. & Stern-Ginossar, N. The RNA modification N(6)-methyladenosine as a novel regulator of the immune system. Nature immunology 21, 501-512, doi:10.1038/s41590-020-0650-4 (2020).
83 Zhang, L. et al. The EMBO journal 42, e113126, (2023).
84 Binnewies, M. et al. Nature medicine 24, 541-550, doi:10.1038/s41591-018-0014-x (2018).
85 Franciszkiewicz, et al Cancer research 72, 6325-6332, doi:10.1158/0008-5472.Can-12-2027 (2012).
86 Kohli, K., Pillarisetty, V. G. & Kim, T. S. Key chemokines direct migration of immune cells in solid tumors.Cancer gene therapy 29, 10-21, doi:10.1038/s41417-021-00303-x (2022).
87 Melssen, M. M., Sheybani, N. D., Leick, K. M. & Slingluff, C. L., Jr. Barriers to immune cell infiltration in tumors. J Immunother Cancer 11, doi:10.1136/jitc-2022-006401 (2023).
88 Ma, S., Caligiuri, M. A. & Yu, J. Harnessing IL-15 signaling to potentiate NK cell-mediated cancer immunotherapy. Trends in immunology 43, 833-847, doi:10.1016/j.it.2022.08.004 (2022).
89 Shan, L., Flavell, R. A. & Herndler-Brandstetter, D. Development of Humanized Mouse Models for Studying Human NK Cells in Health and Disease. Methods in molecular biology (Clifton, N. J.) 2463, 53-66, doi:10.1007/978-1-0716-2160-8_5 (2022).
90 Mansour, A. G. et al. Off-the-shelf CAR-engineered natural killer cells targeting FLT3 enhance killing of acute myeloid leukemia. Blood Advances 7, 6225-6239, doi:10.1182/bloodadvances.2022007405% J Blood Advances (2023).
91 Xiao, S. et al. The tumor-intrinsic role of the m<sup>6</sup>A reader YTHDF2 in regulating immune evasion. 9, eadl2171, doi:doi:10.1126/sciimmunol.adl2171 (2024).
92 Ma, S. et al. An XBPls-PIM-2 positive feedback loop controls IL-15-mediated survival of natural killer cells. Science immunology 8, eabn7993, doi:10.1126/sciimmunol.abn7993 (2023).
93 Dobin, A. et al. STAR: ultrafast universal RNA-seq aligner. Bioinformatics (Oxford, England) 29, 15-21, doi:10.1093/bioinformatics/bts635 (2013).

What is claimed is:

1. A compound comprising a tumor-associated macrophage (TAM) targeting agent attached to a YTHDF2 inhibitor.

2. The compound of claim 1, wherein the TAM targeting agent is a ligand for a TAM receptor.

3. The compound of claim 2, wherein the TAM receptor is TLR9, CD163, CD206, CD14, CD16, CD32, CD64, CD68, CD71, CCR5, or CCR2.

4. The compound of claim 1, wherein the TAM targeting agent is a CpG oligodeoxynucleotide.

5. The compound of claim 1, wherein the YTHDF2 inhibitor inhibits expression of a YTHDF2 protein.

6. The compound of claim 1, wherein the YTHDF2 inhibitor is an antibody, a small molecule, an aptamer, a nucleic acid, a protein, or an enzyme.

7. The compound of claim 1, wherein the YTHDF2 inhibitor is an antisense inhibitor.

8. The compound of claim 7, wherein the antisense inhibitor is shRNA or siRNA.

9. The compound of claim 7, wherein the antisense inhibitor is siRNA comprising SEQ ID NO:2.

10. The compound of claim 1, wherein the TAM targeting agent is SEQ ID NO:1 attached through a covalent linking group to the 5′ end of sense strand RNA having SEQ ID NO:3, wherein the sense strand RNA is hybridized to the YTHDF2 inhibitor having SEQ ID NO:2.

11. The compound of claim 1, wherein the YTHDF2 inhibitor is a small molecule having the structure of Formula (I) or a pharmaceutically acceptable salt thereof: 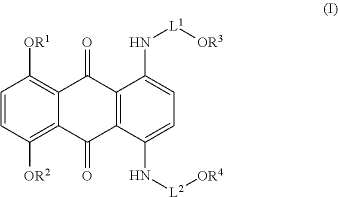
wherein R¹, R², R³, and R⁴ are each independently H or C₁₋₅ alkyl, and L¹ and L² are each independently a 2 to 8 membered heteroalkylene.

12. The compound of claim 1, wherein the YTHDF2 inhibitor is a small molecule having the structure of Formula (II) or a pharmaceutically acceptable salt thereof:

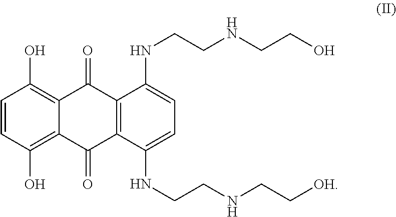 [SMILES: O=C1c2c(O)ccc(O)c2C(=O)c2c(NCCNCCO)ccc(NCCNCCO)c21]

13. The compound of claim 1, wherein the TAM targeting agent is attached to the YTHDF2 inhibitor through a covalent linking group.

14. A pharmaceutical composition comprising the compound of claim 1 and a pharmaceutically acceptable excipient.

15. A method of treating cancer in a patient in need thereof, the method comprising administering to the patient an effective amount of the compound of claim 1, thereby treating cancer.

16. The method of claim 15, wherein the patient has a high YTHDF2 expression in TAMS in the tumor microenvironment relative to a control.

17. A T helper 9 cell, wherein the T helper 9 cell does not express a YTHDF2 protein.

18. A method of treating cancer in a patient in need thereof, the method comprising administering to the patient an effective amount of the T helper 9 cell according to claim 17, thereby treating cancer.

19. A process of making the T helper 9 cell according to claim 17, the process comprising:
(i) modifying expression of a YTHDF2 protein in naïve CD4⁺ T cells, thereby producing naïve CD4⁺ T cells that do not express a YTHDF2 protein;
(ii) culturing and differentiating the naïve CD4⁺ T cells that do not express a YTHDF2 protein in the presence of IL-4, thereby producing T helper 9 cells that do not express a YTHDF2 protein; and
(iii) optionally transducing the T helper 9 cells that do not express a YTHDF2 protein with a chimeric antigen receptor, thereby producing T helper 9 cells that do not express a YTHDF2 protein and that comprises a chimeric antigen receptor.

20. A naïve CD4⁺ T cell, wherein the naïve CD4⁺ T cell does not express a YTHDF2 protein.
